(19) United States
(12) Patent Application Publication
(10) Pub. No.: US 2024/0366611 A1
(43) Pub. Date: Nov. 7, 2024

(54) METHODS OF TREATING A DISEASE OR DISORDER

(71) Applicants: Ascentage Pharma (Suzhou) Co., Ltd., Suzhou, Jiangsu (CN); Ascentage Pharma Group Corp Limited, Hong Kong (CN)

(72) Inventors: Yifan ZHAI, Suzhou, Jiangsu (CN); Dajun YANG, Suzhou, Jiangsu (CN); Saijie ZHU, Suzhou, Jiangsu (CN); Lei YANG, Suzhou, Jiangsu (CN); Douglas Dong FANG, Suzhou, Jiangsu (CN)

(21) Appl. No.: 18/682,742

(22) PCT Filed: Aug. 12, 2022
(86) PCT No.: PCT/CN2022/112286

§ 371 Date: Feb. 9, 2024

(30) Foreign Application Priority Data
Aug. 13, 2021 (WO) PCT/CN2021/112431
Oct. 22, 2021 (WO) PCT/CN2021/125764

(51) Int. Cl. A61K 31/519; A61K 31/5377; A61K 31/541; A61K 31/55; A61K 38/18; A61P 1/00; A61P 7/06

(52) U.S. Cl. CPC A61K 31/519; A61K 31/5377; A61K 31/541; A61K 31/55; A61K 38/1816; A61P 1/00; A61P 7/06

(57) ABSTRACT

Disclosed is a method for preventing and/or treating a disease or disorder associated with a defect in hemoglobin protein activity or expression or innate immune response, type 1 or type 3 immunity related immune disorder, which comprises administering to a subject in need thereof a therapeutically effective amount of a substance X or a pharmaceutical composition comprising the substance X; the substance X is a compound of Formula I, a pharmaceutically acceptable salt or solvate thereof.

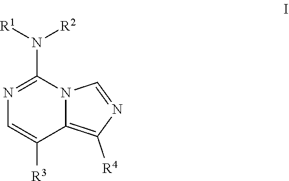

### CROSS-REFERENCE TO RELATED APPLICATIONS

This application is a U.S. National Phase Application, filed under 35 USC § 371, of PCT Application No. PCT/CN2022/112286, filed Aug. 12, 2022, which claims the priority of PCT Application No. PCT/CN2021/112431, filed Aug. 13, 2021, and PCT Application No. PCT/CN2021/125764, filed on Oct. 22, 2021, the contents of each of which are incorporated by reference herein in their entireties.

### FIELD OF INVENTION

The present invention relates to methods of preventing/treating a disease or disorder associated with a defect in hemoglobin protein activity or expression or innate immune response, type 1 or type 3 immunity related immune disorder.

### PRIOR ARTS

Hemoglobin is the critical protein involved in oxygen transport throughout the body of vertebrates. It is found in red blood cells and consists of two a subunits and two β-like subunits. The composition of hemoglobin is developmentally regulated, and the human genome encodes multiple versions of these proteins, which are expressed during distinct stages of development (Blobel et al, Exp Hematol 2015; Stamatoyannopoulos G, Exp Hematol 2005). In general, fetal hemoglobin (HbF) is composed of two subunits of hemoglobin γ (HBγ) and two subunits of hemoglobin α (HBα) and adult hemoglobin (HbA) is composed of two subunits of hemoglobin β (HBO) and two subunits of HBu. Thus, the β-like subunit utilized during the fetal stage of development (HBγ) switches to hemoglobin β (HBβ) after birth.

SCD (Sickle-Cell Disease) is a group of inherited red blood cell disorders and an autosomal recessive disease caused by single homozygous mutations in both copies of the HBB gene (E6V) that result in a mutant hemoglobin protein called HbS (https://ghr.nlm.nih.gov/condition/sickle-cell-disease). Under deoxygenated conditions, the HbS protein polymerizes, which leads to abnormal red blood cell morphology. Healthy red blood cells are round, and they move through small blood vessels to carry oxygen to all parts of the body. In someone who has SCD, the red blood cells become hard and sticky and look like a C-shaped farm tool called a “sickle”. The sickle cells die early, which causes a constant shortage of red blood cells. Also, when they travel through small blood vessels, they get stuck and clog the blood flow. This can cause pain and other serious problems such infection, acute chest syndrome and stroke.

β-thalassemia is caused by mutations in the HBB gene and results in reduced hemoglobin production (https://ghr.nlm.nih.gov/condition/beta-thalassemia). The mutations in the HBB gene typically reduce the production of adult β-globin protein, which leads to low levels of adult hemoglobin, HbA. This leads to a shortage of red blood cells and a lack of oxygen distribution throughout the body. Patients with β-thalassemia can have weakness, fatigue and are at risk of developing abnormal blood clots. Thousands of infants are born with β-thalassemia each year, and symptoms are typically detected within the first two years of life.

Chemotherapy-induced anemia (CIA) is a consequence of malignant invasion of normal tissue leading to blood loss, bone marrow infiltration with disruption of erythropoiesis, and functional iron deficiency as a consequence of inflammation. CIA is a significant consequence of chemotherapy and may delay or limit therapy as well as contribute to both fatigue and diminished quality of life.

Anemia is also a common complication in patients with chronic kidney disease (CKD), developing gradually and increasing in severity as kidney disease progresses. Many factors contribute to declining hemoglobin as CKD progresses, but impaired production of erythropoietin by failing kidneys is a central cause.

The immune system is made up of two parts: the innate (general) immune system and the adaptive (specialized) immune system. The innate immune response consists of physical, chemical and cellular defenses against pathogens. The main purpose of the innate immune response is to immediately prevent the spread and movement of foreign pathogens throughout the body. In the innate immune response, these include monocytes, NK cells, macrophages, neutrophils, eosinophils, basophils, mast cells, and dendritic cells. Adaptive immune responses are carried out by white blood cells called lymphocytes. There are two broad classes of such responses-antibody responses and cell-mediated immune responses, and they are carried out by different classes of lymphocytes, called B cells and T cells, respectively. The innate and adaptive immune systems converge into 3 major kinds of cell mediated effector immunity, which are categorized as type 1, type 2, and type 3. Type 1 immunity consists of T-bet⁺ IFN-g-producing group 1 ILCs (ILC1 and natural killer cells), CD8⁺ cytotoxic T cells (T_{C}1), CD4⁺ Tₕ1 cells, and the effector macrophage, which protects against intracellular microbes through activation of mononuclear phagocytes. Type 2 immunity consists of GATA-3⁺ ILC2s, T_{C}2 cells, and Tₕ2 cells producing IL-4, IL-5, IL-13, etc., which induces mast cell, basophil, and eosinophil activation, as well as IgE antibody production, thus protecting against helminthes, venoms and repairing tissue injury. Type 3 immunity is mediated by retinoic acid-related orphan receptor γδ⁺ ILC3s, T_{C}17 cells, and T_{H}17 cells producing IL-17, IL-22, etc., which recruits neutrophils and induce epithelial antimicrobial responses, thus protecting against extracellular bacteria and fungi (Annunziato F, Romagnani C, Romagnani S. The 3 major types of innate and adaptive cell-mediated effector immunity, Journal of Allergy & Clinical Immunology, 2015, 135(3): 626-635). Dysregulation of innate immune, type 1 and type 3 immunity mediates autoimmune diseases.

IBD (inflammatory bowel disease), classically divided into Crohn's disease (CD) and ulcerative colitis (UC), is a chronic, debilitating condition characterized by relapsing and remitting episodes of gastrointestinal (GI) inflammation. UC affects the superficial mucosa, starting with the rectum, in a continuous pattern and is limited to the colon. CD is characterized by transmural inflammation that can affect any part of the GI tract from mouth to anus. Acute DSS colitis model is caused primarily by disruption of the epithelium and activation of macrophages and neutrophils, which can be induced with the absence of adaptive immunity, so it is mainly recognized as an innate immune induced model. Intrarectal acute TNBS administration to mice induces a transmural colitis mainly driven by a type 1 immune response and characterized by infiltration of the lamina propria with CD4+ T cells, neutrophils, and macrophages (Kiesler P, Fuss I J, Strober W. Experimental Models of Inflammatory Bowel Diseases. Cell Mol Gastroenterol Hepatol. 2015, 1(2): 154-170). Injection of the sorted CD4⁺CD45RB^{high} T cells into recipient lymphopenic mice can produce a Th17 cells dominant model (may combined with Th1 cells). (Cell Mol Gastroenterol Hepatol. 2015, 1(2): 154-170; Am J Physiol Gastrointest Liver Physiol. 2009 February; 296(2): G135-G146). There is ongoing research looking for effective, convenient and tolerable treatments for a disease or disorder associated with a defect in hemoglobin protein activity or expression or innate immune response, type 1 or type 3 immunity related immune disorders.

### Content of the Present Invention

In one aspect, the present invention provides a method for preventing and/or treating disease or disorder which is selected from the group consisting of:
(1) a disease or disorder associated with a defect in hemoglobin protein activity or expression;
(2) innate immune response, type 1 or type 3 immunity related immune disorder (preferably inflammatory bowel disease, including Crohn's disease and ulcerative colitis);
the method comprises administering to a subject in need thereof a therapeutically effective amount of a substance X or a pharmaceutical composition comprising the substance X;

In another aspect, the present invention provides the use of substance X in the manufacture of a medicament for preventing and/or treating disease or disorder which is selected from the group consisting of:
(1) a disease or disorder associated with a defect in hemoglobin protein activity or expression;
(2) innate immune response, type 1 or type 3 immunity related immune disorder (preferably inflammatory bowel disease, including Crohn's disease and ulcerative colitis);

The substance X in present invention is a compound of Formula I, a pharmaceutically acceptable salt or solvate thereof;

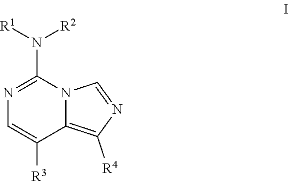

wherein:
R¹ is aralkyl;
R² is selected from the group consisting of hydrogen and C₁-C₄ alkyl;
R³ and R⁴ taken together with the carbon atoms to which they are attached form a radical of Formula I-A, I-B, or I-C:

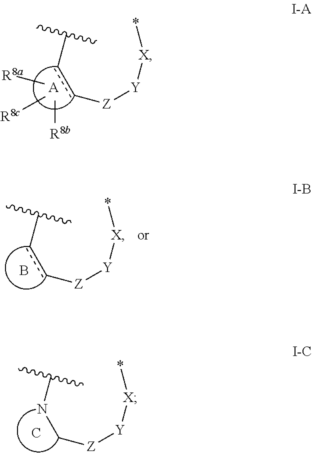

X is selected from the group consisting of —C(R^{5a})(R^{5b})—, —C(═O)—, and —S(═O)₂—;
R^{5a} and R^{5b} are independently selected from the group consisting of hydrogen and C₁-C₄ alkyl;
Y is selected from the group consisting of —C(R^{6a})(R^{6b})—, —S—, —O—, and —N(R⁷)—; or
X and Y taken together form a 5-membered heteroarylenyl;
Z is —C(R^{6c})(R^{6d})ₘ—;
R^{6a} and R^{6b} are independently selected from the group consisting of hydrogen and C₁-C₄ alkyl;
each R^{6c} and R^{6d} is independently selected from the group consisting of hydrogen and C₁-C₄ alkyl;
m is 0, 1, or 2;
R⁷ is selected from the group consisting of hydrogen, C₁-C₆ alkyl, C₁-C₆ haloalkyl, optionally substituted C₃-C₈ cycloalkyl, optionally substituted C₄-C₈ heterocyclo, hydroxyalkyl, (alkoxy)alkyl, (cycloalkyl)alkyl, and (heterocyclo)alkyl;
R^{8a}, R^{8b}, and R^{8c} are independently selected from the group consisting of hydrogen, halo, C₁-C₄ alkyl, C₁-C₄ haloalkyl, C₁-C₄ alkoxy, carboxamido, optionally substituted C₃-C₈ cycloalkyl, optionally substituted 4- to 8-membered heterocyclo, (heterocyclo)C₁-C₄ alkyl, and alkylsulfonyl;

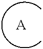

is a fused phenyl, fused 5-membered heteroaryl, or fused 6-membered heteroaryl;

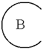

is an optionally substituted fused 3- to 8-membered cycloalkyl or optionally substituted fused 4- to 8-membered heterocyclo;

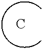

is an optionally substituted fused 4- to 8-membered heterocyclo; and
the bond designated with a “ [symbol] ” is attached at the R³ position of Formula I and the bond designated with an “*” is attached at the R⁴ position of Formula I; or
R³ is R^{3a};
R⁴ is R^{4a};
R^{3a} is selected from group consisting of optionally substituted aryl, optionally substituted 5- to 10-membered heteroaryl, and optionally substituted 4- to 8-membered heterocyclo; and
R^{4a} is selected from the group consisting of hydrogen, halo, C₁-C₄haloalkyl, —S(═O)₂R⁹, —P(═O)(R^{10a})(R^{10b}), —C(═O)OR^{11a}, —C(═O)NR^{11b}R^{11c} and —S(═O)(═NR^{13a})R^{13b};
R⁹ is selected from the group consisting of C₁-C₄ alkyl and C₃-C₆ cycloalkyl;
R^{10a} and R^{10b} are independently C₁-C₄ alkyl;
R^{11a} is selected from the group consisting of hydrogen and C₁-C₄ alkyl;
R^{11b} and R^{11c} are independently selected from the group consisting of hydrogen and C₁-C₄ alkyl; or
R^{11b} and R^{11c} taken together with the nitrogen atom to which they are attached form a 4- to 6-membered optionally substituted heterocyclo;
R^{13a} is selected from the group consisting of hydrogen, C₁-C₆ alkyl, C₃-C₆ cycloalkyl, and hydroxyalkyl;
R^{13b} is selected from the group consisting of C₁-C₆ alkyl and C₃-C₆ cycloalkyl; or
R^{13a} and R^{13b} taken together form a 5- to 7-membered heterocyclo; and
[symbol] is a single or double bond.

In some embodiments, the substance X are compounds of Formula I, wherein:
X is selected from the group consisting of —C(R^{5a})(R^{5b})—, —C(═O)—, and —S(═O)₂—;
Y is selected from the group consisting of —C(R^{6a})(R^{6b})—, —S—, —O—, and —N(R⁷)—; and
R^{8a}, R^{8b}, and R^{8c} are independently selected from the group consisting of hydrogen, halo, C₁-C₄ alkyl, C₁-C₄ haloalkyl, C₁-C₄ alkoxy, and alkylsulfonyl, or a pharmaceutically acceptable salt or solvate thereof.

In some embodiments, the substance X are compounds of Formula I, wherein R³ and R⁴ taken together with the carbon atoms to which they are attached form a radical of Formula I-A, I-B, or I-C, or a pharmaceutically acceptable salt or solvate thereof.

In some embodiments, the substance X are compounds of Formula II:

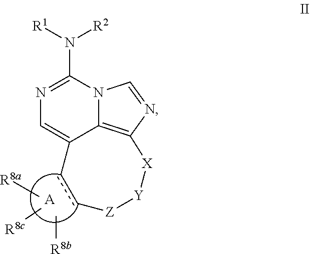

wherein R¹, R², R^{8a}, R^{8b}, R^{8c}, X, Y, Z,

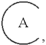

and [symbol] are as defined in connection with Formula I, or a pharmaceutically acceptable salt or solvate thereof.

In some embodiments, the substance X are compounds of Formula III:

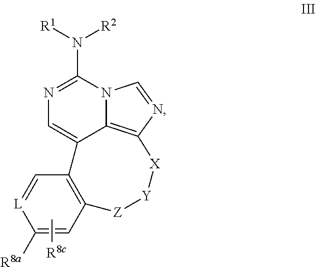

wherein L is selected from the group consisting of —C(R^{8b})═ and —N═; and R¹, R², R^{8a}, R^{8b}, R^{8c}, X, Y, and Z are as defined in connection with Formula I, or a pharmaceutically acceptable salt or solvate thereof.

In some embodiments, the substance X are compounds of Formula IV:

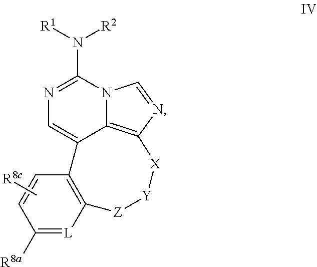

wherein L is selected from the group consisting of —C(R^{8b})═ and —N═; and R¹, R², R^{8a}, R^{8b}, R^{8c}, X, Y, and Z are as defined in connection with Formula I, or a pharmaceutically acceptable salt or solvate thereof.

In some embodiments, the substance X are compounds of Formula V:

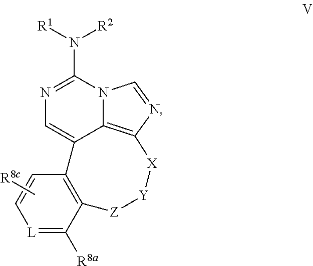

wherein L is selected from the group consisting of —C(R^{8b})═ and —N═; and R¹, R², R^{8a}, R^{8b}, R^{8c}, X, Y, and Z are as defined in connection with Formula I, or a pharmaceutically acceptable salt or solvate thereof.

In some embodiments, the substance X are compounds of Formula VI:

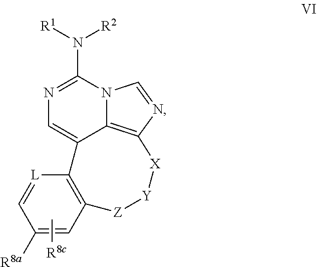

wherein L is selected from the group consisting of —C(R^{8b})═ and —N═; and R¹, R², R^{8a}, R^{8b}, R^{8c}, X, Y, and Z are as defined in connection with Formula I, or a pharmaceutically acceptable salt or solvate thereof.

In some embodiments, the substance X are compounds of any one of Formulae III-VI, wherein L is —C(R^{8b})═, or a pharmaceutically acceptable salt or solvate thereof.

In some embodiments, the substance X are compounds of any one of Formulae III-VI, wherein L is —N═, or a pharmaceutically acceptable salt or solvate thereof.

In some embodiments, the substance X are compounds of any one of Formulae I-VI, wherein R^{8a}, R^{8b}, and R^{8c} are independently selected from the group consisting of hydrogen, C₁-C₄ alkyl, C₁-C₄ haloalkyl, and C₃-C₆ cycloalkyl, or a pharmaceutically acceptable salt or solvate thereof. In another embodiment, R^{8a} is selected from the group consisting of —CHF₂, —CF₃, —CH₃, —CD₃, and cyclopropyl; and R^{8b} and R^{8c} are hydrogen. In another embodiment, R^{8a} is selected from the group consisting of —CF₃ or —CH₃; and R^{8b} and R^{8c} are hydrogen.

In some embodiments, the substance X are compounds of any one of Formulae I-VI, wherein, R^{8a} is selected from the group consisting of C₁-C₄ alkyl, 4- to 8-membered heterocyclo, and (heterocyclo)C₁-C₄ alkyl; and R^{8b} and R^{8c} are hydrogen, or a pharmaceutically acceptable salt or solvate thereof. In another embodiment, R^{8a} is C₁-C₄ alkyl. In another embodiment, R^{8a} is 4- to 8-membered heterocyclo. In another embodiment, R^{8a} is (heterocyclo)C₁-C₄ alkyl. In another embodiment, R^{8a} is selected from the group consisting of:

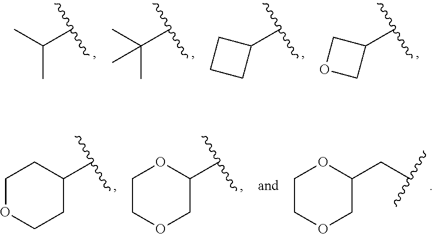

In some embodiments, the substance X are compounds of Formula VII:

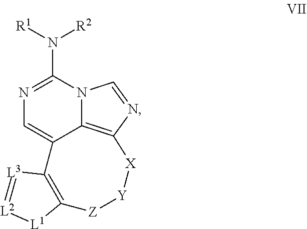

wherein:
L¹ is selected from the group consisting of —S—, —O—, and —N(R^{8a})—;
L² is selected from the group consisting of —C(R^{8b})═ and —N═;
L³ is selected from the group consisting of —C(R^{8c})═ and —N═;
R^{8a} is selected from the group consisting of hydrogen and C₁-C₄ alkyl;
R^{8b} is selected from the group consisting of hydrogen, C₁-C₄ alkyl, and C₁-C₄ haloalkyl;
R^{8c} is selected from the group consisting of hydrogen, C₁-C₄ alkyl, and C₁-C₄ haloalkyl; and
R¹, R², X, Y, and Z are as defined in connection with Formula I, or a pharmaceutically acceptable salt or solvate thereof.

In some embodiments, the substance X are compounds of Formula VIII:

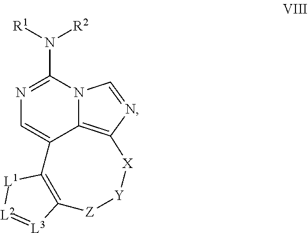

wherein:
L¹ is selected from the group consisting of —S—, —O—, and —N(R^{8a})—;
L² is selected from the group consisting of —C(R^{8b})═ and —N═;
L³ is selected from the group consisting of —C(R^{8c})═ and —N═;
R^{8a} is selected from the group consisting of hydrogen and C₁-C₄ alkyl;
R^{8b} is selected from the group consisting of hydrogen, C₁-C₄ alkyl, and C₁-C₄ haloalkyl;
R^{8c} is selected from the group consisting of hydrogen, C₁-C₄ alkyl, and C₁-C₄ haloalkyl; and
R¹, R², X, Y, and Z are as defined in connection with Formula I, or a pharmaceutically acceptable salt or solvate thereof.

In some embodiments, the substance X are compounds of Formula IX:

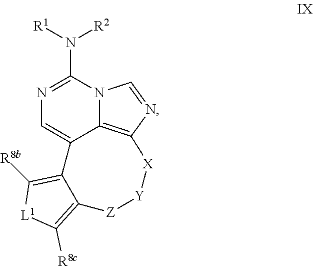

wherein:
L¹ is selected from the group consisting of —S—, —O—, and —N(R^{8a})—;
R^{8a} is selected from the group consisting of hydrogen and C₁-C₄ alkyl;
R^{8b} is selected from the group consisting of hydrogen, C₁-C₄ alkyl, and C₁-C₄ haloalkyl;
R^{8c} is selected from the group consisting of hydrogen, C₁-C₄ alkyl, and C₁-C₄ haloalkyl; and
R¹, R², X, Y, and Z are as defined in connection with Formula I, or a pharmaceutically acceptable salt or solvate thereof.

In some embodiments, the substance X are compounds of Formula X:

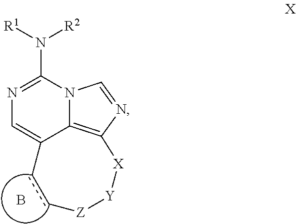

wherein R¹, R², X, Y, Z,

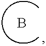

and [symbol] are as defined in connection with Formula I, or a pharmaceutically acceptable salt or solvate thereof.

In some embodiments, the substance X are compounds of Formula XI:

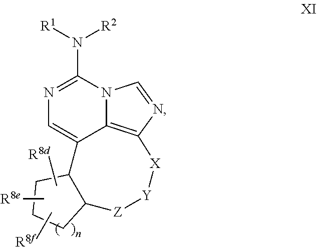

wherein:
R^{8d}, R^{8e}, and R^{8f} are independently selected from the group consisting of hydrogen, halo, and C₁-C₄ alkyl;
n is 1, 2, or 3; and
R¹, R², X, Y, and Z are as defined in connection with Formula I, or a pharmaceutically acceptable salt or solvate thereof.

In some embodiments, the substance X are compounds of Formula XI-A:

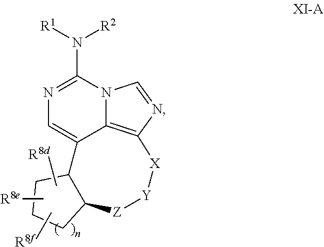

wherein R¹, R², R^{8d}, R^{8e}, R^{8f}, n, X, Y, and Z are as defined in connection with Formula XI, or a pharmaceutically acceptable salt or solvate thereof.

In some embodiments, the substance X are compounds of Formula XI-B:

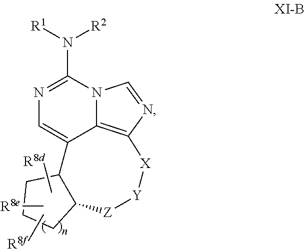

wherein R¹, R², R^{8d}, R^{8e}, R^{8f}, n, X, Y, and Z are as defined in connection with Formula XI, or a pharmaceutically acceptable salt or solvate thereof.

In some embodiments, the substance X are compounds of Formula XII:

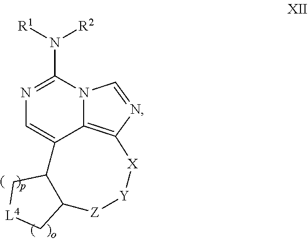

wherein:
L⁴ is selected from the group consisting of —S—, —O—, and —N(R^{8g})—;
R^{8g} is selected from the group consisting of hydrogen, C₁-C₄ alkyl, optionally substituted C₃-C₆ cycloalkyl, and optionally substituted 4- to 8-membered heterocyclo;
o is 0, 1, 2, or 3;
p is 0, 1, 2, or 3;
wherein the sum of o and p is 1, 2, 3, 4, or 5; and
R¹, R², X, Y, and Z are as defined in connection with Formula I, or a pharmaceutically acceptable salt or solvate thereof.

In some embodiments, the substance X are compounds of Formula XII-A:

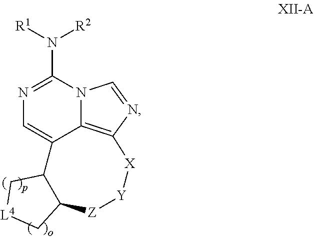

wherein R¹, R², L⁴, o, p, X, Y, and Z are as defined in connection with Formula XII, or a pharmaceutically acceptable salt or solvate thereof.

In some embodiments, the substance X are compounds of Formula XII-B:

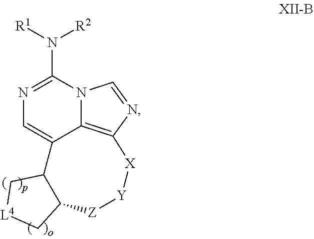

wherein R¹, R², L⁴, o, p, X, Y, and Z are as defined in connection with Formula XII, or a pharmaceutically acceptable salt or solvate thereof.

In some embodiments, the substance X are compounds of Formula XIII:

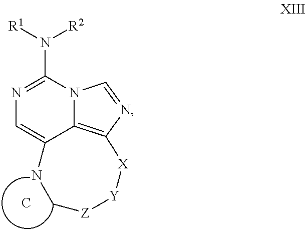

wherein R¹, R², X, Y, Z, and

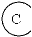

are as defined in connection with Formula I, or a pharmaceutically acceptable salt or solvate thereof.

In some embodiments, the substance X are compounds of Formula XIII-A:

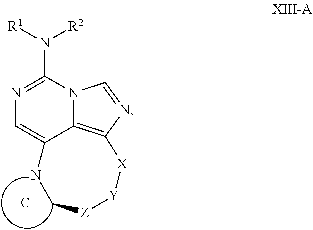

wherein R¹, R², X, Y, Z, and

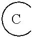

are as defined in connection with Formula I, or a pharmaceutically acceptable salt or solvate thereof.

In some embodiments, the substance X are compounds of Formula XIII-B:

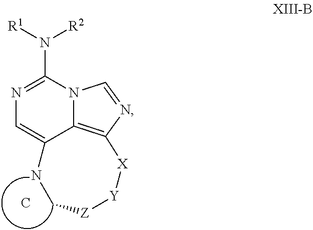

wherein R¹, R², X, Y, Z, and

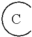

are as defined in connection with Formula I, or a pharmaceutically acceptable salt or solvate thereof.

In some embodiments, the substance X are compounds of Formula XIV:

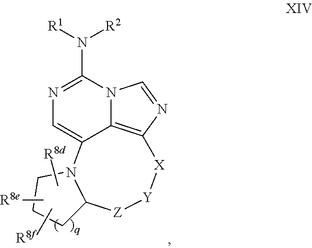

wherein:
R^{8d}, R^{8e}, and R^{8f} are independently selected from the group consisting of hydrogen and C-C₄ alkyl;
q is 1, 2, or 3; and
R¹, R², X, Y, and Z are as defined in connection with Formula I, or a pharmaceutically acceptable salt or solvate thereof.

In some embodiments, the substance X are compounds of Formula XIV-A:

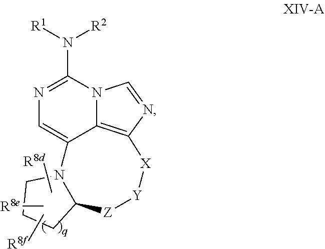

wherein R¹, R², R^{8d}, R^{8e}, R^{8f}, q, X, Y, and Z are as defined in connection with Formula XIV, or a pharmaceutically acceptable salt or solvate thereof.

In some embodiments, the substance X are compounds of Formula XIV-B:

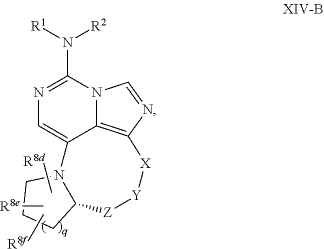

wherein R¹, R², R^{8d}, R^{8e}, R^{8f}, q, X, Y, and Z are as defined in connection with Formula XIV, or a pharmaceutically acceptable salt or solvate thereof.

In some embodiments, the substance X are compounds of Formula XV:

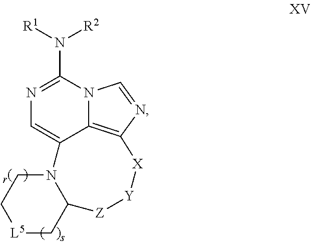

wherein:
L⁵ is selected from the group consisting of —S—, —O—, and —N(R^{8h})—;
R^{8h} is selected from the group consisting of hydrogen, C₁-C₄ alkyl, optionally substituted C₃-C₆ cycloalkyl, optionally substituted 4- to 8-membered heterocyclo, —C(═O)R^{14a}, and —S(═O)₂R^{14b};
R^{14a} and R^{14b} are independently selected from the group consisting of C₁-C₆ alkyl and optionally substituted C₃-C₈ cycloalkyl;
r is 1, 2, or 3;
s is 1, 2, or 3; and
R¹, R², X, Y, and Z are as defined in connection with Formula I, or a pharmaceutically acceptable salt or solvate thereof.

In some embodiments, the substance X are compounds of Formula XV-A:

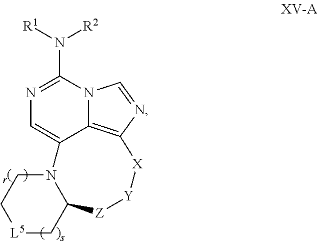

wherein R¹, R², L⁵, r, s, X, Y, and Z are as defined in connection with Formula XV, or a pharmaceutically acceptable salt or solvate thereof.

In some embodiments, the substance X are compounds of Formula XV-B:

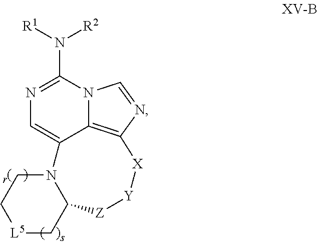

wherein R¹, R², L⁵, r, s, X, Y, and Z are as defined in connection with Formula XV, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, or XV-B, wherein Z is —CH₂—, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, or XV-B, wherein X is —CH₂—, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, or XV-B, wherein X is —C(═O)—, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, or XV-B, wherein X is —S(═O)₂—, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, or XV-B, wherein Y is —O—, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, or XV-B, wherein Y is —N(R⁷)—, or a pharmaceutically acceptable salt or solvate thereof. In another embodiment, R⁷ is selected from the group consisting of C₁-C₆ alkyl, C₁-C₆ haloalkyl, and optionally substituted C₃-C₈ cycloalkyl, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, or XV-B, wherein Z is —CH₂—, X is —C(═O)—, and Y is —N(R⁷)—, or a pharmaceutically acceptable salt or solvate thereof. In another embodiment, R⁷ is selected from the group consisting of C₁-C₆ alkyl, C₁-C₆ haloalkyl, and optionally substituted C₃-C₈cycloalkyl. In another embodiment, R⁷ is C₁-C₄alkyl. In another embodiment, R⁷ is selected from the group consisting of methyl, ethyl, propyl, or isopropyl.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, or XV-B, wherein X-Y taken together form an optionally substituted fused 5- or 6-membered heteroaryl, or a pharmaceutically acceptable salt or solvate thereof. In another embodiment, X and Y taken together form a 5-membered heteroarylenyl.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, or XV-B, wherein X and Y taken together form a 5-membered heteroarylenyl of Formula I-D:

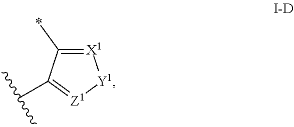

wherein:
X¹ is selected from the group consisting of ═CR^{15a}— and ═N—;
Y¹ is selected from the group consisting of —O—, —S—, and —NR^{15c}—;
Z¹ is selected from the group consisting of ═CR^{15b}— and ═N—;
R^{15a} and R^{15b} are independently selected from the group consisting of hydrogen, C₁-C₄ alkyl, C₁-C₄ haloalkyl, and C₃-C₆ cycloalkyl;
R^{15c} is selected from the group consisting of hydrogen, C₁-C₄ alkyl, and C₃-C₆ cycloalkyl; and
the bond designated with a “ [symbol] ” is attached to Z, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, or XV-B, wherein X and Y taken together form a 5-membered heteroarylenyl of Formula I-E:

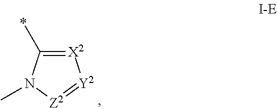

wherein:
X² is selected from the group consisting of ═CR^{16a}— and ═N—;
Y² is selected from the group consisting of ═CR^{16b}— and ═N—;
Z² is selected from the group consisting of ═CR^{16b}— and ═N—; and
R^{16a}, R^{16b}, and R^{16c} are independently selected from the group consisting of hydrogen, C₁-C₄ alkyl, and C₃-C₆ cycloalkyl; and
the bond designated with a “ [symbol] ” is attached to Z, or a pharmaceutically acceptable salt or solvate thereof

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, or XV-B, wherein X and Y taken together form a 5-membered heteroarylenyl selected from the group consisting of:

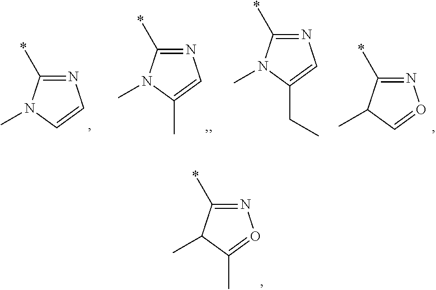

wherein the bond designated with a “ [symbol] ” is attached to Z, or a pharmaceutically acceptable salt or solvate thereof.

In some embodiments, the substance X are compounds of Formula XVI:

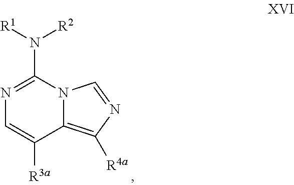

wherein R¹, R², R^{3a}, and R^{4a} are as defined in connection with Formula I, or a pharmaceutically acceptable salt or solvate thereof.

In some embodiments, the substance X are compounds of Formula XVI, wherein R^{3a} is optionally substituted phenyl, or a pharmaceutically acceptable salt or solvate thereof.

In some embodiments, the substance X are compounds of Formula XVI, wherein R^{3a} is optionally substituted 5-membered heteroaryl, or a pharmaceutically acceptable salt or solvate thereof.

In some embodiments, the substance X are compounds of Formula XVI, wherein R^{3a} is optionally substituted 6-membered heteroaryl, or a pharmaceutically acceptable salt or solvate thereof. In another embodiment, R^{3a} is selected from the group consisting of:

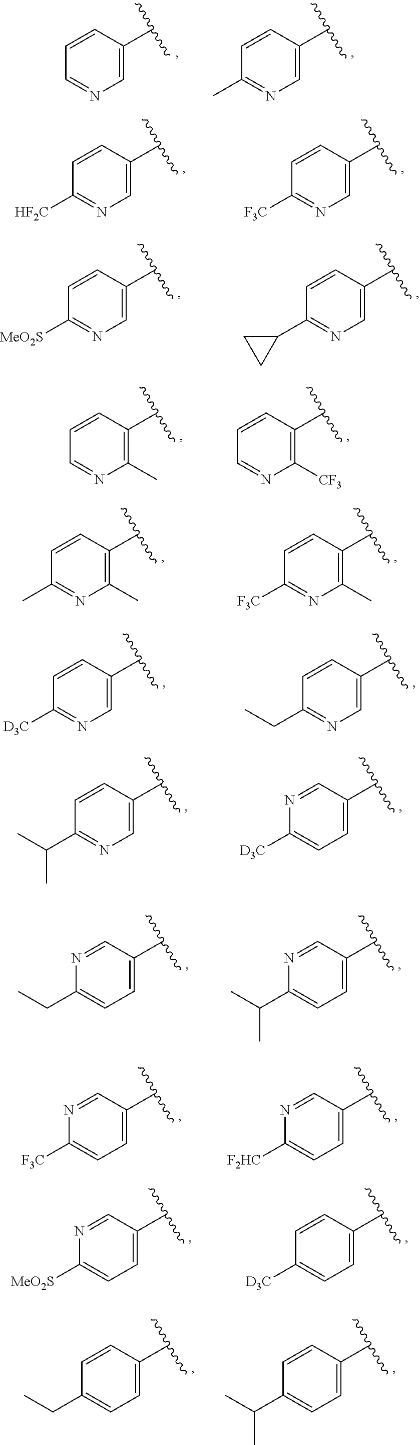

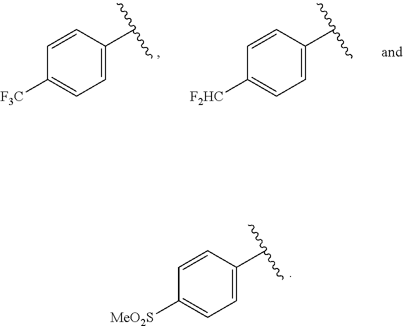 [SMILES: *c1ccc(C(F)(F)F)cc1 ; *c1ccc(C(F)F)cc1 ; *c1ccc(S(C)(=O)=O)cc1]

In some embodiments, the substance X are compounds of Formula XVI, or a pharmaceutically acceptable salt or solvate thereof, wherein R^{3a} is selected from the group consisting of:

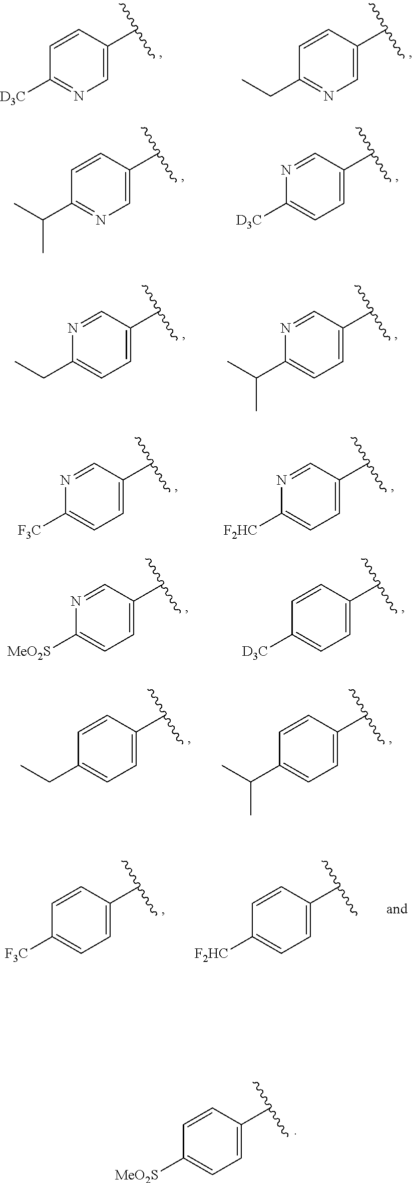

In some embodiments, the substance X are compounds of Formula XVI, or a pharmaceutically acceptable salt or solvate thereof, wherein R^{3a} is selected from the group consisting of:

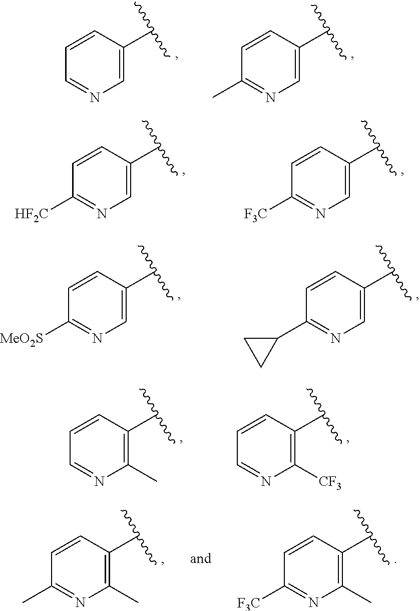

In some embodiments, the substance X are compounds of Formula XVI, wherein R^{3a} is optionally substituted 4- to 6-membered heterocyclo, or a pharmaceutically acceptable salt or solvate thereof.

In some embodiments, the substance X are compounds of Formula XVI, wherein R^{4a} is C₁-C₄haloalkyl, or a pharmaceutically acceptable salt or solvate thereof.

In some embodiments, the substance X are compounds of Formula XVI, wherein R^{4a} is —S(═O)₂R⁹, or a pharmaceutically acceptable salt or solvate thereof.

In some embodiments, the substance X are compounds of Formula XVI, wherein R^{4a} is —P(═O)(R^{10a})(R^{10b}), or a pharmaceutically acceptable salt or solvate thereof.

In some embodiments, the substance X are compounds of Formula XVI, wherein R^{4a} is —C(═O)OR^{11a}, or a pharmaceutically acceptable salt or solvate thereof. In another embodiment, R^{11a} is hydrogen.

In some embodiments, the substance X are compounds of Formula XVI, wherein R^{4a} is —C(═O)NR^{11b}R^{11c}, or a pharmaceutically acceptable salt or solvate thereof.

In some embodiments, the substance X are compounds of Formula XVI, wherein R^{4a} is —S(═O)(═NR^{13a})R^{13b} or a pharmaceutically acceptable salt or solvate thereof. In another embodiment, R^{13a} is selected from the group consisting of hydrogen and C₁-C₄ alkyl and R^{13b} is C₁-C₄ alkyl. In another embodiment, R^{13a} and R^{13b} taken together form a 6-membered heterocyclo, e.g.,

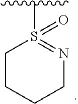 [SMILES: *S1(=O)=NCCCC1]

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, XV-B, or XVI, wherein R² is hydrogen, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, XV-B, or XVI, wherein:
R¹ is R¹-1:

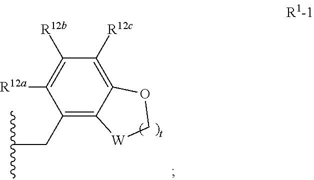

R^{12a}, R^{12b}, and R^{12c} are each independently selected from the group consisting of hydrogen, halo, C₁-C₄ alkyl, C₁-C₄ haloalkyl, and C₁-C₄ alkoxy;
W is selected from the group consisting of —CH₂— and —C(═O)—; and
t is 1 or 2, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, XV-B, or XVI, wherein R¹ is R¹-1, R^{12a} is fluoro; and R^{12b} and R^{12c} are independently selected from the group consisting of hydrogen and fluoro, or a pharmaceutically acceptable salt or solvate thereof. In another embodiment, R^{12a} is fluoro; and R^{12b} and R^{12c} are hydrogen.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, XV-B, or XVI, wherein:
R¹ is R¹-2:

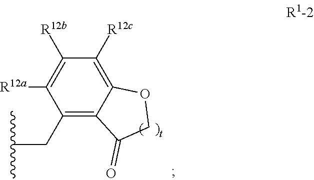

R^{12a}, R^{12b}, and R^{12c} are each independently selected from the group consisting of hydrogen, halo, C₁-C₄ alkyl, C₁-C₄ haloalkyl, and C₁-C₄ alkoxy; and
t is 1 or 2, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, XV-B, or XVI, wherein R¹ is R¹-2, R^{12a} is fluoro; and R^{12b} and R^{12c} are independently selected from the group consisting of hydrogen and fluoro, or a pharmaceutically acceptable salt or solvate thereof. In another embodiment, R^{12a} is fluoro; and R^{12b} and R^{12c} are hydrogen.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, XV-B, or XVI, wherein:
R¹ is R¹-3:

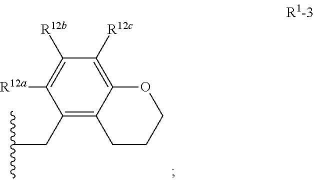

and
R^{12a}, R^{12b}, and R^{12c} are each independently selected from the group consisting of hydrogen, halo, C₁-C₄ alkyl, C₁-C₄ haloalkyl, and C₁-C₄ alkoxy, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, XV-B, or XVI, wherein R¹ is R¹-3, R^{12a} is fluoro; and R^{12b} and R^{12c} are independently selected from the group consisting of hydrogen and fluoro, or a pharmaceutically acceptable salt or solvate thereof. In another embodiment, R^{12a} is fluoro; and R^{12b} and R^{12c} are hydrogen.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, XV-B, or XVI, wherein:
R¹ is R¹-4:

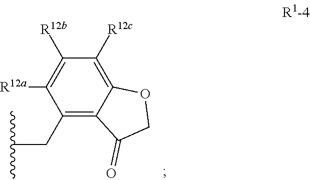

and
R^{12a}, R^{12b}, and R^{12c} are each independently selected from the group consisting of hydrogen, halo, C₁-C₄ alkyl, C₁-C₄ haloalkyl, and C₁-C₄ alkoxy, or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, XV-B, or XVI, wherein R¹ is R¹-4, R^{12a} is fluoro; and R^{12b} and R^{12c} are independently selected from the group consisting of hydrogen and fluoro, or a pharmaceutically acceptable salt or solvate thereof. In another embodiment, R^{12a} is fluoro; and R^{12b} and R^{12c} are hydrogen.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, XV-B, or XVI, wherein R¹ is selected from the group consisting of:

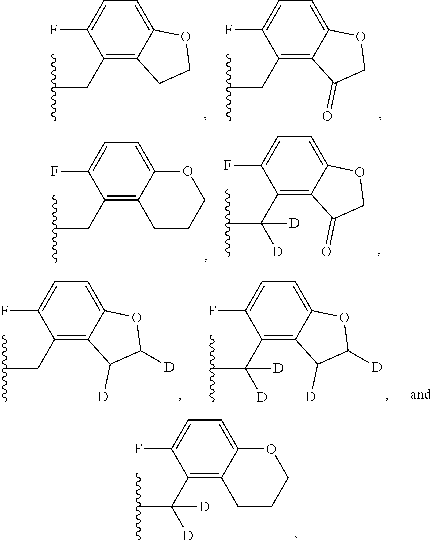

or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, XV-B, or XVI, wherein R¹ is selected from the group consisting of:

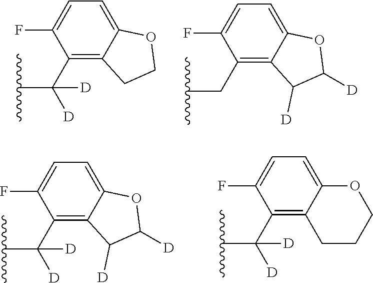

or a pharmaceutically acceptable salt or solvate thereof.

In some embodiments, the substance X are compounds of any one of Formulae I-XI, XI-A, XI-B, XII, XII-A, XII-B, XIII, XIII-A, XIII-B, XIV, XIV-A, XIV-B, XV, XV-A, XV-B, or XVI, wherein R¹ is selected from the group consisting of:

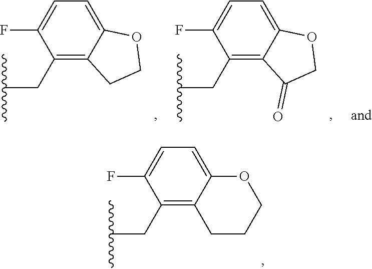

or a pharmaceutically acceptable salt or solvate thereof.

In some embodiments, the substance X are any one or more of the compounds listed in Table 1, or a pharmaceutically acceptable salt or solvate thereof;

TABLE 1

| Cpd. | | |
|---|---|---|
| No. | Structure | Name |
| 1 | 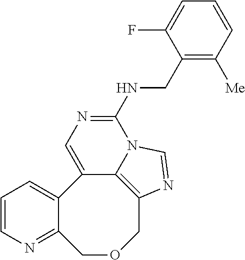 [SMILES: Cc1cccc(F)c1CNc1ncc2c3c(ncn13)COCc1ncccc1-2] | N-(2-fluoro-6-methylbenzyl)-3H,5H-4-oxa- 2,6,11,12a-tetraazabenzo[4,5]cycloocta[1,2,3- cd]inden-12-amine |
| 2 | 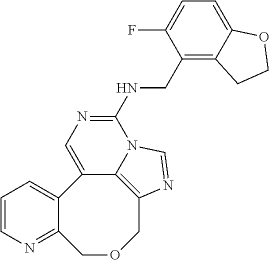 [SMILES: Fc1ccc2c(c1CNc1ncc3c4c(ncn14)COCc1ncccc1-3)CCO2] | N-((5-fluoro-2,3-dihydrobenzofuran-4-yl)methyl)- 3H,5H-4-oxa-2,6,11,12a- tetraazabenzo[4,5]cycloocta[1,2,3-cd]inden-12- amine |
| 3 | 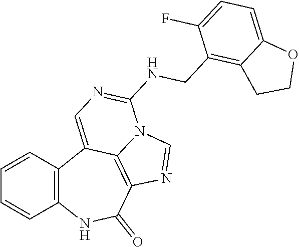 [SMILES: O=C1Nc2ccccc2-c2cnc(NCc3c(F)ccc4c3CCO4)n3cnc1c23] | 11-(((5-fluoro-2,3-dihydrobenzofuran-4- yl)methyl)amino)-2,4,10,11a- tetraazadibenzo[cd,f]azulen-3(4H)-one |
| 4 | 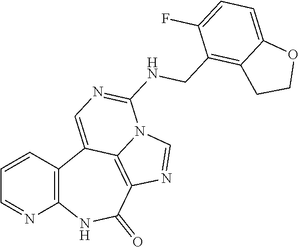 [SMILES: O=C1Nc2ncccc2-c2cnc(NCc3c(F)ccc4c3CCO4)n3cnc1c23] | 11-(((5-fluoro-2,3-dihydrobenzofurna-4- yl)methyl)amino)-2,4,510,11a- pentaazadibenzo[cd,f]azulen-3(4H)-one |
| 5 | 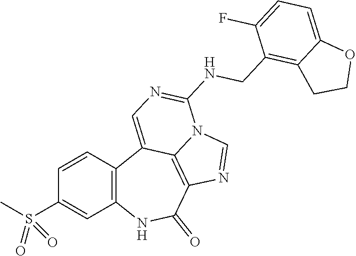 [SMILES: CS(=O)(=O)c1ccc2c(c1)NC(=O)c1ncn3c(NCc4c(F)ccc5c4CCO5)ncc-2c13] | 11-(((5-fluoro-2,3-dihydrobenzofuran-4- yl)methyl)amino)-6-(methylsulfonyl)-2,4,10,11a- tetraazadibenzo[cd,f]azulen-3(4H)-one |
| 6 | 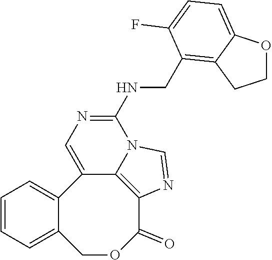 [SMILES: O=C1OCc2ccccc2-c2cnc(NCc3c(F)ccc4c3CCO4)n3cnc1c23] | 12-(((5-fluoro-2,3-dihydrobenzofuran-4- yl)methyl)amino)-3H,5H-4-oxa-2,11,12a- triazabenzo[4,5]cycloocta[1,2,3-cd]inden-3-one |
| 7 | 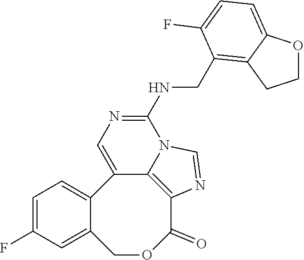 [SMILES: O=C1OCc2cc(F)ccc2-c2cnc(NCc3c(F)ccc4c3CCO4)n3cnc1c23] | 7-fluoro-12-(((5-fluoro-2,3-dihydrobenzofuran-4- yl)methyl)amino)-3H,5H-4-oxa-2,11,12a- triazabenzo[4,5]cycloocta[1,2,3-cd]inden-3-one |
| 8 | 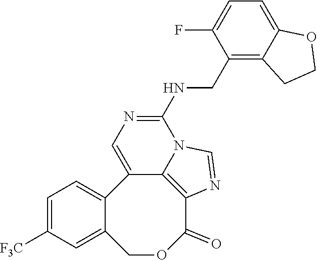 [SMILES: O=C1OCc2cc(C(F)(F)F)ccc2-c2cnc(NCc3c(F)ccc4c3CCO4)n3cnc1c23] | 12-(((5-fluoro-2,3-dihydrobenzofuran-4- yl)methyl)amino)-7-(trifluoromethyl)-3H,5H-4- oxa-2,11,12a-triazabenzo[4,5]cycloocta[1,2,3- cd]inden-3-one |
| 9 | 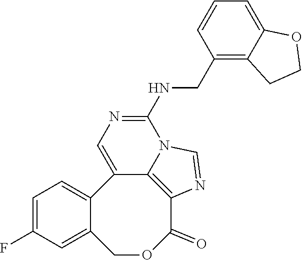 [SMILES: O=C1OCc2cc(F)ccc2-c2cnc(NCc3cccc4c3CCO4)n3cnc1c23] | 12-(((2,3-dihydrobenzofuran-4-yl)methyl)amino)- 7-fluoro-3H,5H-4-oxa-2,11,12a- triazabenzo[4,5]cycloocta[1,2,3-cd]inden-3-one |
| 10 | 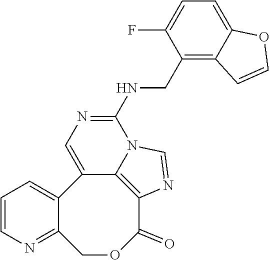 [SMILES: O=C1OCc2ncccc2-c2cnc(NCc3c(F)ccc4occc34)n3cnc1c23] | 12-(((5-fluorobenzofuran-4-yl)methyl)amino)- 3H,5H-4-oxa-2,6,11,12a- tetraazabenzo[4,5]cycloocta[1,2,3-cd]inden-3-one |
| 11 | 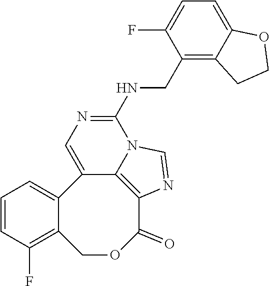 [SMILES: O=C1OCc2c(F)cccc2-c2cnc(NCc3c(F)ccc4c3CCO4)n3cnc1c23] | 6-fluoro-12-(((5-fluoro-2,3-dihydrobenzofuran-4- yl)methyl)amino)-3H,5H-4-oxa-2,11,12a- triazabenzo[4,5]cycloocta[1,2,3-cd]indene-3-one |
| 12 | 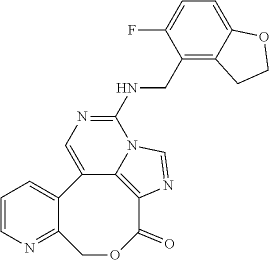 [SMILES: O=C1OCc2ncccc2-c2cnc(NCc3c(F)ccc4c3CCO4)n3cnc1c23] | 12-(((5-fluoro-2,3-dihydrobenzofuran-4- yl)methyl)amino)-3H,5H-4-oxa-2,6,11,12a- tetraazabenzo[4,5]cycloocta[1,2,3-cd]inden-3-one |
| 13 | 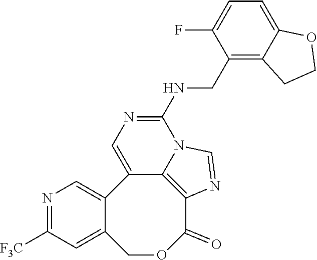 [SMILES: O=C1OCc2cc(C(F)(F)F)ncc2-c2cnc(NCc3c(F)ccc4c3CCO4)n3cnc1c23] | 12-(((5-fluoro-2,3-dihydrobenzofuran-4- yl)methyl)amino)-7-(trifluoromethyl)-3H,5H-4- oxa-2,8,11,12a-tetraazabenzo[4,5]cycloocta[1,2,3- cd]inden-3-one |
| 14 | 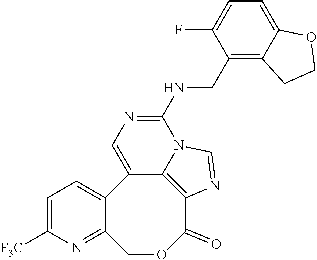 [SMILES: O=C1OCc2nc(C(F)(F)F)ccc2-c2cnc(NCc3c(F)ccc4c3CCO4)n3cnc1c23] | 12-(((5-fluoro-2,3-dihydrobenzofuran-4- yl)methyl)amino)-7-(trifluoromethyl)-3H,5H-4- oxa-2,6,11,12a-tetraazabenzo[4,5]cycloocta[1,2,3- cd]inden-3-one |
| 15 | 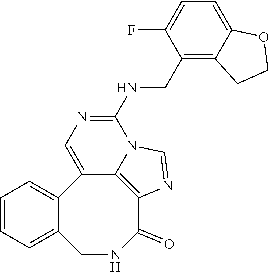 [SMILES: O=C1NCc2ccccc2-c2cnc(NCc3c(F)ccc4c3CCO4)n3cnc1c23] | 12-(((5-fluoro-2,3-dihydrobenzofuran-4- yl)methyl)amino)-4,5-dihydro-3H-2,4,11,12a- tetraazabenzo[4,5]cycloocta[1,2,3-cd]inden-3-one |
| 16 | 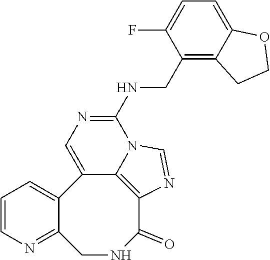 [SMILES: O=C1NCc2ncccc2-c2cnc(NCc3c(F)ccc4c3CCO4)n3cnc1c23] | 12-(((5-fluoro-2,3-dihydrobenzofuran-4- yl)methyl)amino)-4,5-dihydro-3H-2,4,6,11,12a- pentaazabenzo[4,5]cycloocta[1,2,3-cd]inden-3- one |
| 17 | 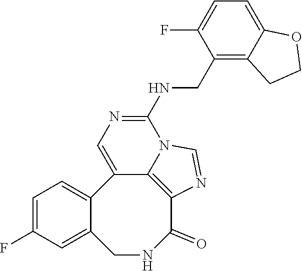 [SMILES: O=C1NCc2cc(F)ccc2-c2cnc(NCc3c(F)ccc4c3CCO4)n3cnc1c23] | 7-fluoro-12-(((5-fluoro-2,3-dihydrobenzofuran-4- yl)methyl)amino)-4,5-dihydro-3H-2,4,11,12a- tetraazabenzo[4,5]cycloocta[1,2,3-cd]inden-3- one |
| 18 | 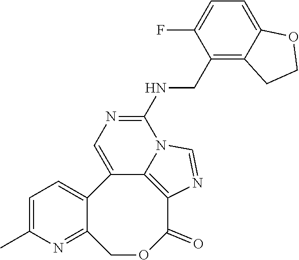 [SMILES: Cc1ccc2c(n1)COC(=O)c1ncn3c(NCc4c(F)ccc5c4CCO5)ncc-2c13] | 12-(((5-fluoro-2,3-dihydrobenzofuran-4- yl)methyl)amino)-7-methyl-3H,5H-4-oxa- 2,6,11,12a-tetraazabenzo[4,5]cycloocta[1,2,3- cd]inden-3-one |
| 19 | 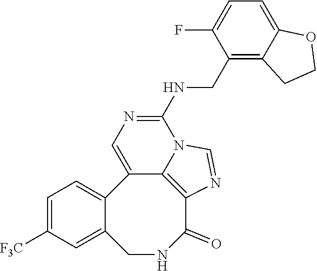 [SMILES: O=C1NCc2cc(C(F)(F)F)ccc2-c2cnc(NCc3c(F)ccc4c3CCO4)n3cnc1c23] | 12-(((5-fluoro-2,3-dihydrobenzofuran-4- yl)methyl)amino)-7-(trifluoromethyl)-4,5- dihydro-3H-2,4,11,12a- tetraazabenzo[4,5]cycloocta[1,2,3-cd]inden-3-one |
| 20 | 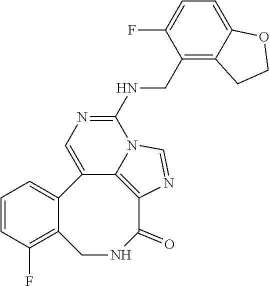 [SMILES: O=C1NCc2c(F)cccc2-c2cnc(NCc3c(F)ccc4c3CCO4)n3cnc1c23] | 6-fluoro-12-(((5-fluoro-2,3-dihydrobenzofuran-4- yl)methyl)amino)-4,5-dihydro-3H-2,4,11,12a- tetraazabenzo[4,5]cycloocta[1,2,3-cd]inden-3-one |
| 21 | 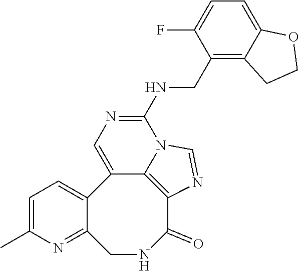 [SMILES: Cc1ccc2c(n1)CNC(=O)c1ncn3c(NCc4c(F)ccc5c4CCO5)ncc-2c13] | 12-(((5-fluoro-2,3-dihydrobenzofuran-4- yl)methyl)amino)-7-methyl-4,5-dihydro-3H- 2,4,6,11,12a-pentaazabenzo[4,5]cycloocta[1,2,3- cd]inden-3-one |
| 22 | 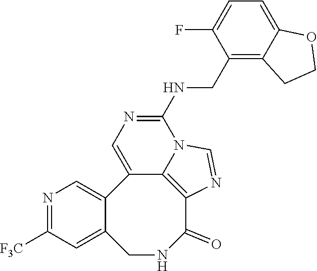 [SMILES: O=C1NCc2cc(C(F)(F)F)ncc2-c2cnc(NCc3c(F)ccc4c3CCO4)n3cnc1c23] | 12-(((5-fluoro-2,3-dihydrobenzofuran-4- yl)methyl)amino)-7-(trifluoromethyl)-4,5- dihydro-3H-2,4,8,11,12a- pentaazabenzo[4,5]cycloocta[1,2,3-cd]inden-3- one |
| 23 | 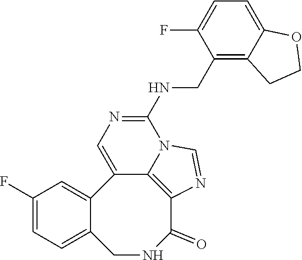 [SMILES: O=C1NCc2ccc(F)cc2-c2cnc(NCc3c(F)ccc4c3CCO4)n3cnc1c23] | 8-fluoro-12-(((5-fluoro-2,3-dihydrobenzofuran-4- yl)methyl)amino)-4,5-dihydro-3H-2,4,11,12a- tetraazabenzo[4,5]cycloocta[1,2,3-cd]inden-3-one |
| 24 | 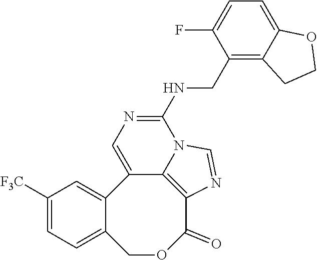 [SMILES: O=C1OCc2ccc(C(F)(F)F)cc2-c2cnc(NCc3c(F)ccc4c3CCO4)n3cnc1c23] | 12-(((5-fluoro-2,3-dihydrobenzofuran-4- yl)methyl)amino)-8-(trifluoromethyl)-3H,5H-4- oxa-2,11,12a-triazabenzo[4,5]cycloocta[1,2,3- cd]inden-3-one |
| 25 | 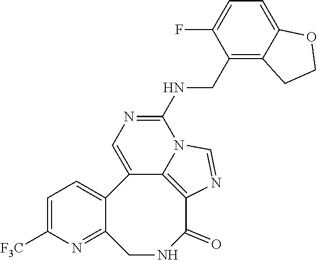 [SMILES: O=C1NCc2nc(C(F)(F)F)ccc2-c2cnc(NCc3c(F)ccc4c3CCO4)n3cnc1c23] | 12-(((5-fluoro-2,3-dihydrobenzofuran-4- yl)methyl)amino)-7-(trifluoromethyl)-4,5- dihydro-3H-2,4,6,11,12a- pentaazabenzo[4,5]cycloocta[1,2,3-cd]inden-3- one |
| 26 | 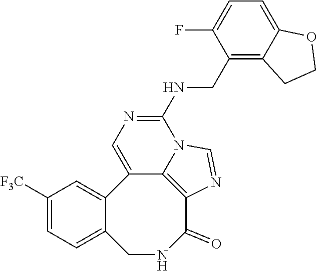 [SMILES: O=C1NCc2ccc(C(F)(F)F)cc2-c2cnc(NCc3c(F)ccc4c3CCO4)n3cnc1c23] | 12-(((5-fluoro-2,3-dihydrobenzofuran-4- yl)methyl)amino)-8-(trifluoromethyl)-4,5- dihydro-3H-2,4,11,12a- tetraaabenzo[4,5]cycloocta[1,2,3-cd]inden-3-one |
| 27 | 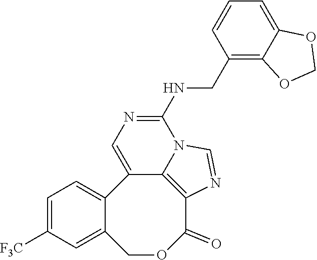 [SMILES: O=C1OCc2cc(C(F)(F)F)ccc2-c2cnc(NCc3cccc4c3OCO4)n3cnc1c23] | 12-((benzo[d][1,3]dioxol-4-ylmethyl)amino)-7- (trifluoromethyl)-3H,5H-4-oxa-2,11,12a- triazabenzo[4,5]cycloocta[1,2,3-cd]inden-3-one |
| 28 | 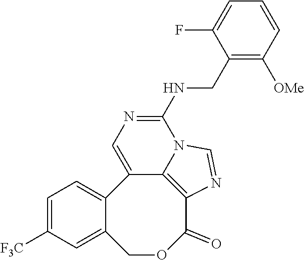 [SMILES: COc1cccc(F)c1CNc1ncc2c3c(ncn13)C(=O)OCc1cc(C(F)(F)F)ccc1-2] | 12-((2-fluoro-6-methoxybenzyl)amino)-7- (trifluoromethyl)-3H,5H-4-oxa-2,11,12a- triazabenzo[4,5]cycloocta[1,2,3-cd]inden-3-one |
| 29 | 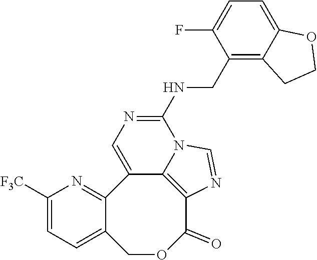 [SMILES: O=C1OCc2ccc(C(F)(F)F)nc2-c2cnc(NCc3c(F)ccc4c3CCO4)n3cnc1c23] | 12-(((5-fluoro-2,3-dihydrobenzofuran-4- yl)methyl)amino)-8-(trifluoromethyl)-3H,5H-4- oxa-2,9,11,12a-tetraazabenzo[4,5]cycloocta[1,2,3- cd]inden-3-one |
| 30 | 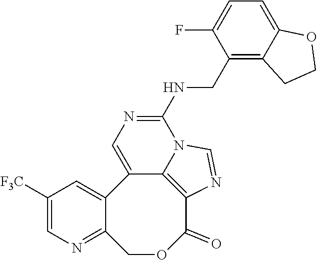 [SMILES: O=C1OCc2ncc(C(F)(F)F)cc2-c2cnc(NCc3c(F)ccc4c3CCO4)n3cnc1c23] | 12-(((5-fluoro-2,3-dihydrobenzofuran-4- yl)methyl)amino)-8-(trifluoromethyl)-3H,5H-4- oxa-2,6,11,12a-tetraazabenzo[4,5]cycloocta[1,2,3- cd]inden-3-one |
| 31 | 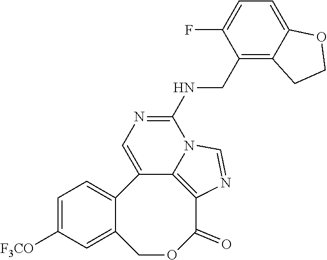 [SMILES: O=C1OCc2cc(OC(F)(F)F)ccc2-c2cnc(NCc3c(F)ccc4c3CCO4)n3cnc1c23] | 12-(((5-fluoro-2,3-dihydrobenzofuran-4- yl)methyl)amino)-7-(trifluoromethoxy)-3H,5H-4- oxa-2,11,12a-triazabenzo[4,5]cycloocta[1,2,3- cd]inden-3-one |
| 32 | 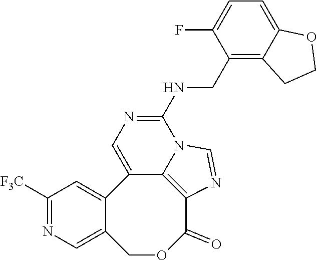 [SMILES: O=C1OCc2cnc(C(F)(F)F)cc2-c2cnc(NCc3c(F)ccc4c3CCO4)n3cnc1c23] | 12-(((5-fluoro-2,3-dihydrobenzofuran-4- yl)methyl)amino)-8-(trifluoromethyl)-3H,5H-4- oxa-2,7,11,12a-tetraazabenzo[4,5]cycloocta[1,2,3- cd]inden-3-one |
| 33 | 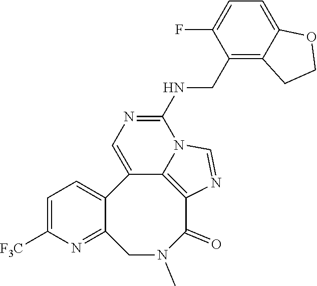 [SMILES: CN1Cc2nc(C(F)(F)F)ccc2-c2cnc(NCc3c(F)ccc4c3CCO4)n3cnc(c23)C1=O] | 12-(((5-fluoro-2,3-dihydrobenzofuran-4- yl)methyl)amino)-4-methyl-7-(trifluoromethyl)- 4,5-dihydro-3H-2,4,6,11,12a- pentaazabenzo[4,5]cycloocta[1,2,3-cd]inden-3- one |
| 34 | 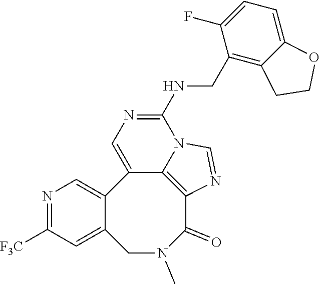 [SMILES: CN1Cc2cc(C(F)(F)F)ncc2-c2cnc(NCc3c(F)ccc4c3CCO4)n3cnc(c23)C1=O] | 12-(((5-fluoro-2,3-dihydrobenzofuran-4- yl)methyl)amino)-4-methyl-7-(trifluoromethyl)- 4,5-dihydro-3H-2,4,8,11,12a- pentaazabenzo[4,5]cycloocta[1,2,3-cd]inden-3- one |
| 35 | 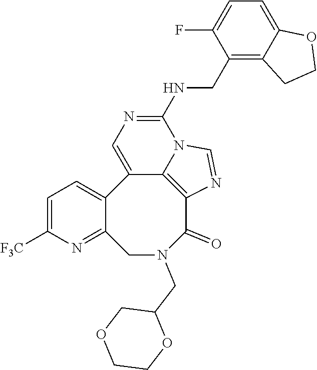 [SMILES: O=C1c2ncn3c(NCc4c(F)ccc5c4CCO5)ncc(c23)-c2ccc(C(F)(F)F)nc2CN1CC1COCCO1] | 4-((1,4-dioxan-2-yl)methyl)-12-(((5-fluoro-2,3- dihydrobenzofuran-4-yl)methyl)amino)-7- (trifluoromethyl)-4,5-dihydro-3H-2,4,6,11,12a- pentaazabenzo[4,5]cycloocta[1,2,3-cd]inden-3- one |
| 36 | 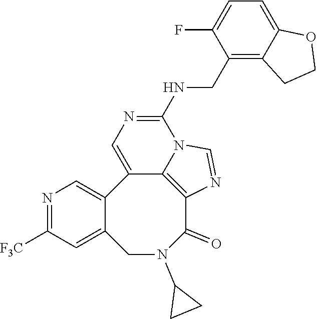 [SMILES: O=C1c2ncn3c(NCc4c(F)ccc5c4CCO5)ncc(c23)-c2cnc(C(F)(F)F)cc2CN1C1CC1] | 4-cyclopropyl-12-(((5-fluoro-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| 37 | 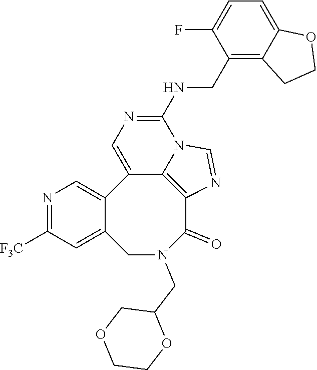 [SMILES: O=C1c2ncn3c(NCc4c(F)ccc5c4CCO5)ncc(c23)-c2cnc(C(F)(F)F)cc2CN1CC1COCCO1] | 4-((1,4-dioxan-2-yl)methyl)-12-(((5-fluoro-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| 38 | 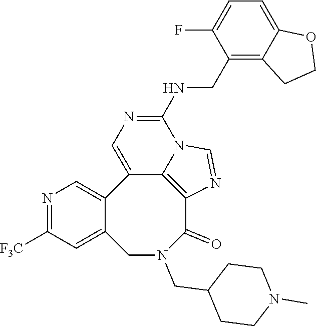 [SMILES: CN1CCC(CN2Cc3cc(C(F)(F)F)ncc3-c3cnc(NCc4c(F)ccc5c4CCO5)n4cnc(c34)C2=O)CC1] | 12-(((5-fluoro-2,3-dihydrobenzofuran-4- yl)methyl)amino)-4-((1-methylpiperidin-4- yl)methyl)-7-(trifluoromethyl)-4,5-dihydro-3H- 2,4,8,11,12a-pentaazabenzo[4,5]cycloocta[1,2,3- cd]inden-3-one |
| 39 | 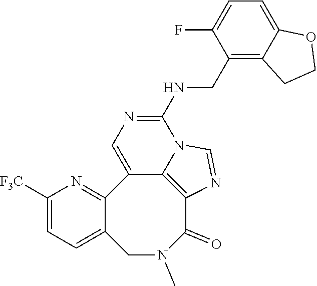 [SMILES: CN1Cc2ccc(C(F)(F)F)nc2-c2cnc(NCc3c(F)ccc4c3CCO4)n3cnc(c23)C1=O] | 12-(((5-fluoro-2,3-dihydrobenzofuran-4- yl)methyl)amino)-4-methyl-8-(trifluoromethyl)- 4,5-dihydro-3H-2,4,9,11,12a- pentaazabenzo[4,5]cycloocta[1,2,3-cd]inden-3- one |
| 40 | 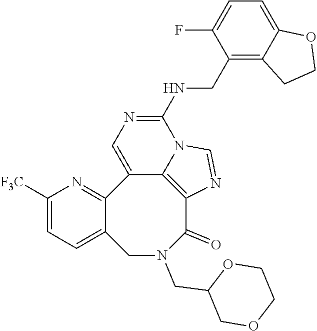 [SMILES: O=C1c2ncn3c(NCc4c(F)ccc5c4CCO5)ncc(c23)-c2nc(C(F)(F)F)ccc2CN1CC1COCCO1] | 4-((1,4-dioxan-2-yl)methyl)-12-(((5-fluoro-2,3- dihydrobenzofuran-4-yl)methyl)amino)-8- (trifluoromethyl)-4,5-dihydro-3H-2,4,9,11,12a- pentaazabenzo[4,5]cycloocta[1,2,3-cd]inden-3- one |
| 41 | 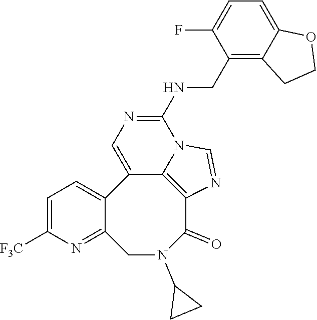 [SMILES: O=C1c2ncn3c(NCc4c(F)ccc5c4CCO5)ncc(c23)-c2ccc(C(F)(F)F)nc2CN1C1CC1] | 4-cyclopropyl-12-(((5-fluoro-2,3- dihydrobenzofuran-4-yl)methyl)amino)-7- (trifluoromethyl)-4,5-dihydro-3H-2,4,6,11,12a- pentaazabenzo[4,5]cycloocta[1,2,3-cd]inden-3- one |
| 42 | 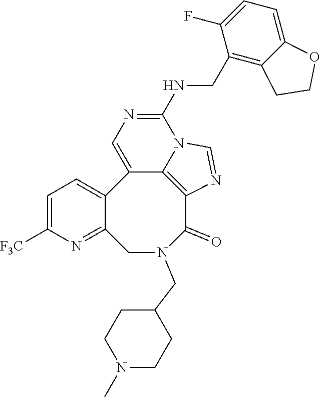 [SMILES: CN1CCC(CN2Cc3nc(C(F)(F)F)ccc3-c3cnc(NCc4c(F)ccc5c4CCO5)n4cnc(c34)C2=O)CC1] | 12-(((5-fluoro-2,3-dihydrobenzofuran-4- yl)methyl)amino)-4-((1-methylpiperidin-4- yl)methyl)-7-(trifluoromethyl)-4,5-dihydro-3H- 2,4,6,11,12a-pentaazabenzo[4,5]cycloocta[1,2,3- cd]inden-3-one |
| 43 | 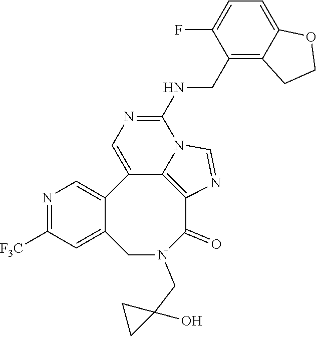 [SMILES: O=C1c2ncn3c(NCc4c(F)ccc5c4CCO5)ncc(c23)-c2cnc(C(F)(F)F)cc2CN1CC1(O)CC1] | 12-(((5-fluoro-2,3-dihydrobenzofuran-4- yl)methyl)amino)-4-((1- hydroxycyclopropyl)methyl)-7-(trifluoromethyl)- 4,5-dihydro-3H-2,4,8,11,12a- pentaazabenzo[4,5]cycloocta[1,2,3-cd]inden-3- one |
| 44 | 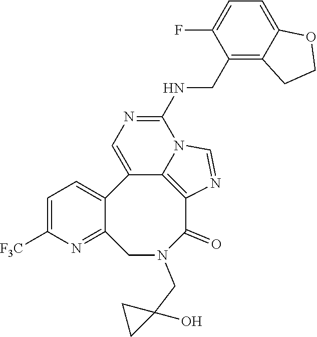 [SMILES: O=C1c2ncn3c(NCc4c(F)ccc5c4CCO5)ncc(c23)-c2ccc(C(F)(F)F)nc2CN1CC1(O)CC1] | 12-(((5-fluoro-2,3-dihydrobenzofuran-4- yl)methyl)amino)-4-((1- hydroxycyclopropyl)methyl)-7-(trifluoromethyl)- 4,5-dihydro-3H-2,4,6,11,12a- pentaazabenzo[4,5]cycloocta[1,2,3-cd]inden-3- one |
| 45 | 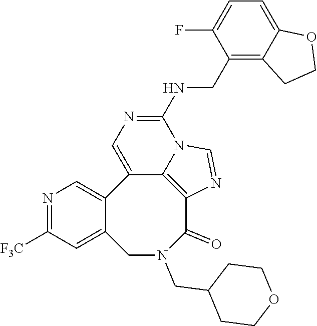 [SMILES: O=C1c2ncn3c(NCc4c(F)ccc5c4CCO5)ncc(c23)-c2cnc(C(F)(F)F)cc2CN1CC1CCOCC1] | 12-(((5-fluoro-2,3-dihydrobenzofuran-4- yl)methyl)amino)-4-((tetrahydro-2H-pyran-4- yl)methyl)-7-(trifluoromethyl)-4,5-dihydro-3H- 2,4,8,11,12a-pentaazabenzo[4,5]cycloocta[1,2,3- cd]inden-3-one |
| 46 | 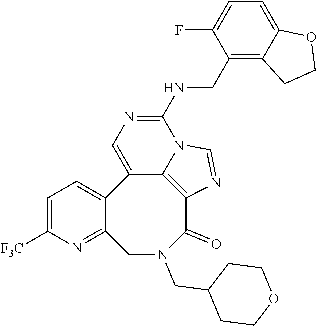 [SMILES: O=C1c2ncn3c(NCc4c(F)ccc5c4CCO5)ncc(c23)-c2ccc(C(F)(F)F)nc2CN1CC1CCOCC1] | 12-(((5-fluoro-2,3-dihydrobenzofuran-4- yl)methyl)amino)-4-((tetrahydro-2H-pyran-4- yl)methyl)-7-(trifluoromethyl)-4,5-dihydro-3H- 2,4,6,11,12a-pentaazabenzo[4,5]cycloocta[1,2,3- cd]inden-3-one |
| 47 | 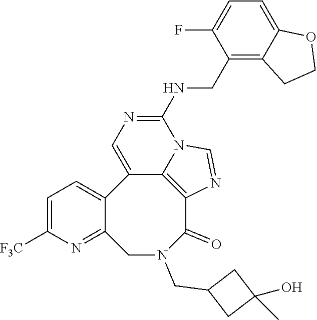 [SMILES: CC1(O)CC(CN2Cc3nc(C(F)(F)F)ccc3-c3cnc(NCc4c(F)ccc5c4CCO5)n4cnc(c34)C2=O)C1] | 12-(((5-fluoro-2,3-dihydrobenzofuran-4- yl)methyl)amino)-4-((3-hydroxy-3- methylcyclobutyl)methyl)-7-(trifluoromethyl)-4,5- dihydro-3H-2,4,6,11,12a- pentaazabenzo[4,5]cycloocta[1,2,3-cd]inden-3- one |
| 48 | 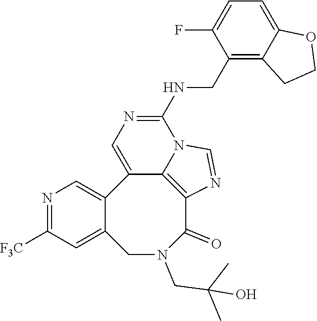 [SMILES: CC(C)(O)CN1Cc2cc(C(F)(F)F)ncc2-c2cnc(NCc3c(F)ccc4c3CCO4)n3cnc(c23)C1=O] | 12-(((5-fluoro-2,3-dihydrobenzofuran-4- yl)methyl)amino)-4-(2-hydroxy-2-methylpropyl)- 7-(trifluoromethyl)-4,5-dihydro-3H-2,4,8,11,12a- pentaazabenzo[4,5]cycloocta[1,2,3-cd]inden-3- one |
| 49 | 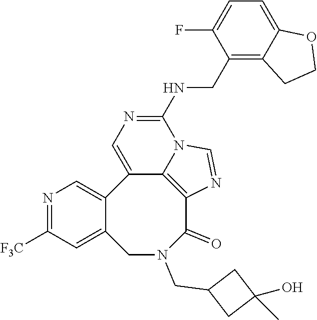 [SMILES: CC1(O)CC(CN2Cc3cc(C(F)(F)F)ncc3-c3cnc(NCc4c(F)ccc5c4CCO5)n4cnc(c34)C2=O)C1] | 12-(((5-fluoro-2,3-dihydrobenzofuran-4- yl)methyl)amino)-4-((3-hydroxy-3- methylcyclobutyl)methyl)-7-(trifluoroemthyl)-4,5- dihydro-3H-2,4,8,11,12a- pentaazabenzo[4,5]cycloocta[1,2,3-cd]inden-3- one |
| 50 | 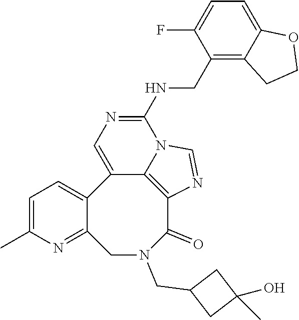 [SMILES: Cc1ccc2c(n1)CN(CC1CC(C)(O)C1)C(=O)c1ncn3c(NCc4c(F)ccc5c4CCO5)ncc-2c13] | 12-(((5-fluoro-2,3-dihydrobenzofuran-4- yl)methyl)amino)-4-((3-hydroxy-3- methylcyclobutyl)methyl)-7-methyl-4,5-dihydro- 3H-2,4,6,11,12a- pentaazabenzo[4,5]cycloocta[1,2,3-cd]inden-3- one |
| 51 | 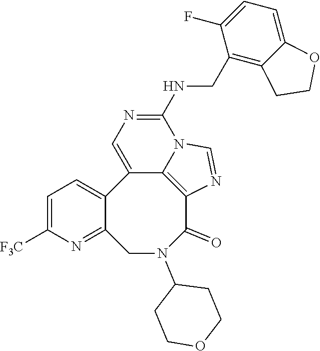 [SMILES: O=C1c2ncn3c(NCc4c(F)ccc5c4CCO5)ncc(c23)-c2ccc(C(F)(F)F)nc2CN1C1CCOCC1] | 12-(((5-fluoro-2,3-dihydrobenzofuran-4- yl)methyl)amino)-4-(tetrahydro-2H-pyran-4-yl)-7- (trifluoromethyl)-4,5-dihydro-3H-2,4,6,11,12a- pentaazabenzo[4,5]cycloocta[1,2,3-cd]inden-3- one |
| 52 | 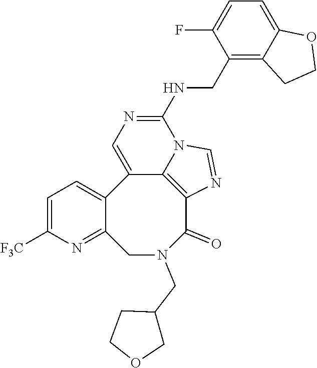 [SMILES: O=C1c2ncn3c(NCc4c(F)ccc5c4CCO5)ncc(c23)-c2ccc(C(F)(F)F)nc2CN1CC1CCOC1] | 12-(((5-fluoro-2,3-dihydrobenzofuran-4- yl)methyl)amino)-4-((tetrahydrofuran-3- yl)methyl)-7-(trifluoromethyl)-4,5-dihydro-3H- 2,4,6,11,12a-pentaazabenzo[4,5]cycloocta[1,2,3- cd]inden-3-one |
| 53 | 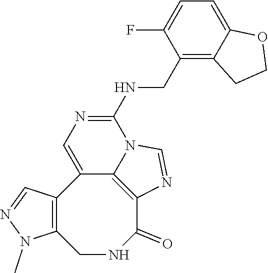 [SMILES: Cn1ncc2c1CNC(=O)c1ncn3c(NCc4c(F)ccc5c4CCO5)ncc-2c13] | 11-(((5-fluoro-2,3-dihydrobenzofuran-4- yl)methyl)amino)-6-methyl-5,6-dihydro- 2,4,6,7,10,11a- hexaazacyclopenta[4,5]cycloocta[1,2,3-cd]inden- 3(4H)-one |
| 54 | 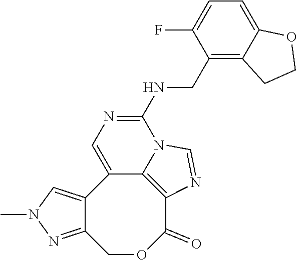 [SMILES: Cn1cc2c(n1)COC(=O)c1ncn3c(NCc4c(F)ccc5c4CCO5)ncc-2c13] | 11-(((5-fluoro-2,3-dihydrobenzofuran-4- yl)methyl)amino)-7-methyl-5,7-dihydro-3H-4- oxa-2,6,7,10,11a- pentaazacyclopenta[4,5]cycloocta[1,2,3-cd]inden- 3-one |
| 55 | 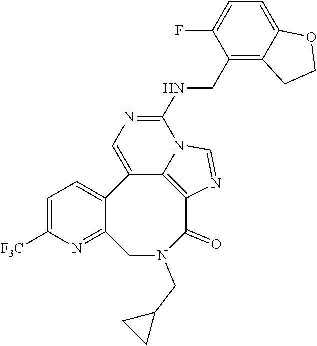 [SMILES: O=C1c2ncn3c(NCc4c(F)ccc5c4CCO5)ncc(c23)-c2ccc(C(F)(F)F)nc2CN1CC1CC1] | 4-(cyclopropylmethyl)-12-(((5-fluoro-2,3- dihydrobenzofuran-4-yl)methyl)amino)-7- (trifluoromethyl)-4,5-dihydro-3H-2,4,6,11,12a- pentaazabenzo[4,5]cycloocta[1,2,3-cd]inden-3- one |
| 56 | 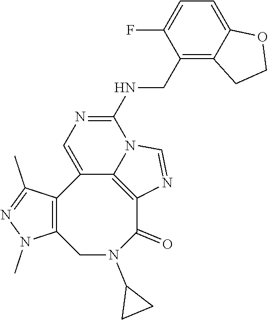 [SMILES: Cc1nn(C)c2c1-c1cnc(NCc3c(F)ccc4c3CCO4)n3cnc(c13)C(=O)N(C1CC1)C2] | 4-cyclopropyl-11-(((5-fluoro-2,3- dihydrobenzofuran-4-yl)methyl)amino)-6,8- dimethyl-5,6-dihydro-2,4,6,7,10,11a- hexaazacyclopenta[4,5]cycloocta[1,2,3-cd]inden- 3(4H)-one |
| 57 | 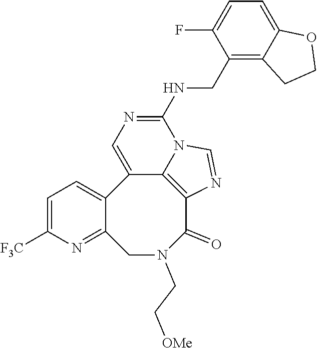 [SMILES: COCCN1Cc2nc(C(F)(F)F)ccc2-c2cnc(NCc3c(F)ccc4c3CCO4)n3cnc(c23)C1=O] | 12-(((5-fluoro-2,3-dihydrobenzofuran-4- yl)methyl)amino)-4-(2-methoxyethyl)-7- (trifluoromethyl)-4,5-dihydro-3H-2,4,6,11,12a- pentaazabenzo[4,5]cycloocta[1,2,3-cd]inden-3- one |
| 58 | 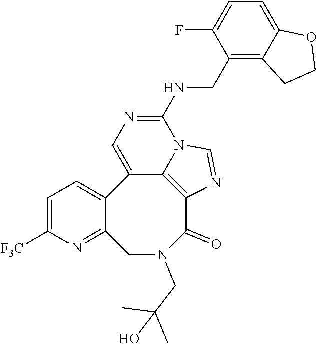 [SMILES: CC(C)(O)CN1Cc2nc(C(F)(F)F)ccc2-c2cnc(NCc3c(F)ccc4c3CCO4)n3cnc(c23)C1=O] | 12-(((5-fluoro-2,3-dihydrobenzofuran-4- yl)methyl)amino)-4-(2-hydroxy-2-methylpropyl)- 7-(trifluoromethyl)-4,5-dihydro-3H-2,4,6,11,12a- pentaazabenzo[4,5]cycloocta[1,2,3-cd]inden-3- one |
| 59 | 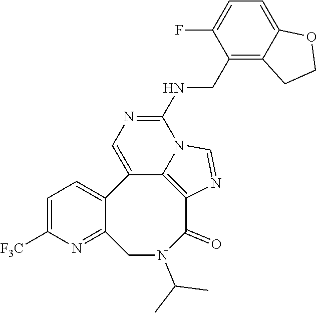 [SMILES: CC(C)N1Cc2nc(C(F)(F)F)ccc2-c2cnc(NCc3c(F)ccc4c3CCO4)n3cnc(c23)C1=O] | 12-(((5-fluoro-2,3-dihydrobenzofuran-4- yl)methyl)amino)-4-isopropyl-7-(trifluoromethyl)- 4,5-dihydro-3H-2,4,6,11,12a- pentaazabenzo[4,5]cycloocta[1,2,3-cd]inden-3- one |
| 60 | 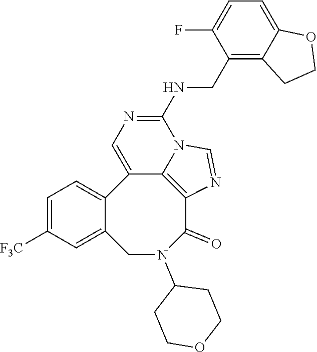 [SMILES: O=C1c2ncn3c(NCc4c(F)ccc5c4CCO5)ncc(c23)-c2ccc(C(F)(F)F)cc2CN1C1CCOCC1] | 12-(((5-fluoro-2,3-dihydrobenzofuran-4- yl)methyl)amino)-4-(tetrahydro-2H-pyran-4-yl)-7- (trifluoromethyl)-4,5-dihydro-3H-2,4,11,12a- tetraazabenzo[4,5]cycloocta[1,2,3-cd]inden-3-one |
| 61 | 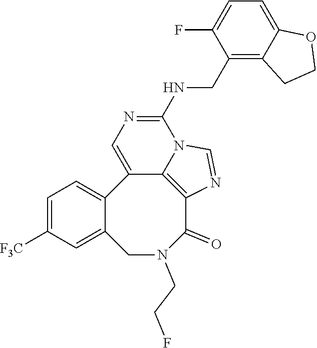 [SMILES: O=C1c2ncn3c(NCc4c(F)ccc5c4CCO5)ncc(c23)-c2ccc(C(F)(F)F)cc2CN1CCF] | 12-(((5-fluoro-2,3-dihydrobenzofuran-4- yl)methyl)amino)-4-(2-fluoroethyl)-7- (trifluoromethyl)-4,5-dihydro-3H-2,4,11,12a- tetraazabenzo[4,5]cycloocta[1,2,3-cd]inden-3-one |
| 62 | 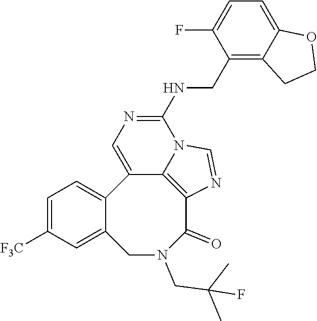 [SMILES: CC(C)(F)CN1Cc2cc(C(F)(F)F)ccc2-c2cnc(NCc3c(F)ccc4c3CCO4)n3cnc(c23)C1=O] | 12-(((5-fluoro-2,3-dihydrobenzofuran-4- yl)methyl)amino)-4-(2-fluoro-2-methylpropyl)-7- (trifluoromethyl)-4,5-dihydro-3H-2,4,11,12a- tetraazabenzo[4,5]cycloocta[1,2,3-cd]inden-3-one |
| 63 | 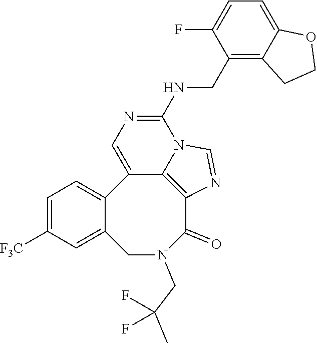 [SMILES: CC(F)(F)CN1Cc2cc(C(F)(F)F)ccc2-c2cnc(NCc3c(F)ccc4c3CCO4)n3cnc(c23)C1=O] | 4-(2,2-difluoropropyl)-12-(((5-fluoro-2,3- dihydrobenzofuran-4-yl)methyl)amino)-7- (trifluoromethyl)-4,5-dihydro-3H-2,4,11,12a- tetraazabenzo[4,5]cycloocta[1,2,3-cd]inden-3-one |
| 64 | 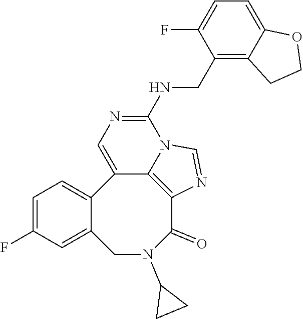 [SMILES: O=C1c2ncn3c(NCc4c(F)ccc5c4CCO5)ncc(c23)-c2ccc(F)cc2CN1C1CC1] | 4-cyclopropyl-7-fluoro-12-(((5-fluoro-2,3- dihydrobenzofuran-4-yl)methyl)amino)-4,5- dihydro-3H-2,4,11,12a- tetraazabenzo[4,5]cycloocta[1,2,3-cd]inden-3-one |
| 65 | 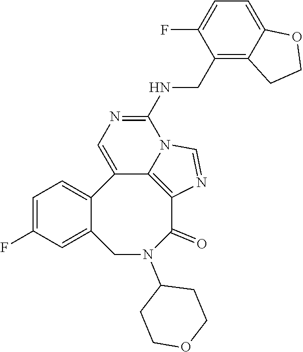 [SMILES: O=C1c2ncn3c(NCc4c(F)ccc5c4CCO5)ncc(c23)-c2ccc(F)cc2CN1C1CCOCC1] | 7-fluoro-12-(((5-fluoro-2,3-dihydrobenzofuran-4- yl)methyl)amino)-4-(tetrahydro-2H-pyran-4-yl)- 4,5-dihydro-3H-2,4,11,12a- tetraazabenzo[4,5]cycloocta[1,2,3-cd]inden-3-one |
| 66 | 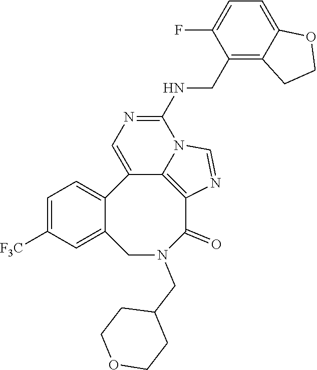 [SMILES: O=C1c2ncn3c(NCc4c(F)ccc5c4CCO5)ncc(c23)-c2ccc(C(F)(F)F)cc2CN1CC1CCOCC1] | 12-(((5-fluoro-2,3-dihydrobenzofuran-4- yl)methyl)amino)-4-((tetrahydro-2H-pyran-4- yl)methyl)-7-(trifluoromethyl)-4,5-dihydro-3H- 2,4,11,12a-tetraazabenzo[4,5]cycloocta[1,2,3- cd]inden-3-one |
| 67 | 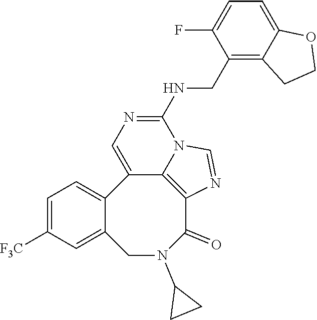 [SMILES: O=C1c2ncn3c(NCc4c(F)ccc5c4CCO5)ncc(c23)-c2ccc(C(F)(F)F)cc2CN1C1CC1] | 4-cyclopropyl-12-(((5-fluoro-2,3- dihydrobenzofuran-4-yl)methyl)amino)-7- (trifluoromethyl)-4,5-dihydro-3H-2,4,11,12a- tetraazabenzo[4,5]cycloocta[1,2,3-cd]inden-3-one |
| 68 | 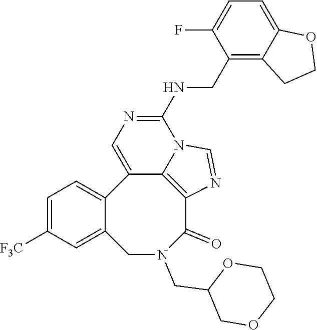 [SMILES: O=C1c2ncn3c(NCc4c(F)ccc5c4CCO5)ncc(c23)-c2ccc(C(F)(F)F)cc2CN1CC1COCCO1] | 4-((1,4-dioxan-2-yl)methyl)-12-(((5-fluoro-2,3- dihydrobenzofuran-4-yl)methyl)amino)-7- (trifluoromethyl)-4,5-dihydro-3H-2,4,11,12a- tetraazabenzo[4,5]cycloocta[1,2,3-cd]inden-3-one |
| 69 | 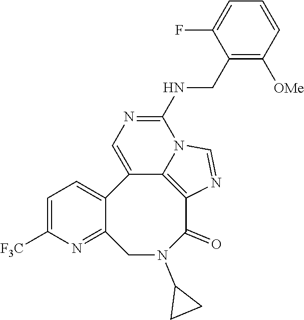 [SMILES: COc1cccc(F)c1CNc1ncc2c3c(ncn13)C(=O)N(C1CC1)Cc1nc(C(F)(F)F)ccc1-2] | 4-cyclopropyl-12-((2-fluoro-6- methoxybenzyl)amino)-7-(trifluoromethyl)-4,5- dihydro-3H-2,4,6,11,12a- pentaazabenzo[4,5]cycloocta[1,2,3-cd]inden-3- one |
| 70 | 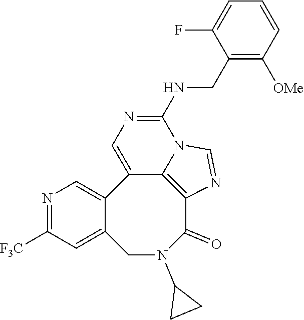 [SMILES: COc1cccc(F)c1CNc1ncc2c3c(ncn13)C(=O)N(C1CC1)Cc1cc(C(F)(F)F)ncc1-2] | 4-cyclopropyl-12-((2-fluoro-6- methoxybenzyl)amino)-7-(trifluoromethyl)-4,5- dihydro-3H-2,4,8,11,12a- pentaazabenzo[4,5]cycloocta[1,2,3-cd]inden-3- one |
| 71 | 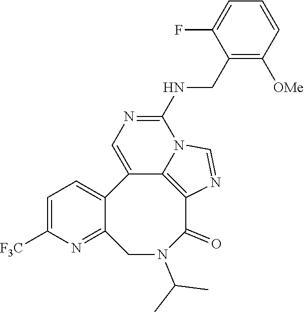 [SMILES: COc1cccc(F)c1CNc1ncc2c3c(ncn13)C(=O)N(C(C)C)Cc1nc(C(F)(F)F)ccc1-2] | 12-((2-fluoro-6-methoxybenzyl)amino)-4- isopropyl-7-(trifluoromethyl)-4,5-dihydro-3H- 2,4,6,11,12a-pentaazabenzo[4,5]cycloocta[1,2,3- cd]inden-3-one |
| 72 | 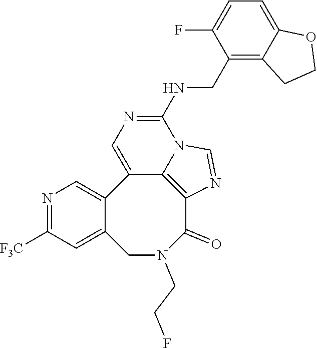 [SMILES: O=C1c2ncn3c(NCc4c(F)ccc5c4CCO5)ncc(c23)-c2cnc(C(F)(F)F)cc2CN1CCF] | 12-(((5-fluoro-2,3-dihydrobenzofuran-4- yl)methyl)amino)-4-(2-fluoroethyl)-7- (trifluoromethyl)-4,5-dihydro-3H-2,4,8,11,12a- pentaazabenzo[4,5]cycloocta[1,2,3-cd]inden-3- one |
| 73 | 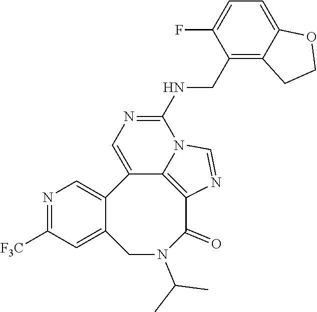 [SMILES: CC(C)N1Cc2cc(C(F)(F)F)ncc2-c2cnc(NCc3c(F)ccc4c3CCO4)n3cnc(c23)C1=O] | 12-(((5-fluoro-2,3-dihydrobenzofuran-4- yl)methyl)amino)-4-isopropyl-7-(trifluoromethyl)- 4,5-dihydro-3H-2,4,8,11,12a- pentaazabenzo[4,5]cycloocta[1,2,3-cd]inden-3- one |
| 74 | 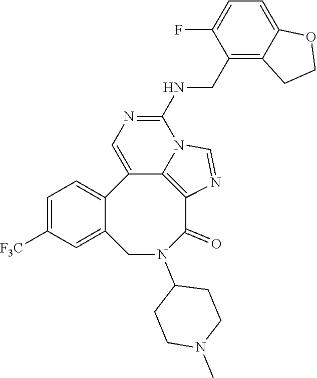 [SMILES: CN1CCC(N2Cc3cc(C(F)(F)F)ccc3-c3cnc(NCc4c(F)ccc5c4CCO5)n4cnc(c34)C2=O)CC1] | 12-(((5-fluoro-2,3-dihydrobenzofuran-4- yl)methyl)amino)-4-(1-methylpiperidin-4-yl)-7- (trifluoromethyl)-4,5-dihydro-3H-2,4,11,12a- tetraazabenzo[4,5]cycloocta[1,2,3-cd]inden-3-one |
| 75 | 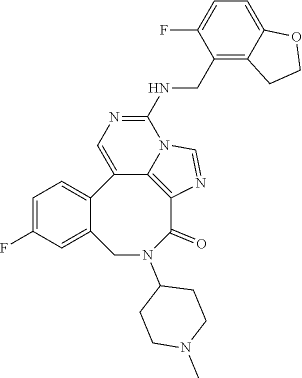 [SMILES: CN1CCC(N2Cc3cc(F)ccc3-c3cnc(NCc4c(F)ccc5c4CCO5)n4cnc(c34)C2=O)CC1] | 7-fluoro-12-(((5-fluoro-2,3-dihydrobenzofuran-4- yl)methyl)amino)-4-(1-methylpiperidin-4-yl)-4,5- dihydro-3H-2,4,11,12a- tetraazabenzo[4,5]cycloocta[1,2,3-cd]inden-3-one |
| 76 | 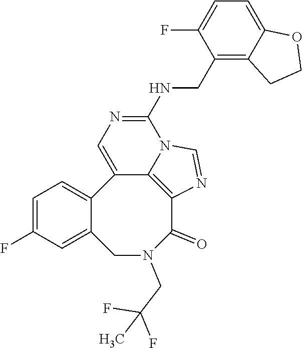 [SMILES: CC(F)(F)CN1Cc2cc(F)ccc2-c2cnc(NCc3c(F)ccc4c3CCO4)n3cnc(c23)C1=O] | 4-(2,2-difluoropropyl)-7-fluoro-12-(((5-fluoro- 2,3-dihydrobenzofuran-4-yl)methyl)amino)-4,5- dihydro-3H-2,4,11,12a- tetraazabenzo[4,5]cycloocta[1,2,3-cd]inden-3-one |
| 77 | 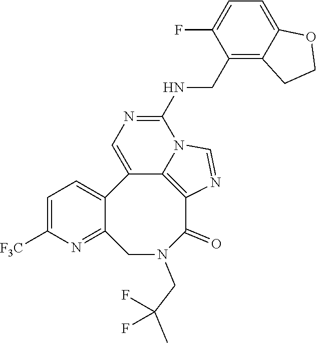 [SMILES: CC(F)(F)CN1Cc2nc(C(F)(F)F)ccc2-c2cnc(NCc3c(F)ccc4c3CCO4)n3cnc(c23)C1=O] | 4-(2,2-difluoropropyl)-12-(((5-fluoro-2,3- dihydrobenzofuran-4-yl)methyl)amino)-7- (trifluoromethyl)-4,5-dihydro-3H-2,4,6,11,12a- pentaazabenzo[4,5]cycloocta[1,2,3-cd]inden-3- one |
| 78 | 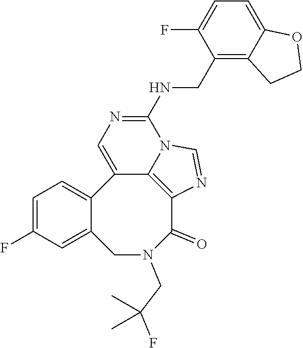 [SMILES: CC(C)(F)CN1Cc2cc(F)ccc2-c2cnc(NCc3c(F)ccc4c3CCO4)n3cnc(c23)C1=O] | 7-fluoro-12-(((5-fluoro-2,3-dihydrobenzofuran-4- y)methyl)amino)-4-(2-fluoro-2-methylpropyl)- 4,5-dihydro-3H-2,4,11,12a- tetraazabenzo[4,5]cycloocta[1,2,3-cd]inden-3-one |
| 79 | 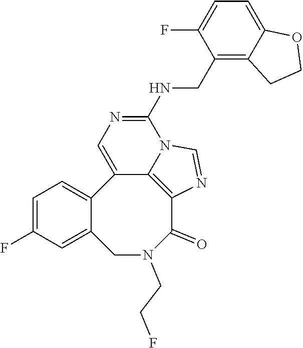 [SMILES: O=C1c2ncn3c(NCc4c(F)ccc5c4CCO5)ncc(c23)-c2ccc(F)cc2CN1CCF] | 7-fluoro-12-(((5-fluoro-2,3-dihydrobenzofuran-4- yl)methyl)amino)-4-(2-fluoroethyl)-4,5-dihydro- 3H-2,4,11,12a-tetraazabenzo[4,5]cycloocta[1,2,3- cd]inden-3-one |
| 80 | 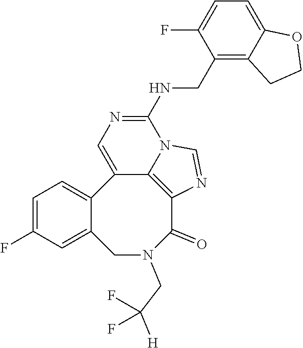 [SMILES: O=C1c2ncn3c(NCc4c(F)ccc5c4CCO5)ncc(c23)-c2ccc(F)cc2CN1CC(F)F] | 4-(2,2-difluoroethyl)-7-fluoro-12-(((5-fluoro-2,3- dihydrobenzofuran-4-yl)methyl)amino)-4,5- dihydro-3H-2,4,11,12a- tetraazabenzo[4,5]cycloocta[1,2,3-cd]inden-3-one |
| 81 | 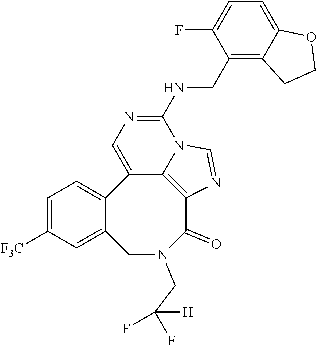 [SMILES: O=C1c2ncn3c(NCc4c(F)ccc5c4CCO5)ncc(c23)-c2ccc(C(F)(F)F)cc2CN1CC(F)F] | 4-(2,2-difluoroethyl)-12-(((5-fluoro-2,3- dihydrobenzofuran-4-yl)methyl)amino)7- (trifluoromethyl)-4,5-dihydro-3H-2,4,11,12a- tetraazabenzo[4,5]cycloocta[1,2,3-cd]inden-3-one |
| 82 | 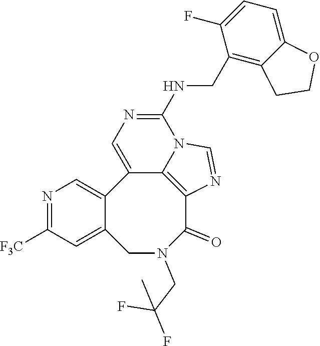 [SMILES: CC(F)(F)CN1Cc2cc(C(F)(F)F)ncc2-c2cnc(NCc3c(F)ccc4c3CCO4)n3cnc(c23)C1=O] | 4-(2,2-difluoropropyl)-12-(((5-fluoro-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| 83 | 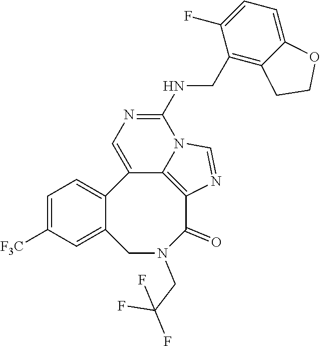 [SMILES: O=C1c2ncn3c(NCc4c(F)ccc5c4CCO5)ncc(c23)-c2ccc(C(F)(F)F)cc2CN1CC(F)(F)F] | 12-(((5-fluoro-2,3-dihydrobenzofuran-4- yl)methyl)amino)-4-(2,2,2-trifluoroethyl)-7- (trifluoromethyl)-4,5-dihydro-3H-2,4,11,12a- tetraazabenzo[4,5]cycloocta[1,2,3-cd]inden-3-one |
| 84 | 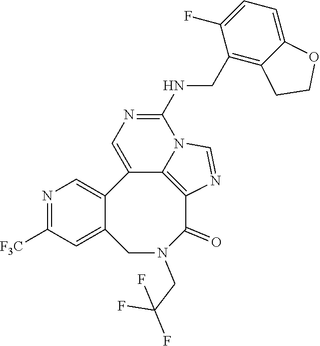 [SMILES: O=C1c2ncn3c(NCc4c(F)ccc5c4CCO5)ncc(c23)-c2cnc(C(F)(F)F)cc2CN1CC(F)(F)F] | 12-(((5-fluoro-2,3-dihydrobenzofuran-4- yl)methyl)amino)-4-(2,2,2-trifluoroethyl)-7- (trifluoromethyl)-4,5-dihydro-3H-2,4,8,11,12a- pentaazabenzo[4,5]cycloocta[1,2,3-cd]inden-3- one |
| 85 | 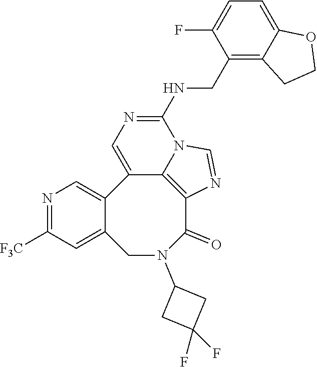 [SMILES: O=C1c2ncn3c(NCc4c(F)ccc5c4CCO5)ncc(c23)-c2cnc(C(F)(F)F)cc2CN1C1CC(F)(F)C1] | 4-(3,3-difluorocyclobutyl)-12-(((5-fluoro-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| 86 | 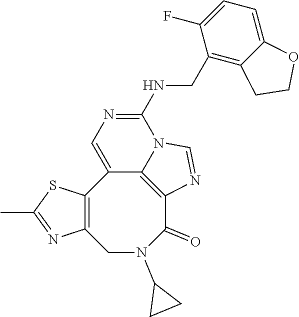 [SMILES: Cc1nc2c(s1)-c1cnc(NCc3c(F)ccc4c3CCO4)n3cnc(c13)C(=O)N(C1CC1)C2] | 4-cyclopropyl-11-(((5-fluoro-2,3- dihydrobenzofuran-4-yl)methyl)amino)-7-methyl- 4,5-dihydro-3H-8-thia-2,4,6,10,11a- pentaazacyclopenta[4,5]cycloocta[1,2,3-cd]inden- 3-one |
| 87 | 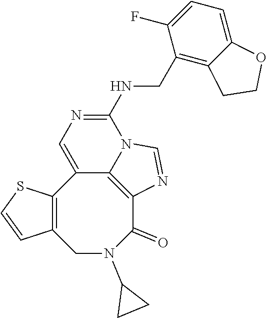 [SMILES: O=C1c2ncn3c(NCc4c(F)ccc5c4CCO5)ncc(c23)-c2sccc2CN1C1CC1] | 4-cyclopropyl-11-(((5-fluoro-2,3- dihydrobenzofuran-4-yl)methyl)amino)-4,5- dihydro-3H-8-thia-2,4,10,11a- tetraazacyclopenta[4,5]cycloocta[1,2,3-cd]inden- 3-one |
| 88 | 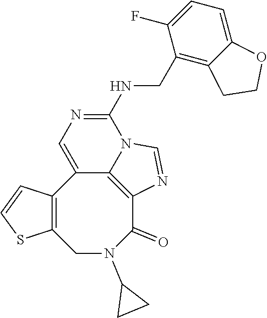 [SMILES: O=C1c2ncn3c(NCc4c(F)ccc5c4CCO5)ncc(c23)-c2ccsc2CN1C1CC1] | 4-cyclopropyl-11-(((5-fluoro-2,3- dihydrobenzofuran-4-yl)methyl)amino)-4,5- dihydro-3H-6-thia-2,4,10,11a- tetraazacyclopenta[4,5]cycloocta[1,2,3-cd]inden- 3-one |
| 89 | 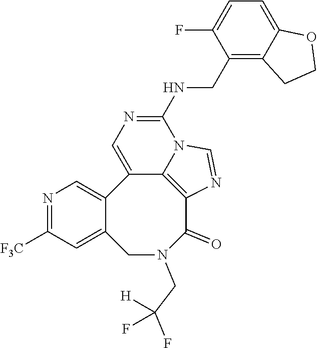 [SMILES: O=C1c2ncn3c(NCc4c(F)ccc5c4CCO5)ncc(c23)-c2cnc(C(F)(F)F)cc2CN1CC(F)F] | 4-(2,2-difluoroethyl)-12-(((5-fluoro-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| 90 | 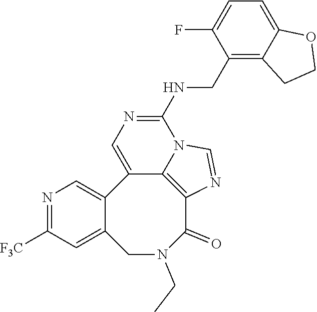 [SMILES: CCN1Cc2cc(C(F)(F)F)ncc2-c2cnc(NCc3c(F)ccc4c3CCO4)n3cnc(c23)C1=O] | 4-ethyl-12-(((5-fluoro-2,3-dihydrobenzofuran-4- yl)methyl)amino)-7-(trifluoromethyl)-4,5- dihydro-3H-2,4,8,11,12a- pentaazabenzo[4,5]cycloocta[1,2,3-cd]inden-3- one |
| 91 | 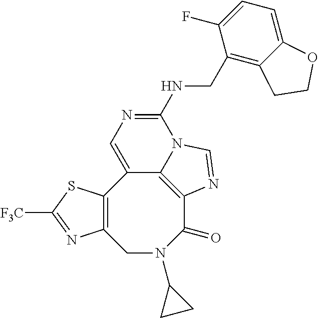 [SMILES: O=C1c2ncn3c(NCc4c(F)ccc5c4CCO5)ncc(c23)-c2sc(C(F)(F)F)nc2CN1C1CC1] | 4-cyclopropyl-11-(((5-fluoro-2,3- dihydrobenzofuran-4-yl)methyl)amino)-7- (trifluoromethyl)-4,5-dihydro-3H-8-thia- 2,4,6,10,11a- pentaazacyclopenta[4,5]cycloocta[1,2,3-cd]inden- 3-one |
| 92 | 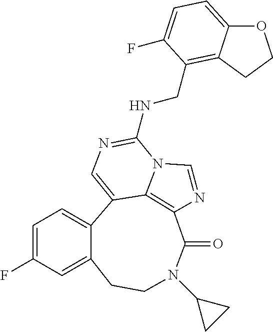 [SMILES: O=C1c2ncn3c(NCc4c(F)ccc5c4CCO5)ncc(c23)-c2ccc(F)cc2CCN1C1CC1] | 4-cycloproyl-8-fluoro-13-(((5-fluoro-2,3- dihydrobenzofuran-4-yl)methyl)amino)-5,6- dihydro-2,4,12,13a- tetraazabenzo[4,5]cyclonona[1,2,3-cd]inden- 3(4H)-one |
| 93 | 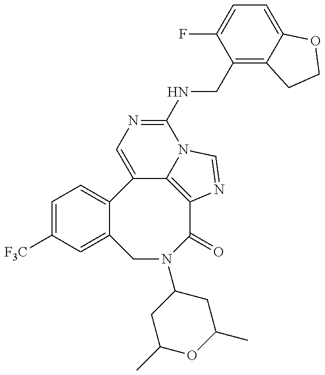 [SMILES: CC1CC(N2Cc3cc(C(F)(F)F)ccc3-c3cnc(NCc4c(F)ccc5c4CCO5)n4cnc(c34)C2=O)CC(C)O1] | 4-(2,6-dimethyltetrahydro-2H-pyran-4-yl)-12- (((5-fluoro-2,3-dihydrobenzofuran-4- yl)methyl)amino)-7-(trifluoromethyl)-4,5- dihydro-3H-2,4,11,12a- tetraazabenzo[4,5]cycloocta[1,2,3-cd]inden-3-one |
| 94 | 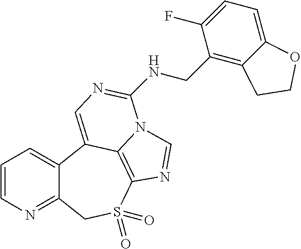 [SMILES: O=S1(=O)Cc2ncccc2-c2cnc(NCc3c(F)ccc4c3CCO4)n3cnc1c23] | 11-(((5-fluoro-2,3-dihydrobenzofuran-4- yl)methyl)amino)-4H-3-thia-2,5,10,11a- tetraazadibenzo[cd,f]azulene 3,3-dioxide |
| 95 | 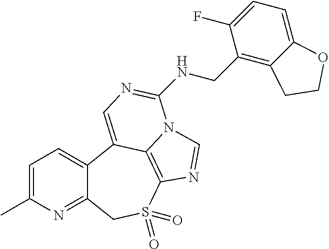 [SMILES: Cc1ccc2c(n1)CS(=O)(=O)c1ncn3c(NCc4c(F)ccc5c4CCO5)ncc-2c13] | 11-(((5-fluoro-2,3-dihydrobenzofuran-4- yl)methyl)amino)-6-methyl-4H-3-thia-2,5,10,11a- tetraazadibenzo[cd,f]azulene 3,3-dioxide |
| 96 | 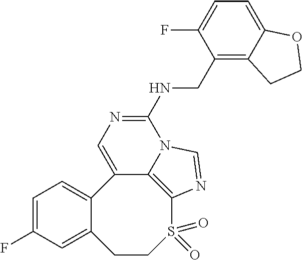 [SMILES: O=S1(=O)CCc2cc(F)ccc2-c2cnc(NCc3c(F)ccc4c3CCO4)n3cnc1c23] | 7-fluoro-12-(((5-fluoro-2,3-dihydrobenzofuran-4- yl)methyl)amino)-4,5-dihydro-3-thia-2,11,12a- triazabenzo[4,5]cycloocta[1,2,3-cd]indene-3,3- dioxide |
| 97 | 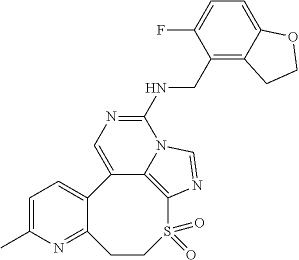 [SMILES: Cc1ccc2c(n1)CCS(=O)(=O)c1ncn3c(NCc4c(F)ccc5c4CCO5)ncc-2c13] | 12-(((5-fluoro-2,3-dihydrobenzofuran-4- yl)methyl)amino)-7-methyl-4,5-dihydro-3-thia- 2,6,11,12a-tetraazabenzo[4,5]cycloocta[1,2,3- cd]indene 3,3-dioxide |
| 98 | 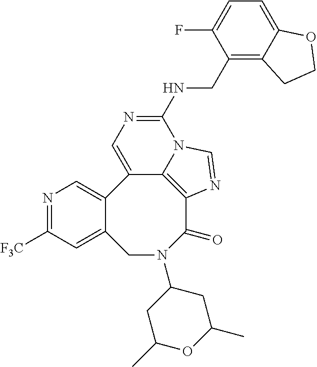 [SMILES: CC1CC(N2Cc3cc(C(F)(F)F)ncc3-c3cnc(NCc4c(F)ccc5c4CCO5)n4cnc(c34)C2=O)CC(C)O1] | 4-(2,6-dimethyltetrahydro-2H-pyran-4-yl)-12- (((5-fluoro-2,3-dihydrobenzofuran-4- yl)methyl)amino)-7-(trifluoromethyl)-4,5- dihydro-3H-2,4,8,11,12a- pentaazabenzo[4,5]cycloocta[1,2,3-cd]inden-3- one |
| 99 | 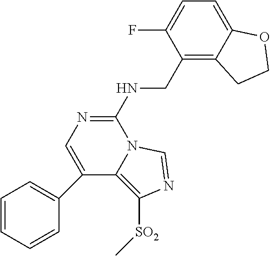 [SMILES: CS(=O)(=O)c1ncn2c(NCc3c(F)ccc4c3CCO4)ncc(-c3ccccc3)c12] | N-((5-fluoro-2,3-dihydrobenzofuran-4-yl)methyl)- 1-(methylsulfonyl)-8-phenylimidazo[1,5- c]pyrimidin-5-amine |
| 100 | 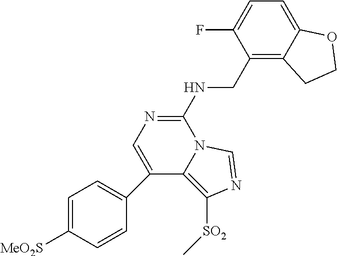 [SMILES: CS(=O)(=O)c1ccc(-c2cnc(NCc3c(F)ccc4c3CCO4)n3cnc(S(C)(=O)=O)c23)cc1] | N-((5-fluoro-2,3-dihydrobenzofuran-4-yl)methyl)- 1-(methylsulfonyl)-8-(4 (methyslulfonyl)phenyl)imidazo[1,5-c]pyrimidin- 5-amine |
| 101 | 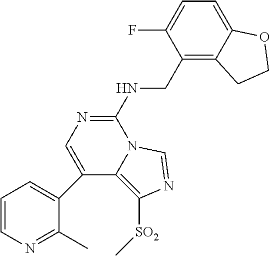 [SMILES: Cc1ncccc1-c1cnc(NCc2c(F)ccc3c2CCO3)n2cnc(S(C)(=O)=O)c12] | N-((5-fluoro-2,3-dihydrobenzofuran-4-yl)methyl)- 8-(2-methylpyridin-3-yl)-1- (methylsulfonyl)imidazo[1,5-c]pyrimidin-5-amine |
| 102 | 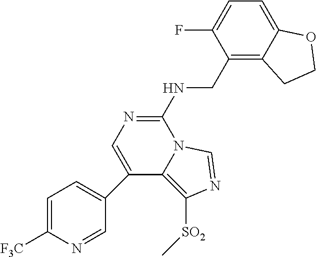 [SMILES: CS(=O)(=O)c1ncn2c(NCc3c(F)ccc4c3CCO4)ncc(-c3ccc(C(F)(F)F)nc3)c12] | N-((5-fluoro-2,3-dihydrobenzofuran-4-yl)methyl)- 1-(methylsulfonyl)-8-(6-(trifluoromethyl)pyridin- 3-yl)imidazo[1,5-c]pyrimidin-5-amine |
| 103 | 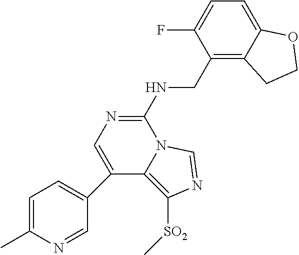 [SMILES: Cc1ccc(-c2cnc(NCc3c(F)ccc4c3CCO4)n3cnc(S(C)(=O)=O)c23)cn1] | N-((5-fluoro-2,3-dihydrobenzofuran-4-yl)methyl)- 8-(6-methylpyridin-3-yl)-1- (methylsulfonyl)imidazo[1,5-c]pyrimidin-5-amine |
| 104 | 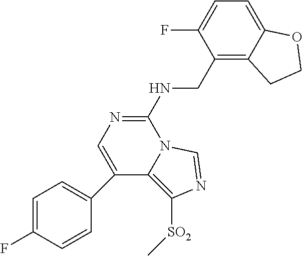 [SMILES: CS(=O)(=O)c1ncn2c(NCc3c(F)ccc4c3CCO4)ncc(-c3ccc(F)cc3)c12] | N-((5-fluoro-2,3-dihydrobenzofuran-4-yl)methyl)- 8-(4-fluorophenyl)-1 (methylsulfonyl)imidazo[1,5-c]pyrimidin-5-amine |
| 105 | 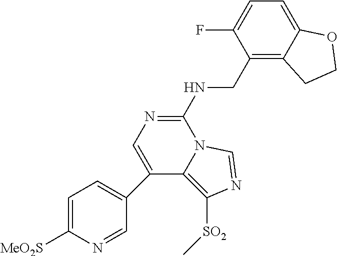 [SMILES: CS(=O)(=O)c1ccc(-c2cnc(NCc3c(F)ccc4c3CCO4)n3cnc(S(C)(=O)=O)c23)cn1] | N-((5-fluoro-2,3-dihydrobenzofuran-4-yl)methyl)- 1-(methylsulfonyl)-8-(6-(methylsulfonyl)pyridin- 3-yl)imidazo[1,5-c]pyrimidin-5-amine |
| 106 | 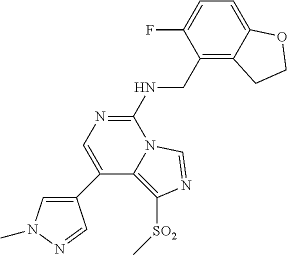 [SMILES: Cn1cc(-c2cnc(NCc3c(F)ccc4c3CCO4)n3cnc(S(C)(=O)=O)c23)cn1] | N-((5-fluoro-2,3-dihydrobenzofuran-4-yl)methyl)- 8-(1-methyl-1H-pyrazol-4-yl)-1- (methylsulfonyl)imidazo[1,5-c]pyrimidin-5-amine |
| 107 | 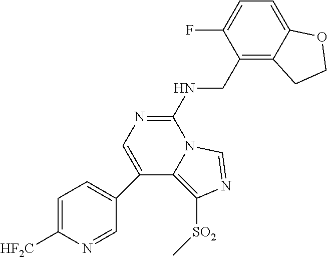 | 8-(6-(difluoromethyl)pyridin-3-yl)-N-((5-fluoro- 2,3-dihydrobenzofuran-4-yl)methyl)-1- (methylsulfonyl)imidazo[1,5-c]pyrimidin-5-amine |
| 108 | 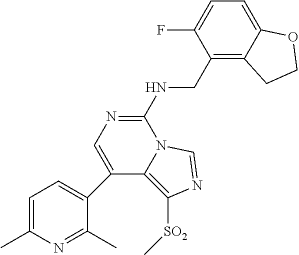 [SMILES: Cc1ccc(-c2cnc(NCc3c(F)ccc4c3CCO4)n3cnc(S(C)(=O)=O)c23)c(C)n1] | 8-(2,6-dimethylpyridin-3-yl)-N-((5-fluoro-2,3- dihydrobenzofuran-4-yl)methyl)-1- (methylsulfonyl)imidazo[1,5-c]pyrimidin-5-amine |
| 109 | 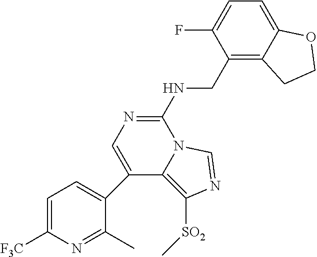 [SMILES: Cc1nc(C(F)(F)F)ccc1-c1cnc(NCc2c(F)ccc3c2CCO3)n2cnc(S(C)(=O)=O)c12] | N-((5-fluoro-2,3-dihydrobenzofuran-4-yl)methyl)- 8-(2-methyl-6-(trifluoromethyl)pyridin-3-yl)-1- (methylsulfonyl)imidazo[1,5-c]pyrimidin-5-amine |
| 110 | 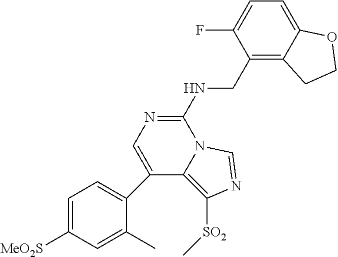 [SMILES: Cc1cc(S(C)(=O)=O)ccc1-c1cnc(NCc2c(F)ccc3c2CCO3)n2cnc(S(C)(=O)=O)c12] | N-((5-fluoro-2,3-dihydrobenzofuran-4-yl)methyl)- 8-(2-methyl-4-(methylsulfonyl)phenyl)-1- (methylsulfonyl)imidazo[1,5-c]pyrimidin-5-amine |
| 111 | 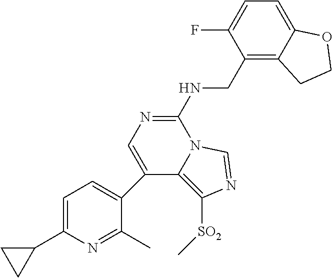 [SMILES: Cc1nc(C2CC2)ccc1-c1cnc(NCc2c(F)ccc3c2CCO3)n2cnc(S(C)(=O)=O)c12] | 8-(6-cyclopropylpyridin-3-yl)-N-((5-fluoro-2,3- dihydrobenzofuran-4-yl)methyl)-1- (methylsulfonyl)imidazo[1,5-c]pyrimidin-5-amine |
| 112 | 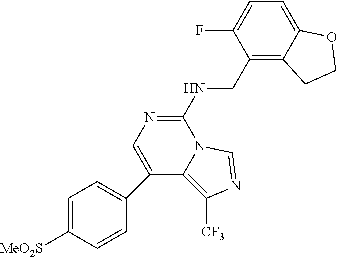 [SMILES: CS(=O)(=O)c1ccc(-c2cnc(NCc3c(F)ccc4c3CCO4)n3cnc(C(F)(F)F)c23)cc1] | N-((5-fluoro-2,3-dihydrobenzofuran-4-yl)methyl)- 8-(4-(methylsulfonyl)phenyl)-1- (trifluoromethyl)imidazo[1,5-c]pyrimidin-5-amine |
| 113 | 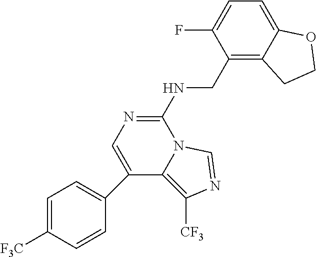 [SMILES: Fc1ccc2c(c1CNc1ncc(-c3ccc(C(F)(F)F)cc3)c3c(C(F)(F)F)ncn13)CCO2] | N-((5-fluoro-2,3-dihydrobenzofuran-4-yl)methyl)- 1-(trifluoromethyl)-8-(4- (trifluroomethyl)phenyl)imidazo[1,5-c]pyrimidin- 5-amine |
| 114 | 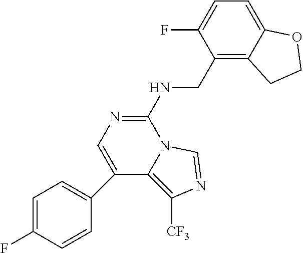 [SMILES: Fc1ccc(-c2cnc(NCc3c(F)ccc4c3CCO4)n3cnc(C(F)(F)F)c23)cc1] | N-((5-fluoro-2,3-dihydrobenzofuran-4-yl)methyl)- 8-(4-fluorophenyl)-1- (trifluoromethyl)imidazo[1,5-c]pyrimidin-5-amine |
| 115 | 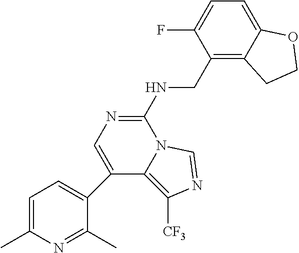 [SMILES: Cc1ccc(-c2cnc(NCc3c(F)ccc4c3CCO4)n3cnc(C(F)(F)F)c23)c(C)n1] | 8-(2,6-dimethylpyridin-3-yl)-N-((5-fluoro-2,3- dihydrobenzofuran-4-yl)methyl)-1- (trifluoromethyl)imidazo[1,5-c]pyrimidin-5-amine |
| 116 | 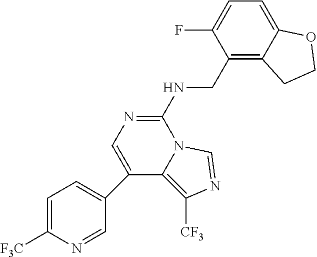 [SMILES: Fc1ccc2c(c1CNc1ncc(-c3ccc(C(F)(F)F)nc3)c3c(C(F)(F)F)ncn13)CCO2] | N-((5-fluoro-2,3-dihydrobenzofuran-4-yl)methyl)- 1-(trifluoromethyl)-8-(6-(trifluoromethyl)pyridin- 3-yl)imidazo[1,5-c]pyrimidin-5-amine |
| 117 | 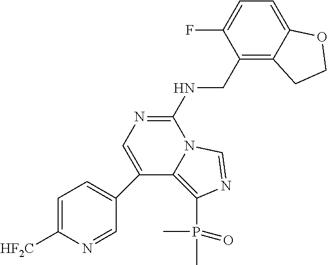 | (8-(6-(difluroomethyl)pyridin-3-yl)-5-(((5-fluoro- 2,3-dihydrobenzofuran-4- yl)methyl)amino)imidazo[1,5-c]pyrimidin-1- yl)dimethylphosphine oxide |
| 118 | 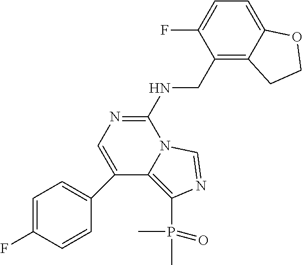 [SMILES: CP(C)(=O)c1ncn2c(NCc3c(F)ccc4c3CCO4)ncc(-c3ccc(F)cc3)c12] | (5-(((5-fluoro-2,3-dihydrobenzofuran-4- yl)methyl)amino)-8-(4-fluorophenyl)imidazo[1,5- c]pyrimidin-1-yl)dimethylphosphine oxide |
| 119 | 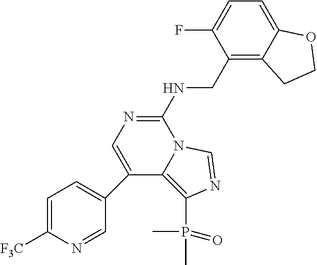 [SMILES: CP(C)(=O)c1ncn2c(NCc3c(F)ccc4c3CCO4)ncc(-c3ccc(C(F)(F)F)nc3)c12] | (5-(((5-fluoro-2,3-dihydrobenzofuran-4- yl)methyl)amino)-8-(6-(trifluoromethyl)pyridin-3- yl)imidazo[1,5-c]pyrimidin-1- yl)dimethylphosphine oxide |
| 120 | 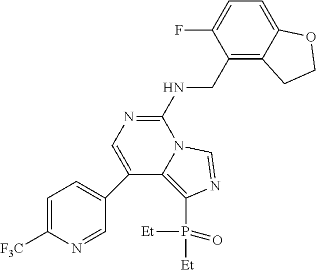 [SMILES: CCP(=O)(CC)c1ncn2c(NCc3c(F)ccc4c3CCO4)ncc(-c3ccc(C(F)(F)F)nc3)c12] | diethyl(5-(((5-fluoro-2,3-dihydrobenzofuran-4- yl)methyl)amino)-8-(6-(trifluoromethyl)pyridin-3- yl)imidazo[1,5-c]pyrimidin-1-yl)phosphine oxide |
| 121 | 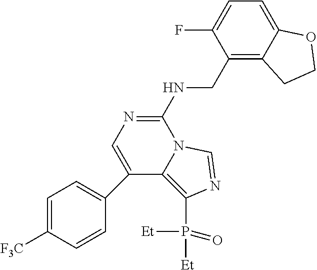 [SMILES: CCP(=O)(CC)c1ncn2c(NCc3c(F)ccc4c3CCO4)ncc(-c3ccc(C(F)(F)F)cc3)c12] | diethyl(5-(((5-fluoro-2,3-dihydrobenzofuran-4- yl)methyl)amino)-8-(4- (trifluoromethyl)phenyl)imidazo[1,5-c]pyrimidin- 1-yl)phosphine oxide |
| 122 | 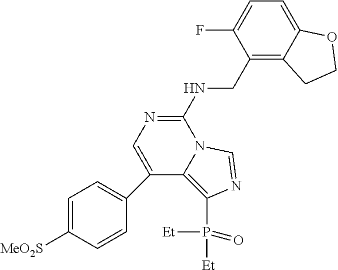 [SMILES: CCP(=O)(CC)c1ncn2c(NCc3c(F)ccc4c3CCO4)ncc(-c3ccc(S(C)(=O)=O)cc3)c12] | diethyl(5-(((5-fluoro-2,3-dihydrobenzofuran-4- yl)methyl)amino)-8-(4- (methylsulfonyl)phenyl)imidazo[1,5-c]pyrimidin- 1-yl)phosphine oxide |
| 123 | 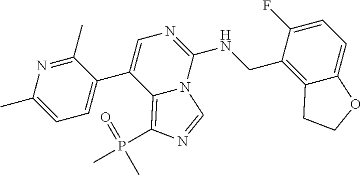 [SMILES: Cc1ccc(-c2cnc(NCc3c(F)ccc4c3CCO4)n3cnc(P(C)(C)=O)c23)c(C)n1] | (8-(2,6-dimethylpyridin-3-yl)-5-(((5-fluoro-2,3- dihydrobenzofuran-4- yl)methyl)amino)imidazo[1,5-c]pyrimidin-1- yl)dimethylphosphine oxide |
| 124 | 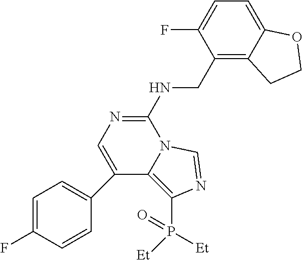 [SMILES: CCP(=O)(CC)c1ncn2c(NCc3c(F)ccc4c3CCO4)ncc(-c3ccc(F)cc3)c12] | diethyl(5-(((5-fluoro-2,3-dihydrobenzofuran-4- yl)methyl)amino)-8-(4-fluorophenyl)imidazo[1,5- c]pyrimidin-1-yl)phosphine oxide |
| 125 | 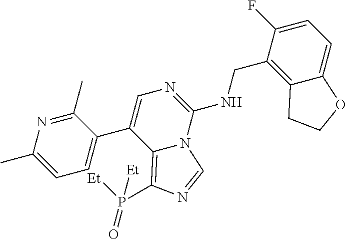 [SMILES: CCP(=O)(CC)c1ncn2c(NCc3c(F)ccc4c3CCO4)ncc(-c3ccc(C)nc3C)c12] | (8-(2,6-dimethylpyridin-3-yl)-5-(((5-fluoro-2,3- dihydrobenzofuran-4- yl)methyl)amino)imidazo[1,5-c]pyrimidin-1- yl)diethylphosphine oxide |
| 126 | 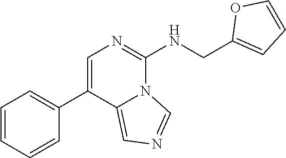 [SMILES: c1ccc(-c2cnc(NCc3ccco3)n3cncc23)cc1] | N-(furan-2-ylmethyl)-8-phenylimidazo[1,5- c]pyrimidin-5-amine |
| 127 | 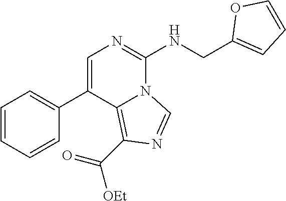 [SMILES: CCOC(=O)c1ncn2c(NCc3ccco3)ncc(-c3ccccc3)c12] | ethyl 5-((furan-2-ylmethyl)amino)-8- phenylimidazo[1,5-c]pyrimidine-1-carboxylate |
| 128 | 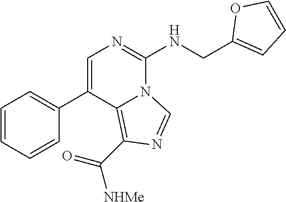 [SMILES: CNC(=O)c1ncn2c(NCc3ccco3)ncc(-c3ccccc3)c12] | 5-((furan-2-ylmethyl)amino)-N-methyl-8- phenylimidazo[1,5-c]pyrimidine-1-carboxamide |
| 129 | 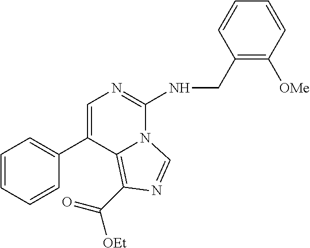 [SMILES: CCOC(=O)c1ncn2c(NCc3ccccc3OC)ncc(-c3ccccc3)c12] | ethyl 5-((2-methoxybenzyl)amino)-8- phenylimidazo[1,5-c]pyrimidine-1-carboxylate |
| 130 | 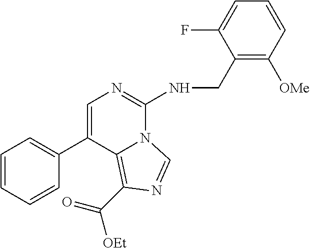 [SMILES: CCOC(=O)c1ncn2c(NCc3c(F)cccc3OC)ncc(-c3ccccc3)c12] | ethyl 5-((2-fluoro-6-methoxybenzyl)amino)-8- phenylimidazo[1,5-c]pyrimidine-1-carboxylate |
| 131 | 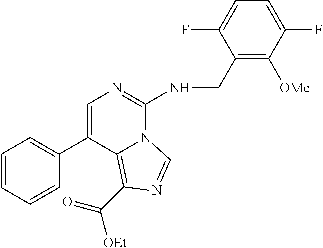 [SMILES: CCOC(=O)c1ncn2c(NCc3c(F)ccc(F)c3OC)ncc(-c3ccccc3)c12] | ethyl 5-((2,6-difluoro-3-methoxybenzyl)amino)-8- phenylimidazo[1,5-c]pyrimidine-1-carboxylate |
| 132 | 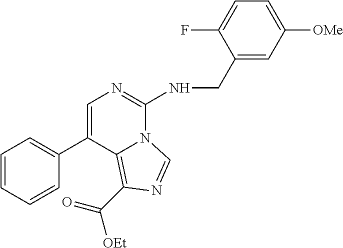 [SMILES: CCOC(=O)c1ncn2c(NCc3cc(OC)ccc3F)ncc(-c3ccccc3)c12] | ethyl 5-((2-fluoro-5-methoxybenzyl)amino)-8- phenylimidazo[1,5-c]pyrimidine-1-carboxylate |
| 133 | 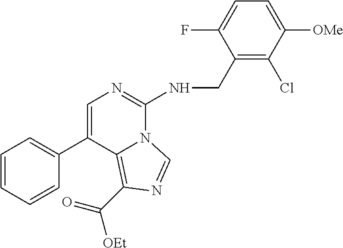 [SMILES: CCOC(=O)c1ncn2c(NCc3c(F)ccc(OC)c3Cl)ncc(-c3ccccc3)c12] | ethyl 5-((2-chloro-6-fluoro-3- methoxybenzyl)amino)-8-phenylimidazo[1,5- c]pyrimidine-1-carboxylate |
| 134 | 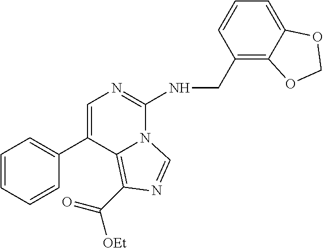 [SMILES: CCOC(=O)c1ncn2c(NCc3cccc4c3OCO4)ncc(-c3ccccc3)c12] | ethyl 5-((benzo[d][1,3]dioxol-4-ylmethyl)amino)- 8-phenylimidazo[1,5-c]pyrimidine-1-carboxylate |
| 135 | 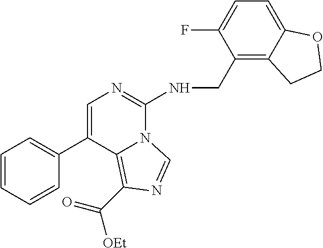 [SMILES: CCOC(=O)c1ncn2c(NCc3c(F)ccc4c3CCO4)ncc(-c3ccccc3)c12] | ethyl 5-(((5-fluoro-2,3-dihydrobenzofuran-4- yl)methyl)amino)-8-phenylimidazo[1,5- c]pyrimidine-1-carboxylate |
| 136 | 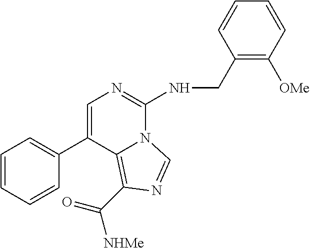 [SMILES: CNC(=O)c1ncn2c(NCc3ccccc3OC)ncc(-c3ccccc3)c12] | 5-((2-methoxybenzyl)amino)-N-methyl-8- phenylimidazo[1,5-c]pyrimidine-1-carboxamide |
| 137 | 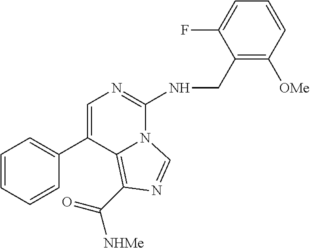 [SMILES: CNC(=O)c1ncn2c(NCc3c(F)cccc3OC)ncc(-c3ccccc3)c12] | 5-((2-fluoro-6-methoxybenzyl)amino)-N-methyl- 8-phenylimidazo[1,5-c]pyrimidine-1-carboxamide |
| 138 | 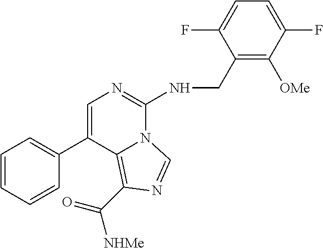 [SMILES: CNC(=O)c1ncn2c(NCc3c(F)ccc(F)c3OC)ncc(-c3ccccc3)c12] | 5-((3,6-difluoro-2-methoxybenzyl)amino)-N- methyl-8-phenylimidazo[1,5-c]pyrimidine-1- carboxamide |
| 139 | 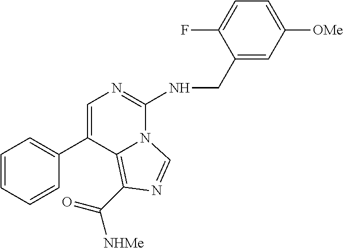 [SMILES: CNC(=O)c1ncn2c(NCc3cc(OC)ccc3F)ncc(-c3ccccc3)c12] | 5-((2-fluoro-5-methoxybenzyl)amino)-N-methyl- 8-phenylimidazo[1,5-c]pyrimidine-1-carboxamide |
| 140 | 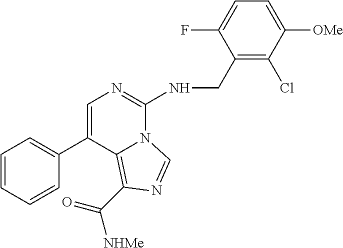 [SMILES: CNC(=O)c1ncn2c(NCc3c(F)ccc(OC)c3Cl)ncc(-c3ccccc3)c12] | 5-((2-chloro-6-fluoro-3-methoxybenzyl)amino)- N-methyl-8-phenylimidazo[1,5-c]pyrimidine-1- carboxamide |
| 141 | 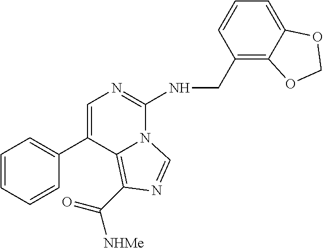 [SMILES: CNC(=O)c1ncn2c(NCc3cccc4c3OCO4)ncc(-c3ccccc3)c12] | 5-((benzo[d][1,3]dioxol-4-ylmethyl)amino)-N- methyl-8-phenylimidazo[1,5-c]pyrimidine-1- carboxmaide |
| 142 | 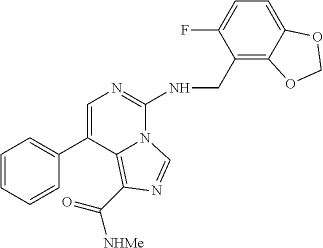 [SMILES: CNC(=O)c1ncn2c(NCc3c(F)ccc4c3OCO4)ncc(-c3ccccc3)c12] | 5-(((5-fluoro-2,3-dihydrobenzofuran-4- yl)methyl)amino)-N-methyl-8-phenylimidazo[1,5- c]pyrimidine-1-carboxamide |
| 143 | 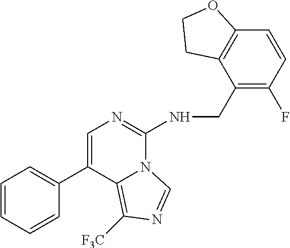 [SMILES: Fc1ccc2c(c1CNc1ncc(-c3ccccc3)c3c(C(F)(F)F)ncn13)CCO2] | N-((5-fluoro-2,3-dihydrobenzofuran-4-yl)methyl)- 8-phenyl-1-(trifluoromethyl)imidazo[1,5- c]pyrimidin-5-amine |
| 144 | 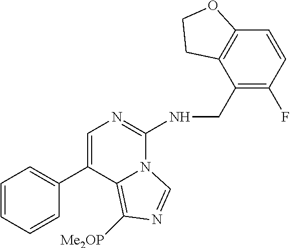 | (5-(((5-fluoro-2,3-dihydrobenzofuran-4- yl)methyl)amino)-8-phenylimidazo[1,5- c]pyrimidin-1-yl)dimethylphosphine oxide |
| 146 | 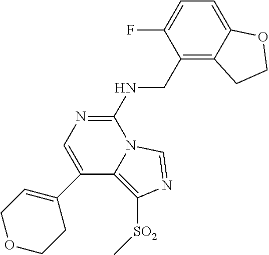 [SMILES: CS(=O)(=O)c1ncn2c(NCc3c(F)ccc4c3CCO4)ncc(C3=CCOCC3)c12] | 8-(3,6-dihydro-2H-pyran-4-yl)-N-((5-fluoro-2,3- dihydrobenzofuran-4-yl)methyl)-1- (methylsulfonyl)imidzo[1,5-c]pyrimidin-5-amine |
| 147 | 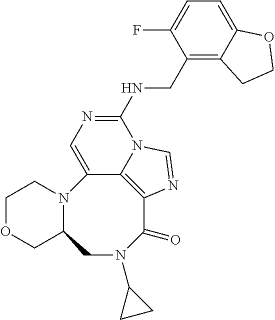 [SMILES: O=C1c2ncn3c(NCc4c(F)ccc5c4CCO5)ncc(c23)N2CCOC[C@@H]2CN1C1CC1] | (S)-4-cyclopropyl-12-(((5-fluoro-2,3- dihydrobenzofuran-4-yl)methyl)amino)- 4,5,5a,6,8,9-hexahydro-3H-7-oxa-2,4,9a,11,12a- pentaazabenzo[4,5]cycloocta[1,2,3-cd]inden-3- one |
| 148 | 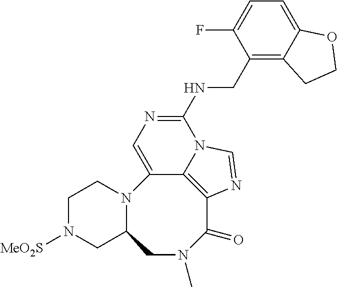 [SMILES: CN1C[C@H]2CN(S(C)(=O)=O)CCN2c2cnc(NCc3c(F)ccc4c3CCO4)n3cnc(c23)C1=O] | (S)-12-(((5-fluoro-2,3-dihydrobenzofuran-4- yl)methyl)amino)-4-mehtyl-7-(methylsulfonyl)- 5,5a,6,7,8,9-hexahydro-2,4,7,9a,11,12a- hexaazabenzo[4,5]cycloocta[1,2,3-cd]inden- 3(4H)-one |
| 149 | 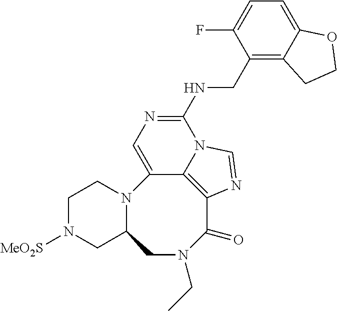 [SMILES: CCN1C[C@H]2CN(S(C)(=O)=O)CCN2c2cnc(NCc3c(F)ccc4c3CCO4)n3cnc(c23)C1=O] | (S)-4-ethyl-12-(((5-fluoro-2,3-dihydrobenzofuran- 4-yl)methyl)amino)-7-(methylsulfonyl)- 5,5a,6,7,8,9-hexahydro-2,4,7,9a,11,12a- hexaazabenzo[4,5]cycloocta[1,2,3-cd]inden- 3(4H)-one |
| 150 | 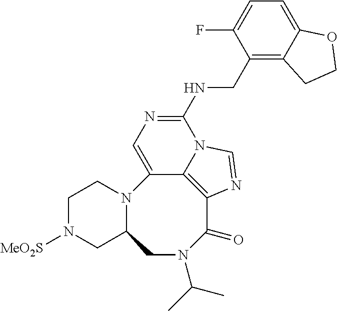 [SMILES: CC(C)N1C[C@H]2CN(S(C)(=O)=O)CCN2c2cnc(NCc3c(F)ccc4c3CCO4)n3cnc(c23)C1=O] | (S)-12-(((5-fluoro-2,3-dihydrobenzofuran-4- yl)methyl)amino)-4-isopropyl-7-(methylsulfonyl)- 5,5a,6,7,8,9-hexahydro-2,4,7,9a,11,12a- hexaazabenzo[4,5]cycloocta[1,2,3-cd]inden- 3(4H)-one |
| 151 | 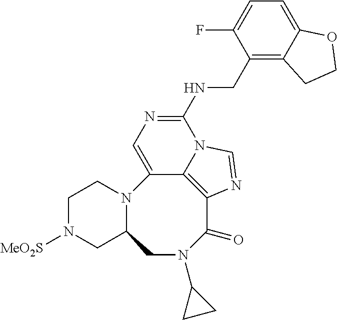 [SMILES: CS(=O)(=O)N1CCN2c3cnc(NCc4c(F)ccc5c4CCO5)n4cnc(c34)C(=O)N(C3CC3)C[C@H]2C1] | (S)-4-cyclopropyl-12-(((5-fluoro-2,3- dihydrobenzofuran-4-yl)methyl)amino)-7- (methylsulfonyl)-5,5a,6,7,8,9-hexahydro- 2,4,7,9a,11,12a- hexaazabenzo[4,5]cycloocta[1,2,3-cd]inden- 3(4H)-one |
| 152 | 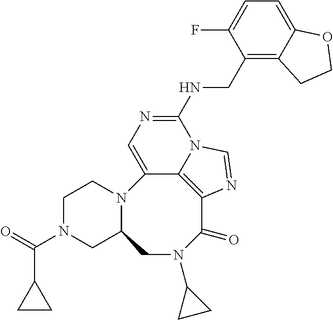 [SMILES: O=C(C1CC1)N1CCN2c3cnc(NCc4c(F)ccc5c4CCO5)n4cnc(c34)C(=O)N(C3CC3)C[C@H]2C1] | (R)-7-(cyclopropanecarbonyl)-4-cyclopropyl-12- (((5-fluoro-2,3-dihydrobenzofuran-4- yl)methyl)amino)-5,5a,6,7,8,9-hexahydro- 2,4,7,9a,11,12a- hexaazabenzo[4,5]cycloocta[1,2,3-cd]inden- 3(4H)-one |
| 153 | 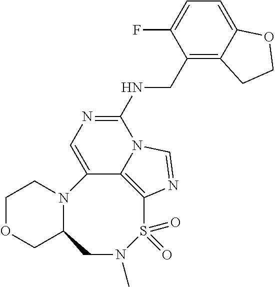 [SMILES: CN1C[C@H]2COCCN2c2cnc(NCc3c(F)ccc4c3CCO4)n3cnc(c23)S1(=O)=O] | (S)-12-(((5-fluoro-2,3-dihydrobenzofuran-4- yl)methyl)amino)-4-methyl-4,5,5a,6,8,9- hexahydro-7-oxa-3-thia-2,4,9a,11,12a- pentaazabenzo[4,5]cycloocta[1,2,3-cd]indene 3,3- dioxide |
| 154 | 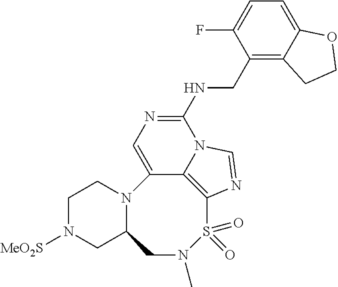 [SMILES: CN1C[C@H]2CN(S(C)(=O)=O)CCN2c2cnc(NCc3c(F)ccc4c3CCO4)n3cnc(c23)S1(=O)=O] | (S)-12-(((5-fluoro-2,3-dihydrobenzofuran-4- yl)methyl)amino)-4-methyl-7-(methylsulfonyl)- 5,5a,6,7,8,9-hexahydro-4H-3-thia-2,4,7,9a,11,12a- hexaazabenzo[4,5]cycloocta[1,2,3-cd]indene 3,3- dioxide |
| 155 | 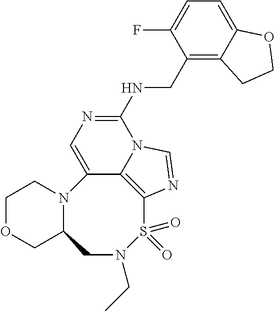 [SMILES: CCN1C[C@H]2COCCN2c2cnc(NCc3c(F)ccc4c3CCO4)n3cnc(c23)S1(=O)=O] | (S)-4-ethyl-12-(((5-fluoro-2,3-dihydrobenzofuran- 4-yl)methyl)amino)-4,5,5a,6,8,9-hexahydro-7- oxa-3-thia-2,4,9a,11,12a- pentaazabenzo[4,5]cycloocta[1,2,3-cd]indene 3,3- dioxide |
| 156 | 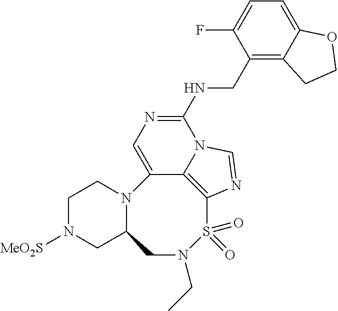 [SMILES: CCN1C[C@H]2CN(S(C)(=O)=O)CCN2c2cnc(NCc3c(F)ccc4c3CCO4)n3cnc(c23)S1(=O)=O] | (R)-4-ethyl-12-(((5-fluoro-2,3-dihydrobenzofuran- 4-yl)methyl)amino)-7-(methyslulfonyl)- 5,5a,6,7,8,9-hexahydro-4H-3-thia-2,4,7,9a,11,12a- hexaazabenzo[4,5]cycloocta[1,2,3-cd]indene 3,3- dioxide |
| 157 | 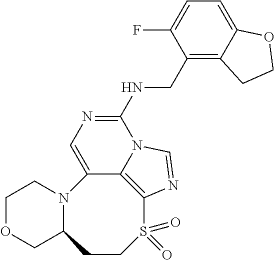 [SMILES: O=S1(=O)CC[C@H]2COCCN2c2cnc(NCc3c(F)ccc4c3CCO4)n3cnc1c23] | (S)-12-(((5-fluoro-2,3-dihydrobenzofuran-4- yl)methyl)amino)-4,5,5a,6,8,9-hexahydro-7-oxa- 3-thia-2,9a,11,12a- tetraazabenzo[4,5]cycloocta[1,2,3-cd]indene 3,3- dioxide |
| 158 | 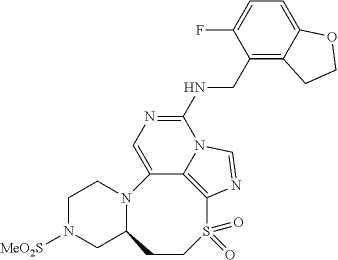 [SMILES: CS(=O)(=O)N1CCN2c3cnc(NCc4c(F)ccc5c4CCO5)n4cnc(c34)S(=O)(=O)CC[C@H]2C1] | (S)-12-(((5-fluoro-2,3-dihydrobenzofurna-4- yl)methyl)amino)-7-(methylsulfonyl)-5,5a,6,7,8,9- hexahydro-4H-3-thia-2,7,9a,11,12a- pentaazabenzo[4,5]cycloocta[1,2,3-cd]indene 3,3- dioxide |
| 169 | 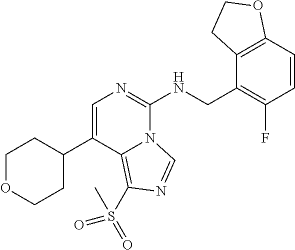 [SMILES: CS(=O)(=O)c1ncn2c(NCc3c(F)ccc4c3CCO4)ncc(C3CCOCC3)c12] | N-((5-fluoro-2,3-dihydrobenzofuran-4-yl)methyl)- 1-(methylsulfonyl)-8-(tetrahydro-2H-pyran-4- yl)imidazo[1,5-c]pyrimidin-5-amine |
| 160 | 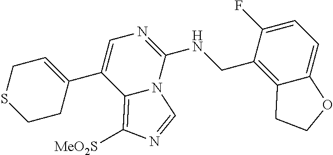 [SMILES: CS(=O)(=O)c1ncn2c(NCc3c(F)ccc4c3CCO4)ncc(C3=CCSCC3)c12] | 8-(3,6-dihydro-2H-thiopyran-4-yl)-N-((5-fluoro- 2,3-dihydrobenzofuran-4-yl)methyl)-1- (methylsulfonyl)imidazo[1,5-c]pyrimidin-5-amine |
| 161 | 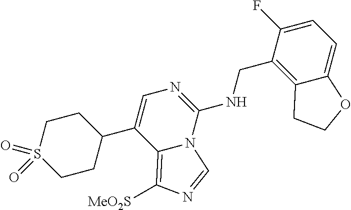 [SMILES: CS(=O)(=O)c1ncn2c(NCc3c(F)ccc4c3CCO4)ncc(C3CCS(=O)(=O)CC3)c12] | 4-(5-(((5-fluoro-2,3-dihydrobenzofuran-4- yl)methyl)amino)-1-(methylsulfonyl)imidazo[1,5- c]pyrimidin-8-yl)tetrahydro-2H-thiopyran 1,1- dioxide |
| 162 | 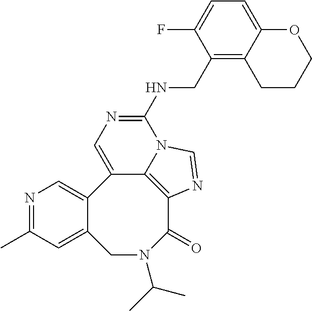 [SMILES: Cc1cc2c(cn1)-c1cnc(NCc3c(F)ccc4c3CCCO4)n3cnc(c13)C(=O)N(C(C)C)C2] | 12-(((6-fluorochroman-5-yl)methyl)amino)-4- isopropyl-7-methyl-4,5-dihydro-3H-2,4,8,11,12a- pentaazabenzo[4,5]cycloocta[1,2,3-cd]inden-3- one |
| 163 | 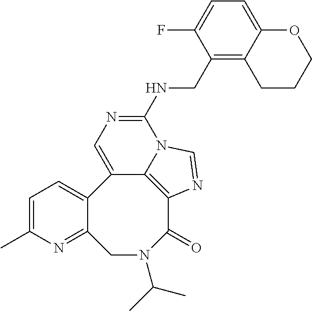 [SMILES: Cc1ccc2c(n1)CN(C(C)C)C(=O)c1ncn3c(NCc4c(F)ccc5c4CCCO5)ncc-2c13] | 12-(((6-fluorochroman-5-yl)methyl)amino)-4- isopropyl-7-methyl-4,5-dihydro-3H-2,4,6,11,12a- pentaazabenzo[4,5]cyclocta[1,2,3-cd]inden-3- one |
| 164 | 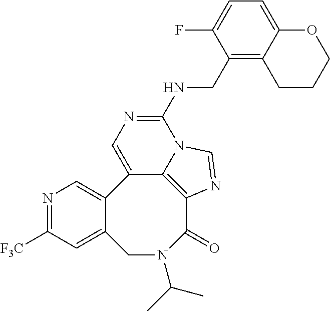 [SMILES: CC(C)N1Cc2cc(C(F)(F)F)ncc2-c2cnc(NCc3c(F)ccc4c3CCCO4)n3cnc(c23)C1=O] | 12-(((6-fluorochroman-5-yl)methyl)amino)-4- isopropyl-7-(trifluoromethyl)-4,5-dihydro-3H- 2,4,8,11,12a-pentaazabenzo[4,5]cycloocta[1,2,3- cd]inden-3-one |
| 165 | 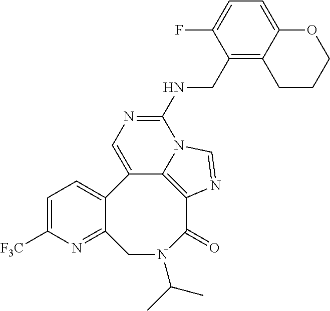 [SMILES: CC(C)N1Cc2nc(C(F)(F)F)ccc2-c2cnc(NCc3c(F)ccc4c3CCCO4)n3cnc(c23)C1=O] | 12-(((6-fluorochroman-5-yl)methyl)amino)-4- isopropyl-7-(trifluoromethyl)-4,5-dihydro-3H- 2,4,6,11,12a-pentaazabenzo[4,5]cycloocta[1,2,3- cd]inden-3-one |
| 166 | 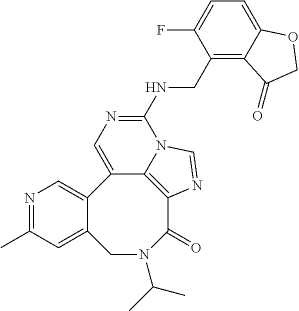 [SMILES: Cc1cc2c(cn1)-c1cnc(NCc3c(F)ccc4c3C(=O)CO4)n3cnc(c13)C(=O)N(C(C)C)C2] | 12-(((5-fluoro-3-oxo-2,3-dihydrobenzofuran-4- yl)methyl)amino)-4-isopropyl-7-methyl-4,5- dihydro-3H-2,4,8,11,12a- pentaazabenzo[4,5]cycloocta[1,2,3-cd]inden-3- one |
| 167 | 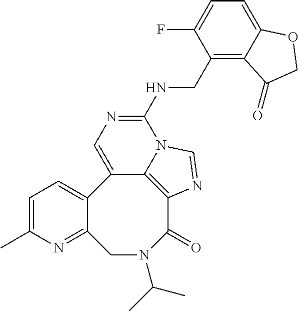 [SMILES: Cc1ccc2c(n1)CN(C(C)C)C(=O)c1ncn3c(NCc4c(F)ccc5c4C(=O)CO5)ncc-2c13] | 12-(((5-fluoro-3-oxo-2,3-dihydrobenzofuran-4- yl)methyl)amino)-4-isopropyl-7-methyl-4,5- dihydro-3H-2,4,6,11,12a- pentaazabenzo[4,5]cycloocta[1,2,3-cd]inden-3- one |
| 168 | 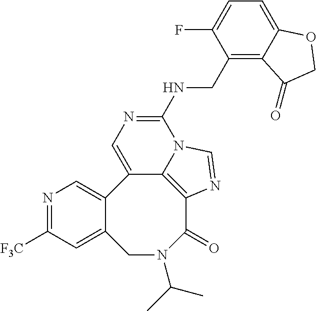 [SMILES: CC(C)N1Cc2cc(C(F)(F)F)ncc2-c2cnc(NCc3c(F)ccc4c3C(=O)CO4)n3cnc(c23)C1=O] | 12-(((5-fluoro-3-oxo-2,3-dihydrobenzofuran-4- yl)methyl)amino)-4-isopropyl-7-(trifluoromethyl)- 4,5-dihydro-3H-2,4,8,11,12a- pentaazabenzo[4,5]cycloocta[1,2,3-cd]inden-3- one |
| 169 | 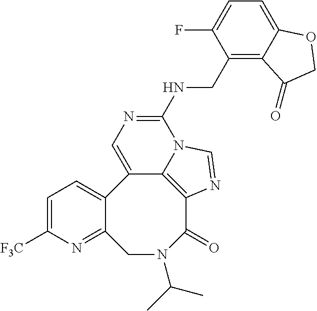 [SMILES: CC(C)N1Cc2nc(C(F)(F)F)ccc2-c2cnc(NCc3c(F)ccc4c3C(=O)CO4)n3cnc(c23)C1=O] | 12-(((5-fluoro-3-oxo-2,3-dihydrobenzofuran-4- yl)methyl)amino)-4-isoporopyl-7-(trifluoromethyl)- 4,5-dihydro-3H-2,4,6,11,12a- pentaazabenzo[4,5]cycloocta[1,2,3-cd]inden-3- one |
| 170 | 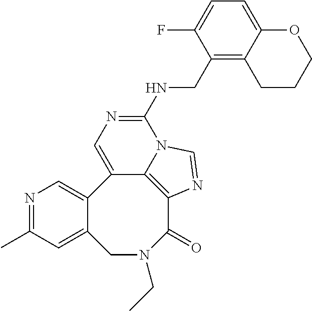 [SMILES: CCN1Cc2cc(C)ncc2-c2cnc(NCc3c(F)ccc4c3CCCO4)n3cnc(c23)C1=O] | 4-ethyl-12-(((6-fluorochroman-5- yl)methyl)amino)-7-methyl-4,5-dihydro-3H- 2,4,8,11,12a-pentaazabenzo[4,5]cycloocta[1,2,3- cd]inden-3-one |
| 171 | 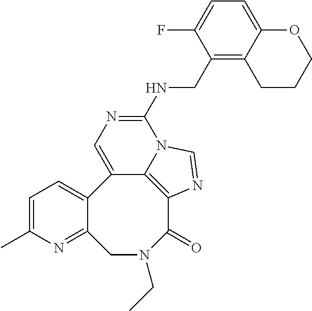 [SMILES: CCN1Cc2nc(C)ccc2-c2cnc(NCc3c(F)ccc4c3CCCO4)n3cnc(c23)C1=O] | 4-ethyl-12-(((6-fluorochroman-5- yl)methyl)amino)-7-methyl-4,5-dihydro-3H- 2,4,6,11,12a-pentaazabenzo[4,5]cycloocta[1,2,3- cd]inden-3-one |
| 172 | 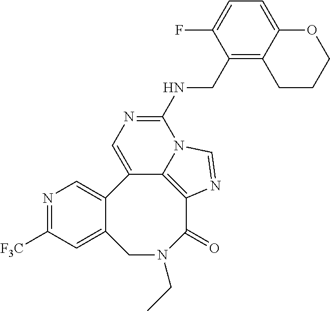 [SMILES: CCN1Cc2cc(C(F)(F)F)ncc2-c2cnc(NCc3c(F)ccc4c3CCCO4)n3cnc(c23)C1=O] | 4-ethyl-12-(((6-fluorochroman-5- yl)methyl)amino)-7-(trifluoromethyl)-4,5- dihydro-3H-2,4,8,11,12a- pentaazabenzo[4,5]cycloocta[1,2,3-cd]inden-3- one |
| 173 | 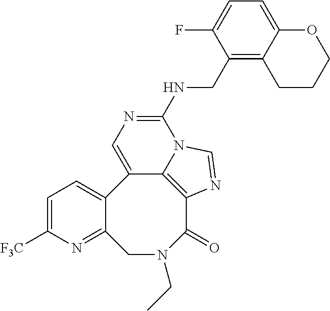 [SMILES: CCN1Cc2nc(C(F)(F)F)ccc2-c2cnc(NCc3c(F)ccc4c3CCCO4)n3cnc(c23)C1=O] | 4-ethyl-12-(((6-fluorochroman-5- yl)methyl)amino)-7-(trifluoromethyl)-4,5- dihydro-3H-2,4,6,11,12a- pentaazabenzo[4,5]cycloocta[1,2,3-cd]inden-3- one |
| 174 | 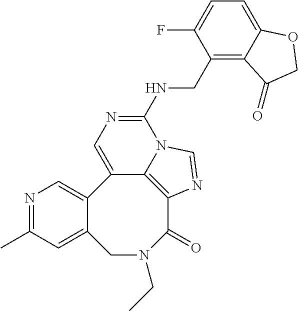 [SMILES: CCN1Cc2cc(C)ncc2-c2cnc(NCc3c(F)ccc4c3C(=O)CO4)n3cnc(c23)C1=O] | 4-ethyl-12-(((5-fluoro-3-oxo-2,3- dihydrobenzofuran-4-yl)methyl)amino)-7-methyl- 4,5-dihydro-3H-2,4,8,11,12a- pentaazabenzo[4,5]cycloocta[1,2,3-cd]inden-3- one |
| 175 | 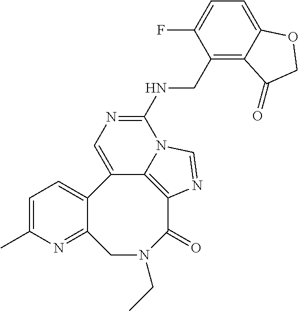 [SMILES: CCN1Cc2nc(C)ccc2-c2cnc(NCc3c(F)ccc4c3C(=O)CO4)n3cnc(c23)C1=O] | 4-ethyl-12-(((5-fluoro-3-oxo-2,3- dihydrobenzofuran-4-yl)methyl)amino)-7-methyl- 4,5-dihydro-3H-2,4,6,11,12a- pentaazabenzo[4,5]cycloocta[1,2,3-cd]inden-3- one |
| 176 | 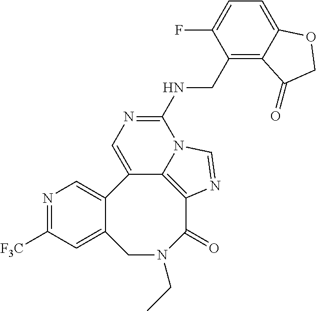 [SMILES: CCN1Cc2cc(C(F)(F)F)ncc2-c2cnc(NCc3c(F)ccc4c3C(=O)CO4)n3cnc(c23)C1=O] | 4-ethyl-12-(((5-fluoro-3-oxo-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| 177 | 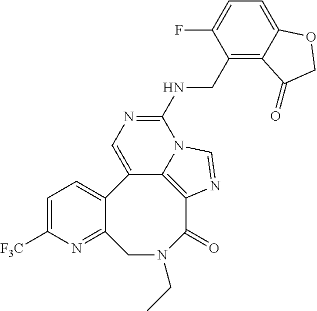 [SMILES: CCN1Cc2nc(C(F)(F)F)ccc2-c2cnc(NCc3c(F)ccc4c3C(=O)CO4)n3cnc(c23)C1=O] | 4-ethyl-12-(((5-fluoro-3-oxo-2,3- dihydrobenzofuran-4-yl)methyl)amino)-7- (trifluoromethyl)-4,5-dihydro-3H-2,4,6,11,12a- pentaazabenzo[4,5]cycloocta[1,2,3-cd]inden-3- one |
| 178 | 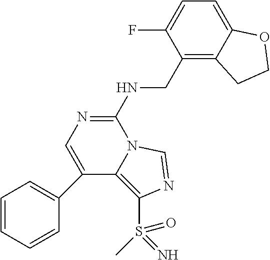 [SMILES: CS(=N)(=O)c1ncn2c(NCc3c(F)ccc4c3CCO4)ncc(-c3ccccc3)c12] | (5-(((5-fluoro-2,3-dihydrobenzofuran-4- yl)methyl)amino)-8-phenylimidazo[1,5- c]pyrimidin-1-yl)(imino)(methyl)-16-sulfanone |
| 179 | 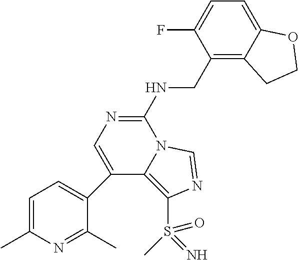 [SMILES: Cc1ccc(-c2cnc(NCc3c(F)ccc4c3CCO4)n3cnc(S(C)(=N)=O)c23)c(C)n1] | (8-(2,6-dimethylpyridin-3-yl)-5-(((5-fluoro-2,3- dihydrobenzofuran-4- yl)methyl)amino)imidazo[1,5-c]pyrimidin-1- yl)(imino)(methyl)-sulfanone |
| 180 | 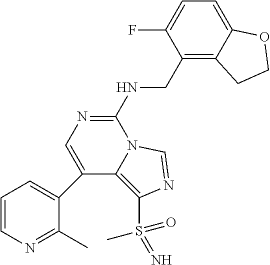 [SMILES: Cc1ncccc1-c1cnc(NCc2c(F)ccc3c2CCO3)n2cnc(S(C)(=N)=O)c12] | (5-(((5-fluoro-2,3-dihydrobenzofuran-4- yl)methyl)amino)-8-(2-methylpyridin-3- yl)imidazo[1,5-c]pyrimidin-1-yl)(imino)(methyl)- sulfanone |
| 181 | 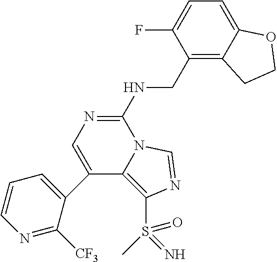 [SMILES: CS(=N)(=O)c1ncn2c(NCc3c(F)ccc4c3CCO4)ncc(-c3cccnc3C(F)(F)F)c12] | (5-(((5-fluoro-2,3-dihydrobenzofuran-4- yl)methyl)amino)-8-(2-(trifluoromethyl)pyridin-3- yl)imidazo[1,5-c]pyrimidin-1-yl)(imino)(methyl)- 16-sulfanone |
| 182 | 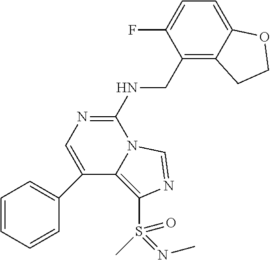 [SMILES: CN=S(C)(=O)c1ncn2c(NCc3c(F)ccc4c3CCO4)ncc(-c3ccccc3)c12] | (5-(((5-fluoro-2,3-dihydrobenzofuran-4- yl)methyl)amino)-8-phenylimidazo[1,5- c]pyrimidin-1-yl)(methyl)(methylimino)-1,6- sulfanone |
| 183 | 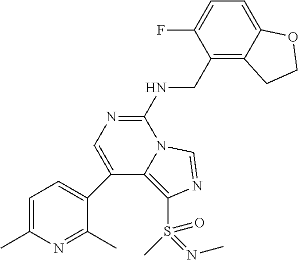 [SMILES: CN=S(C)(=O)c1ncn2c(NCc3c(F)ccc4c3CCO4)ncc(-c3ccc(C)nc3C)c12] | (8-(2,6-dimethylpyridin-3-yl)-5-(((5-fluoro-2,3- dihydrobenzofuran-4- yl)methyl)amino)imidazo[1,5-c]pyrimidin-1- yl)(methyl)(methylimino)-16-sulfanone |
| 184 | 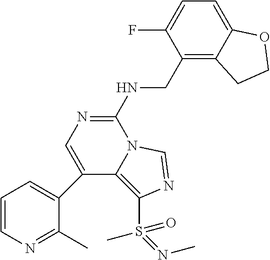 [SMILES: CN=S(C)(=O)c1ncn2c(NCc3c(F)ccc4c3CCO4)ncc(-c3cccnc3C)c12] | (5-(((5-fluoro-2,3-dihydrobenzofuran-4- yl)methyl)amino)-8-(2-methylpyridin-3- yl)imidazo[1,5-c]pyrimidin-1- yl)(methyl)(methylimino)-16-sulfanone |
| 185 | 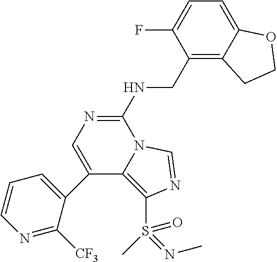 [SMILES: CN=S(C)(=O)c1ncn2c(NCc3c(F)ccc4c3CCO4)ncc(-c3cccnc3C(F)(F)F)c12] | (5-(((5-fluoro-2,3-dihydrobenzofuran-4- yl)methyl)amino)-8-(2-(trifluoromethyl)pyridin-3- yl)imidazo[1,5-c]pyrimidin-1- yl)(methyl)(methylimino)-16-sulfanone |
| 186 | 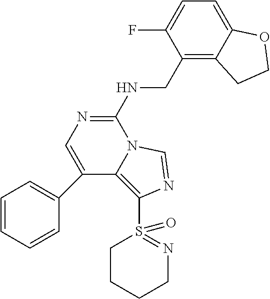 [SMILES: O=S1(c2ncn3c(NCc4c(F)ccc5c4CCO5)ncc(-c4ccccc4)c23)=NCCCC1] | 1-(5-(((5-fluoro-2,3-dihydrobenzofuran-4- yl)methyl)amino)-8-phenylimidazo[1,5- c]pyrimidin-1-yl)-3,4,5,6-tetrahydro-1,2-thiazine 1-oxide |
| 187 | 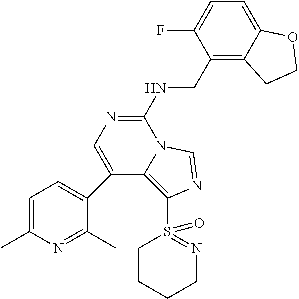 [SMILES: Cc1ccc(-c2cnc(NCc3c(F)ccc4c3CCO4)n3cnc(S4(=O)=NCCCC4)c23)c(C)n1] | 1-(8-(2,6-dimethylpyridin-3-yl)-5-(((5-fluoro-2,3- dihydrobenzofuran-4- yl)methyl)amino)imidazo[1,5-c]pyrimidin-1-yl)- 3,4,5,6-tetrahydro-1,2-thiazine 1-oxide |
| 188 | 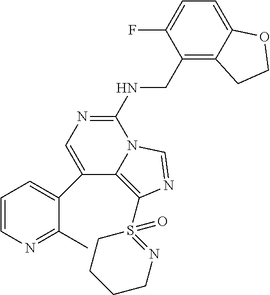 [SMILES: Cc1ncccc1-c1cnc(NCc2c(F)ccc3c2CCO3)n2cnc(S3(=O)=NCCCC3)c12] | 1-(5-(((5-fluoro-2,3-dihydrobenzofuran-4- yl)methyl)amino)-8-(2-methylpyridin-3- yl)imidazo[1,5-c]pyrimidin-1-yl)-3,4,5,6- tetrahydro-1,2-thiazine 1-oxide |
| 189 | 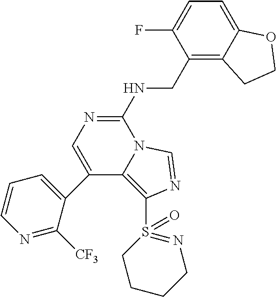 [SMILES: O=S1(c2ncn3c(NCc4c(F)ccc5c4CCO5)ncc(-c4cccnc4C(F)(F)F)c23)=NCCCC1] | 1-(5-(((5-fluoro-2,3-dihydrobenzofuran-4- yl)methyl)amino)-8-(2-(trifluoromethyl)pyridin-3- yl)imidazo[1,5-c]pyrimidin-1-yl)-3,4,5,6- tetrahydro-1,2-thiazine 1-oxide |
| E1 | 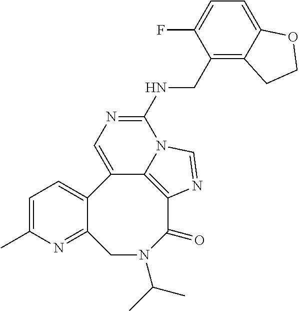 [SMILES: Cc1ccc2c(n1)CN(C(C)C)C(=O)c1ncn3c(NCc4c(F)ccc5c4CCO5)ncc-2c13] | 12-(((5-fluoro-2,3-dihydrobenzofuran-4- yl)methyl)amino)-4-isopropyl-7-methyl-4,5- dihydro-3H-2,4,6,11,12a- pentaazabenzo[4,5]cycloocta[1,2,3-cd]inden-3- one |
| E2 | 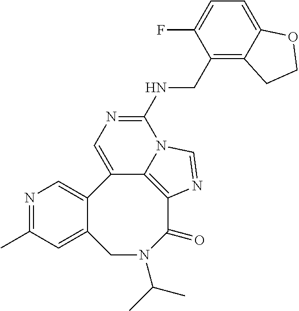 [SMILES: Cc1cc2c(cn1)-c1cnc(NCc3c(F)ccc4c3CCO4)n3cnc(c13)C(=O)N(C(C)C)C2] | 12-(((5-fluoro-2,3-dihydrobenzofuran-4- yl)methyl)amino)-4-isopropyl-7-methyl-4,5- dihydro-3H-2,4,8,11,12a- pentaazabenzo[4,5]cycloocta[1,2,3-cd]inden-3- one |
| E3 | 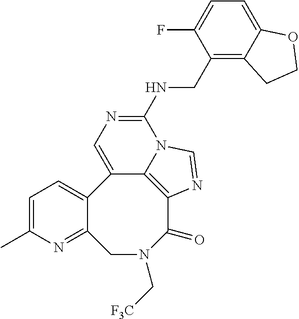 [SMILES: Cc1ccc2c(n1)CN(CC(F)(F)F)C(=O)c1ncn3c(NCc4c(F)ccc5c4CCO5)ncc-2c13] | 12-(((5-fluoro-2,3-dihydrobenzofuran-4- yl)methyl)amino)-7-methyl-4-(2,2,2- trifluoroethyl)-4,5-dihydro-3H-2,4,6,11,12a- pentaazabenzo[4,5]cycloocta[1,2,3-cd]inden-3- one |
| E4 | 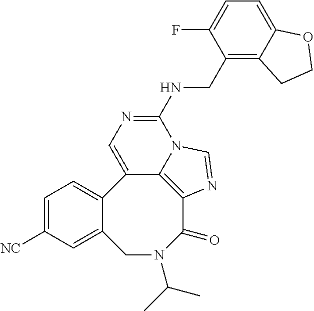 [SMILES: CC(C)N1Cc2cc(C#N)ccc2-c2cnc(NCc3c(F)ccc4c3CCO4)n3cnc(c23)C1=O] | 12-(((5-fluoro-2,3-dihydrobenzofuran-4- yl)methyl)amino)-4-isopropyl-3-oxo-4,5-dihydro- 3H-2,4,11,12a-tetraazabenzo[4,5]cycloocta[1,2,3- cd]indene-7-carbonitrile |
| E5 | 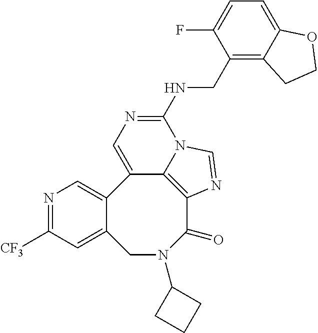 [SMILES: O=C1c2ncn3c(NCc4c(F)ccc5c4CCO5)ncc(c23)-c2cnc(C(F)(F)F)cc2CN1C1CCC1] | 4-cyclobutyl-12-(((5-fluoro-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| E6 | 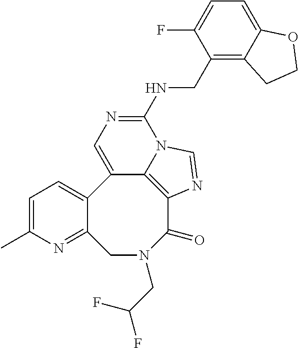 [SMILES: Cc1ccc2c(n1)CN(CC(F)F)C(=O)c1ncn3c(NCc4c(F)ccc5c4CCO5)ncc-2c13] | 4-(2,2-difluoroethyl)-12-(((5-fluoro-2,3- dihydrobenzofuran-4-yl)methyl)amino)-7-methyl- 4,5-dihydro-3H-2,4,6,11,12a- pentaazabenzo[4,5]cycloocta[1,2,3-cd]inden-3- one |
| E7 | 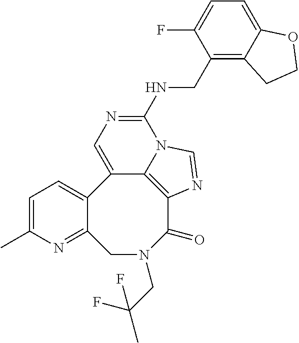 [SMILES: Cc1ccc2c(n1)CN(CC(C)(F)F)C(=O)c1ncn3c(NCc4c(F)ccc5c4CCO5)ncc-2c13] | 4-(2,2-difluoropropyl)-12-(((5-fluoro-2,3- dihydrobenzofuran-4-yl)methyl)amino)-7-methyl- 4,5-dihydro-3H-2,4,6,11,12a- pentaazabenzo[4,5]cycloocta[1,2,3-cd]inden-3- one |
| E8 | 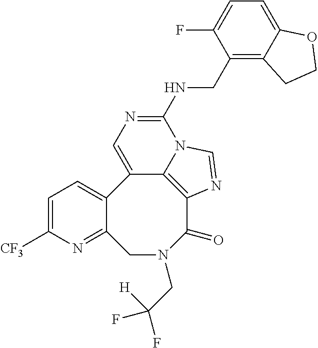 [SMILES: O=C1c2ncn3c(NCc4c(F)ccc5c4CCO5)ncc(c23)-c2ccc(C(F)(F)F)nc2CN1CC(F)F] | 4-(2,2-difluoroethyl)-12-(((5-fluoro-2,3- dihydrobenzofuran-4-yl)methyl)amino)-7- (trifluoromethyl)-4,5-dihydro-3H-2,4,6,11,12a- pentaazabenzo[4,5]cycloocta[1,2,3-cd]inden-3- one |
| E9 | 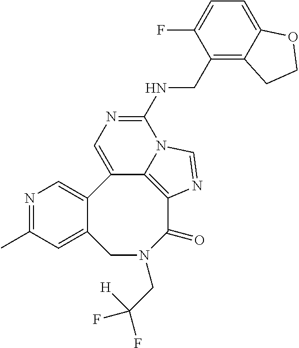 [SMILES: Cc1cc2c(cn1)-c1cnc(NCc3c(F)ccc4c3CCO4)n3cnc(c13)C(=O)N(CC(F)F)C2] | 4-(2,2-difluoroethyl)-12-(((5-fluoro-2,3- dihydrobenzofuran-4-yl)methyl)amino)-7-methyl- 4,5-dihydro-3H-2,4,8,11,12a- pentaazabenzo[4,5]cycloocta[1,2,3-cd]inden-3- one |
| E10 | 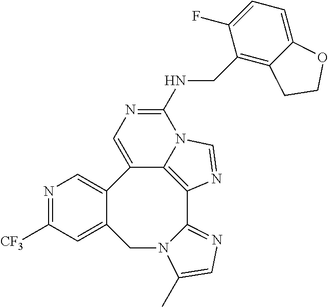 [SMILES: Cc1cnc2n1Cc1cc(C(F)(F)F)ncc1-c1cnc(NCc3c(F)ccc4c3CCO4)n3cnc-2c13] | N-((5-fluoro-2,3-dihydrobenzofuran-4-yl)methyl)- 5-methyl-8-(trifluoromethyl)-6H-2,3,5a,9,12,13a- hexaazabenzo[4,5]cyclopenta[7,8]cycloocta[1,2,3- cd]inden-13-amine |
| E11 | 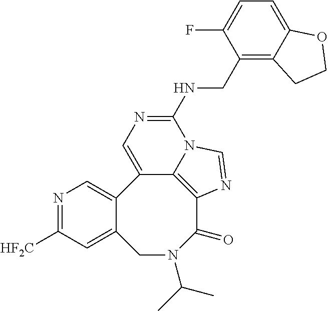 | 7-(difluoromethyl)-12-(((5-fluoro-2,3- dihydrobenzofuran-4-yl)methyl)amino)-4- isopropyl-4,5-dihydro-3H-2,4,8,11,12a- pentaazabenzo[4,5]cycloocta[1,2,3-cd]inden-3- one |
| E12 | 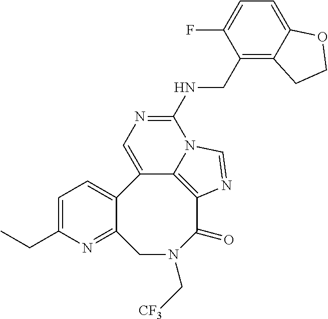 [SMILES: CCc1ccc2c(n1)CN(CC(F)(F)F)C(=O)c1ncn3c(NCc4c(F)ccc5c4CCO5)ncc-2c13] | 7-ethyl-12-(((5-fluoro-2,3-dihydrobenzofuran-4- yl)methyl)amino)-4-(2,2,2-trifluoroethyl)-4,5- dihydro-3H-2,4,6,11,12a- pentaazabenzo[4,5]cycloocta[1,2,3-cd]inden-3- one |
| E13 | 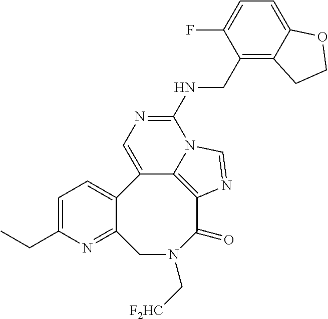 [SMILES: CCc1ccc2c(n1)CN(CC(F)F)C(=O)c1ncn3c(NCc4c(F)ccc5c4CCO5)ncc-2c13] | 4-(2,2-difluoroethyl)-7-ethyl-12-(((5-fluoro-2,3- dihydrobenzofuran-4-yl)methyl)amino)-4,5- dihydro-3H-2,4,6,11,12a- pentaazabenzo[4,5]cycloocta[1,2,3-cd]inden-3- one |
| E14 | 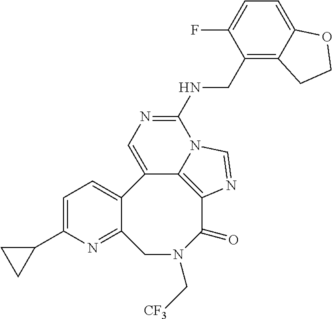 [SMILES: O=C1c2ncn3c(NCc4c(F)ccc5c4CCO5)ncc(c23)-c2ccc(C3CC3)nc2CN1CC(F)(F)F] | 7-cyclopropyl-12-(((5-fluoro-2,3- dihydrobenzofuran-4-yl)methyl)amino)-4-(2,2,2- trifluoroethyl)-4,5-dihydro-3H-2,4,6,11,12a- pentaazabenzo[4,5]cycloocta[1,2,3-cd]inden-3- one |
| E15 | 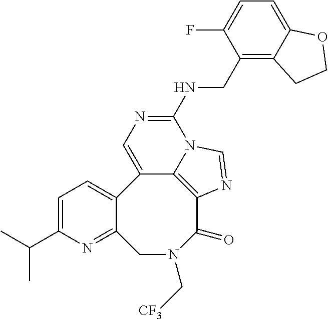 [SMILES: CC(C)c1ccc2c(n1)CN(CC(F)(F)F)C(=O)c1ncn3c(NCc4c(F)ccc5c4CCO5)ncc-2c13] | 12-(((5-fluoro-2,3-dihydrobenzofuran-4- yl)methyl)amino)-7-isopropyl-4-(2,2,2- trifluoroethyl)-4,5-dihydro-3H-2,4,6,11,12a- pentaazabenzo[4,5]cycloocta[1,2,3-cd]inden-3- one |
| E16 | 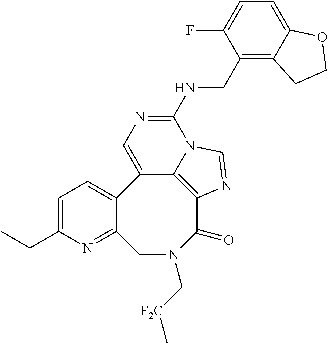 [SMILES: CCc1ccc2c(n1)CN(CC(C)(F)F)C(=O)c1ncn3c(NCc4c(F)ccc5c4CCO5)ncc-2c13] | 4-(2,2-difluoropropyl)-7-ethyl-12-(((5-fluoro-2,3- dihydrobenzofuran-4-yl)methyl)amino)-4,5- dihydro-3H-2,4,6,11,12a- pentaazabenzo[4,5]cycloocta[1,2,3-cd]inden-3- one |
| E17 | 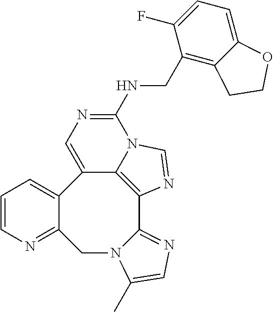 [SMILES: Cc1cnc2n1Cc1ncccc1-c1cnc(NCc3c(F)ccc4c3CCO4)n3cnc-2c13] | N-((5-fluoro-2,3-dihydrobenzofuran-4-yl)methyl)- 5-methyl-6H-2,3,5a,7,12,13a- hexaazabenzo[4,5]cyclopenta[7,8]cycloocta[1,2,3- cd]inden-13-amine |
| E18 | 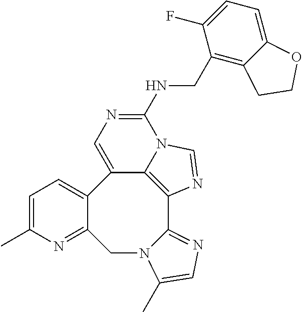 [SMILES: Cc1ccc2c(n1)Cn1c(C)cnc1-c1ncn3c(NCc4c(F)ccc5c4CCO5)ncc-2c13] | N-((5-fluoro-2,3-dihydrobenzofuran-4-yl)methyl)- 5,8-dimethyl-6H-2,3,5a,7,12,13a- hexaazabenzo[4,5]cyclopenta[7,8]cycloota[1,2,3- cd]inden-13-amine |
| E19 | 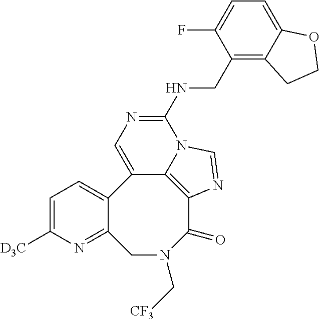 [SMILES: [2H]C([2H])([2H])c1ccc2c(n1)CN(CC(F)(F)F)C(=O)c1ncn3c(NCc4c(F)ccc5c4CCO5)ncc-2c13] | 12-(((5-fluoro-2,3-dihydrobenzofuran-4- yl)methyl)amino)-7-(methyl-d3)-4-(2,2,2- trifluoroethyl)-4,5-dihydro-3H-2,4,6,11,12a- pentaazabenzo[4,5]cycloocta[1,2,3-cd]inden-3- one |
| E20 | 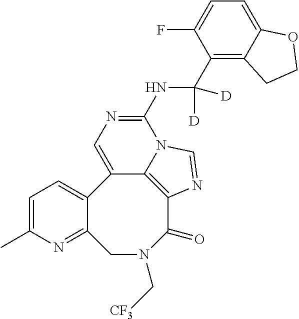 [SMILES: [2H]C([2H])(Nc1ncc2c3c(ncn13)C(=O)N(CC(F)(F)F)Cc1nc(C)ccc1-2)c1c(F)ccc2c1CCO2] | 12-(((5-fluoro-2,3-dihydrobenzofuran-4- yl)methyl-d2)amino)-7-methyl-4-(2,2,2- trifluoroethyl)-4,5-dihydro-3H-2,4,6,11,12a- pentaazabenzo[4,5]cyclocta[1,2,3-cd]inden-3- one |
| E21 | 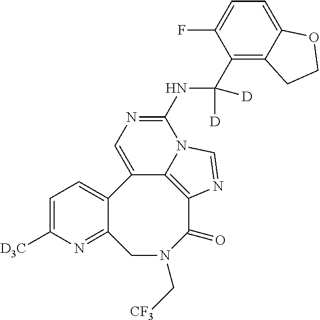 [SMILES: [2H]C([2H])([2H])c1ccc2c(n1)CN(CC(F)(F)F)C(=O)c1ncn3c(NC([2H])([2H])c4c(F)ccc5c4CCO5)ncc-2c13] | 12-(((5-fluoro-2,3-dihydrobenzofuran-4- yl)methyl-d2)amino)-7-(methyl-d3)-4-(2,2,2- trifluoroethyl)-4,5-dihydro-3H-2,4,6,11,12a- pentaazabenzo[4,5]cycloocta[1,2,3-cd]inden-3- one |
| E22 | 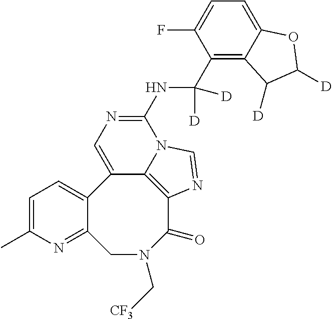 [SMILES: [2H]C1Oc2ccc(F)c(C([2H])([2H])Nc3ncc4c5c(ncn35)C(=O)N(CC(F)(F)F)Cc3nc(C)ccc3-4)c2C1[2H]] | 12-(((5-fluoro-2,3-dihydrobenzofuran-4-yl-2,3- d2)methyl-d2)amino)-7-methyl-4-(2,2,2- trifluoroethyl)-4,5-dihydro-3H-2,4,611,12a- pentaazabenzo[4,5]cycloocta[1,2,3-cd]inden-3- one |
| E23 | 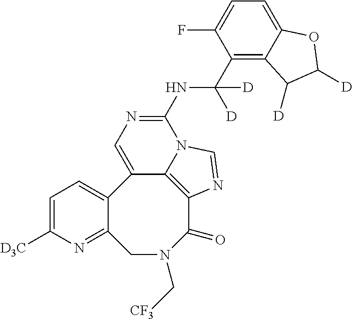 [SMILES: [2H]C1Oc2ccc(F)c(C([2H])([2H])Nc3ncc4c5c(ncn35)C(=O)N(CC(F)(F)F)Cc3nc(C([2H])([2H])[2H])ccc3-4)c2C1[2H]] | 12-(((5-fluoro-2,3-dihydrobenzofuran-4-yl-2,3- d2)methyl-d2)amino)-7-(methyl-d3)-4-(2,2,2- trifluoroethyl)-4,5-dihydro-3H-2,4,6,11,12a- pentaazabenzo[4,5]cycloocta[1,2,3-cd]inden-3- one |
| E24 | 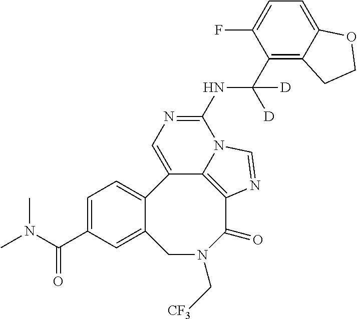 [SMILES: [2H]C([2H])(Nc1ncc2c3c(ncn13)C(=O)N(CC(F)(F)F)Cc1cc(C(=O)N(C)C)ccc1-2)c1c(F)ccc2c1CCO2] | 12-(((5-fluoro-2,3-dihydrobenzofuran-4- yl)methyl-d2)amino)-N,N-dimethyl-3-oxo-4- (2,2,2-trifluoroethyl)-4,5-dihydro-3H-2,4,11,12a- tetraazabenzo[4,5]cycloocta[1,2,3-cd]indene-7- carboxamide |
| E25 | 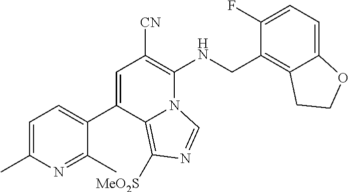 [SMILES: Cc1ccc(-c2cc(C#N)c(NCc3c(F)ccc4c3CCO4)n3cnc(S(C)(=O)=O)c23)c(C)n1] | 8-(2,6-dimethylpyridin-3-yl)-5-(((5-fluoro-2,3- dihdryobenzofurna-4-yl)methyl)amino)-1- (methylsulfonyl)imidazo[1,5-a]pyridine-6- carbonitrile |
| E26 | 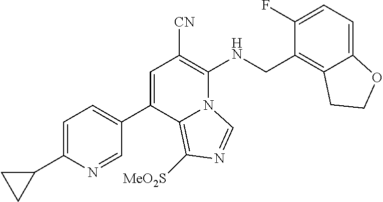 [SMILES: CS(=O)(=O)c1ncn2c(NCc3c(F)ccc4c3CCO4)c(C#N)cc(-c3ccc(C4CC4)nc3)c12] | 8-(6-cyclopropylpyridin-3-yl)-5-(((5-fluoro-2,3- dihydrobenzofuran-4-yl)methyl)amino)-1- (methylsulfonyl)imidazo[1,5-a]pyridine-6- carbonitrile |
| E27 | 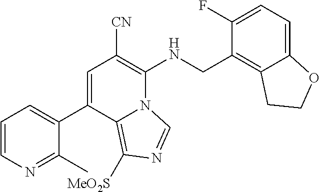 [SMILES: Cc1ncccc1-c1cc(C#N)c(NCc2c(F)ccc3c2CCO3)n2cnc(S(C)(=O)=O)c12] | 5-(((5-fluoro-2,3-dihydrobenzofuran-4- yl)methyl)amino)-8-(2-methylpyridin-3-yl)-1- (methylsulfonyl)imidazo[1,5-a]pyridine-6- carbonitrile |
| E28 | 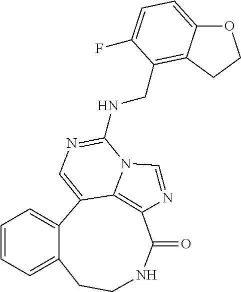 [SMILES: O=C1NCCc2ccccc2-c2cnc(NCc3c(F)ccc4c3CCO4)n3cnc1c23] | 13-(((5-fluoro-2,3-dihydrobenzofuran-4- yl)methyl)amino)-5,6-dihydro-2,4,12,13a- tetraazabenzo[4,5]cyclonona[1,2,3-cd]inden- 3(4H)-one |
| E29 | 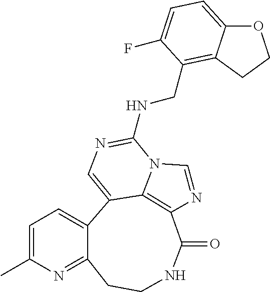 [SMILES: Cc1ccc2c(n1)CCNC(=O)c1ncn3c(NCc4c(F)ccc5c4CCO5)ncc-2c13] | 13-(((5-fluoro-2,3-dihydrobenzofuran-4- yl)methyl)amino)-8-methyl-5,6-dihydro- 2,4,7,12,13a-pentaazabenzo[4,5]cyclonona[1,2,3- cd]inden-3(4H)-one |
| E30 | 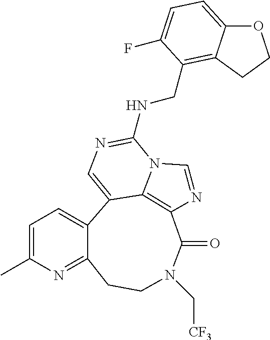 [SMILES: Cc1ccc2c(n1)CCN(CC(F)(F)F)C(=O)c1ncn3c(NCc4c(F)ccc5c4CCO5)ncc-2c13] | 13-(((5-fluoro-2,3-dihydrobenzofurna-4- yl)methyl)amino)-8-methyl-4-(2,2,2- trifluoroethyl)-5,6-dihydro-2,4,7,12,13a- pentaazabenzo[4,5]cyclonona[1,2,3-cd]inden- 3(4H)-one |
| E31 | 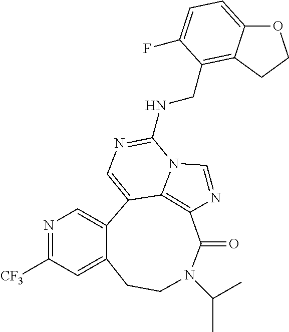 [SMILES: CC(C)N1CCc2cc(C(F)(F)F)ncc2-c2cnc(NCc3c(F)ccc4c3CCO4)n3cnc(c23)C1=O] | 13-(((5-fluoro-2,3-dihydrobenzofuran-4- yl)methyl)amino)-4-isopropyl-8-(trifluoromethyl)- 5,6-dihydro-2,4,9,12,13a- pentaazabenzo[4,5]cyclonona[1,2,3-cd]inden- 3(4H)-one |
| E32 | 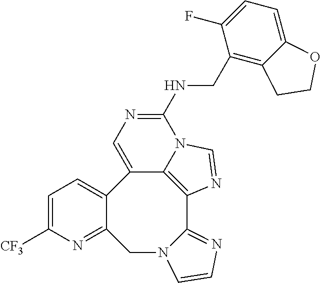 [SMILES: Fc1ccc2c(c1CNc1ncc3c4c(ncn14)-c1nccn1Cc1nc(C(F)(F)F)ccc1-3)CCO2] | N-((5-fluoro-2,3-dihydrobenzofuran-4-yl)methyl)- 8-(trifluoromethyl)-6H-2,3,5a,7,12,13a- hexaazabenzo[4,5]cyclopenta[7,8]cycloocta[1,2,3- cd]inden-13-amine |
| E33 | 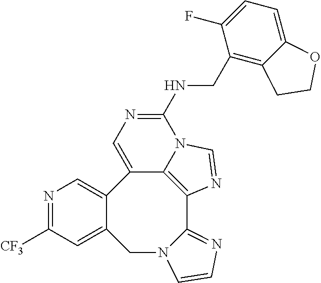 [SMILES: Fc1ccc2c(c1CNc1ncc3c4c(ncn14)-c1nccn1Cc1cc(C(F)(F)F)ncc1-3)CCO2] | N-((5-fluoro-2,3-dihydrobenzofuran-4-yl)methyl)- 8-(trifluoromethyl)-6H-2,3,5a,9,12,13a- hexaazabenzo[4,5]cyclopenta[7,8]cycloocta[1,2,3- cd]inden-13-amine |
| E34 | 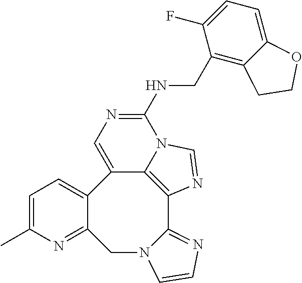 [SMILES: Cc1ccc2c(n1)Cn1ccnc1-c1ncn3c(NCc4c(F)ccc5c4CCO5)ncc-2c13] | N-((5-fluoro-2,3-dihydrobenzofuran-4-yl)methyl)- 8-methyl-6H-2,3,5a,7,12,13a- hexaazabenzo[4,5]cyclopenta[7,8]cycloocta[1,2,3- cd]inden-13-amine |
| E35 | 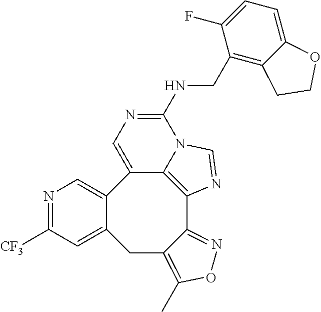 [SMILES: Cc1onc2c1Cc1cc(C(F)(F)F)ncc1-c1cnc(NCc3c(F)ccc4c3CCO4)n3cnc-2c13] | N-((5-fluoro-2,3-dihydrobenzofuran-4-yl)methyl)- 5-methyl-8-(trifluoromethyl)-6H-4-oxa- 2,3,9,12,13a- pentaazabenzo[4,5]cyclopenta[7,8]cycloocta[1,2,3- cd]inden-13-amine |
| E36 | 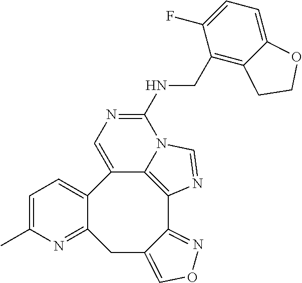 [SMILES: Cc1ccc2c(n1)Cc1conc1-c1ncn3c(NCc4c(F)ccc5c4CCO5)ncc-2c13] | N-((5-fluoro-2,3-dihydrobenzofurna-4-yl)methyl)- 8-methyl-6H-4-oxa-2,3,7,12,13a- pentaazabenzo[4,5]cyclopenta[7,8]cycloocta[1,2,3- cd]inden-13-amine |
| E37 | 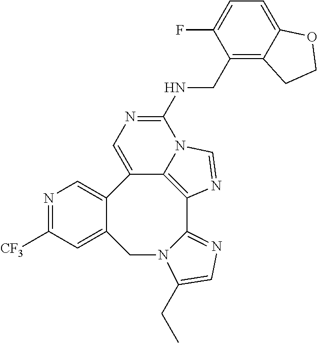 [SMILES: CCc1cnc2n1Cc1cc(C(F)(F)F)ncc1-c1cnc(NCc3c(F)ccc4c3CCO4)n3cnc-2c13] | 5-ethyl-N-((5-fluoro-2,3-dihydrobenzofuran-4- yl)methyl)-8-(trifluoromethyl)-6H- 2,3,5a,9,12,13a- hexaazabenzo[4,5]cyclopenta[7,8]cycloocta[1,2,3- cd]inden-13-amine |
| E38 | 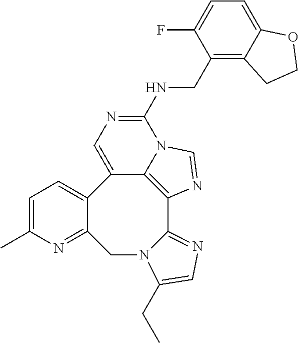 [SMILES: CCc1cnc2n1Cc1nc(C)ccc1-c1cnc(NCc3c(F)ccc4c3CCO4)n3cnc-2c13] | 5-ethyl-N-((5-fluoro-2,3-dihydrobenzofuran-4- yl)methyl)-8-methyl-6H-2,3,5a,7,12,13a- hexaazabenzo[4,5]cyclopenta[7,8]cycloocta[1,2,3- cd]inden-13-amine |
| E39 | 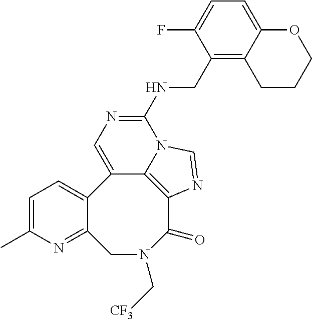 [SMILES: Cc1ccc2c(n1)CN(CC(F)(F)F)C(=O)c1ncn3c(NCc4c(F)ccc5c4CCCO5)ncc-2c13] | 12-(((6-fluorochroman-5-yl)methyl)amino)-7- methyl-4-(2,2,2-trifluoroethyl)-4,5-dihydro-3H- 2,4,6,11,12a-pentaazabenzo[4,5]cycloocta[1,2,3- cd]inden-3-one |
| E40 | 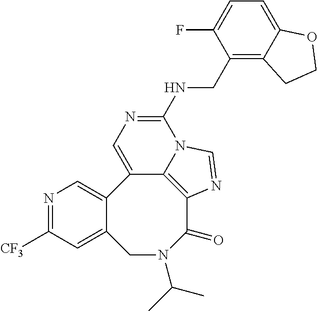 [SMILES: CC(C)N1Cc2cc(C(F)(F)F)ncc2-c2cnc(NCc3c(F)ccc4c3CCO4)n3cnc(c23)C1=O] | 12-(((5-fluoro-2,3-dihydrobenzofuran-4- yl)methyl)amino)-4-isopropyl-7-(trifluoromethyl)- 4,5-dihydro-3H-2,4,8,11,12a- pentaazabenzo[4,5]cycloocta[1,2,3-cd]inden-3- one |
| E41 | 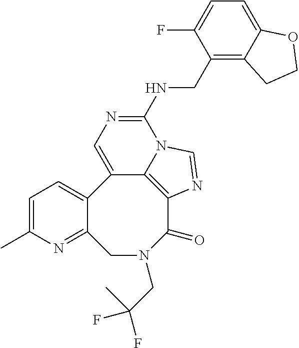 [SMILES: Cc1ccc2c(n1)CN(CC(C)(F)F)C(=O)c1ncn3c(NCc4c(F)ccc5c4CCO5)ncc-2c13] | 4-(2,2-difluoropropyl)-12-(((5-fluoro-2,3- dihydrobenzofuran-4-yl)methyl)amino)-7-methyl- 4,5-dihydro-3H-2,4,6,11,12a- pentaazabenzo[4,5]cycloocta[1,2,3-cd]inden-3- one |
| E42 | 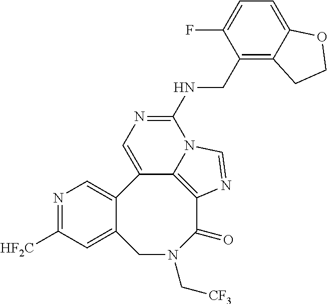 | 7-(difluoromethyl)-12-(((5-fluoro-2,3- dihydrobenzofuran-4-yl)methyl)amino)-4-(2,2,2- trifluoroethyl)-4,5-dihydro-3H-2,4,8,11,12a- pentaazabenzo[4,5]cycloocta[1,2,3-cd]inden-3- one |
| E43 | 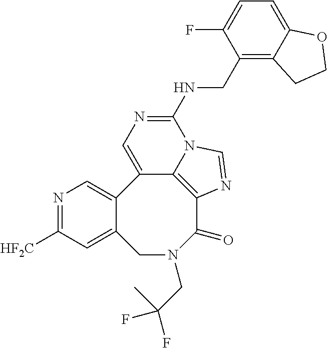 | 7-(difluoromethyl)-4-(2,2-difluoropropyl)-12-(((5- fluoro-2,3-dihydrobenzofuran-4- yl)methyl)amino)-4,5-dihydro-3H-2,4,8,11,12a- pentaazabenzo[4,5]cycloocta[1,2,3-cd]inden-3- one |
| E44 | 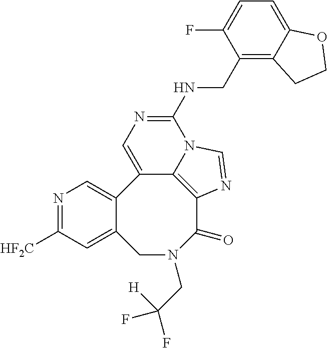 | 4-(2,2-difluoroethyl)-7-(difluoromethyl)-12-(((5- fluoro-2,3-dihydrobenzofuran-4- yl)methyl)amino)-4,5-dihydro-3H-2,4,8,11,12a- pentaazabenzo[4,5]cycloocta[1,2,3-cd]inden-3- one |
| E45 | 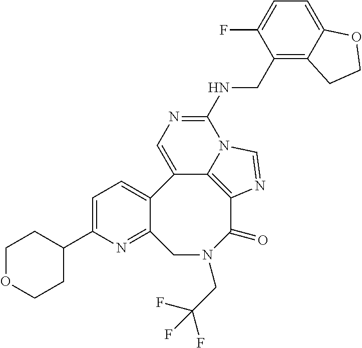 [SMILES: O=C1c2ncn3c(NCc4c(F)ccc5c4CCO5)ncc(c23)-c2ccc(C3CCOCC3)nc2CN1CC(F)(F)F] | 12-(((5-fluoro-2,3-dihydrobenzofuran-4- yl)methyl)amino)-7-(tetrahydro-2H-pyran-4-yl)-4- (2,2,2-trifluoroethyl)-4,5-dihydro-3H- 2,4,6,11,12a-pentaazabenzo[4,5]cycloocta[1,2,3- cd]inden-3-one |
| E46 | 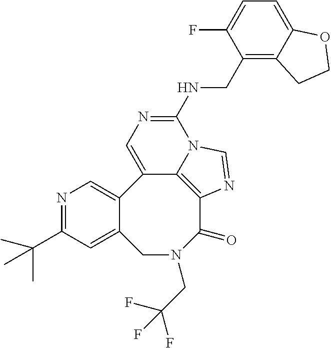 [SMILES: CC(C)(C)c1cc2c(cn1)-c1cnc(NCc3c(F)ccc4c3CCO4)n3cnc(c13)C(=O)N(CC(F)(F)F)C2] | 7-(tert-butyl)-12-(((5-fluoro-2,3- dihydrobenzofuran-4-yl)methyl)amino)-4-(2,2,2- trifluoroethyl)-4,5-dihydro-3H-2,4,8,11,12a- pentaazabenzo[4,5]cycloocta[1,2,3-cd]inden-3- one |
| E47 | 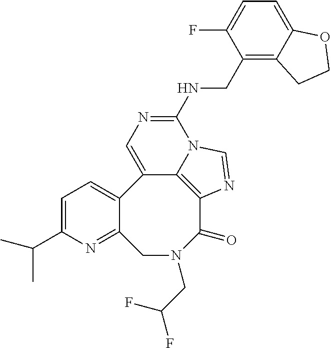 [SMILES: CC(C)c1ccc2c(n1)CN(CC(F)F)C(=O)c1ncn3c(NCc4c(F)ccc5c4CCO5)ncc-2c13] | 4-(2,2-difluoroethyl)-12-(((5-fluoro-2,3- dihydrobenzofuran-4-yl)methyl)amino)-7- isopropyl-4,5-dihydro-3H-2,4,6,11,12a- pentaazabenzo[4,5]cycloocta[1,2,3-cd]inden-3- one |
| E48 | 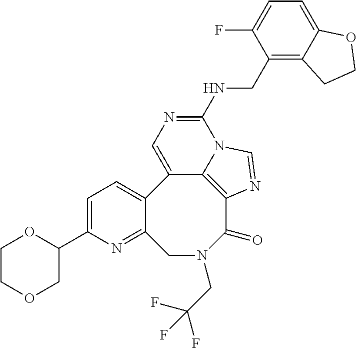 [SMILES: O=C1c2ncn3c(NCc4c(F)ccc5c4CCO5)ncc(c23)-c2ccc(C3COCCO3)nc2CN1CC(F)(F)F] | 7-(1,4-dioxan-2-yl)-12-(((5-fluoro-2,3- dihydrobenzofuran-4-yl)methyl)amino)-4-(2,2,2- trifluoroethyl)-4,5-dihydro-3H-2,4,6,11,12a- pentaazabenzo[4,5]cycloocta[1,2,3-cd]inden-3- one |
| E49 | 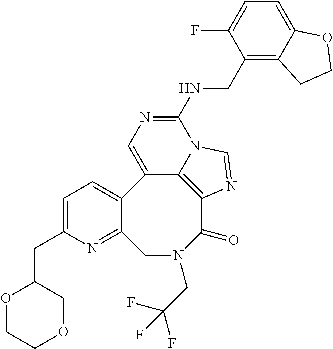 [SMILES: O=C1c2ncn3c(NCc4c(F)ccc5c4CCO5)ncc(c23)-c2ccc(CC3COCCO3)nc2CN1CC(F)(F)F] | 7-((1,4-dioxan-2-yl)methyl)-12-(((5-fluoro-2,3- dihydrobenzofuran-4-yl)methyl)amino)-4-(2,2,2- trifluoroethyl)-4,5-dihydro-3H-2,4,6,11,12a- pentaazabenzo[4,5]cycloocta[1,2,3-cd]inden-3- one |
| E50 | 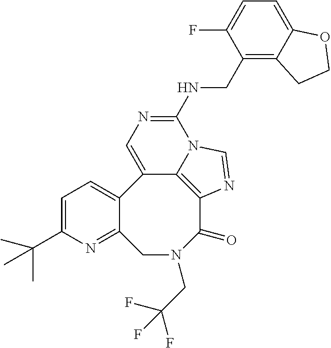 [SMILES: CC(C)(C)c1ccc2c(n1)CN(CC(F)(F)F)C(=O)c1ncn3c(NCc4c(F)ccc5c4CCO5)ncc-2c13] | 7-(tert-butyl)-12-(((5-fluoro-2,3- dihydrobenzofuran-4-yl)methyl)amino)-4-(2,2,2- trifluoroethyl)-4,5-dihydro-3H-2,4,6,11,12a- pentaazabenzo[4,5]cycloocta[1,2,3-cd]inden-3- one |
| E51 | 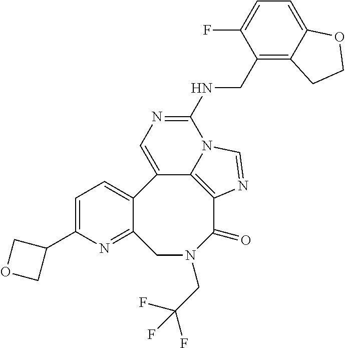 [SMILES: O=C1c2ncn3c(NCc4c(F)ccc5c4CCO5)ncc(c23)-c2ccc(C3COC3)nc2CN1CC(F)(F)F] | 12-(((5-fluoro-2,3-dihydrobenzofuran-4- yl)methyl)amino)-7-(oxetan-3-yl)-4-(2,2,2- trifluoroethyl)-4,5-dihydro-3H-2,4,6,11,12a- pentaazabenzo[4,5]cycloocta[1,2,3-cd]inden-3- one |
| E52 | 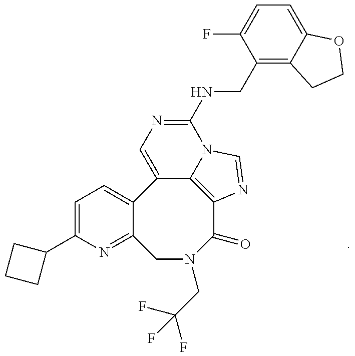 [SMILES: O=C1c2ncn3c(NCc4c(F)ccc5c4CCO5)ncc(c23)-c2ccc(C3CCC3)nc2CN1CC(F)(F)F] | 7-cyclobutyl-12-(((5-fluoro-2,3- dihydrobenzofuran-4-yl)methyl)amino)-4-(2,2,2- trifluoroethyl)-4,5-dihydro-3H-2,4,6,11,12a- pentaazabenzo[4,5]cycloocta[1,2,3-cd]inden-3- one |

In some embodiments, the substance X are compounds of Formula I selected from group consisting of:
4-ethyl-12-(((5-fluoro-2,3-dihydrobenzofuran-4-yl)methyl)amino)-7-(trifluoromethyl)-4,5-dihydro-3H-2,4,8,11,12a-pentaazabenzo[4,5]cycloocta[1,2,3-cd]inden-3-one;
12-(((5-fluoro-2,3-dihydrobenzofuran-4-yl)methyl)amino)-4-(2,2,2-trifluoroethyl)-7-(trifluoromethyl)-4,5-dihydro-3H-2,4,8,11,12a-pentaazabenzo[4,5]cycloocta[1,2,3-cd]inden-3-one;
4-cyclopropyl-12-(((5-fluoro-2,3-dihydrobenzofuran-4-yl)methyl)amino)-7-(trifluoromethyl)-4,5-dihydro-3H-2,4,8,11,12a-pentaazabenzo[4,5]cycloocta[1,2,3-cd]inden-3-one;
12-(((5-fluoro-2,3-dihydrobenzofuran-4-yl)methyl)amino)-4-isopropyl-7-(trifluoromethyl)-4,5-dihydro-3H-2,4,8,11,12a-pentaazabenzo[4,5]cycloocta[1,2,3-cd]inden-3-one; and
11-(((5-fluoro-2,3-dihydrobenzofuran-4-yl)methyl)amino)-6-methyl-4H-3-thia-2,5,10,11a-tetraazadibenzo[cd,f]azulene 3,3-dioxide,
   or a pharmaceutically acceptable salt or solvate thereof.

In some embodiments, the substance X is Cpd. 73

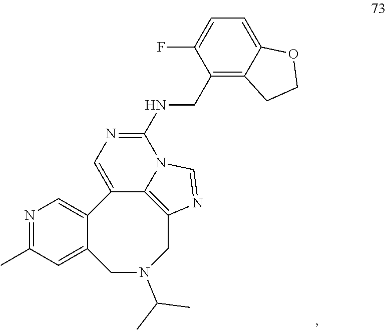 [SMILES: Cc1cc2c(cn1)-c1cnc(NCc3c(F)ccc4c3CCO4)n3cnc(c13)CN(C(C)C)C2]

or a pharmaceutically acceptable salt or solvate thereof.

In some embodiments, the subject may be a mammal.

In some embodiments, the subject may be a mice or a human.

In some embodiments, the pharmaceutical composition may comprise the substance X and an excipient and/or pharmaceutically acceptable carrier.

In some embodiments, the substance X or the pharmaceutical composition may be administered orally.

In some embodiments, the substance X or the pharmaceutical composition may be administered one or more times daily.

In some embodiments, the substance X or the pharmaceutical composition may be administered one or more times weekly.

In some embodiments, the substance X may be administered in an amount of 3 mg/kg to 90 mg/kg per time to the subject in need thereof.

In some embodiments, the substance X may be administered in an amount of 3 mg/kg, 10 mg/kg, 30 mg/kg or 90 mg/kg per time to the subject in need thereof.

In some embodiments, the substance X or the pharmaceutical composition may be administered continuously for at least 5 days, at least 1 week, at least 2 weeks, at least 3 weeks or at least 4 weeks.

In some embodiments, the disease or disorder associated with a defect in hemoglobin protein activity or expression is a blood disorder; Preferably anemia.

In some embodiments, the disease or disorder associated with a defect in hemoglobin protein activity or expression is selected from a group consisting of: Sickle cell disease, β-thalassemia, β-thalessemia intermedia, β-thalessemia major, β-thalessemia minor, chemotherapy-induced anemia, chronic kidney disease related anemia, and Cooley's anemia. Preferably, the disease or disorder is Sickle cell disease or β-thalassemia.

In another aspect, the present invention provides a method of decreasing the H3K27me3 level in erythroid lineage cells or the H3K27me3 level in PBMC monocytes, which comprises administering to a subject in need thereof a therapeutically effective amount of a substance X or a pharmaceutical composition comprising the substance X; the substance X is as defined above; the pharmaceutical composition is as defined above.

In some embodiments, the erythroid lineage cells may be the erythroid lineage cells from bone marrow.

In some embodiments, the erythroid lineage cells may be the TER119+ erythroid lineage cells from bone marrow.

In some embodiments, the subject may suffer from a disease or disorder associated with a defect in hemoglobin protein activity or expression.

In some embodiments, the disease or disorder associated with a defect in hemoglobin protein activity or expression is a blood disorder; Preferably anemia.

In some embodiments, the disease or disorder associated with a defect in hemoglobin protein activity or expression is selected from a group consisting of: Sickle cell disease, β-thalassemia, β-thalessemia intermedia, β-thalessemia major, β-thalessemia minor, chemotherapy-induced anemia, chronic kidney disease related anemia, and Cooley's anemia.

Preferably, the disease or disorder is Sickle cell disease or β-thalassemia.

In some embodiments, the subject may be a mammal.

In some embodiments, the subject may be a mice or a human.

In some embodiments, the pharmaceutical composition may comprise the substance X and an excipient and/or pharmaceutically acceptable carrier.

In some embodiments, the substance X or the pharmaceutical composition may be administered orally.

In some embodiments, the substance X or the pharmaceutical composition may be administered one or more times daily.

In some embodiments, the substance X or the pharmaceutical composition may be administered one or more times weekly.

In some embodiments, the substance X may be administered in an amount of 3 mg/kg to 90 mg/kg per time to the subject in need thereof.

In some embodiments, the substance X may be administered in an amount of 3 mg/kg, 10 mg/kg, 30 mg/kg or 90 mg/kg per time to the subject in need thereof.

In some embodiments, the substance X or the pharmaceutical composition may be administered continuously for at least 5 days, at least 1 week, at least 2 weeks, at least 3 weeks or at least 4 weeks.

In another aspect, the present invention provides a method of increasing Hbb-bh1 mRNA level, HBG gene level (HBG1, HBG2), γ hemoglobin level or HbF level, which comprises administering to a subject in need thereof a therapeutically effective amount of a substance X or a pharmaceutical composition comprising the substance X; the substance X is as defined above; the pharmaceutical composition is as defined above.

In some embodiments, the Hbb-bh1 mRNA level may be the Hbb-bh1 mRNA level in blood, e.g., peripheral blood.

In some embodiments, the Hbb-bh1 mRNA level may be the Hbb-bh1 mRNA level in the blood of CD-1 mice.

In some embodiments, the HBG1 gene level may be the HBG1 gene level in blood, e.g., peripheral blood.

In some embodiments, the HBG1 gene level may be the HBG1 gene level in the blood of human.

In some embodiments, the HBG2 gene level may be the HBG2 gene level in blood, e.g., peripheral blood.

In some embodiments, the HBG2 gene level may be the HBG2 gene level in the blood of human.

In some embodiments, the γ hemoglobin level may be the γ hemoglobin level in blood, e.g., peripheral blood.

In some embodiments, the γ hemoglobin level may be the γ hemoglobin level in the blood of human.

In some embodiments, the HbF level may be the HbF level in blood, e.g., peripheral blood.

In some embodiments, the HbF level may be the HbF level in the blood of human.

In some embodiments, the subject may suffer from a disease or disorder associated with a defect in hemoglobin protein activity or expression.

In some embodiments, the subject may be a mammal.

In some embodiments, the subject may be a mice or a human.

In some embodiments, the pharmaceutical composition may comprise the substance X and an excipient and/or pharmaceutically acceptable carrier.

In some embodiments, the substance X or the pharmaceutical composition may be administered orally.

In some embodiments, the substance X or the pharmaceutical composition may be administered one or more times daily.

In some embodiments, the substance X or the pharmaceutical composition may be administered one or more times weekly.

In some embodiments, the substance X may be administered in an amount of 3 mg/kg to 90 mg/kg per time to the subject in need thereof.

In some embodiments, the substance X may be administered in an amount of 3 mg/kg, 10 mg/kg, 30 mg/kg or 90 mg/kg per time to the subject in need thereof.

In some embodiments, the substance X or the pharmaceutical composition may be administered continuously for at least 5 days, at least 1 week, at least 2 weeks, at least 3 weeks or at least 4 weeks.

In some embodiments, the disease or disorder associated with a defect in hemoglobin protein activity or expression is a blood disorder; Preferably anemia.

In some embodiments, the disease or disorder associated with a defect in hemoglobin protein activity or expression is selected from a group consisting of: Sickle cell disease, β-thalassemia, β-thalessemia intermedia, β-thalessemia major, β-thalessemia minor, chemotherapy-induced anemia, chronic kidney disease related anemia, and Cooley's anemia. Preferably, the disease or disorder is Sickle cell disease or β-thalassemia.

In some embodiments, the method comprising using a second therapeutic agent, preferably Erythropoietin (EPO).

In some embodiments, the innate immune response, type 1 or type 3 immunity related immune disorder is inflammatory bowel diseases, including Crohn's disease and ulcerative colitis.

In some embodiments, the innate immune response, type 1 or type 3 immunity related immune disorder is selected from a group consisting of: acute disseminated encephalomyelitis (ADEM), Addison disease, ankylosing spondylitis, antiphospholipid syndrome (APGS), aplastic anemia, American Industrial Hygiene Association (AIHA), autoimmune hepatitis (AIH), autoimmune hypoparathyroidism, Autoimmune hypophysitis, autoimmune myocardioptis, autoimmune oophoritis, autoimmune orchitis, Autoimmune thrombocytopenic purpura (AITP), Behcet's disease, bullous pemphigoid, Chronic inflammatory demyelinating polyneuropathy, Churg-Strauss syndrome, Crohn's disease, dermatomyositis, familial dysautonomia, epidermolysis bullosa, Pemphigoid during pregnancy, giant cell arteritis, Goodpasture syndrome, Granulomatous disease with polyvasculitis, Graves' disease, Guillain-barre syndrome, Hashimoto Disease, Immunoglobulin A (IgA) neurological disease, ulcerative colitis, interstitial cystitis (IC), Kawasaki Disease, Lambert-Eaton myasthenic syndrome (LEMS), Chronic Lyme disease, Mooren's ulcer, morphea, myasthenia gravis, neuromyotonia, multiple sclerosis, Clonic syndrome of strabismus, optic neuritis, Ord thyroiditis, pemphigus, pernicious anemia, polyarteritis, polyarthritis, Polyglandular autoimmune syndrome, primary biliary cirrhosis, psoriasis, Reiter's syndrome, Sarcoidosis, rheumatic arthritis, Sjogren's syndrome, stiff-man syndrome, systemic lupus erythematosus, Takayasu arthritis, and Vogt-Kovangai-Harada disease.

In another aspect, the present invention provides a pharmaceutical composition comprising (i) the substance X; (ii) erythropoietin.

In some embodiments, the substance X may be administered in an amount of 10-30 mg/kg, preferably 15 mg/kg.

In some embodiments, the Erythropoietin (EPO) may be administered in an amount of 10-100 U/kg, preferably 50 U/kg.

In some embodiments, the innate immune response, type 1 or type 3 immunity related immune disorder is inflammatory bowel diseases, including Crohn's disease and ulcerative colitis.

In some embodiments, the innate immune response, type 1 or type 3 immunity related immune disorder is selected from a group consisting of: acute disseminated encephalomyelitis (ADEM), Addison disease, ankylosing spondylitis, antiphospholipid syndrome (APGS), aplastic anemia, American Industrial Hygiene Association (AIHA), autoimmune hepatitis (AIH), autoimmune hypoparathyroidism, Autoimmune hypophysitis, autoimmune myocardioptis, autoimmune oophoritis, autoimmune orchitis, Autoimmune thrombocytopenic purpura (AITP), Behcet's disease, bullous pemphigoid, Chronic inflammatory demyelinating polyneuropathy, Churg-Strauss syndrome, Crohn's disease, dermatomyositis, familial dysautonomia, epidermolysis bullosa, Pemphigoid during pregnancy, giant cell arteritis, Goodpasture syndrome, Granulomatous disease with polyvasculitis, Graves' disease, Guillain-barre syndrome, Hashimoto Disease, Immunoglobulin A (IgA) neurological disease, ulcerative colitis, interstitial cystitis (IC), Kawasaki Disease, Lambert-Eaton myasthenic syndrome (LEMS), Chronic Lyme disease, Mooren's ulcer, morphea, myasthenia gravis, neuromyotonia, multiple sclerosis, Clonic syndrome of strabismus, optic neuritis, Ord thyroiditis, pemphigus, pernicious anemia, polyarteritis, polyarthritis, Polyglandular autoimmune syndrome, primary biliary cirrhosis, psoriasis, Reiter's syndrome, Sarcoidosis, rheumatic arthritis, Sjogren's syndrome, stiff-man syndrome, systemic lupus erythematosus, Takayasu arthritis, and Vogt-Kovangai-Harada disease.

In some embodiments, the substance X may be administered in an amount of 10-100 mg/kg, preferably 50 mg/kg or 90 mg/kg.

The term “preventing” refers to a method of preventing the onset of a disease or condition and/or its attendant symptoms or barring a subject from acquiring a disease. As used herein, “preventing” also includes delaying the onset of a disease and/or its attendant symptoms and reducing a subject's risk of acquiring a disease. The terms “preventing” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition

The term “treating” refers to eliminating, reducing, or ameliorating a disease or condition, and/or symptoms associated therewith. Although not precluded, treating a disease or condition does not require that the disease, condition, or symptoms associated therewith be completely eliminated. The term “treating” and synonyms contemplate administering a therapeutically effective amount of a compound to a subject in need of such treatment. The treatment can be orientated symptomatically, for example, to suppress symptoms. It can be effected over a short period, be oriented over a medium term, or can be a long-term treatment, for example within the context of a maintenance therapy.

The term “subject” refers to any animal, including mammals, such as mice, rats, other rodents, rabbits, dogs, cats, pigs, cattle, sheep, horses, primates or humans. The preferred subjects are humans.

The term “therapeutically effective amount” of a substance or a pharmaceutical composition refers to an amount sufficient to cure, alleviate or partially arrest the clinical manifestations of a given disease or disorder and its complications. The amount that is effective for a particular therapeutic purpose will depend on the severity of the disease or injury as well as on the weight and general state of the subject. It will be understood that determination of an appropriate dosage may be achieved, using routine experimentation, by constructing a matrix of values and testing different points in the matrix, all of which is within the ordinary skills of a trained physician or clinical scientist.

The term “pharmaceutically acceptable salt” refers to salts or zwitterionic forms of Compound. Salts of Compound can be prepared during the final isolation and purification of the compounds or separately by reacting the compound with a suitable acid. The pharmaceutically acceptable salts of Compound can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of compound include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compound can be quatemized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides.

The term “solvate” refers to a combination, physical association and/or solvation of a compound of the present invention with a solvent molecule, e.g., a disolvate, monosolvate or hemisolvate, where the ratio of solvent molecule to compound of the present invention is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds can be present as solvated forms with a pharmaceutically acceptable solvent, such as water, methanol, ethanol, and the like. In some embodiment, the solvate is a hydrate. A “hydrate” relates to a particular subgroup of solvates where the solvent molecule is water. Solvates typically can function as pharmacological equivalents. Preparation of solvates is known in the art. A typical, non-limiting, process of preparing a solvate would involve dissolving a compound in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

The term “chronic kidney disease related anemia” and “chronic kidney disease induced anemia” are used interchangeably herein.

The use of the terms “a”, “an”, “the”, and similar referents in the context of describing the invention (especially in the context of the claims) are to be construed to cover both the singular and the plural, unless otherwise indicated.

The term “halo” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one embodiment, the alkyl is a C₁-C₁₀ alkyl. In another embodiment, the alkyl is a C₁-C₆ alkyl. In another embodiment, the alkyl is a C₁-C₄ alkyl. In another embodiment, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl. In another embodiment, one or more of the hydrogen atoms of the alkyl group are replaced by deuterium atoms, i.e., the alkyl group is isotopically-labeled with deuterium. A non-limiting exemplarly deteuterated alkyl group is —CD₃.

The term “optionally substituted alkyl” as used herein by itself or as part of another group refers to an alkyl group that is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy, alkoxycarbonyl, carboxyalkyl, —N(R^{56a})C(═O)R^{56b}, —N(R^{56c})S(═O)₂R^{56d}, —C(═O)R⁵⁷, —S(═O)R^{56e}, or —S(═O)₂R⁵⁸; wherein:
R^{56a} is hydrogen or alkyl;
R^{56b} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R^{56c} is hydrogen or alkyl;
R^{86d} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R^{56e} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R⁵⁷ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl; and
R⁵⁸ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl. Non-limiting exemplary optionally substituted alkyl groups include —CH(CO₂Me)CH₂CO₂Me and —CH(CH₃)CH₂N(H)C(═O)O(CH₃)₃.

The term “alkenyl” as used herein by itself or as part of another group refers to an alkyl group containing one, two, or three carbon-to-carbon double bonds. In one embodiment, the alkenyl group is a C₂-C₆ alkenyl group. In another embodiment, the alkenyl group is a C₂-C₄ alkenyl group. In another embodiment, the alkenyl group has one carbon-to-carbon double bond. Non-limiting exemplary alkenyl groups include ethenyl, propenyl, isopropenyl, butenyl, sec-butenyl, pentenyl, and hexenyl.

The term “optionally substituted alkenyl” as used herein by itself or as part of another refers to an alkenyl group that is either unsubstituted or substituted with one, two or three substituents, wherein each substituent is independently halo, nitro, cyano, hydroxy, amino (e.g., alkylamino, dialkylamino), haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkenyl groups include —CH═CHPh.

The term “alkynyl” as used herein by itself or as part of another group refers to an alkyl group containing one, two, or three carbon-to-carbon triple bonds. In one embodiment, the alkynyl is a C₂-C₆ alkynyl. In another embodiment, the alkynyl is a C₂-C₄ alkynyl. In another embodiment, the alkynyl has one carbon-to-carbon triple bond. Non-limiting exemplary alkynyl groups include ethynyl, propynyl, butynyl, 2-butynyl, pentynyl, and hexynyl groups.

The term “optionally substituted alkynyl” as used herein by itself or as part of another group refers to an alkynyl group that is either unsubstituted or substituted with one, two or three substituents, wherein each substituent is independently halo, nitro, cyano, hydroxy, amino, e.g., alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkynyl groups include —C≡CPh and —CH(Ph)C≡CH.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl. In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In one embodiment, the alkyl is a C₁-C₆ alkyl. In another embodiment, the alkyl is a C₁-C₄ alkyl. In another embodiment, the alkyl is a C₁ or C₂ alkyl. In another embodiment, the hydroxyalkyl is a monohydroxyalkyl group, i.e., substituted with one hydroxy group. In another embodiment, the hydroxyalkyl group is a dihydroxyalkyl group, i.e., substituted with two hydroxy groups. Non-limiting exemplary (hydroxyl)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group attached to a terminal oxygen atom. In one embodiment, the alkyl is a C₁-C₆ alkyl and resulting alkoxy is thus referred to as a “C₁-C₆ alkoxy.” In another embodiment, the alkyl is a C₁-C₄ alkyl group. Non-limiting exemplary alkoxy groups include methoxy, ethoxy, and tert-butoxy.

The term “haloalkoxy” as used herein by itself or as part of another group refers to a haloalkyl group attached to a terminal oxygen atom. In one embodiment, the haloalkyl group is a C₁-C₆ haloalkyl. In another embodiment, the haloalkyl group is a C₁-C₄haloalkyl group. Non-limiting exemplary haloalkoxy groups include fluoromethoxy, difluoromethoxy, trifluoromethoxy, and 2,2,2-trifluoroethoxy.

The term “alkylthio” as used herein by itself or as part of another group refers to an alkyl group attached to a terminal sulfur atom. In one embodiment, the alkyl group is a C₁-C₄ alkyl group. Non-limiting exemplary alkylthio groups include —SCH₃, and —SCH₂CH₃.

The terms “alkoxyalkyl” or “(alkoxy)alkyl” as used herein by themselves or as part of another group refers to an alkyl group substituted with one alkoxy group. In one embodiment, the alkoxy is a C₁-C₆ alkoxy. In another embodiment, the alkoxy is a C₁-C₄ alkoxy. In another embodiment, the alkyl is a C₁-C₆ alkyl. In another embodiment, the alkyl is a C₁-C₄ alkyl. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

The term “heteroalkyl” as used by itself or part of another group refers to unsubstituted straight- or branched-chain aliphatic hydrocarbons containing from three to twenty chain atoms, i.e., 3- to 20-membered heteroalkyl, or the number of chain atoms designated, wherein at least one —CH₂— is replaced with at least one of —O—, —N(H)—, —N(C₁-C₄ alkyl)-, or —S—. The —O—, —N(H)—, —N(C₁-C₄ alkyl)-, or —S— can independently be placed at any interior position of the aliphatic hydrocarbon chain so long as each —O—, —N(H)—, —N(C₁-C₄ alkyl)-, and —S— group is separated by at least two —CH₂— groups. In one embodiment, one —CH₂— group is replaced with one —O— group. In another embodiment, two —CH₂— groups are replaced with two —O— groups. In another embodiment, three —CH₂— groups are replaced with three —O— groups. In another embodiment, four —CH₂— groups are replaced with four —O— groups. Non-limiting exemplary heteroalkyl groups include —CH₂OCH₃, —CH₂OCH₂CH₂CH₃, —CH₂CH₂CH—₂OCH₃, —CH₂CH₂OCH₂CH₂OCH₂CH₃, —CH₂CH₂OCH₂CH₂OCH₂CH₂OCH₂CH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In one embodiment, the cycloalkyl is bicyclic, i.e., it has two rings. In another embodiment, the cycloalkyl is monocyclic, i.e., it has one ring. In another embodiment, the cycloalkyl is a C₃₋₈ cycloalkyl. In another embodiment, the cycloalkyl is a C₃-6 cycloalkyl, i.e., cyclopropyl, cyclobutyl, cyclopentyl, or cyclohexyl. In another embodiment, the cycloalkyl is a C₅ cycloalkyl, i.e., cyclopentyl. In another embodiment, the cycloalkyl is a C₆ cycloalkyl, i.e., cyclohexyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl group that is either unsubstituted or substituted with one, two, or three substituents, wherein each substituent is independently halo, nitro, cyano, hydroxy, amino (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{86a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, and R⁵⁸ are as defined in connection with the term “optionally substituted alkyl” and R⁵⁹ is (hydroxy)alkyl or (amino)alkyl. The term optionally substituted cycloalkyl also includes cycloalkyl groups having fused optionally substituted aryl or optionally substituted heteroaryl groups such as

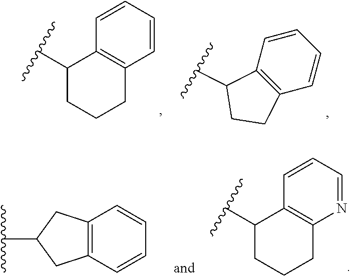 [SMILES: *C1CCCc2ccccc21 ; *C1CCc2ccccc21 ; *C1Cc2ccccc2C1 ; *C1CCCc2ncccc21]

Non-limiting exemplary optionally substituted cycloalkyl groups include:

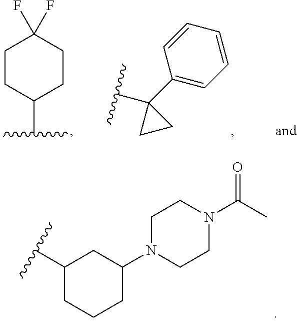 [SMILES: *C1CCC(F)(F)CC1 ; *C1(c2ccccc2)CC1 ; *C1CCCC(N2CCN(C(C)=O)CC2)C1]

The term “heterocyclo” as used herein by itself or as part of another group refers to saturated and partially unsaturated, e.g., containing one or two double bonds, monocyclic, bicyclic, or tricyclic groups containing three to fourteen ring members, i.e., a 3- to 14-membered heterocyclo, comprising one, two, three, or four heteroatoms. Each heteroatom is independently oxygen, sulfur, or nitrogen. Each sulfur atom is independently oxidized to give a sulfoxide, i.e., S(═O), or sulfone, i.e., S(═O)₂.

The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one.

The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In one embodiment, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In another embodiment, the heterocyclo group is a 5- to 8-membered cyclic group containing one ring and one or two nitrogen atoms and, optionally, one —CH₂— group is replaced with one —C(═O)— group. In another embodiment, the heterocyclo group is a 5- or 6-membered cyclic group containing one ring and one or two nitrogen atoms and, optionally, one —CH₂— group is replaced with one —C(═O)— group. In another embodiment, the heterocyclo group is a 8- to 12-membered cyclic group containing two rings and one or two nitrogen atoms. The heterocyclo can be linked to the rest of the molecule through any available carbon or nitrogen atom. Non-limiting exemplary heterocyclo groups include:

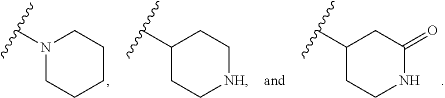

The term “optionally substituted heterocyclo” as used herein by itself or part of another group refers to a heterocyclo group that is either unsubstituted or substituted with one to four substituents, wherein each substituent is independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.” Substitution may occur on any available carbon or nitrogen atom of the heterocyclo group. Non-limiting exemplary optionally substituted heterocyclo groups include:

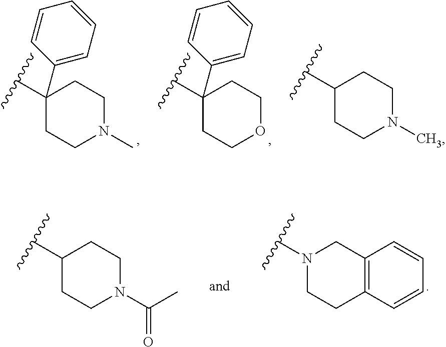

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “optionally substituted aryl” as used herein by itself or as part of another group refers to aryl that is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In one embodiment, the optionally substituted aryl is an optionally substituted phenyl. In another embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl includes aryl groups having fused optionally substituted cycloalkyl groups and fused optionally substituted heterocyclo groups. Non-limiting examples include: 2,3-dihydro-1H-inden-1-yl, 1,2,3,4-tetrahydronaphthalen-1-yl, 1,3,4,5-tetrahydro-2H-benzo[c]azepin-2-yl, 1,2,3,4-tetrahydroisoquinolin-1-yl, and 2-oxo-2,3,4,5-tetrahydro-1H-benzo[d]azepin-1-yl.

The term “heteroaryl” as used herein by itself or as part of another group refers to monocyclic and bicyclic aromatic ring systems having five to 14 fourteen ring members, i.e., a 5- to 14-membered heteroaryl, comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In one embodiment, the optionally substituted heteroaryl has two substituents. In another embodiment, the optionally substituted heteroaryl has one substituent. Any available carbon or nitrogen atom can be substituted.

The term “5-membered heteroarylenyl” as used herein by itself or part of another group refers to a divalent form of an optionally substituted 5-membered heteroaryl group. In one embodiment, the heteroarylenyl is a substituted 5-membered heteroarylenyl. In one embodiment, the heteroarylenyl is an unsubstituted 5-membered heteroarylenyl. Non-limiting exemplary 5-membered heteroarylenyls include:

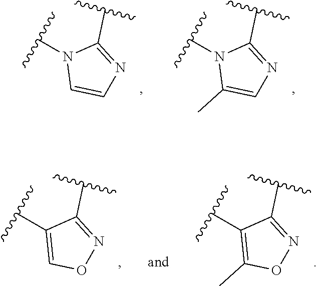

The term “aryloxy” as used herein by itself or as part of another group refers to an optionally substituted aryl attached to a terminal oxygen atom. A non-limiting exemplary aryloxy group is PhO—.

The term “heteroaryloxy” as used herein by itself or as part of another group refers to an optionally substituted heteroaryl attached to a terminal oxygen atom. A non-limiting exemplary aryloxy group is pyridyl-O—.

The term “aralkyloxy” as used herein by itself or as part of another group refers to an aralkyl attached to a terminal oxygen atom. A non-limiting exemplary aralkyloxy group is PhCH₂O—.

The term “(cyano)alkyl” as used herein by itself or as part of another group refers to an alkyl substituted with one, two, or three cyano groups. In one embodiment, the alkyl is substituted with one cyano group. In another embodiment, the alkyl is a C₁-C₆ alkyl In another embodiment, the alkyl is a C₁-C₄ alkyl. Non-limiting exemplary (cyano)alkyl groups include —CH₂CH₂CN and —CH₂CH₂CH₂CN.

The term “(cycloalkyl)alkyl” as used herein by itself or as part of another group refers to an alkyl substituted with one or two optionally substituted cycloalkyl groups. In one embodiment, the cycloalkyl group(s) is an optionally substituted C₃-C₆ cycloalkyl. In another embodiment, the alkyl is a C₁-C₆ alkyl. In another embodiment, the alkyl is a C₁-C₄ alkyl. In another embodiment, the alkyl is a C₁ or C₂ alkyl. In another embodiment, the alkyl is substituted with one optionally substituted cycloalkyl group. In another embodiment, the alkyl is substituted with two optionally substituted cycloalkyl groups. Non-limiting exemplary (cycloalkyl)alkyl groups include:

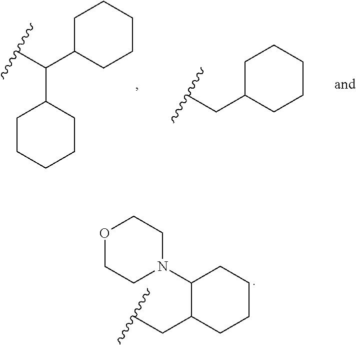 [SMILES: *C(C1CCCCC1)C1CCCCC1 ; *CC1CCCCC1 ; *CC1CCCCC1N1CCOCC1]

The term “sulfonamido” as used herein by itself or as part of another group refers to a radical of the formula —SO₂NR^{50a}R^{50b}, wherein R^{50a} and R^{50b} are each independently hydrogen, alkyl, optionally substituted cycloalkyl, optionally substituted heterocyclo, optionally substituted aryl, or optionally substituted heteroaryl; or R^{50a} and R^{50b} taken together with the nitrogen to which they are attached form a 3- to 8-membered optionally substituted heterocyclo group. Non-limiting exemplary sulfonamido groups include —SO₂NH₂, —SO₂N(H)CH₃, and —SO₂N(H)Ph.

The term “carboxamido” as used herein by itself or as part of another group refers to a radical of the formula —C(═O)NR^{50c}R^{50d}, wherein R^{50c} and R^{50d} are each independently hydrogen, alkyl, optionally substituted cycloalkyl, optionally substituted heterocyclo, optionally substituted aryl, or optionally substituted heteroaryl; or R^{50c} and R^{50d} taken together with the nitrogen to which they are attached form a 3- to 8-membered optionally substituted heterocyclo group. Non-limiting exemplary carboxamido groups include —C(═O)NH₂, —C(═O)(H)CH₃, and —C(═O)N(CH₃)₂.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A non-limiting exemplary alkylcarbonyl group is —COCH₃.

The term “arylcarbonyl” as used herein by itself or as part of another group refers to a carbonyl group, i.e., —C(═O)—, substituted by an optionally substituted aryl group. A non-limiting exemplary arylcarbonyl group is —COPh.

The term “alkylsulfonyl” as used herein by itself or as part of another group refers to a sulfonyl group, i.e., —SO₂—, substituted by an alkyl group. A non-limiting exemplary alkylsulfonyl group is —SO₂CH₃.

The term “arylsulfonyl” as used herein by itself or as part of another group refers to a sulfonyl group, i.e., —SO₂—, substituted by an optionally substituted aryl group. A non-limiting exemplary arylsulfonyl group is —SO₂Ph.

The term “mercaptoalkyl” as used herein by itself or as part of another group refers to an alkyl substituted by a —SH group.

The term “carboxy” as used by itself or as part of another group refers to a radical of the formula —C(═O)OH.

The term “ureido” as used herein by itself or as part of another group refers to a radical of the formula —NR^{51a}—C(═O)—NR^{51b}R^{51c}, wherein R^{51a} is hydrogen or alkyl; and R^{51b} and R^{51c} are each independently hydrogen, alkyl, optionally substituted cycloalkyl, optionally substituted heterocyclo, optionally substituted aryl, or optionally substituted heteroaryl, or R^{51b} and R^{51c} taken together with the nitrogen to which they are attached form a 4- to 8-membered optionally substituted heterocyclo group. Non-limiting exemplary ureido groups include —NH—C(C═O)—NH₂ and —NH—C(C═O)—NHCH₃.

The term “guanidino” as used herein by itself or as part of another group refers to a radical of the formula —NR^{52a}—C(═NR⁵³)—NR^{52b}R^{52c}, wherein R^{52a} is hydrogen or alkyl; R^{52b} and R^{53c} are each independently hydrogen, alkyl, optionally substituted cycloalkyl, optionally substituted heterocyclo, optionally substituted aryl, or optionally substituted heteroaryl; or R^{52b} and R^{52c} taken together with the nitrogen to which they are attached form a 4- to 8-membered optionally substituted heterocyclo group; and R⁵³ is hydrogen, alkyl, cyano, alkylsulfonyl, alkylcarbonyl, carboxamido, or sulfonamido. Non-limiting exemplary guanidino groups include —NH—C(C═NH)—NH₂, —NH—C(C═NCN)—NH₂, and —NH—C(C═NH)—NHCH₃.

The term “(heterocyclo)alkyl” as used herein by itself or as part of another group refers to an alkyl substituted with one, two, or three optionally substituted heterocyclo groups. In one embodiment, the alkyl is substituted with one optionally substituted 5- to 8-membered heterocyclo group. In another embodiment, alkyl is a C₁-C₆ alkyl. In another embodiment, alkyl is a C₁-C₄ alkyl. The heterocyclo group can be linked to the alkyl group through a carbon or nitrogen atom. Non-limiting exemplary (heterocyclo)alkyl groups include:

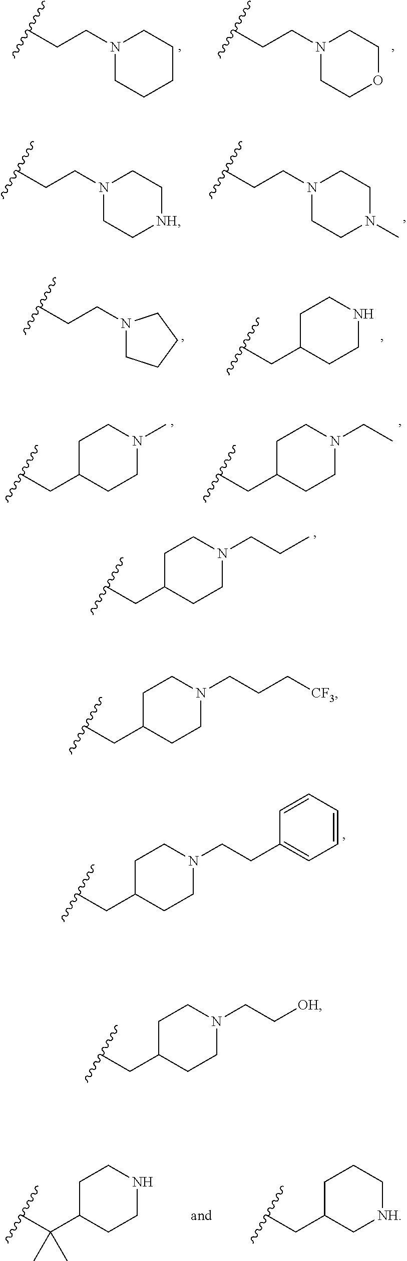

The term “carbamate” as used herein by itself or as part of another group refers to a radical of the formula —NR^{54a}—C(═O)—OR^{54b}, wherein R^{54a} is hydrogen or alkyl, and R^{54b} is hydrogen, alkyl, optionally substituted cycloalkyl, optionally substituted heterocyclo, optionally substituted aryl, or optionally substituted heteroaryl. A non-limiting exemplary carbamate group is —NH—(C═O)—OtBu.

The term “(heteroaryl)alkyl” as used herein by itself or as part of another group refers to an alkyl substituted with one or two optionally substituted heteroaryl groups. In one embodiment, the alkyl group is substituted with one optionally substituted 5- to 14-membered heteroaryl group. In another embodiment, the alkyl group is substituted with two optionally substituted 5- to 14-membered heteroaryl groups. In another embodiment, the alkyl group is substituted with one optionally substituted 5- to 9-membered heteroaryl group. In another embodiment, the alkyl group is substituted with two optionally substituted 5- to 9-membered heteroaryl groups. In another embodiment, the alkyl group is substituted with one optionally substituted 5- or 6-membered heteroaryl group. In another embodiment, the alkyl group is substituted with two optionally substituted 5- or 6-membered heteroaryl groups. In one embodiment, the alkyl group is a C₁-C₆ alkyl. In another embodiment, the alkyl group is a C₁-C₄ alkyl. In another embodiment, the alkyl group is a C₁ or C₂ alkyl. Non-limiting exemplary (heteroaryl)alkyl groups include:

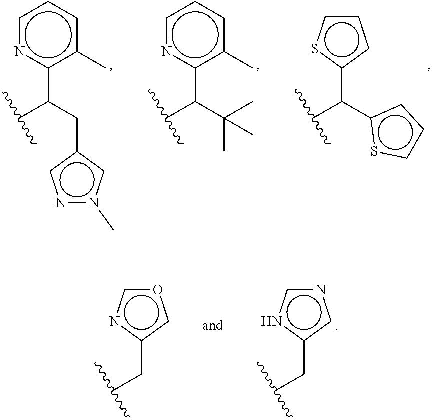

The terms “aralkyl” or “(aryl)alkyl” as used herein by themselves or as part of another group refers to an alkyl substituted with one, two, or three optionally substituted aryl groups. In one embodiment, the alkyl is substituted with one optionally substituted aryl group. In another embodiment, the alkyl is substituted with two optionally substituted aryl groups. In one embodiment, the aryl is an optionally substituted phenyl or optionally substituted naphthyl. In another embodiment, the aryl is an optionally substituted phenyl. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (aryl)alkyl groups include benzyl, phenethyl, —CHPh₂, and —CH(4-F-Ph)₂.

The term “amido” as used herein by itself or as part of another group refers to a radical of formula —C(═O)NR^{60a}R^{60b}, wherein R^{60a} and R^{60b} are each independently hydrogen, optionally substituted alkyl, optionally substituted alkenyl, optionally substituted alkynyl, haloalkyl, (alkoxy)alkyl, (hydroxy)alkyl, (cyano)alkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl; or R^{60a} and R^{60b} taken together with the nitrogen to which they are attached from a 4- to 8-membered optionally substituted heterocyclo group. In one embodiment, R^{60a} and R^{60b} are each independently hydrogen or C₁-C₆ alkyl.

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In one embodiment, the amino is —NH₂.

In another embodiment, the amino is an “alkylamino,” i.e., an amino group wherein R^{55a} is C₁₋₆ alkyl and R^{55b} is hydrogen. In one embodiment, R^{55a} is C₁-C₄ alkyl. Non-limiting exemplary alkylamino groups include —N(H)CH₃ and —N(H)CH₂CH₃.

In another embodiment, the amino is a “dialkylamino,” i.e., an amino group wherein R^{55a} and R^{55b} are each independently C₁₋₆ alkyl. In one embodiment, R^{55a} and R^{55b} are each independently C₁-C₄ alkyl. Non-limiting exemplary dialkylamino groups include —N(CH₃)₂ and —N(CH₃)CH₂CH(CH₃)₂.

In another embodiment, the amino is a “hydroxyalkylamino,” i.e., an amino group wherein R^{55a} is (hydroxyl)alkyl and R^{55b} is hydrogen or C₁-C₄ alkyl.

In another embodiment, the amino is a “cycloalkylamino,” i.e., an amino group wherein R^{55a} is optionally substituted cycloalkyl and R^{55b} is hydrogen or C₁-C₄ alkyl.

In another embodiment, the amino is a “aralkylamino,” i.e., an amino group wherein R^{55a} is aralkyl and R^{55b} is hydrogen or C₁-C₄ alkyl. Non-limiting exemplary aralkylamino groups include —N(H)CH₂Ph, —N(H)CHPh₂, and —N(CH₃)CH₂Ph.

In another embodiment, the amino is a “(cycloalkyl)alkylamino,” i.e., an amino group wherein R^{55a} is (cycloalkyl)alkyl and R^{55b} is hydrogen or C₁-C₄ alkyl. Non-limiting exemplary (cycloalkyl)alkylamino groups include:

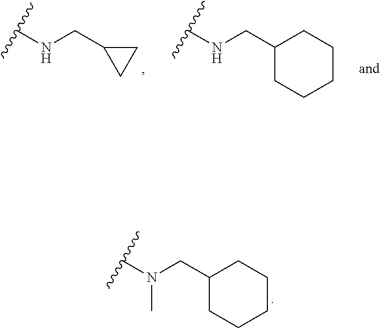

In another embodiment, the amino is a “(heterocyclo)alkylamino,” i.e., an amino group wherein R^{55a} is (heterocyclo)alkyl and R^{55b} is hydrogen or C₁-C₄ alkyl. Non-limiting exemplary (heterocyclo)alkylamino groups include:

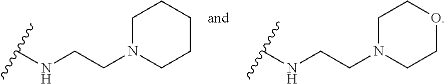 [SMILES: *NCCN1CCCCC1 ; "and" ; *NCCN1CCOCC1]

The term “(amino)alkyl” as used herein by itself or as part of another group refers to an alkyl substituted with one amino group. In one embodiment, the amino group is —NH₂. In one embodiment, the amino group is an alkylamino. In another embodiment, the amino group is a dialkylamino. In another embodiment, the alkyl is a C₁-C₆ alkyl. In another embodiment, the alkyl is a C₁-C₄ alkyl. Non-limiting exemplary (amino)alkyl groups include —CH₂NH₂, CH₂CH₂N(H)CH₃, —CH₂CH₂N(CH₃)₂, CH₂N(H)cyclopropyl, —CH₂N(H)cyclobutyl, and —CH₂N(H)cyclohexyl, and —CH₂CH₂CH₂N(H)CH₂Ph and —CH₂CH₂CH₂N(H)CH₂(4-CF₃-Ph).

The present disclosure encompasses any of the compounds of Formula I being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, C, ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. In one embodiment, provided is a compound wherein substantially all of the atoms at a position within the Substance X are replaced by an atom having a different atomic mass or mass number. In another embodiment, provided is a compound wherein substantially all of the atoms at a position within the Substance X are replaced by deuterium atoms, e.g., all of the hydrogen atoms of a —CH₃ group are replaced by deuterium atoms to give a —CD₃ group. In another embodiment, provided is a compound wherein a portion of the atoms at a position within the Substance X are replaced, i.e., the Substance X is enriched at a position with an atom having a different atomic mass or mass number. In another embodiment, provided is a compound wherein none of the atoms of the Substance X are replaced by an atom having a different atomic mass or mass number. Isotopically-labelled compounds of Formula I can be prepared by methods known in the art.

compounds of Formula I may contain one or more asymmetric centers and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, compounds of Formula I are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The term “about,” as used herein, includes the recited number±10%. Thus, “about 10” means 9 to 11.

The pharmaceutical composition can be manufactured, for example, by conventional mixing, dissolving, granulating, dragee-making, emulsifying, encapsulating, entrapping, or lyophilizing processes. Proper formulation is dependent upon the route of administration chosen. The pharmaceutical composition typically is in the form of a tablet, capsule, powder, solution, or elixir. When administered in tablet form, the pharmaceutical composition additionally can contain a solid carrier, such as a gelatin or an adjuvant. The tablet, capsule, and powder contain about 0.01% to about 95%, and preferably from about 1% to about 50%, of a substance X. When administered in liquid form, a liquid carrier, such as water, petroleum, or oils of animal or plant origin, can be added. The liquid form of the pharmaceutical composition can further contain physiological saline solution, dextrose or other saccharide solutions, or glycols. When administered in liquid form, the pharmaceutical composition contains about 0.1% to about 90%, and preferably about 1% to about 50%, by weight, of a substance X.

When the pharmaceutical composition is administered by intravenous, cutaneous, or subcutaneous injection, the pharmaceutical composition is in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, cutaneous, or subcutaneous injection typically contains, an isotonic vehicle.

The substance X can be readily combined with pharmaceutically acceptable carriers well-known in the art. Standard pharmaceutical carriers are described in Remington's Pharmaceutical Sciences, Mack Publishing Co., Easton, PA, 19th ed. 1995. Such carriers enable the active agents to be formulated as tablets, pills, dragees, capsules, liquids, gels, syrups, slurries, suspensions and the like, for oral ingestion by a subject to be treated. Pharmaceutical preparations for oral use can be obtained by adding the Substance X to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

The pharmaceutical composition can be formulated for parenteral administration by injection, e.g., by bolus injection or continuous infusion. Formulations for injection can be presented in unit dosage form, e.g., in ampules or in multidose containers, with an added preservative. The pharmaceutical composition can take such forms as suspensions, solutions, or emulsions in oily or aqueous vehicles, and can contain formulatory agents such as suspending, stabilizing, and/or dispersing agents.

The pharmaceutical composition for parenteral administration include aqueous solutions of the active agent in water-soluble form. Additionally, suspensions of a Substance X can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, the pharmaceutical composition can be in powder form for constitution with a suitable vehicle, e.g., sterile pyrogen-free water, before use.

The pharmaceutical composition also can be formulated in rectal compositions, such as suppositories or retention enemas, e.g., containing conventional suppository bases. In addition to the formulations described previously, the pharmaceutical composition also can be formulated as a depot preparation. Such long-acting formulations can be administered by implantation (for example, subcutaneously or intramuscularly) or by intramuscular injection. Thus, for example, the Substance X can be formulated with suitable polymeric or hydrophobic materials (for example, as an emulsion in an acceptable oil) or ion exchange resins.

In particular, the pharmaceutical composition can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Substance X also can be injected parenterally, for example, intravenously, intramuscularly, subcutaneously, or intracoronarily. For parenteral administration, the Substance X are typically used in the form of a sterile aqueous solution which can contain other substances, for example, salts or monosaccharides, such as mannitol or glucose, to make the solution isotonic with blood.

The use of any and all examples, or exemplary language (e.g., “in some embodiments”) provided herein, is intended to better illustrate the invention and is not a limitation on the scope of the invention unless otherwise claimed. No language in the specification should be construed as indicating any non-claimed element as essential to the practice of the invention.

### BRIEF DESCRIPTION OF THE DRAWINGS

FIG. 1. Cpd. 73 increased the HBG mRNA level in human CD34+ HSC.

FIG. 2. Cpd. 73 increased the HbF concentration in human CD34+ HSC.

FIG. 3. Cpd. 73 increased the % of HbF+ cells in human CD34+ HSC.

FIG. 4A-4B. Cpd. 73 significantly reduced DAI and improved body weight loss in TNBS induced IBD mice model.

FIG. 5A-5B. Cpd. 73 significantly decreased colon weight and increased colon length in TNBS induced IBD mice model.

FIG. 6A-6D. Cpd. 73 significantly decreased the elevated neutrophils and monocytes and improved anemia in peripheral blood in TNBS induced IBD mice model.

FIG. 7A-7E. Cpd. 73 significantly reduced the elevated neutrophils, NK cells, activated NK cells, Th1 (IFN-γ secreting CD4+ T) cells and Macrophage cells in mesenteric lymph nodes (MLN) in TNBS induced IBD mice model.

FIG. 8. Cpd. 73 significantly decreased pathological score in TNBS induced IBD mice model.

FIG. 9. Cpd. 73 significantly reduced the fibrosis score in TNBS induced IBD mice model.

FIG. 10A-10B. Cpd. 73 significantly reduced DAI and improved body weight loss in T cell transfer induced IBD mice model.

FIG. 11A-11B. Cpd. 73 significantly decreased colon weight and increased colon length in T cell transfer induced IBD mice model.

FIG. 12. Cpd. 73 significantly decreased pathological score in T cell transfer induced IBD mice model.

FIG. 13. Cpd. 73 significantly decreased colon density (colon weight/colon length) in DSS induced IBD mice model.

FIG. 14. Cpd. 73 significantly decreased pathological score in DSS induced IBD mice model.

FIG. 15. Cpd. 73 dose-dependently improved the body weight of rat with chronic kidney disease-induced anemia.

FIG. 16. Cpd. 73 dose-dependently improved RBC of rat with chronic kidney disease-induced anemia.

FIG. 17. Cpd. 73 dose-dependently improved HGB of rat with chronic kidney disease-induced anemia.

FIG. 18. Cpd. 73 dose-dependently improved HCT of rat with chronic kidney disease-induced anemia.

FIG. 19. Cpd. 73 dose-dependently improved RET of rat with chronic kidney disease-induced anemia.

FIG. 20. Combination of Cpd. 73 and EPO further improved the body weight of rat with chronic kidney disease-induced anemia.

FIG. 21. Combination of Cpd. 73 and EPO further improved RBC of rat with chronic kidney disease-induced anemia.

FIG. 22. Combination of Cpd. 73 and EPO further improved HGB of rat with chronic kidney disease-induced anemia.

FIG. 23. Combination of Cpd. 73 and EPO further improved HCT of rat with chronic kidney disease-induced anemia.

FIG. 24. Combination of Cpd. 73 and EPO further improved RET of rat with chronic kidney disease-induced anemia.

### DETAILED DESCRIPTION OF THE PREFERRED EMBODIMENT

The following examples further illustrate the present invention, but the present invention is not limited thereto.

The Cpd. 73 used in the following Embodiment is

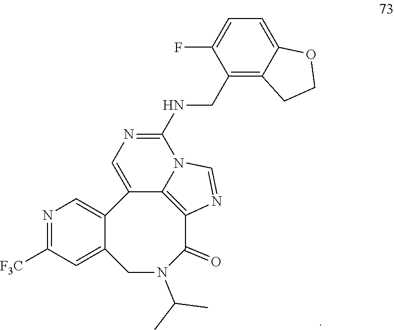 [SMILES: CC(C)N1Cc2cc(C(F)(F)F)ncc2-c2cnc(NCc3c(F)ccc4c3CCO4)n3cnc(c23)C1=O]

### Embodiment 1

### 1. Animals

Female CD-1 mice (8 weeks old) were housed and handled in a temperature-controlled environment with a 12-h light/12-h dark cycle. A total of 25 mice were assigned to 5 groups by randomization based on body weight, followed by 5 days of treatment with vehicle (po, qd), 3 mg/kg Cpd. 73 (po, qd), 10 mg/kg Cpd. 73 (po, qd), 30 mg/kg Cpd. 73 (po, qd), or 90 mg/kg Cpd. 73 (po, qd). The protocols and procedures involving the care and use of animals were approved by the Institutional Animal Care and Use Committee (IACUC) of WuXiAppTec (Shanghai) Co., Ltd. (Shanghai, China).

### 2. Determination of H3K27Me3 Level in Bone Marrow Cells and PBMC Monocytes.

Mice were euthanatized 4 hours post the last dose. Whole blood and bone marrow were collected. Bone marrow cells were flushed from femur and tibia with IMDM+/+ (IMDM, 10% heat inactivated FBS, 100 U/mL penicillin and 100 μg/mL streptomycin). Aggregate was removed by passing through 40 μm cell strainer. 5 mL DPBS was used to pellet the cells after centrifugation, and the cell number was counted. Whole blood was added with 1×RBC lysis buffer remove RBC (whole blood: 1×RBC lysis buffer=1:9), and mononuclear cells were collected by centrifugation. The cells were then washed with 1×DPBS for once and resuspended in 3 ml DPBS after centrifugation followed by cell number counting.

Bone marrow cells were stained with FITC-conjugated anti-mouse TER-119 antibody (Thermo Fisher, 11-5921-82) and eFluor 506-conjugated anti-mouse CD45 antibody (Thermo Fisher, 69-0451-82). TER-119+ cells were erythroid lineage cells. PBMC were stained with eFlour 450-conjugated anti-mouse CD11b antibody (Invitrogen, 48-0112-82), PerCP-eFluor710-conjugated anti-mouse CD3 antibody (Invitrogen, 46-0032-82), eFluor 506-conjugated anti-mouse CD45 antibody (Thermo Fisher, 69-0451-82). CD45+CD3−CD11b+ cells were monocytes from PBMC.

After washing, centrifugation and fixation, the cells were permeabilized and further incubated with antibodies of H3K27m3 (Tri-Methyl-Histone H3 (Lys27) (C36B11) Rabbit mAb (PE Conjugate), CST, 40724S) and H3 (Histone H3 (D1H2) XP® Rabbit mAb (Alexa Fluor®647 Conjugate), CST, 12230S), followed by FACS analysis to quantify the mean fluorescence intensity of target cells. The ratio between the fluorescence intensity of H3K27me3 and H3 were used to reflect the level of H3K27me3 in the cells.

Cpd. 73 was supposed to inhibit H3K27me3 of erythroid lineage cells in the bone marrow. Compared with vehicle group, Cpd. 73 treatment decreased the H3K27me3 level in TER119+ erythroid lineage cells from the bone marrow of CD-1 mice. 21.2%, 18.7% and 21.6% of decrease in H3K27me3 level was observed when the mice were treated with 3 mg/kg, 10 mg/kg and 30 mg/kg respectively (qd×5 days), and a more profound inhibition was observed at 90 mg/kg (qd×5 days), with 39.7% of decrease in H3K27me3 level (p<0.01, vs. vehicle).

The H3K27me3 level in PBMC monocytes was used as another PD marker. Compared with vehicle group, Cpd. 73 treatment decreased the H3K27me3 level in PBMC monocytes. The percent of decrease were 17.9%, 40.5%, 8.8% and 41.2% when Cpd. 73 were dosed at 3 mg/kg, 10 mg/kg, 30 mg/kg and 90 mg/kg respectively.

3. Determination of Hbb-Bh1 mRNA Level in Whole Blood

Mouse Hbb-bh1 gene, the homolog to human HBG1 gene encoding γ hemoglobin, is used as an efficacy biomarker in wide-type CD-1 mice. One hundred and twenty μL whole blood was collected for RNA isolation following the instructions of Trizol LS reagent (Invitrogen, 10296028). The relative expression of Hbb-bh1 mRNA to GAPDH mRNA were quantified by qPCR. The relative expression of Hbb-bh1 mRNA in the whole blood of CD-1 mice increased with increasing dose of Cpd. 73 from 3 mg/kg (qd×5 days) to 90 mg/kg (qd×5 days). The Hbb-bh1 mRNA levels were 82.2%, 114.6% and 178.3% of the vehicle control group when Cpd. 73 was dosed at 3 mg/kg, 10 mg/kg and 30 mg/kg respectively, and a 3.04-fold of increase was observed at 90 mg/kg Cpd. 73 (p<0.05, vs. vehicle). The increased expression of Hbb-bh1 mRNA may result in increased expression of mouse βh1 hemoglobin. In human, a similarly increased expression of HBG1 gene and HBG2 gene is expected which will ultimately bring therapeutic benefits to SCD patients by increasing the % of HbF in peripheral blood.

4. Determination of HBG mRNA Level, HbF Concentration and HbF+ Cell % in Human CD34+ HSC

### Cell Culture and Drug Treatment

Human cord blood CD34+ hematopoietic stem cells (HSC) were first expanded in the expansion medium (StemSpan SFEM II+StemSpan CD34 Expansion Supplement) to achieve the desired number of cells, and then differentiated in differentiation medium (StemSpan SFEM II+StemSpan Erythroid Expansion Supplement (100×)) for three days without drug treatment. Various concentrations of Cpd. 73 were added on the fourth day of differentiation and further incubated with the cells for another 7 days. 33 mM Hydroxyurea was used as the control.

4.1 Determination of HBG mRNA Level

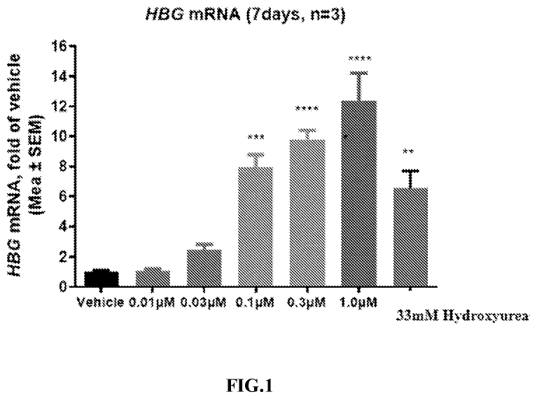

After 7 days of treatment, the cells were collected. Total RNA was isolated using Trizol LS reagent (Invitrogen-10296028) and 1 μg RNA of each sample was reverse transcribed into cDNA using High Capacity cDNA Reverse Transcription Kit (AB (Applied Biosystems)-4374966), according to the manufacturer's instructions. Quantitative RT-PCR (qRT-PCR) was performed using an Applied Biosystems QuantStudio 7 Flex system with the primer pairs shown as below: hHBG-mRNA-F1 (5′-TGGCAAGAAGGTGCTGACTTC-3′) and hHBG-mRNA-R1 (5′-TCACTCAGCTGGGCAAAGG-3′). As seen in FIG. 1, Cpd. 73 showed a concentration-dependent induction effect on HBG mRNA in human CD34+ HSC. The HBG mRNA level were 1.09-, 2.49-, 7.84-, 9.69- and 12.2-folds of the vehicle group when the cells were treated with 0.01 μM, 0.03 μM, 0.1 μM, 0.3 μM and 1.0 μM Cpd. 73 respectively. The maximal effect was observed at 1.0 μM Cpd. 73 which increased HBG mRNA by 12.2 folds (p<0.0001, vs. vehicle). The HBG mRNA level of cells treated with 33 mM Hydroxyurea, an approved medicine for SCD, was 6.46-folds of the vehicle group.

### 4.2 Determination of HbF Concentration by ELISA

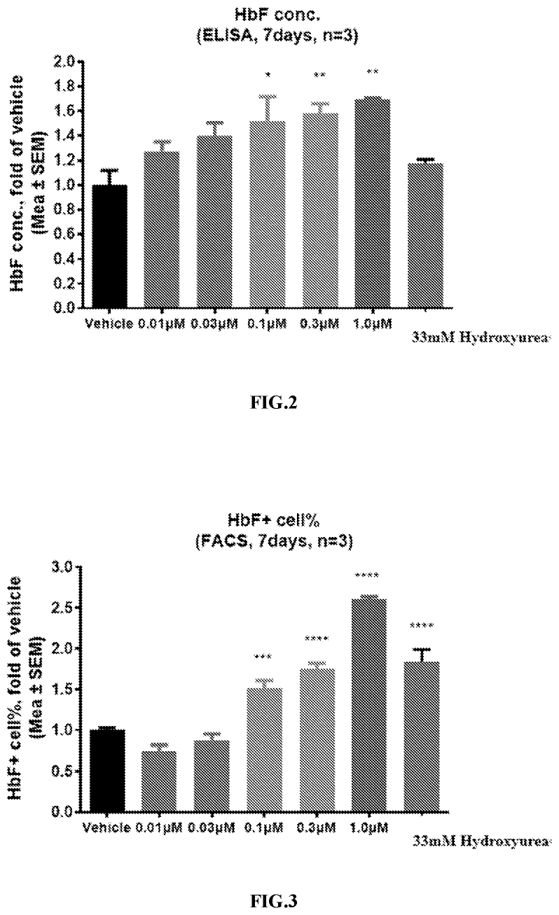

After 7 days of treatment, the cells were collected and lysed. The concentration of HbF in the cell lysate was determined using a Human Fetal Hemoglobin (HBF) ELISA Kit (MyBioSource, MBS2024474). As seen in FIG. 2, Cpd. 73 showed a concentration-dependent induction effect on HbF production in human CD34+ HSC. The cellular HbF concentration were 1.27-, 1.39-, 1.52-, 1.58- and 1.69-folds of the vehicle group when the cells were treated with 0.01 μM, 0.03 μM, 0.1 μM, 0.3 μM and 1.0 μM Cpd. 73 respectively. The maximal effect was observed at 1.0 μM Cpd. 73 where the cellular HbF concentration was 1.69-folds of the vehicle group (p<0.01). HbF concentration of cells treated with 33 mM Hydroxyurea was 1.18-folds of the vehicle group. Clinically, small increases of HbF concentration (1-5%) have the potential to provide clinical benefits to all SCD patients.

### 4.3 Determination of HbF+ Cell % by FACS

After 7 days of treatment, the cells were collected and stained with PE-conjugated Mouse Anti-Human Fetal Hemoglobin (BD, 560041). The percentage of HbF+ cells were quantified by FACS. As seen in FIG. 3, the % of HbF+ cells were 0.73-, 0.86-, 1.50-, 1.73- and 2.58-folds of the vehicle group when the cells were treated with 0.01 μM, 0.03 μM, 0.1 μM, 0.3 μM and 1.0 μM Cpd. 73 respectively. The maximal effect was observed at 1.0 μM Cpd. 73 where HbF+ cell % was 2.58-folds of the vehicle group (p<0.0001). The % of HbF+ cells was 1.82-folds of the vehicle group when the cells were treated with 33 mM Hydroxyurea.

### Embodiment 2

### Example in TNBS Induced IBD Model

### 1. Experimental Protocol for TNBS Induced Inflammatory Bowel Disease (IBD) Model in Balb/c Mice

There are two kinds of IBD: Crohn's disease (CD) and ulcerative colitis (UC). Acute TNBS administration results in a preclinical type 1 immunity induced mouse model replicating clinical Crohn's disease.

Female Balb/c mice (8 weeks old) were obtained from Beijing Vital River Laboratory Animal Co. Ltd. Animals, and then housed and handled in a temperature-controlled environment with a 12-h light/12-h dark cycle. A total of 30 mice were assigned to 3 groups by randomization based on body weight.

On Day 0, the mice weighing 18-20 g were anesthetized with Avidin (Easycheck, M2910), and then further intra-rectally injected 100 μL 1.5% TNBS solution (final concentration in 50% ethanol) in the vehicle group and treatment groups. For the Sham group, the mice were intra-rectally injected with 50% ethanol at the same volume.

Mice in Group 1 and Group 2 were treated with vehicle for 8 days (po, qd, from day −1 to day 6). Mice in Group 3 were treated with 90 mg/kg Cpd. 73 for 8 days (po, qd, from day −1 to day 6). The protocols and procedures involving the care and use of animals were approved by the Institutional Animal Care and Use Committee (IACUC) at WuxiApptec (Shanghai, China). The grouping is shown in table 2.

TABLE 2

| | | | Number |
|---|---|---|---|
| Group | Treatment | Dosage | of mice |
| Group 1 (G1) | Sham | po, qd, from day −1 to day 6 (8 D) | 10 |
| Group 2 (G2) | Vehicle | po, qd, from day −1 to day 6 (8 D) | 10 |
| Group 3 (G3) | Cpd. 73 | 90 mg/kg, po, qd, from day −1 to | 10 |
| | | day 6 (8 D) | |

### 2. Clinical Scores

Clinical signs of IBD were assessed every day based on the score of disease activity index (DAI) which was evaluated from three parameters using a scoring system from 0 to 4: stool consistency (0, normal stool; 1, soft but still formed stool; 2, soft and not formed stool; 3, very soft and wet stool; 4, watery diarrhea), bleeding score (0, negative hemoccult; 1, weak positive hemoccult; 2, positive hemoccult; 3, blood trace in stool visible; 4, gross rectal bleeding) and body weight loss (0, no body weight loos; 1, 1-5% body weight loss; 2, 6-10% body weight loss; 3, 11-20% body weight loss; 4, >20% body weight loss).

TABLE 3

| DAI | BW | | |
|---|---|---|---|
| score | loss % | Stoll consistency | Bleeding |
| 0 | 0 | Normal | Negative hemoccult |
| 1 | 1-5 | Soft but still formed | Weak positive |
| | | | hemoccult |
| 2 | 6-10 | Soft | Positive hemoccult |
| 3 | 11-20 | Very soft; wet | Blood trace in stool |
| | | | visible |
| 4 | >20 | Watery diarrhea | Gross rectal bleeding |

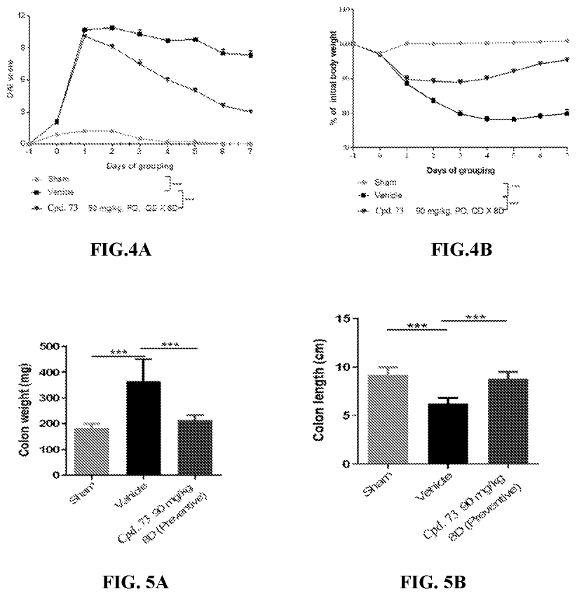

As seen in FIG. 4A-4B, mice from Group 1 (sham control) showed almost no symptoms of DAI (DAI score less than 2 during the entire study). Compared with G1 sham control group, after TNBS intrarectal instillation, there was a higher DAI score in the G2 group reaching the maximal clinical score of 10.70±0.14 on day 1. Cpd. 73 showed efficacy by significantly reducing the DAI score of IBD and improving body weight loss. These results indicated that treatment of Cpd. 73 could ameliorate the progression TNBS-induced IBD in mice.

### 3. Colon Weight and Colon Length Measurement

At the end of experiment (day 7), the animals were euthanized, dissected and the entire colon was quickly removed and gently cleared of feces.

The entire colon was weighed, and the total length was measured. Colon weight gain and shortening is an indirect marker of inflammation. As expected, TNBS induced significant increase of colon weight to 336.8±26.68 mg. Treatment with Cpd. 73 significantly decreased TNBS-induced colon weight increasement to 213.8±6.08 mg (FIG. 5A).

For the colon length, TNBS induced significant decrease of the colon length to 6.26±0.17 cm. Treatment with 90 mg/kg Cpd. 73 significantly improved the colon length to 8.79±0.22 cm (FIG. 5B).

These results indirectly indicate that treatment of Cpd. 73 improved the inflammation in colon in TNBS-induced colitis mice.

### 4. Whole Blood Cell Counting

At the end of experiment, the animals were euthanized, and blood was collected immediately by the heart punctures and used for whole blood cell analysis.

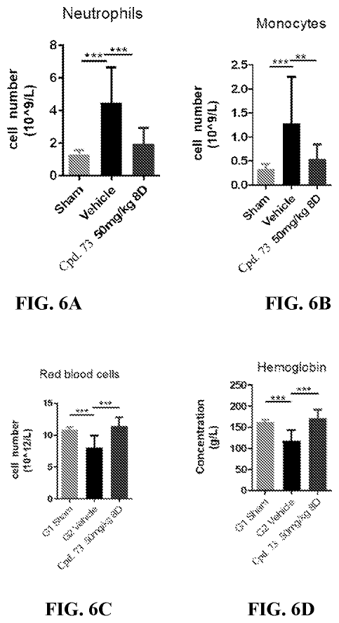

Neutrophils and monocytes as the important components of the innate immune response, are key regulators of intestinal microenvironment homeostasis which promote the development of IBD. Most intestinal macrophages are derived from monocytes in peripheral blood. As shown in FIG. 6A-6B, TNBS induced significant increase of neutrophils and monocytes in peripheral blood compared to sham control. Treatments with Cpd. 73 significantly reduced the elevated cell number of neutrophils and monocytes in peripheral blood. Anemia is the most common extraintestinal manifestation of IBD. TNBS induction leaded to reduction of red blood cells and hemoglobin in peripheral blood, and treatment with Cpd. 73 improved anemia in TNBS induced IBD mice model (FIG. 6C-6D).

### 5. Analysis of MLN Cell Populations by Flow Cytometry

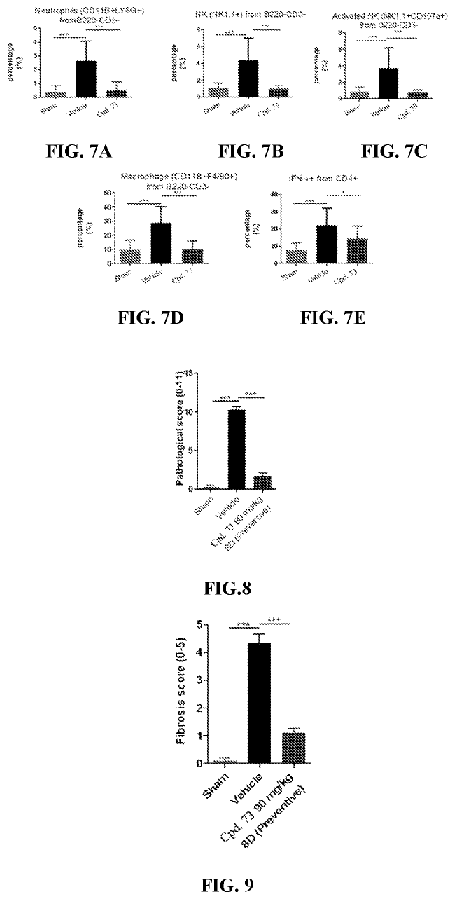

At the end of the study (day 7), the cell suspensions from sham control, model control and Cpd. 73 treatment groups were obtained from mesenteric lymph nodes (MLN). Cells were stained with the following florescence-labelled antibodies: APC-Cy7-conjugated anti-mouse CD45, BV510-conjugated anti-mouse CD3e, AF700-conjugated anti-mouse CD8a, BUV395-conjugated anti-mouse CD4, BV421-conjugated anti-mouse CD25, FITC-conjugated anti-mouse Foxp3, BV650-conjugated anti-mouse IFN-γ, APC-conjugated anti-mouse B220, BV395-conjugated anti-mouse CD3e, BV605-conjugated anti-mouse CD11b, BB700-conjugated anti-mouse CD11c, AF488-conjugated anti-mouse MHCII, BV421-conjugated anti-mouse NK1.1, BV510-conjugated anti-mouse Ly6G, PE-Cy7-conjugated anti-mouse CD107a and PE-CF594-conjugated anti-mouse F4/80. All cells were primarily gated on single and live lymphocytes based on forward scatter (FCS), side scatter (SSC) and live/dead staining buffer. Samples were analyzed on a flow cytometer (BD LSRFortessa) to count the percentage of each subtype of lymphocytes. As seen in FIG. 7A-7E, after treatment with 90 mg/kg Cpd. 73, the elevated percentages of neutrophils (CD45⁺CD3⁻B220⁻CD11B⁺LY6G⁺), NK cells (CD45⁺CD3⁻B220⁻NK1.1⁺), activated NK cells (CD45⁺CD3⁻B220⁻NK1.1⁺CD107a⁺), IFN-γ secreting CD4+ T cells (CD45⁺CD3⁺CD8⁺, Th1 cells), and macrophages (CD45⁺CD3⁻B220⁻CD11b+F4/80⁺) were significantly reduced when compared with vehicle control.

### 6. Histopathologic Assessment

At the end of the study (day 7), all the animals were sacrificed by CO2. The colon was Swiss-rolled and fixed with neutralized PFA followed by H&E staining. After that, the pathologists from WuXi clinical pathological analysis platform, who were blinded to animal ID, reviewed the H&E staining and scored. The pathological scoring standards were as follows: crypt architecture (normal, 0; severe crypt distortion with loss of entire crypts, 3), degree of inflammatory cell infiltration (normal, 0; dense inflammatory infiltrate, 3), muscle thickening (normal, 0; marked muscle thickening present, 3), goblet cell depletion (absent, 0; present, 1) and crypt abscess (absent, 0; present, 1).

As seen in FIG. 8, mice in the vehicle group (G2) showed a pathological score of 10.33±0.35, indicating a large range of inflammatory cell infiltration in the colon, while treatment with Cpd. 73 significantly reduced the score to 1.70±0.37, which was in agreement with the alleviation of clinical symptoms as indicated by the clinical scores.

### 7. Masson's Trichrome Staining

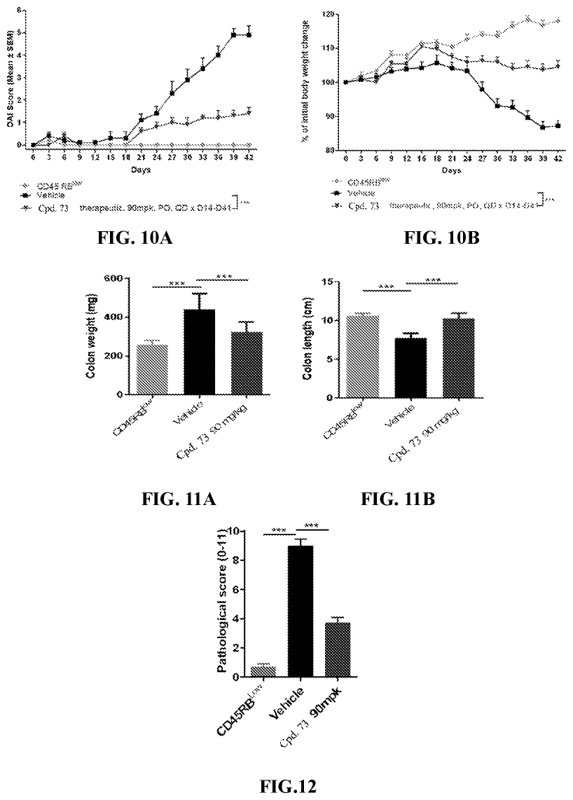

The fixed colon was stained with Masson's Trichrome to assess the collagen fibers in colon tissue. The Masson's Trichrome staining procedures was followed the standard protocol. After that, the pathological doctors from WuXi clinical pathological analysis platform reviewed the whole slices and scored blinded with animal information. The pathological scoring standards were as follows: No increase—0, Increased in the submucosa—1; Increased in the mucosa—2; Increased in the muscularis mucosa with thickening/disorganization of the muscularis mucosa-3; Increased in the muscularis propria (evident increases in collagen fibrils for Sirius red)—4; Gross disorganization of the muscularis propria—5. As seen in FIG. 9, results of Masson's Trichrome staining showed marked evidence of fibrosis in vehicle control group (4.33±0.30), compared to G1 sham control. Treatment with Cpd. 73 significantly reduced the fibrosis score to 1.10±0.17.

### Example in T Cell Transfer Induced IBD Model

### 1. Experimental Protocol for T Cell Transfer Induced Inflammatory Bowel Disease (IBD) Model in CB17 Mice

The T cell transfer model of colitis recapitulates the clinical pathology (colitis and small bowel inflammation) observed in human intestinal inflammatory diseases.

Female CB17 mice (8 weeks old) were obtained from Zhejiang Vital River Laboratory Animal Co. Ltd. Animals were housed and handled in a temperature-controlled environment with a 12-h light/12-h dark cycle. A total of 30 mice were assigned to 3 groups by randomization based on body weight.

To generate T cell transfer induced colitis, 95 female Balb/c mice, aged 8-10 weeks, were used to prepare naive CD4⁺CD45RB^{high} T and CD4⁺CD45RB^{low} T cells. The mouse spleens were harvested in precooled DPBS, grinded into cell suspension and passed through a 70 μm cell filter after erythrocytes lysed by ACK. Cells were collected and counted by centrifugation. Then, CD4 positive T cells were isolated by using negative magnetic bead separation kit. CD4⁺CD45RB^{high} naive T cells were selected by flow cytometry and used for model construction. 20 CB17 model mice were intraperitoneally injected with 5×10⁵ naive CD4⁺CD45RB^{high} T cells each on Day 0. 10 mice in negative control group were intraperitoneally injected with 5×10⁵ CD4⁺CD45RB^{low} T cells on Day 0.

Mice in Group 2 were treated with vehicle for 28 days (po, qd, from day 14 to day 41). Mice in Group 3 were treated with 90 mg/kg Cpd. 73 for 28 days (therapeutic regimen, po, qd, from day 14 to day 41). The protocols and procedures involving the care and use of animals were approved by the Institutional Animal Care and Use Committee (IACUC) at WuxiApptec (Shanghai, China). The grouping is shown in table 4.

TABLE 4

| | | | Number |
|---|---|---|---|
| Group | Treatment | Dosage | of mice |
| Group 1 (G1) | CD45RB^{low} | / | 10 |
| Group 2 (G2) | Vehicle | po, qd, from day 14 to day 41 | 10 |
| Group 3 (G3) | Cpd. 73 | 90 mg/kg, po, qd, from day 14 | 10 |
| | | to day 41 | |

### 2. Clinical Scores

Clinical signs of IBD were assessed every day based on the score of disease activity index (DAI) which was evaluated from two parameters using a scoring system from 0 to 4: stool consistency (0, normal stool; 1, soft but still formed stool; 2, soft and not formed stool; 3, very soft and wet stool; 4, watery diarrhea), and body weight loss (0, no body weight loos; 1, 1-5% body weight loss; 2, 6-10% body weight loss; 3, 11-20% body weight loss; 4, >20% body weight loss).

TABLE 5

| DAI | BW | |
|---|---|---|
| score | loss % | Stoll consistency |
| 0 | 0 | Normal |
| 1 | 1-5 | Soft but still formed |
| 2 | 6-10 | Soft |
| 3 | 11-20 | Very soft; wet |
| 4 | >20 | Watery diarrhea |

As seen in FIG. 10A-10B, mice from Group 1 (CD45RB^{low}) showed almost no symptoms of DAI change (DAI score less than 1 during the entire study). Compared with G1 CD45RB^{low} control group, there was an increased DAI score in the G2 group reaching the maximal DAI score of 4.90±0.41 on day 42. Treatment with Cpd. 73 significantly reduced the DAI to 1.4±0.27 on day 42 and improved body weight loss. These results indicated that treatment of Cpd. 73 could ameliorate the progression of CD4⁺CD45RB^{high} T-cell transfer-induced IBD in mice.

### 3. Colon Weight and Colon Length

At the end of the study (day 42), the animals were euthanized, dissected and the entire colon was quickly removed and gently cleared of feces.

The entire colon was weighed, and the total length was measured. Colon weight gain and shortening is an indirect marker of inflammation. As expected, CD4⁺CD45RB^{high} T-cell transfer induced significant increase of colon weight to 439.10±25.46 mg. Treatment with Cpd. 73 significantly reduced the increasement of colon weight to 323.40±16.06 mg (FIG. 11A).

For the colon length, the CD4⁺CD45RB^{high} T-cell transfer induced significant decrease of colon length to 7.72±0.18 cm. Treatment with 90 mg/kg Cpd. 73 significantly improved the colon length to 10.26±0.22 cm (FIG. 11B).

These results indirectly indicate that treatment of Cpd. 73 improved the inflammation in colon in CD4⁺CD45RB^{high} T-cell transfer-induced colitis mice.

### 4. Histopathologic Assessment

At the end of the study (day 42), all the animals were sacrificed by CO2. The colon was Swiss-rolled and fixed with neutralized PFA followed by H&E staining. After that, the pathologists from WuXi clinical pathological analysis platform, who were blinded to animal ID, reviewed the H&E staining and scored. The pathological scoring standards were as follows: crypt architecture (normal, 0; severe crypt distortion with loss of entire crypts, 3), degree of inflammatory cell infiltration (normal, 0; dense inflammatory infiltrate, 3), muscle thickening (normal, 0; marked muscle thickening present, 3), goblet cell depletion (absent, 0; present, 1) and crypt abscess (absent, 0; present, 1).

As seen in FIG. 12, mice in the vehicle group (G2) showed a pathological score of 9.00±0.47, indicating a large range of inflammatory cell infiltration in the colon, while treatment with Cpd. 73 significantly reduced the score to 3.70±0.37, which was in agreement with the alleviation of clinical symptoms as indicated by the clinical scores.

### Example of DSS Induced Inflammatory Bowel Disease (IBD) Model

### 1. Experimental Protocol for DSS Induced Inflammatory Bowel Disease (IBD) Model in C57BL/6 Mice

DSS-induced colitis shows clinical and histological similarities to ulcerative colitis.

Female C57BL/6 mice (8 weeks old) were obtained from Beijing Vital River Laboratory Animal Co. Ltd. Animals were housed and handled in a temperature-controlled environment with a 12-h light/12-h dark cycle. A total of 30 mice were assigned to 3 groups by randomization based on body weight.

On Day 0, colitis was induced by administration of 3% DSS (dextran sodium sulfate, molecular weight 36,000-50,000) in drinking water ad libitum for 8 days. 8-week-old mice were divided into 3 groups: Group 2, DSS treatment group (vehicle, po, qd, from day 0 to day 7); Group 3, DSS with 90 mg/kg Cpd. 73 (po, qd, from day 0 to day 7); and the Group 1 (G1) Naive group, the mice were provided drinking water without DSS. The protocols and procedures involving the care and use of animals were approved by the Institutional Animal Care and Use Committee (IACUC) at Wuxi Apptec (Shanghai, China). The grouping is shown in table 6.

TABLE 6

| | | | Number |
|---|---|---|---|
| Group | Treatment | Dosage | of mice |
| Group 1 (G1) | Naive | po, qd, from day 0 to day 7 (8 D) | 10 |
| Group 2 (G2) | Vehicle | po, qd, from day 0 to day 7 (8 D) | 10 |
| Group 3 (G3) | Cpd. 73 | 90 mg/kg, po, qd, from day 0 to | 10 |
| | | day 7 (8 D) | |

### 2. Colon Weight and Colon Length

At the end of the study, the animals were euthanized, dissected and the entire colon was quickly removed and gently cleared of feces.

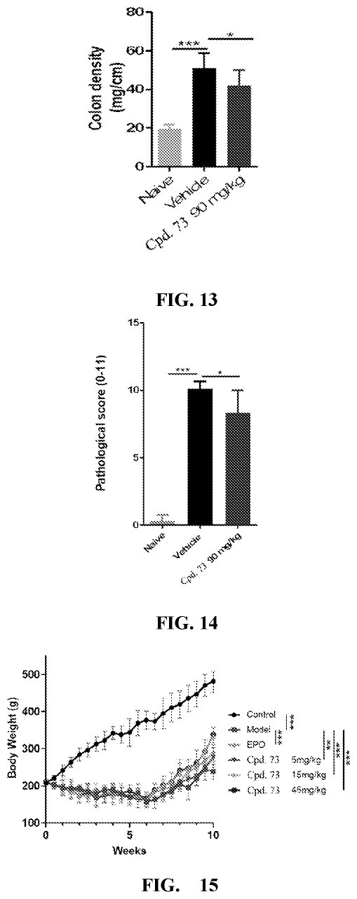

The entire colon was weighed, and the total length was measured. Increased colon density (colon weight/colon length) is an indirect marker of inflammation. As expected, DSS induced increasement of colon density to 50.88±2.53. Treatment with Cpd. 73 significantly decreased the colon density to 41.86±2.57 (FIG. 13).

This result indirectly indicates that treatment of Cpd. 73 improved the inflammation in colon in DSS-induced colitis mice.

### 3. Histopathologic Assessment

At the end of the study (day 8), all the animals were sacrificed by CO2. The colon was Swiss-rolled and fixed with neutralized PFA followed by H&E staining. After that, the pathologists from WuXi clinical pathological analysis platform, who were blinded to animal ID, reviewed the H&E staining and scored. The pathological scoring standards were as follows: crypt architecture (normal, 0; severe crypt distortion with loss of entire crypts, 3), degree of inflammatory cell infiltration (normal, 0; dense inflammatory infiltrate, 3), muscle thickening (normal, 0; marked muscle thickening present, 3), goblet cell depletion (absent, 0; present, 1) and crypt abscess (absent, 0; present, 1).

As seen in FIG. 14, mice in the vehicle group (G2) showed an increasement of pathological score to 10.10±0.18, while treatment with 90 mg/kg Cpd. 73 significantly reduced the score to 8.30±0.54.

These results indicated that treatment with Cpd. 73 plays a therapeutic role in DSS-induced IBD in mice.

### Embodiment 3

### 1. Materials and Methods

SPF-grade male SD rats (200±20 g, 8 weeks) were purchased from Zhejiang Vital River Laboratory Animal Technology Co., Ltd. After acclimating for a week, blood samples were collected and analyzed to exclude anormal animals. The normal animals were randomized, 10 animals were assigned into Control group which were left untreated until the end of the study, and the rest animals received 300 mg/kg adenine by oral gavage (QD×6 weeks). Compared to rats from Control group, adenine-treated rats showed significantly increased serum creatine and BUN levels and significantly decreased HGB level, indicating the successful model establishment. Then 60 adenine-treated rats with successful model establishment were further randomized into the following six groups: Model group (treated with vehicle), EPO group (50 U/kg), Cpd. 73 5 mg/kg group, Cpd. 73 15 mg/kg group, Cpd. 73 45 mg/kg group and CPD. 73+EPO group (15 mg/kg+50 U/kg). Each group contained 10 animals. Cpd. 73 was administered to rats by oral gavage (QD×4 weeks), while EPO was subcutaneously administered (TIW×4 weeks).

Body weight, mortality and health condition of rats were recorded twice a week. After model establishment, blood samples were take from the orbit and a few parameters were determined including RBC, HGB, HCT, RET, BUN and Creatine. Blood samples were taken once a week during drug treatment and RBC, HGB, HCT, and RET were analyzed.

2. Cpd. 73 Significantly Improved the Body Weight, RBC, HGB, HCT and RET of Rat with Chronic Kidney Disease-Induced Anemia

As shown in FIG. 15, at the end of the study, the body weight of SD rats from Model group (6 weeks of adenine plus 4 weeks of vehicle) was significantly lower than that from the Control group (untreated) (p<0.001). Treatment with 50 U/kg EPO (TIW×4 weeks) significantly improved the body weight of rats (p<0.001, vs. Model). Treatment with Cpd. 73 dose-dependently improved the body weight of adenine-treated rats, and significant difference was observed at the end of the study (p<0.001 or 0.01, vs. Model). It should be noted that 45 mg/kg Cpd. 73 improved the body weight of rat to the similar level with 50 U/kg EPO. Overall, Cpd. 73 was effective in improving the body weight in rat model of chronic kidney disease-induced anemia.

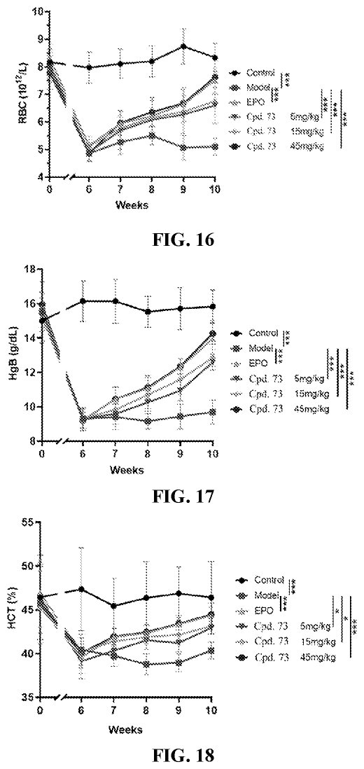

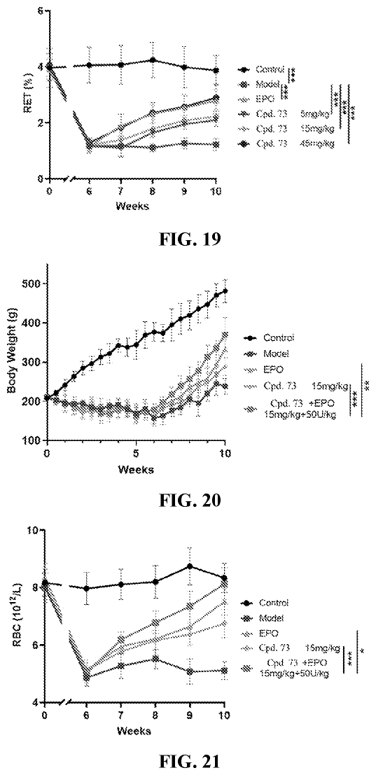

As shown in FIG. 16, FIG. 17, FIG. 18 and FIG. 19, at the end of the study, RBC, HGB, HCT, and RET values of SD rats from Model group (6 weeks of adenine plus 4 weeks of vehicle) was significantly lower than that from the Control group (untreated) (p<0.001). Treatment with 50 U/kg EPO (TIW×4 weeks) significantly improved these parameters (p<0.001, vs. Model), suggesting the therapeutic effect of EPO in the rat model of chronic kidney disease-induced anemia. Treatment with Cpd. 73 dose-dependently improved these parameters in adenine-treated rats, and significant difference was observed at the end of the study (p<0.001 or 0.05, vs. Model). It should be noted that 45 mg/kg Cpd. 73 improved these parameters to the similar level with 50 U/kg EPO. Overall, Cpd. 73 showed dose-dependent therapeutic effect in the rat model of chronic kidney disease-induced anemia.

3. Combination Treatment of Cpd. 73 and EPO Further Improved the Body Weight, RBC, HGB, HCT and RET of Rat with Chronic Kidney Disease-Induced Anemia

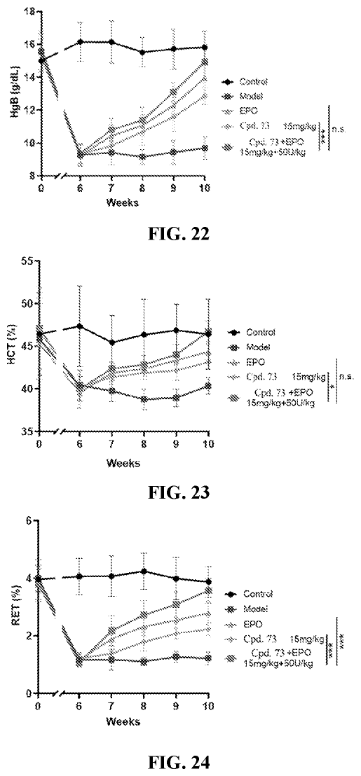

As shown in FIG. 20, combination of 15 mg/kg Cpd. 73 and EPO further improved the body weight of adenine-treated rats, achieving significant difference at the end of the study comparing with each single agent. As shown in FIG. 21, FIG. 22, FIG. 23 and FIG. 24, combination of 15 mg/kg Cpd. 73 and EPO further increased the values of RBC, HGB, HCT, and RET. At the end of the study, all the parameters recovered to normal level, and were higher than each single agent. Significant difference in all parameters was observed when compared with 15 mg/kg Cpd. 73 single agent group, and significant difference in RBC and RET was observed when compared with EPO single agent group. Overall, combination treatment of Cpd. 73 and EPO demonstrated enhanced therapeutic effect in the rat model of chronic kidney disease-induced anemia.

It is to be understood that the foregoing description of embodiments is intended to be purely illustrative of the principles of the invention, rather than exhaustive thereof, and that changes and variations will be apparent to those skilled in the art, and that the present invention is not intended to be limited other than expressly set forth in the following claims.

1. A method for preventing and/or treating a disease or disorder associated with a defect in hemoglobin protein activity or expression or innate immune response, type 1 or type 3 immunity related immune disorder, which comprises administering to a subject in need thereof a therapeutically effective amount of a substance X or a pharmaceutical composition comprising the substance X;
the substance X is a compound of Formula I, a pharmaceutically acceptable salt or solvate thereof; 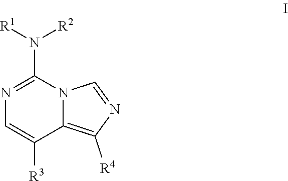
wherein:
R¹ is aralkyl;
R² is selected from the group consisting of hydrogen and C₁-C₄ alkyl;
R³ and R⁴ taken together with the carbon atoms to which they are attached form a radical of Formula I-A, I-B, or I-C: 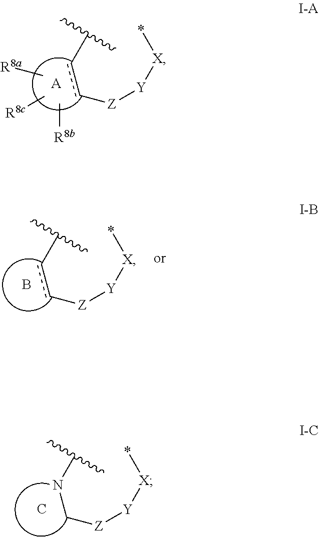
X is selected from the group consisting of —C(R^{5a})(R^{5b})—, —C(═O)—, and —S(═O)₂—;
R^{5a} and R^{5b} are independently selected from the group consisting of hydrogen and C₁-C₄ alkyl;
Y is selected from the group consisting of —C(R^{6a})(R^{6b})—, —S—, —O—, and —N(R⁷)—; or
X and Y taken together form a 5-membered heteroarylenyl;
Z is —C(R^{6c})(R^{6d})ₘ—;
R^{6a} and R^{6b} are independently selected from the group consisting of hydrogen and C₁-C₄ alkyl;
each R^{6c} and R^{6d} is independently selected from the group consisting of hydrogen and C₁-C₄ alkyl;
m is 0, 1, or 2;
R⁷ is selected from the group consisting of hydrogen, C₁-C₆ alkyl, C₁-C₆ haloalkyl, optionally substituted C₃-C₈ cycloalkyl, optionally substituted C₄-C₈ heterocyclo, hydroxyalkyl, (alkoxy)alkyl, (cycloalkyl)alkyl, and (heterocyclo)alkyl;
R^{8a}, R^{8b}, and R^{8c} are independently selected from the group consisting of hydrogen, halo, C₁-C₄ alkyl, C₁-C₄ haloalkyl, C₁-C₄ alkoxy, carboxamido, optionally substituted C₃-C₅ cycloalkyl, optionally substituted 4- to 8-membered heterocyclo, (heterocyclo)C₁-C₄ alkyl, and alkylsulfonyl; 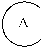
is a fused phenyl, fused 5-membered heteroaryl, or fused 6-membered heteroaryl; 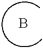
is an optionally substituted fused 3- to 8-membered cycloalkyl or optionally substituted fused 4- to 8-membered heterocyclo; 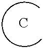
is an optionally substituted fused 4- to 8-membered heterocyclo; and
the bond designated with [symbol] is attached at the R³ position of Formula I and the bond designated with an “*” is attached at the R⁴ position of Formula I; or
R³ is R^{3a};
R⁴ is R^{4a};
R^{3a} is selected from group consisting of optionally substituted aryl, optionally substituted 5- to 10-membered heteroaryl, and optionally substituted 4- to 8-membered heterocyclo; and
R^{4a} is selected from the group consisting of hydrogen, halo, C₁-C₄haloalkyl, —S(═O)₂R⁹, —P(═O)(R^{10a})(R^{10b}), —C(═O)OR^{11a}, —C(═O)NR^{11b}R^{11c}, and —S(═O)(═NR^{13a})R^{13b};
R⁹ is selected from the group consisting of C₁-C₄ alkyl and C₃-C₆ cycloalkyl;
R^{10a} and R^{10b} are independently C₁-C₄ alkyl;
R^{11a} is selected from the group consisting of hydrogen and C₁-C₄ alkyl;
R^{11b} and R^{11c} are independently selected from the group consisting of hydrogen and C₁-C₄ alkyl; or
R^{11b} and R^{11c} taken together with the nitrogen atom to which they are attached form a 4- to 6-membered optionally substituted heterocyclo;
R^{13a} is selected from the group consisting of hydrogen, C₁-C₆ alkyl, C₃-C₆ cycloalkyl, and hydroxyalkyl;
R^{13b} is selected from the group consisting of C₁-C₆ alkyl and C₃-C₆ cycloalkyl; or
R^{13a} and R^{13b} taken together form a 5- to 7-membered heterocyclo; and [symbol]
is a single or double bond.

2. The method of claim 1, wherein R³ and R⁴ taken together with the carbon atoms to which they are attached form a radical of Formula I-A, I-B, or I-C, or a pharmaceutically acceptable salt or solvate thereof.

3. The method of claim 2, wherein the substance X are compounds of Formula II: 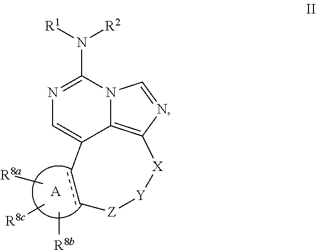
or a pharmaceutically acceptable salt or solvate thereof.

4. The method of claim 3, wherein the substance X are compounds of Formula III: 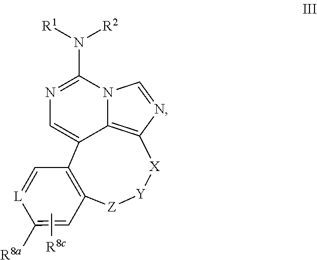
wherein L is selected from the group consisting of —C(R^{8b})═ and —N═, or a pharmaceutically acceptable salt or solvate thereof.

5. The method of claim 3, wherein the substance X are compounds of Formula IV: 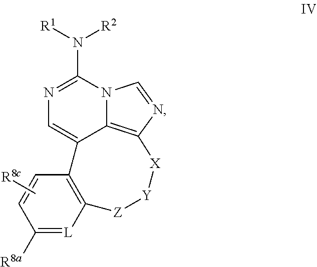
wherein L is selected from the group consisting of —C(R^{8b})═ and —N═, or a pharmaceutically acceptable salt or solvate thereof.

6. The method of claim 3, wherein the substance X are compounds of Formula V: 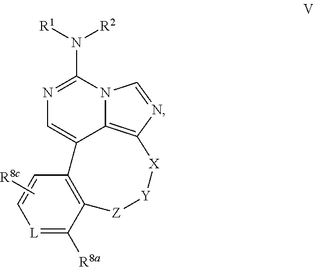
wherein L is selected from the group consisting of —C(R^{8b})═ and —N═, or a pharmaceutically acceptable salt or solvate thereof.

7. The method of claim 3, wherein the substance X are compounds of Formula VI: 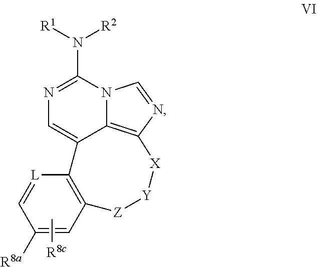
wherein L is selected from the group consisting of —C(R^{8b})═ and —N═, or a pharmaceutically acceptable salt or solvate thereof.

8. The method of claim 4, wherein L is —C(R^{8b})═, or a pharmaceutically acceptable salt or solvate thereof.

9. The method of any one of claims 3-7, wherein L is —N═, or a pharmaceutically acceptable salt or solvate thereof.

10. The method of any one of claims 1-9, wherein R^{8a}, R^{8b}, and R^{8c} are independently selected from the group consisting of hydrogen, C₁-C₄ alkyl, C₁-C₄ haloalkyl, C₃-C₆ cycloalky, optionally substituted 4- to 8-membered heterocyclo, and (heterocyclo)C₁-C₄ alkyl, or a pharmaceutically acceptable salt or solvate thereof.

11. The method of claim 10, wherein:
R^{8a} is selected from the group consisting of —CF₃, —CH₃, —CHF₂, —CD₃, and cyclopropyl; and
R^{8b} and R^{8c} are hydrogen, or a pharmaceutically acceptable salt or solvate thereof.

12. The method of claim 3, wherein the substance X are compounds of Formula VII: 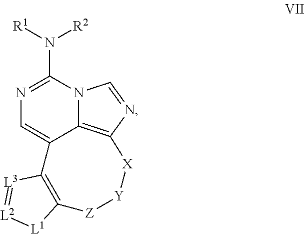
wherein:
L¹ is selected from the group consisting of —S—, —O—, and —N(R^{8a})—;
L² is selected from the group consisting of —C(R^{8b})═ and —N═;
L³ is selected from the group consisting of —C(R^{8c})═ and —N═;
R^{8a} is selected from the group consisting of hydrogen and C₁-C₄ alkyl;
R^{8b} is selected from the group consisting of hydrogen, C₁-C₄ alkyl, and C₁-C₄ haloalkyl; and
R^{8c} is selected from the group consisting of hydrogen, C₁-C₄ alkyl, and C₁-C₄ haloalkyl,
or a pharmaceutically acceptable salt or solvate thereof.

13. The method of claim 3, wherein the substance X are compounds of Formula VIII: 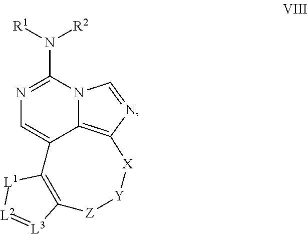
wherein:
L¹ is selected from the group consisting of —S—, —O—, and —N(R^{8a})—;
L² is selected from the group consisting of —C(R^{8b})═ and —N═;
L³ is selected from the group consisting of —C(R^{8c})═ and —N═;
R^{8a} is selected from the group consisting of hydrogen and C₁-C₄ alkyl;
R^{8b} is selected from the group consisting of hydrogen, C₁-C₄ alkyl, and C₁-C₄ haloalkyl; and
R^{8c} is selected from the group consisting of hydrogen, C₁-C₄ alkyl, and C₁-C₄ haloalkyl,
or a pharmaceutically acceptable salt or solvate thereof.

14. The method of claim 3, wherein the substance X are compounds of Formula IX: 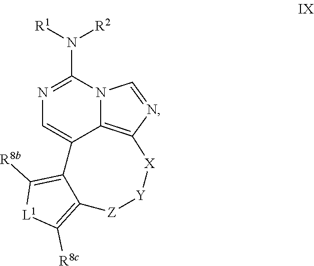
wherein:
L¹ is selected from the group consisting of —S—, —O—, and —N(R^{8a})—;
R^{8a} is selected from the group consisting of hydrogen and C₁-C₄ alkyl;
R^{8b} is selected from the group consisting of hydrogen, C₁-C₄ alkyl, and C₁-C₄ haloalkyl; and
R^{8c} is selected from the group consisting of hydrogen, C₁-C₄ alkyl, and C₁-C₄ haloalkyl,
or a pharmaceutically acceptable salt or solvate thereof.

15. The method of claim 2, wherein the substance X are compounds of Formula X: 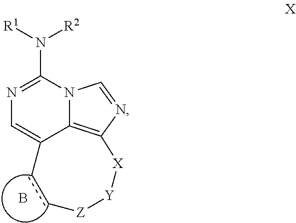
or a pharmaceutically acceptable salt or solvate thereof.

16. The method of claim 15, wherein the substance X are compounds of Formula XI: 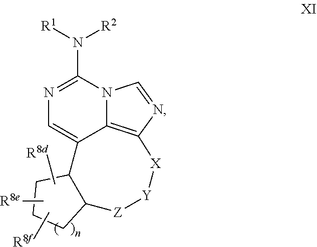
wherein:
R^{8d}, R^{8e}, and R^{8f} are independently selected from the group consisting of hydrogen, halo, and C₁-C₄ alkyl; and
n is 1, 2, or 3, or a pharmaceutically acceptable salt or solvate thereof.

17. The method of claim 15, wherein the substance X are compounds of Formula XII: 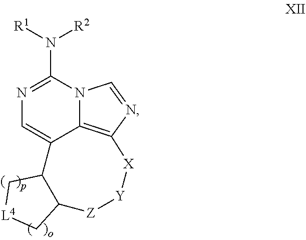
wherein:
L⁴ is selected from the group consisting of —S—, —O—, and —N(R^{8g})—;
R^{8g} is selected from the group consisting of hydrogen, C₁-C₄ alkyl, optionally substituted C₃-C₆ cycloalkyl, and optionally substituted 4- to 8-membered heterocyclo;
o is 0, 1, 2, or 3; and
p is 0, 1, 2, or 3;
wherein the sum of o and p is 1, 2, 3, 4, or 5,
or a pharmaceutically acceptable salt or solvate thereof.

18. The method of claim 2, wherein the substance X are compounds of Formula XIII: 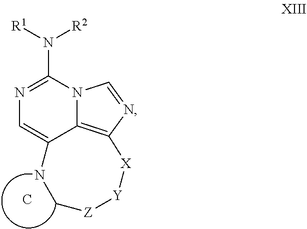
or a pharmaceutically acceptable salt or solvate thereof.

19. The method of claim 18, wherein the substance X are compounds of Formula XIV: 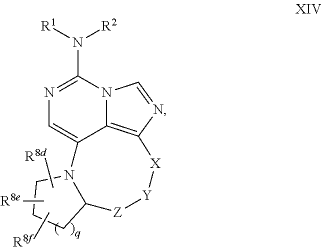
wherein:
R^{8d}, R^{8e}, and R^{8f} are independently selected from the group consisting of hydrogen and C₁-C₄ alkyl; and
q is 1, 2, or 3, or a pharmaceutically acceptable salt or solvate thereof.

20. The method of claim 18, wherein the substance X are compounds of Formula XV: 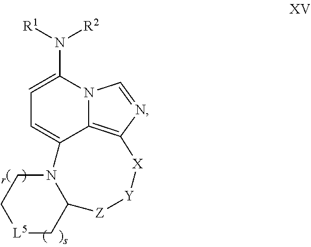
wherein:
L⁵ is selected from the group consisting of —S—, —O—, and —N(R^{8h})—;
R^{8h} is selected from the group consisting of hydrogen, C₁-C₄ alkyl, optionally substituted C₃-C₆ cycloalkyl, optionally substituted 4- to 8-membered heterocyclo, —C(═O)R^{14a}, and —S(═O)₂R^{14b};
R^{14a} and R^{14b} are independently selected from the group consisting of C₁-C₆ alkyl and optionally substituted C₃-C₈ cycloalkyl;
r is 1, 2, or 3; and
s is 1, 2, or 3,
or a pharmaceutically acceptable salt or solvate thereof.

21. The method of any one of claims 1-20, wherein Z is —CH₂—, or a pharmaceutically acceptable salt or solvate thereof.

22. The method of any one of claims 1-21, wherein:
X is selected from the group consisting of —C(R^{5a})(R^{5b})—, —C(═O)—, and —S(═O)₂—; and
Y is selected from the group consisting of —C(R^{6a})(R^{6b})—, —S—, —O—, and —N(R⁷)—, or a pharmaceutically acceptable salt or solvate thereof.

23. The method of any one of claims 1-22, wherein X is —CH₂—, or a pharmaceutically acceptable salt or solvate thereof.

24. The method of any one of claims 1-22, wherein X is —C(═O)—, or a pharmaceutically acceptable salt or solvate thereof.

25. The method of any one of claims 1-22, wherein X is —S(═O)₂—, or a pharmaceutically acceptable salt or solvate thereof.

26. The method of any one of claims 1-25, wherein Y is —O—, or a pharmaceutically acceptable salt or solvate thereof.

27. The method of any one of claims 1-25, wherein Y is —N(R⁷)—, or a pharmaceutically acceptable salt or solvate thereof.

28. The method of claim 27, wherein R⁷ is selected from the group consisting of C₁-C₆ alkyl, C₁-C₆ haloalkyl, and optionally substituted C₃-C₅ cycloalkyl, or a pharmaceutically acceptable salt or solvate thereof.

29. The method of any one of claims 1-21, wherein X and Y taken together form a 5-membered heteroarylenyl, or a pharmaceutically acceptable salt or solvate thereof.

30. The method of claim 1, wherein the substance X are compounds of Formula XVI: 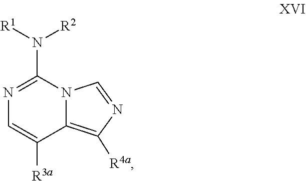
or a pharmaceutically acceptable salt or solvate thereof.

31. The method of any one of claims 1-30, wherein R² is hydrogen, or a pharmaceutically acceptable salt or solvate thereof.

32. The method of any one of claims 1-31, wherein:
R¹ is R¹-1: 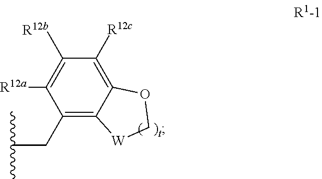
R^{12a}, R^{12b}, and R^{12c} are each independently selected from the group consisting of hydrogen, halo, C₁-C₄ alkyl, C₁-C₄ haloalkyl, and C₁-C₄ alkoxy;
W is selected from the group consisting of —CH₂— and —C(═O)—; and
t is 1 or 2,
or a pharmaceutically acceptable salt or solvate thereof.

33. The method of claim 32, wherein W is —C(═O)— and t is 1, or a pharmaceutically acceptable salt or solvate thereof.

34. The method of claim 32, wherein W is —CH₂— and t is 2, or a pharmaceutically acceptable salt or solvate thereof.

35. The method of claim 32, wherein W is —CH₂— and t is 1, or a pharmaceutically acceptable salt or solvate thereof.

36. The method of any one of claims 32-35, wherein:
R^{12a} is fluoro; and
R^{12b} and R^{12c} are independently selected from the group consisting of hydrogen and fluoro, or a pharmaceutically acceptable salt or solvate thereof.

37. The method of claim 36, wherein:
R^{12a} is fluoro; and
R^{12b} and R^{12c} are hydrogen,
or a pharmaceutically acceptable salt or solvate thereof.

38. The method of claim 1, wherein the substance X are selected from group consisting of:

| Cpd. | | |
|---|---|---|
| No. | Structure | Name |
| 1 | 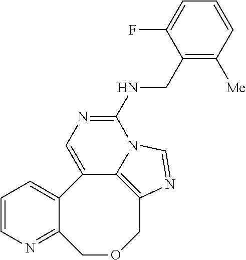 [SMILES: Cc1cccc(F)c1CNc1ncc2c3c(ncn13)COCc1ncccc1-2] | N-(2-fluoro-6-methylbenzyl)-3H,5H-4-oxa- 2,6,11,12a-tetraazabenzo[4,5]cyclooctan[1,2,3- cd]inden-12-amine |
| 2 | 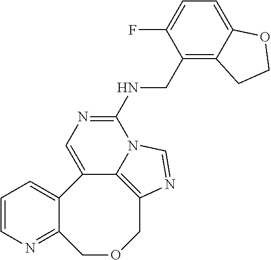 [SMILES: Fc1ccc2c(c1CNc1ncc3c4c(ncn14)COCc1ncccc1-3)CCO2] | N-((5-fluoro-2,3-dihydrobenzofuran-4-yl)methyl)- 3H,5H-4-oxa-2,6,11,12a- tetraazabenzo[4,5]cycloocta[1,2,3-cd]inden-12- amine |
| 3 | 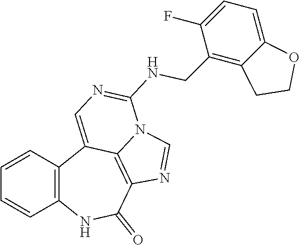 [SMILES: O=C1Nc2ccccc2-c2cnc(NCc3c(F)ccc4c3CCO4)n3cnc1c23] | 11-(((5-fluoro-2,3-dihydrobenzofuran-4- yl)methyl)amino)-2,4,10,11a- tetraazadibenzo[cd,f]azulen-3(4H)-one |
| 4 | 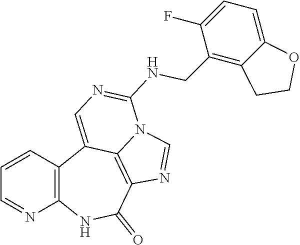 [SMILES: O=C1Nc2ncccc2-c2cnc(NCc3c(F)ccc4c3CCO4)n3cnc1c23] | 11-(((5-fluoro-2,3-dihydrobenzofuran-4- yl)methyl)amino)-2,4,5,10,11a- pentaazadibenzo[cd,f]azulen-3(4H)-one |
| 5 | 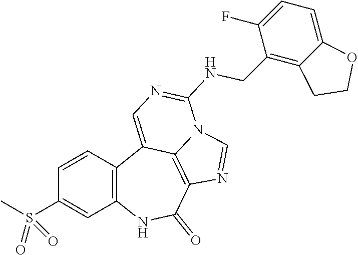 [SMILES: CS(=O)(=O)c1ccc2c(c1)NC(=O)c1ncn3c(NCc4c(F)ccc5c4CCO5)ncc-2c13] | 11-(((5-fluoro-2,3-dihydrobenzofuran-4- yl)methyl)amino)-6-(methylsulfonyl)-2,4,10,11a- tetraazadibenzo[cd,f]azulen-3(4H)-one |
| 6 | 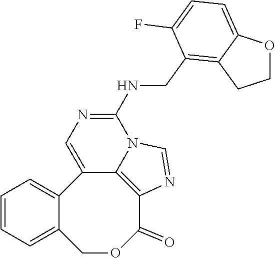 [SMILES: O=C1OCc2ccccc2-c2cnc(NCc3c(F)ccc4c3CCO4)n3cnc1c23] | 12-(((5-fluoro-2,3-dihydrobenzofuran-4- yl)methyl)amino)-3H,5H-4-oxa-2,11,12a- triazabenzo[4,5]cycloocta[1,2,3-cd]inden-3-one |
| 7 | 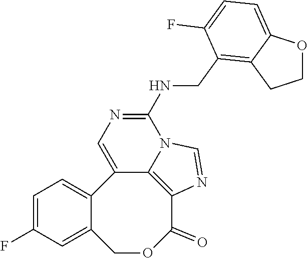 [SMILES: O=C1OCc2cc(F)ccc2-c2cnc(NCc3c(F)ccc4c3CCO4)n3cnc1c23] | 7-fluoro-12-(((5-fluoro-2,3-dihydrobenzofuran-4- yl)methyl)amino)-3H,5H-4-oxa-2,11,12a- triazabenzo[4,5]cycloocta[1,2,3-cd]inden-3-one |
| 8 | 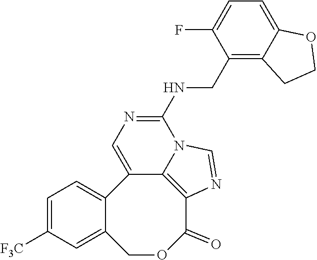 [SMILES: O=C1OCc2cc(C(F)(F)F)ccc2-c2cnc(NCc3c(F)ccc4c3CCO4)n3cnc1c23] | 12-(((5-fluoro-2,3-dihydrobenzofuran-4- yl)methyl)amino)-7-(trifluoromethyl)-3H,5H-4- oxa-2,11,12a-triazabenzo[4,5]cycloocta[1,2,3- cd]inden-3-one |
| 9 | 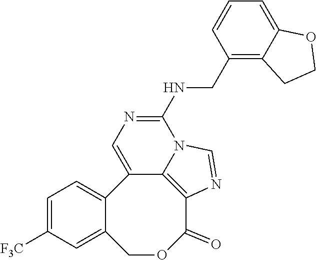 [SMILES: O=C1OCc2cc(C(F)(F)F)ccc2-c2cnc(NCc3cccc4c3CCO4)n3cnc1c23] | 12-(((2,3-dihydrobenzofuran-4-yl)methyl)amino)- 7-fluoro-3H,5H--4-oxa-2,11,12a- triazabenzo[4,5]cycloocta[1,2,3-cd]inden-3-one |
| 10 | 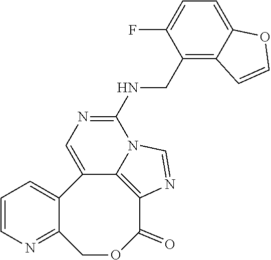 [SMILES: O=C1OCc2ncccc2-c2cnc(NCc3c(F)ccc4occc34)n3cnc1c23] | 12-(((5-fluorobenzofuran-4-yl)methyl)amino)- 3H,5H-4-oxa-2,6,11,12a- tetraazabenzo[4,5]cycloocta[1,2,3-cd]inden-3-one |
| 11 | 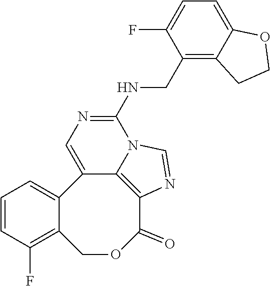 [SMILES: O=C1OCc2c(F)cccc2-c2cnc(NCc3c(F)ccc4c3CCO4)n3cnc1c23] | 6-fluoro-12-(((5-fluoro-2,3-dihydrobenzofuran-4- yl)methyl)amino)-3H,5H-4-oxa-2,11,12a- triazabenzo[4,5]cycloocta[1,2,3-cd]inden-3-one |
| 12 | 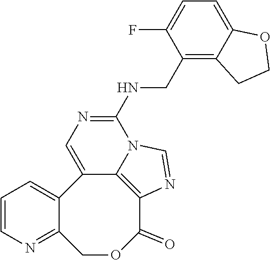 [SMILES: O=C1OCc2ncccc2-c2cnc(NCc3c(F)ccc4c3CCO4)n3cnc1c23] | 12-(((5-fluoro-2,3-dihydrobenzofuran-4- yl)methyl)amino)-3H,5H-4-oxa-2,6,11,12a- tetraazabenzo[4,5]cycloocta[1,2,3-cd]inden-3-one |
| 13 | 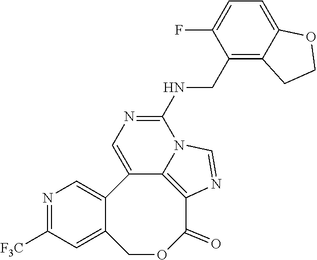 [SMILES: O=C1OCc2cc(C(F)(F)F)ncc2-c2cnc(NCc3c(F)ccc4c3CCO4)n3cnc1c23] | 12-(((5-fluoro-2,3-dihydrobenzofuran-4- yl)methyl)amino)-7-(trifluoromethyl)-3H,5H-4- oxa-2,8,11,12a-tetraazabenzo[4,5]cycloocta[1,2,3- cd]indene-3-one |
| 14 | 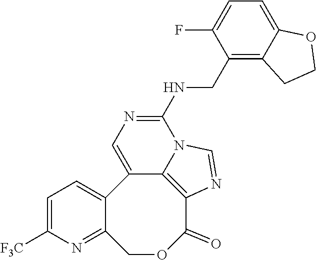 [SMILES: O=C1OCc2nc(C(F)(F)F)ccc2-c2cnc(NCc3c(F)ccc4c3CCO4)n3cnc1c23] | 12-((5-fluoro-2,3-dihydrobenzofuran-4- yl)methyl)amino)-7-(trifluoromethyl)-3H,5H-4- oxa-2,6,11,12a-tetraazabenzo[4,5]cycloocta[1,2,3- cd]inden-3-one |
| 15 | 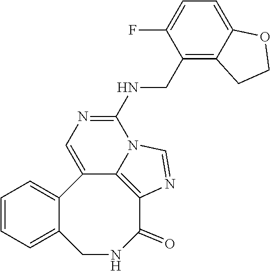 [SMILES: O=C1NCc2ccccc2-c2cnc(NCc3c(F)ccc4c3CCO4)n3cnc1c23] | 12-(((5-fluoro-2,3-dihydrobenzofuran-4- yl)methyl)amino)-4,5-dihydro-3H-2,4,11,12a- tetraazabenzo[4,5]cycloocta[1,2,3-cd]inden-3-one |
| 16 | 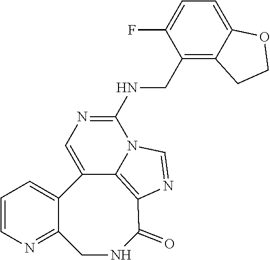 [SMILES: O=C1NCc2ncccc2-c2cnc(NCc3c(F)ccc4c3CCO4)n3cnc1c23] | 12-(((5-fluoro-2,3-dihydrobenzofuran-4- yl)methyl)amino)-4,5-dihydro-3H-2,4,6,11,12a- pentaazabenzo[4,5]cycloocta[1,2,3-cd]inden-3- one |
| 17 | 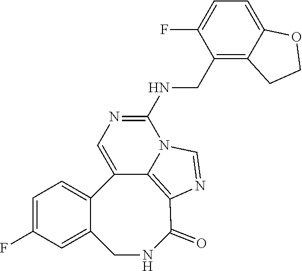 [SMILES: O=C1NCc2cc(F)ccc2-c2cnc(NCc3c(F)ccc4c3CCO4)n3cnc1c23] | 7-fluoro-12-(((5-fluoro-2,3-dihydrobenzofuran-4- yl)methyl)amino)-4,5-dihydro-3H-2,4,11,12a- tetraazabenzo[4,5]cycloocta[1,2,3-cd]inden-3-one |
| 18 | 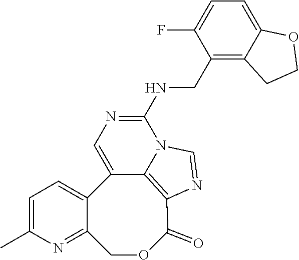 [SMILES: Cc1ccc2c(n1)COC(=O)c1ncn3c(NCc4c(F)ccc5c4CCO5)ncc-2c13] | 12-(((5-fluoro-2,3-dihydrobenzofuran-4- yl)methyl)amino)-7-methyl-3H,5H-4-oxa- 2,6,11,12a-tetraazabenzo[4,5]cycloocta[1,2,3- cd]indene-3-one |
| 19 | 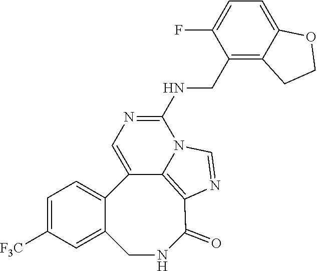 [SMILES: O=C1NCc2cc(C(F)(F)F)ccc2-c2cnc(NCc3c(F)ccc4c3CCO4)n3cnc1c23] | 12-(((5-fluoro-2,3-dihydrobenzofuran-4- yl)methyl)amino)-7-(trifluoromethyl)-4,5- dihydro-3H-2,4,11,12a- tetraazabenzo[4,5]cycloocta[1,2,3-cd]inden-3-one |
| 20 | 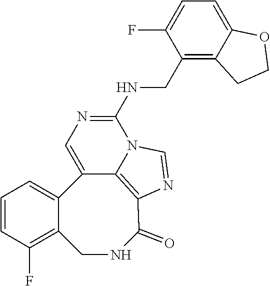 [SMILES: O=C1NCc2c(F)cccc2-c2cnc(NCc3c(F)ccc4c3CCO4)n3cnc1c23] | 6-fluoro-12-(((5-fluoro-2,3-dihydrobenzofuran-4- yl)methyl)amino)-4,5-dihydro-3H-2,4,11,12a- tetraazabenzo[4,5]cycloocta[1,2,3-cd]inden-3-one |
| 21 | 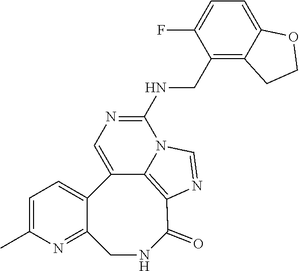 [SMILES: Cc1ccc2c(n1)CNC(=O)c1ncn3c(NCc4c(F)ccc5c4CCO5)ncc-2c13] | 12-(((5-fluoro-2,3-dihydrobenzofuran-4- yl)methyl)amino)-7-methyl-4,5-dihydro-3H- 2,4,6,11,12a-pentaazabenzo[4,5]cycloocta[1,2,3- cd]indene-3-one |
| 22 | 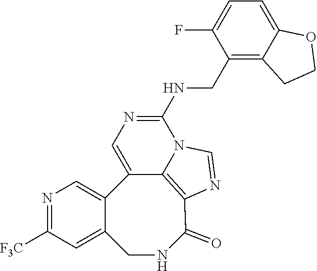 [SMILES: O=C1NCc2cc(C(F)(F)F)ncc2-c2cnc(NCc3c(F)ccc4c3CCO4)n3cnc1c23] | 12-(((5-fluoro-2,3-dihydrobenzofuran-4- yl)methyl)amino)-7-(trifluoromethyl)-4,5- dihydro-3H-2,4,8,11,12a- pentaazabenzo[4,5]cycloocta[1,2,3-cd]inden-3- one |
| 23 | 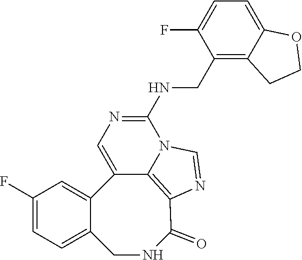 [SMILES: O=C1NCc2ccc(F)cc2-c2cnc(NCc3c(F)ccc4c3CCO4)n3cnc1c23] | 8-fluoro-12-(((5-fluoro-2,3-dihydrobenzofuran-4- yl)methyl)amino)-4,5-dihydro-3H-2,4,11,12a- tetraazabenzo[4,5]cycloocta[1,2,3-cd]inden-3-one |
| 24 | 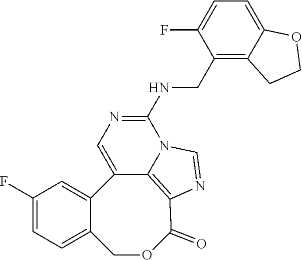 [SMILES: O=C1OCc2ccc(F)cc2-c2cnc(NCc3c(F)ccc4c3CCO4)n3cnc1c23] | 12-(((5-fluoro-2,3-dihydrobenzofuran-4- yl)methyl)amino)-8-(trifluoromethyl)-3H,5H-4- oxa-2,11,12a-triazabenzo[4,5]cycloocta[1,2,3- cd]inden-3-one |
| 25 | 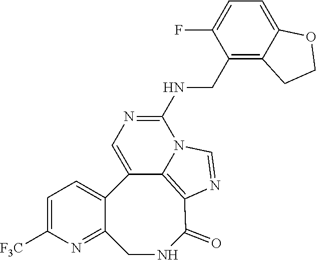 [SMILES: O=C1NCc2nc(C(F)(F)F)ccc2-c2cnc(NCc3c(F)ccc4c3CCO4)n3cnc1c23] | 12-(((5-fluoro-2,3-dihydrobenzofuran-4- yl)methyl)amino)-7-(trifluoromethyl)-4,5- dihydro-3H-2,4,6,11,12a- pentaazabenzo[4,5]cycloocta[1,2,3-cd]inden-3- one |
| 26 | 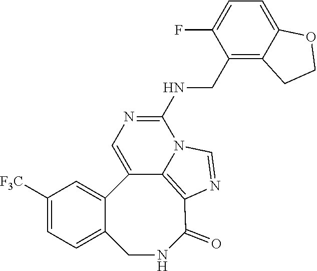 [SMILES: O=C1NCc2ccc(C(F)(F)F)cc2-c2cnc(NCc3c(F)ccc4c3CCO4)n3cnc1c23] | 12-(((5-fluoro-2,3-dihydrobenzofuran-4- yl)methyl)amino)-8-(trifluoromethyl)-4,5- dihydro-3H-2,4,11,12a- tetraazabenzo[4,5]cycloocta[1,2,3-cd]inden-3-one |
| 27 | 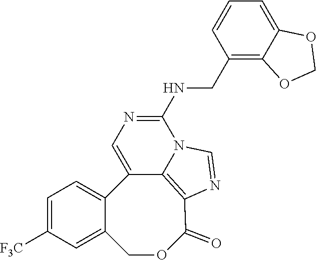 [SMILES: O=C1OCc2cc(C(F)(F)F)ccc2-c2cnc(NCc3cccc4c3OCO4)n3cnc1c23] | 12-((benzo[d][1,3]dioxol-4-ylmethyl)amino)-7- (trifluoromethyl)-3H,5H-4-oxa-2,11,12a- triazabenzo[4,5]cycloocta[1,2,3-cd]inden-3-one |
| 28 | 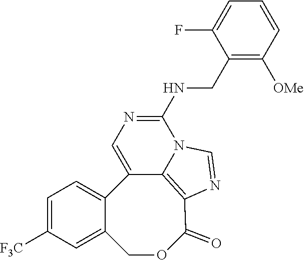 [SMILES: COc1cccc(F)c1CNc1ncc2c3c(ncn13)C(=O)OCc1cc(C(F)(F)F)ccc1-2] | 12-((2-fluoro-6-methoxybenzyl)amino)-7- (trifluoromethyl)-3H,5H-4-oxa-2,11,12a- triazabenzo[4,5]cycloocta[1,2,3-cd]inden-3-one |
| 29 | 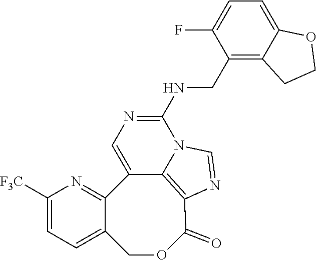 [SMILES: O=C1OCc2ccc(C(F)(F)F)nc2-c2cnc(NCc3c(F)ccc4c3CCO4)n3cnc1c23] | 12-(((5-fluoro-2,3-dihydrobenzofuran-4- yl)methyl)amino)-8-(trifluoromethyl)-3H,5H-4- oxa-2,9,11,12a-tetraazabenzo[4,5]cycloocta[1,2,3- cd]inden-3-one |
| 30 | 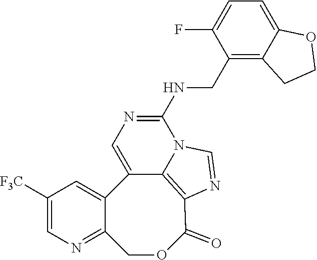 [SMILES: O=C1OCc2ncc(C(F)(F)F)cc2-c2cnc(NCc3c(F)ccc4c3CCO4)n3cnc1c23] | 12-(((5-fluoro-2,3-dihydrobenzofuran-4- yl)methyl)amino)-8-(trifluoromethyl)-3H,5H-4- oxa-2,6,11,12a-tetraazabenzo[4,5]cycloocta[1,2,3- cd]inden-3-one |
| 31 | 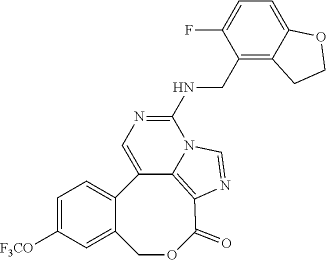 [SMILES: O=C1OCc2cc(OC(F)(F)F)ccc2-c2cnc(NCc3c(F)ccc4c3CCO4)n3cnc1c23] | 12-(((5-fluoro-2,3-dihydrobenzofuran-4- yl)methyl)amino)-7-(trifluoromethoxy)-3H,5H-4- oxa-2,11,12a-triazabenzo[4,5]cycloocta[1,2,3- cd]inden-3-one |
| 32 | 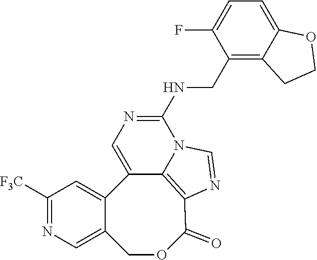 [SMILES: O=C1OCc2cnc(C(F)(F)F)cc2-c2cnc(NCc3c(F)ccc4c3CCO4)n3cnc1c23] | 12-(((5-fluoro-2,3-dihydrobenzofuran-4- yl)methyl)amino)-8-(trifluoromethyl)-3H,5H-4- oxa-2,7,11,12a-tetraazabenzo[4,5]cycloocta[1,2,3- cd]inden-3-one |
| 33 | 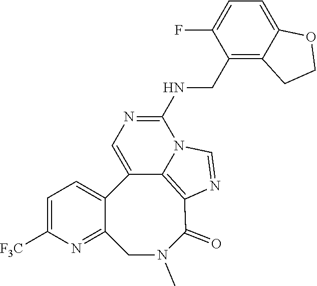 [SMILES: CN1Cc2nc(C(F)(F)F)ccc2-c2cnc(NCc3c(F)ccc4c3CCO4)n3cnc(c23)C1=O] | 12-(((5-fluoro-2,3-dihydrobenzofuran-4- yl)methyl)amino)-4-methyl-7-(trifluoromethyl)- 4,5-dihydro-3H-2,4,6,11,12a- pentaazabenzo[4,5]cycloocta[1,2,3-cd]inden-3- one |
| 34 | 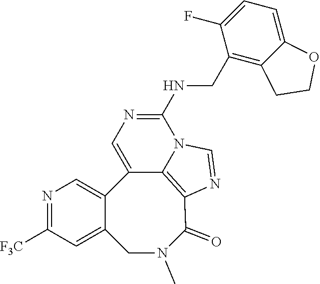 [SMILES: CN1Cc2cc(C(F)(F)F)ncc2-c2cnc(NCc3c(F)ccc4c3CCO4)n3cnc(c23)C1=O] | 12-(((5-fluoro-2,3-dihydrobenzofuran-4- yl)methyl)amino)-4-methyl-7-(trifluoromethyl)- 4,5-dihydro-3H-2,4,8,11,12a- pentaazabenzo[4,5]cycloocta[1,2,3-cd]inden-3- one |
| 35 | 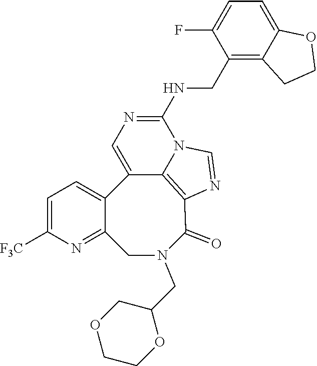 [SMILES: O=C1c2ncn3c(NCc4c(F)ccc5c4CCO5)ncc(c23)-c2ccc(C(F)(F)F)nc2CN1CC1COCCO1] | 4-((1,4-dioxan-2-yl)methyl)-12-(((5-fluoro-2,3- dihydrobenzofuran-4-yl)methyl)amino)-7- (trifluoromethyl)-4,5-dihydro-3H-2,4,6,11,12a- pentaazabenzo[4,5]cycloocta[1,2,3-cd]inden-3- one |
| 36 | 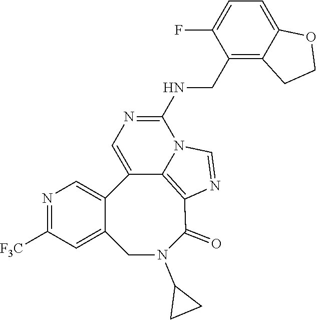 [SMILES: O=C1c2ncn3c(NCc4c(F)ccc5c4CCO5)ncc(c23)-c2cnc(C(F)(F)F)cc2CN1C1CC1] | 4-cyclopropyl-12-(((5-fluoro-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| 37 | 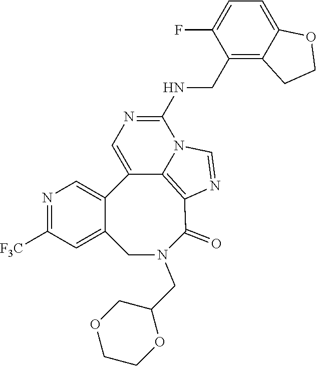 [SMILES: O=C1c2ncn3c(NCc4c(F)ccc5c4CCO5)ncc(c23)-c2cnc(C(F)(F)F)cc2CN1CC1COCCO1] | 4-((1,4-dioxan-2-yl)methyl)-12-(((5-fluoro-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| 38 | 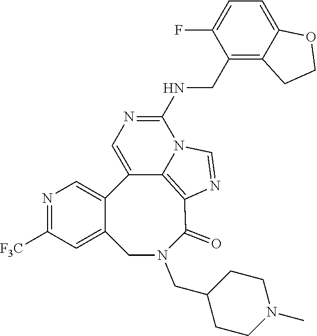 [SMILES: CN1CCC(CN2Cc3cc(C(F)(F)F)ncc3-c3cnc(NCc4c(F)ccc5c4CCO5)n4cnc(c34)C2=O)CC1] | 12-(((5-fluoro-2,3-dihydrobenzofurna-4- yl)methyl)amino)-4-((1-methylpiperidin-4- yl)methyl)-7-(trifluoromethyl)-4,5-dihydro-3H- 2,4,8,11,12a-pentaazabenzo[4,5]cycloocta[1,2,3- cd]inden-3-one |
| 39 | 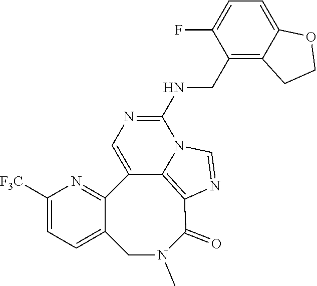 [SMILES: CN1Cc2ccc(C(F)(F)F)nc2-c2cnc(NCc3c(F)ccc4c3CCO4)n3cnc(c23)C1=O] | 12-(((5-fluoro-2,3-dihydrobenzofuran-4- yl)methyl)amino)-4-methyl-8-(trifluoromethyl)- 4,5-dihydro-3H-2,4,9,11,12a- pentaazabenzo[4,5]cycloocta[1,2,3-cd]inden-3- one |
| 40 | 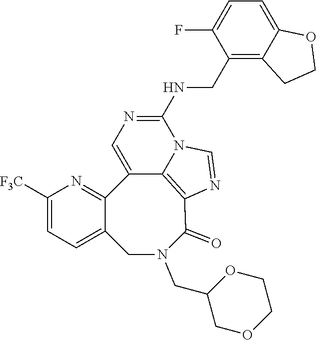 [SMILES: O=C1c2ncn3c(NCc4c(F)ccc5c4CCO5)ncc(c23)-c2nc(C(F)(F)F)ccc2CN1CC1COCCO1] | 4-((1,4-dioxan-2-yl)methyl)-12-(((5-fluoro-2,3- dihydrobenzofuran-4-yl)methyl)amino)-8- (trifluoromethyl)-4,5-dihydro-3H-2,4,9,11,12a- pentaazabenzo[4,5]cycloocta[1,2,3-cd]inden-3- one |
| 41 | 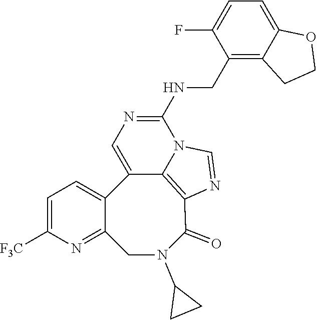 [SMILES: O=C1c2ncn3c(NCc4c(F)ccc5c4CCO5)ncc(c23)-c2ccc(C(F)(F)F)nc2CN1C1CC1] | 4-cyclopropyl-12-(((5-fluoro-2,3- dihydrobenzofuran-4-yl)methyl)amino)-7- (trifluoromethyl)-4,5-dihydro-3H-2,4,6,11,12a- pentaazabenzo[4,5]cycloocta[1,2,3-cd]inden-3- one |
| 42 | 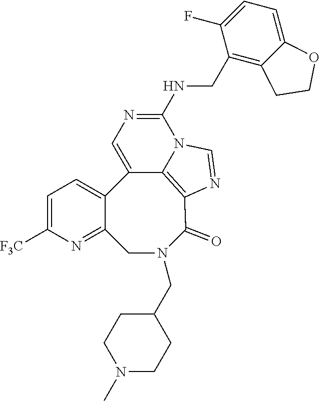 [SMILES: CN1CCC(CN2Cc3nc(C(F)(F)F)ccc3-c3cnc(NCc4c(F)ccc5c4CCO5)n4cnc(c34)C2=O)CC1] | 12-(((5-fluoro-2,3-dihydrobenzofuran-4- yl)methyl)amino)-4-((1-methylpiperidin-4- yl)methyl)-7-(trifluoromethyl)-4,5-dihydro-3H- 2,4,6,11,12a-pentaazabenzo[4,5]cycloocta[1,2,3- cd]inden-3-one |
| 43 | 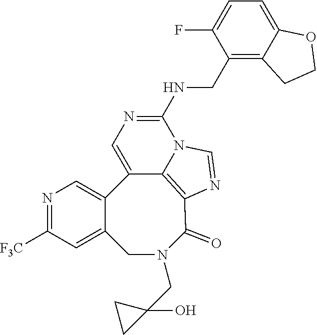 [SMILES: O=C1c2ncn3c(NCc4c(F)ccc5c4CCO5)ncc(c23)-c2cnc(C(F)(F)F)cc2CN1CC1(O)CC1] | 12-(((5-fluoro-2,3-dihydrobenzofuran-4- yl)methyl)amino)-4-((1- hydroxycyclopropyl)methyl)-7-(trifluoromethyl)- 4,5-dihydro-3H-2,4,8,11,12a- pentaazabenzo[4,5]cylcoocta[1,2,3-cd]inden-3- one |
| 44 | 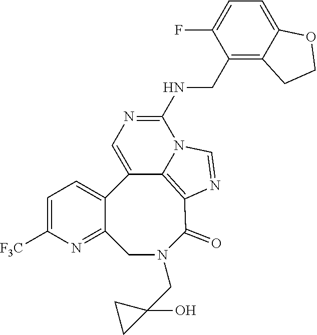 [SMILES: O=C1c2ncn3c(NCc4c(F)ccc5c4CCO5)ncc(c23)-c2ccc(C(F)(F)F)nc2CN1CC1(O)CC1] | 12-(((5-fluoro-2,3-dihydrobenzofuran-4- yl)methyl)amino)-4-((1- hydroxycyclopropyl)methyl)-7-(trifluoromethyl)- 4,5-dihydro-3H-2,4,6,11,12a- pentaazabenzo[4,5]cycloocta[1,2,3-cd]inden-3- one |
| 45 | 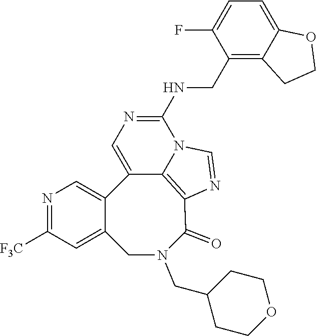 [SMILES: O=C1c2ncn3c(NCc4c(F)ccc5c4CCO5)ncc(c23)-c2cnc(C(F)(F)F)cc2CN1CC1CCOCC1] | 12-(((5-fluoro-2,3-dihydrobenzofuran-4- yl)methyl)amino)-4-((tetrahydro-2H-pyran-4- yl)methyl)-7-(trifluoromethyl)-4,5-dihydro-3H- 2,4,8,11,12a-pentaazabenzo[4,5]cycloocta[1,2,3- cd]inden-3-one |
| 46 | 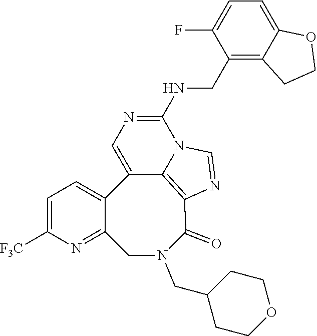 [SMILES: O=C1c2ncn3c(NCc4c(F)ccc5c4CCO5)ncc(c23)-c2ccc(C(F)(F)F)nc2CN1CC1CCOCC1] | 12-(((5-fluroo-2,3-dihydrobenzofuran-4- yl)methyl)amino)-4-((tetrahydro-2H-pyran-4- yl)methyl)-7-(trifluoromethyl)-4,5-dihydro-3H- 2,4,6,11,12a-pentaazabenzo[4,5]cycloocta[1,2,3- cd]indene-3-one |
| 47 | 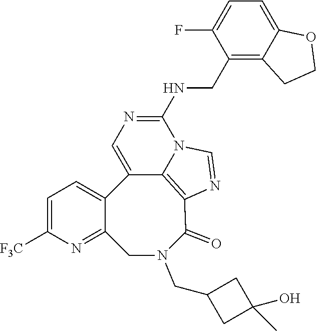 [SMILES: CC1(O)CC(CN2Cc3nc(C(F)(F)F)ccc3-c3cnc(NCc4c(F)ccc5c4CCO5)n4cnc(c34)C2=O)C1] | 12-(((5-fluoro-2,3-dihydrobenzofuran-4- yl)methyl)amino)-4-((3-hydroxy-3- methylcyclobutyl)methyl)-7-(trifluoromethyl)-4,5- dihydro-3H-2,4,6,11,12a- pentaazabenzo[4,5]cycloocta[1,2,3-cd]inden-3- one |
| 48 | 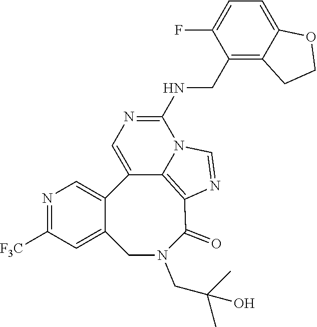 [SMILES: CC(C)(O)CN1Cc2cc(C(F)(F)F)ncc2-c2cnc(NCc3c(F)ccc4c3CCO4)n3cnc(c23)C1=O] | 12-(((5-fluoro-2,3-dihydrobenzofuran-4- yl)methyl)amino)-4-(2-hydroxy-2-methylprpoyl)- 7-(trifluoromethyl)-4,5-dihydro-3H-2,4,8,11,12a- pentaazabenzo[4,5]cycloocta[1,2,3-cd]inden-2- one |
| 49 | 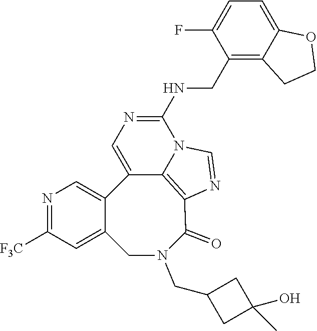 [SMILES: CC1(O)CC(CN2Cc3cc(C(F)(F)F)ncc3-c3cnc(NCc4c(F)ccc5c4CCO5)n4cnc(c34)C2=O)C1] | 12-(((5-fluoro-2,3-dihydrobenzofuran-4- yl)methyl)amino)-4-((3-hydroxy-3- methylcyclobutyl)methyl)-7-(trifluoromethyl)-4,5- dihydro-3H-2,4,8,11,12a- pentaazabenzo[4,5]cycloocta[1,2,3-cd]inden-3- one |
| 50 | 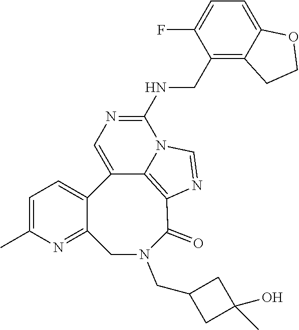 [SMILES: Cc1ccc2c(n1)CN(CC1CC(C)(O)C1)C(=O)c1ncn3c(NCc4c(F)ccc5c4CCO5)ncc-2c13] | 12-(((5-fluoro-2,3-dihydrobenzofuran-4- yl)methyl)amino)-4-((3-hydroxy-3- methylcyclobutyl)methyl)-7-methyl-4,5-dihydro- 3H-2,4,6,11,12a- pentaazabenzo[4,5]cycloocta[1,2,3-cd]inden-3- one |
| 51 | 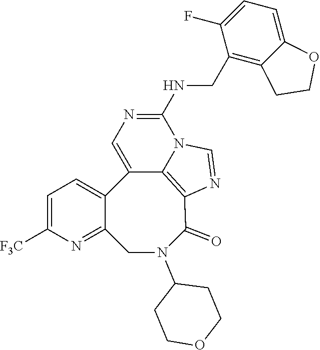 [SMILES: O=C1c2ncn3c(NCc4c(F)ccc5c4CCO5)ncc(c23)-c2ccc(C(F)(F)F)nc2CN1C1CCOCC1] | 12-(((5-fluoro-2,3-dihydrobenzofuran-4- yl)methyl)amino)-4-(tetrahydro-2H-pyran-4-yl)-7- (trifluoromethyl)-4,5-dihydro-3H-2,4,6,11,12a- pentaazabenzo[4,5]cycloocta[1,2,3-cd]inden-3- one |
| 52 | 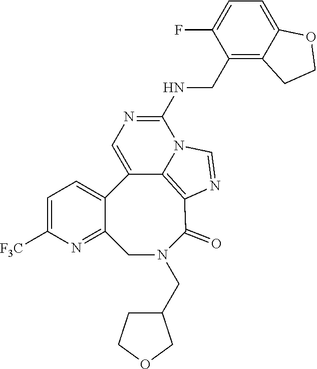 [SMILES: O=C1c2ncn3c(NCc4c(F)ccc5c4CCO5)ncc(c23)-c2ccc(C(F)(F)F)nc2CN1CC1CCOC1] | 12-(((5-fluoro-2,3-dihydrobenzofuran-4- yl)methyl)amino)-4-((tetrahydrofuran-3- yl)methyl)-7-(trifluoromethyl)-4,5-dihydro-3H- 2,4,6,11,12a-pentaazabenzo[4,5]cycloocta[1,2,3- cd]indene-2-one |
| 53 | 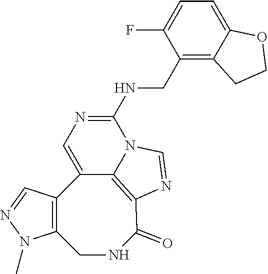 [SMILES: Cn1ncc2c1CNC(=O)c1ncn3c(NCc4c(F)ccc5c4CCO5)ncc-2c13] | 11-(((5-fluoro-2,3-dihydrobenzofuran-4- yl)methyl)amino)-6-methyl-5,6-dihydro- 2,4,6,7,10,11a- hexaazabyclopenta[4,5]cycloocta[1,2,3-cd]inde- 3(4H)-one |
| 54 | 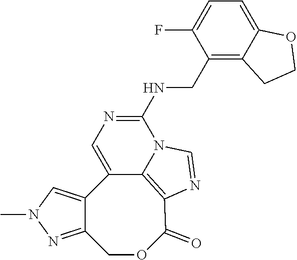 [SMILES: Cn1cc2c(n1)COC(=O)c1ncn3c(NCc4c(F)ccc5c4CCO5)ncc-2c13] | 11-(((5-fluoro-2,3-dihydrobenzofuran-4- yl)methyl)amino)-7-methyl-5,7-dihydro-3H-4- oxa-2,6,7,10,11a- pentaazacyclopenta[4,5]cycloocta[1,2,3-cd]inden- 3-one |
| 55 | 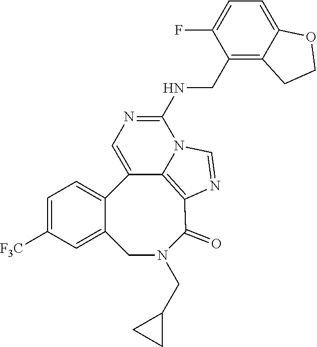 [SMILES: O=C1c2ncn3c(NCc4c(F)ccc5c4CCO5)ncc(c23)-c2ccc(C(F)(F)F)cc2CN1CC1CC1] | 4-(cyclopropylmethyl)-12-(((5-fluoro-2,3- dihydrobenzofuran-4-yl)methyl)amino)-7- (trifluoromethyl)-4,5-dihydro-3H-2,4,6,11,12a- pentaazabenzo[4,5]cycloocta[1,2,3-cd]inden-3- one |
| 56 | 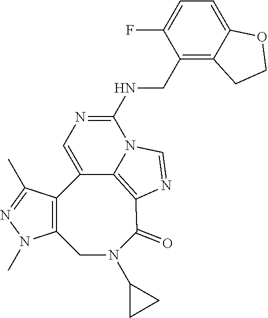 [SMILES: Cc1nn(C)c2c1-c1cnc(NCc3c(F)ccc4c3CCO4)n3cnc(c13)C(=O)N(C1CC1)C2] | 4-cyclopropyl-11-(((5-fluoro-2,3- dihydrobenzofuran-4-yl)methyl)amino)-6,8- dimethyl-5,6-dihydro-2,4,6,7,10,11a- hexaazacyclopenta[4,5]cycloocta[1,2,3-cd]inden- 3(4H)-one |
| 57 | 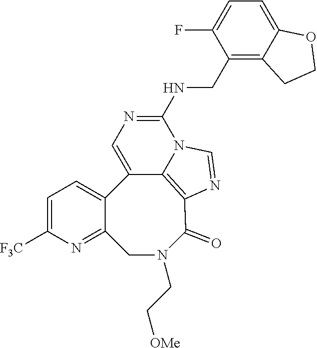 [SMILES: COCCN1Cc2nc(C(F)(F)F)ccc2-c2cnc(NCc3c(F)ccc4c3CCO4)n3cnc(c23)C1=O] | 12-(((5-fluoro-2,3-dihydrobenzofuran-4- yl)methyl)amino)-4-(2-methoxyethyl)-7- (trifluoromethyl)-4,5-dihydro-3H-2,4,6,11,12a- pentaazabenzo[4.5]cycloocta[1,2,3-cd]inden-3- one |
| 58 | 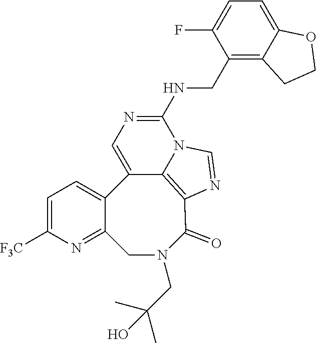 [SMILES: CC(C)(O)CN1Cc2nc(C(F)(F)F)ccc2-c2cnc(NCc3c(F)ccc4c3CCO4)n3cnc(c23)C1=O] | 12-(((5-fluoro-2,3-dihydrobenzofuran-4- yl)methyl)amino)-4-(2-hydroxy-2-methylpropyl)- 7-(trifluoromethyl)-4,5-dihydro-3H-2,4,6,11,12a- pentaazabenzo[4,5]cycloocta[1,2,3-cd]inden-3- one |
| 59 | 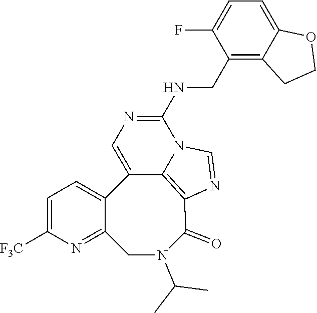 [SMILES: CC(C)N1Cc2nc(C(F)(F)F)ccc2-c2cnc(NCc3c(F)ccc4c3CCO4)n3cnc(c23)C1=O] | 12-(((5-fluoro-2,3-dihydrobenzofuran-4- yl)methyl)amino)-4-isopropyl-7-(trifluoromethyl)- 4,5-dihydro-3H-2,4,6,11,12a- pentaazabenzo[4,5]cycloocta[1,2,3-cd]inden-3- one |
| 60 | 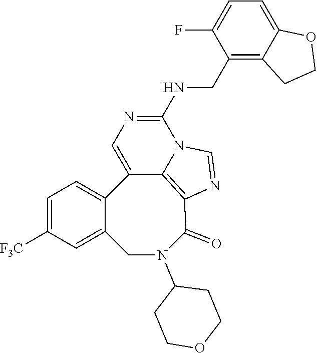 [SMILES: O=C1c2ncn3c(NCc4c(F)ccc5c4CCO5)ncc(c23)-c2ccc(C(F)(F)F)cc2CN1C1CCOCC1] | 12-(((5-fluoro-2,3-dihydrobenzofuran-4- yl)methyl)amino)-4-(tetrahydro-2H-pyran-4-yl)-7- (trifluoromethyl)-4,5-dihydro-3H-2,4,11,12a- tetraazabenzo[4,5]cycloocta[1,2,3-cd]inden-3-one |
| 61 | 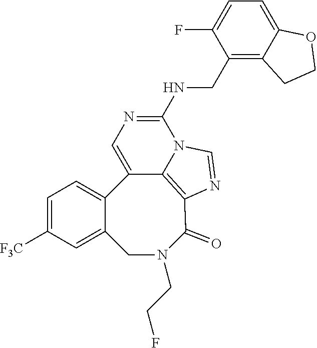 [SMILES: O=C1c2ncn3c(NCc4c(F)ccc5c4CCO5)ncc(c23)-c2ccc(C(F)(F)F)cc2CN1CCF] | 12-(((5-fluoro-2,3-dihydrobenzofuran-4- yl)methyl)amino)-4-(2-fluoroethyl)-7- (trifluoromethyl)-4,5-dihydro-3H-2,4,11,12a- tetraazabenzo[4,5]cycloocta[1,2,3-cd]inden-3-one |
| 62 | 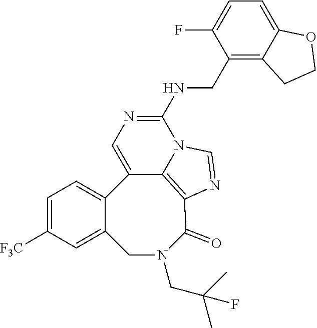 [SMILES: CC(C)(F)CN1Cc2cc(C(F)(F)F)ccc2-c2cnc(NCc3c(F)ccc4c3CCO4)n3cnc(c23)C1=O] | 12-(((5-fluoro-2,3-dihydrobenzofuran-4- yl)methyl)amino)-4-(2-fluoro-2-methylpropyl)-7- (trifluoromethyl)-4,5-dihydro-3H-2,4,11,12a- tetraazabenzo[4,5]cycloocta[1,2,3-cd]inden-3-one |
| 63 | 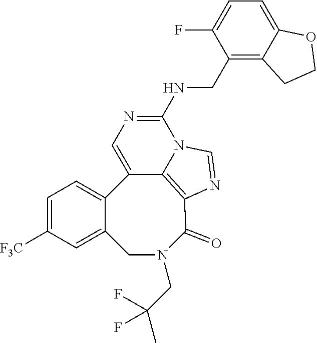 [SMILES: CC(F)(F)CN1Cc2cc(C(F)(F)F)ccc2-c2cnc(NCc3c(F)ccc4c3CCO4)n3cnc(c23)C1=O] | 4-(2,2-difluoropropyl)-12-(((5-fluoro-2,3- dihydrobenzofuran-4-yl)methyl)amino)-7- (trifluoromethyl)-4,5-dihydro-3H-2,4,11,12a- tetraazabenzo[4,5]cycloocta[1,2,3-cd]inden-3-one |
| 64 | 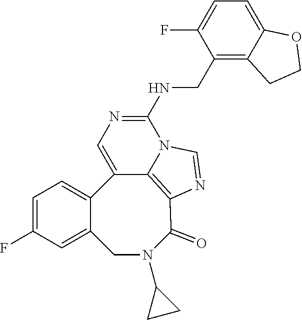 [SMILES: O=C1c2ncn3c(NCc4c(F)ccc5c4CCO5)ncc(c23)-c2ccc(F)cc2CN1C1CC1] | 4-cyclopropyl-7-fluoro-12-(((5-fluoro-2,3- dihydrobenzofuran-4-yl)methyl)amino)-4,5- dihydro-3H-2,4,11,12a- tetraazabenzo[4,5]cycloocta[1,2,3-cd]inden-3-one |
| 65 | 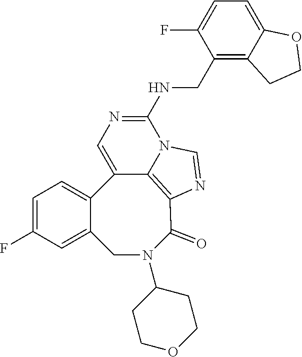 [SMILES: O=C1c2ncn3c(NCc4c(F)ccc5c4CCO5)ncc(c23)-c2ccc(F)cc2CN1C1CCOCC1] | 7-fluoro-12-(((5-fluoro-2,3-dihydrobenzofuran-4- yl)methyl)amino)-4-(tetrahydro-2H-pyran-4-yl)- 4,5-dihydro-3H-2,4,11,12a- tetraazabenzo[4,5]cycloocta[1,2,3-cd]inden-3-one |
| 66 | 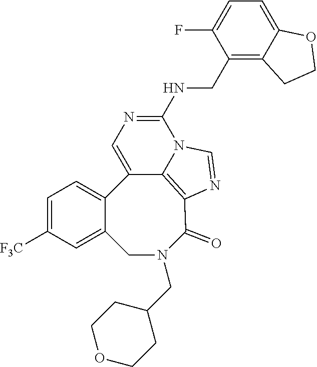 [SMILES: O=C1c2ncn3c(NCc4c(F)ccc5c4CCO5)ncc(c23)-c2ccc(C(F)(F)F)cc2CN1CC1CCOCC1] | 12-(((5-fluoro-2,3-dihydrobenzofuran-4- yl)methyl)amino)-4-((tetrahydro-2H-pyran-4- yl)methyl)-7-(trifluoromethyl)-4,5-dihydro-3H- 2,4,11,12a-tetraazabenzo[4,5]cycloocta[1,2,3- cd]inden-3-one |
| 67 | 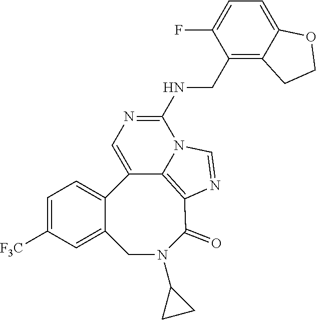 [SMILES: O=C1c2ncn3c(NCc4c(F)ccc5c4CCO5)ncc(c23)-c2ccc(C(F)(F)F)cc2CN1C1CC1] | 4-cyclopropyl-12-(((5-fluoro-2,3- dihydrobenzofuran-4-yl)methyl)amino)-7- (trifluoromethyl)-4,5-dihydro-3H-2,4,11,12a- tetraazabenzo[4,5]cycloocta[1,2,3-cd]inden-3-one |
| 68 | 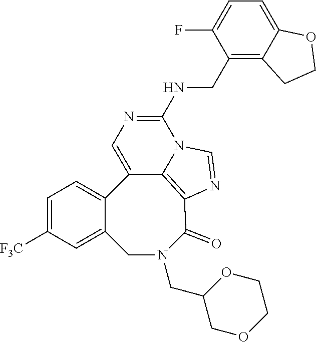 [SMILES: O=C1c2ncn3c(NCc4c(F)ccc5c4CCO5)ncc(c23)-c2ccc(C(F)(F)F)cc2CN1CC1COCCO1] | 4-((1,4-dioxan-2-yl)methyl)-12-(((5-fluoro-2,3- dihydrobenzofuran-4-yl)methyl)amino)-7- (trifluoromethyl)-4,5-dihydro-3H-2,4,11,12a- tetraazabenzo[4,5]cycloocta[1,2,3-cd]inden-3-one |
| 69 | 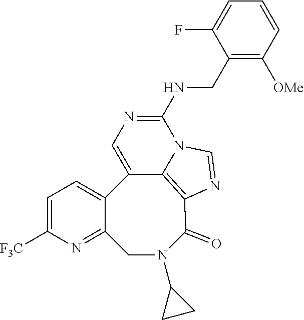 [SMILES: COc1cccc(F)c1CNc1ncc2c3c(ncn13)C(=O)N(C1CC1)Cc1nc(C(F)(F)F)ccc1-2] | 4-cyclopropyl-12-((2-fluoro-6- methoxybenzyl)amino)-7-(trifluoromethyl)-4,5- dihydro-3H-2,4,6,11,12a- pentaazabenzo[4,5]cycloocta[1,2,3-cd]inden-3- one |
| 70 | 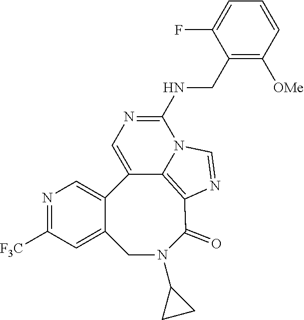 [SMILES: COc1cccc(F)c1CNc1ncc2c3c(ncn13)C(=O)N(C1CC1)Cc1cc(C(F)(F)F)ncc1-2] | 4-cyclopropyl-12-((2-fluoro-6- methoxybenzyl)amino)-7-(trifluoromethyl)-4,5- dihydro-3H-2,4,8,11,12a- pentaazabenzo[4,5]cycloocta[1,2,3-cd]inden-3- one |
| 71 | 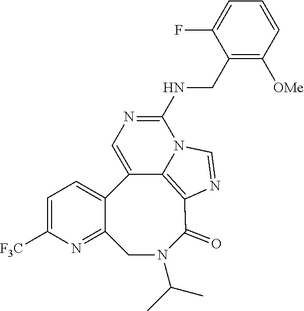 [SMILES: COc1cccc(F)c1CNc1ncc2c3c(ncn13)C(=O)N(C(C)C)Cc1nc(C(F)(F)F)ccc1-2] | 12-((2-fluoro-6-methoxybenzyl)amino)-4- isopropyl-7-(trifluoromethyl)-4,5-dihydro-3H- 2,4,6,11,12a-pentaazabenzo[4,5]cycloocta[1,2,3- cd]inden-3-one |
| 72 | 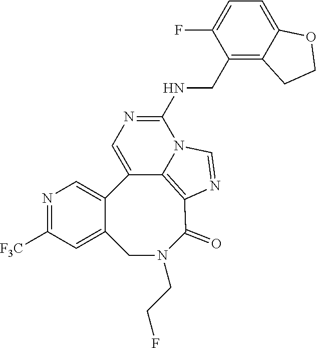 [SMILES: O=C1c2ncn3c(NCc4c(F)ccc5c4CCO5)ncc(c23)-c2cnc(C(F)(F)F)cc2CN1CCF] | 12-(((5-fluoro-2,3-dihydrobenzofuran-4- yl)methyl)amino)-4-(2-fluoroethyl)-7- (trifluoromethyl)-4,5-dihydro-3H-2,4,8,11,12a- pentaazabenzo[4,5]cycloocta[1,2,3-cd]inden-3- one |
| 73 | 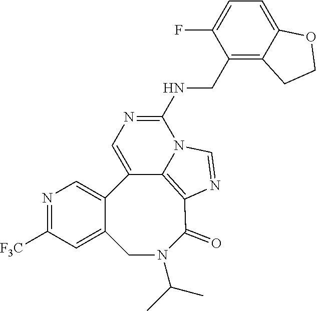 [SMILES: CC(C)N1Cc2cc(C(F)(F)F)ncc2-c2cnc(NCc3c(F)ccc4c3CCO4)n3cnc(c23)C1=O] | 12-(((5-fluoro-2,3-dihydrobenzofuran-4- yl)methyl)amino)-4-isopropyl-7-(trifluoromethyl)- 4,5-dihydro-3H-2,4,8,11,12a- pentaazabenzo[4,5]cycloocta[1,2,3-cd]inden-3- one |
| 74 | 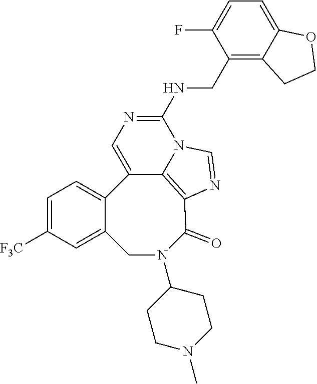 [SMILES: CN1CCC(N2Cc3cc(C(F)(F)F)ccc3-c3cnc(NCc4c(F)ccc5c4CCO5)n4cnc(c34)C2=O)CC1] | 12-(((5-fluoro-2,3-dihydrobenzofuran-4- yl)methyl)amino)-4-(1-methylpiperidin-4-yl)-7- (trifluoromethyl)-4,5-dihydro-3H-2,4,11,12a- tetraazabenzo[4,5]cycloocta[1,2,3-cd]inden-3-one |
| 75 | 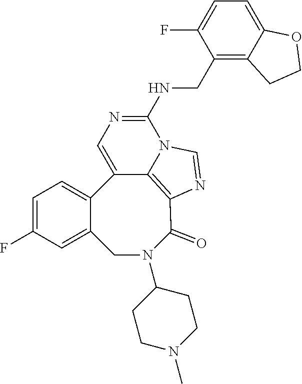 [SMILES: CN1CCC(N2Cc3cc(F)ccc3-c3cnc(NCc4c(F)ccc5c4CCO5)n4cnc(c34)C2=O)CC1] | 7-fluoro-12-(((5-fluoro-2,3-dihydrobenzofuran-4- yl)methyl)amino)-4-(1-methylpiperidin-4-yl)-4,5- dihydro-3H-2,4,11,12a- tetraazabenzo[4,5]cycloocta[1,2,3-cd]inden-3-one |
| 76 | 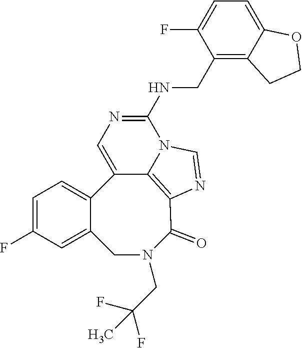 [SMILES: CC(F)(F)CN1Cc2cc(F)ccc2-c2cnc(NCc3c(F)ccc4c3CCO4)n3cnc(c23)C1=O] | 4-(2,2-difluoropropyl)-7-fluoro-12-(((5-fluoro- 2,3-dihydrobenzofuran-4-yl)methyl)amino)-4,5- dihydro-3H-2,4,11,12a- tetraazabenzo[4.5]cycloocta[1,2,3-cd]inden-3-one |
| 77 | 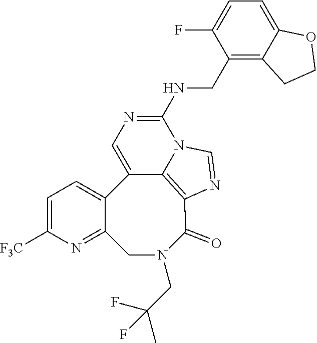 [SMILES: CC(F)(F)CN1Cc2nc(C(F)(F)F)ccc2-c2cnc(NCc3c(F)ccc4c3CCO4)n3cnc(c23)C1=O] | 4-(2,2-difluoropropyl)-12-(((5-fluoro-2,3- dihydrobenzofuran-4-yl)methyl)amino)-7- (trifluoromethyl)-4,5-dihydro-3H-2,4,6,11,12a- pentaazabenzo[4,5]cycloocta[1,2,3-cd]inden-3- one |
| 78 | 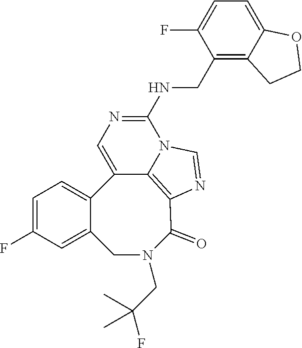 [SMILES: CC(C)(F)CN1Cc2cc(F)ccc2-c2cnc(NCc3c(F)ccc4c3CCO4)n3cnc(c23)C1=O] | 7-fluro-12-(((5-fluoro-2,3-dihydrobenzofuran-4- yl)methyl)amino)-4-(2-fluoro-2-methylpropyl)- 4,5-dihydro-3H-2,4,11,12a- tetraazabenzo[4,5]cycloocta[1,2,3-cd]inden-3-one |
| 79 | 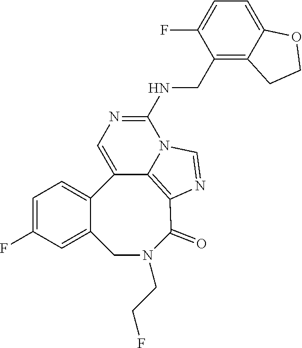 [SMILES: O=C1c2ncn3c(NCc4c(F)ccc5c4CCO5)ncc(c23)-c2ccc(F)cc2CN1CCF] | 7-fluoro-12-(((5-fluoro-2,3-dihydrobenzofuran-4- yl)methyl)amino)-4-(2-fluoroethyl)-4,5-dihydro- 3H-2,4,11,12a-tetraazabenzo[4,5]cycloocta[1,2,3- cd]indene-3-one |
| 80 | 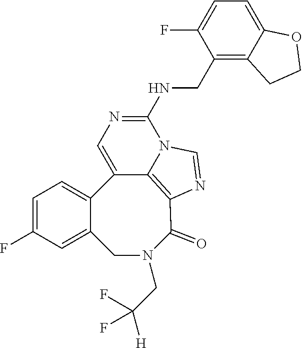 [SMILES: O=C1c2ncn3c(NCc4c(F)ccc5c4CCO5)ncc(c23)-c2ccc(F)cc2CN1CC(F)F] | 4-(2,2-diflurooethyl)-7-fluoro-12-(((5-fluoro-2,3- dihydrobenzofuran-4-yl)methyl)amino)-4,5- dihydro-3H-2,4,11,12a- tetraazabenzo[4,5]cycloocta[1,2,3-cd]inden-3-one |
| 81 | 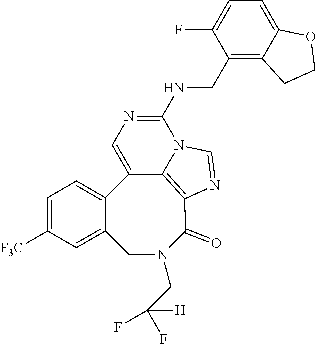 [SMILES: O=C1c2ncn3c(NCc4c(F)ccc5c4CCO5)ncc(c23)-c2ccc(C(F)(F)F)cc2CN1CC(F)F] | 4-(2,2-difluoroethyl)-12-(((5-fluoro-2,3- dihydrobenzofuran-4-yl)methyl)amino)-7- (trifluoromethyl)-4,5-dihydro-3H-2,4,11,12a- tetraazabenzo[4,5]cycloocta[1,2,3-cd]inden-3-one |
| 82 | 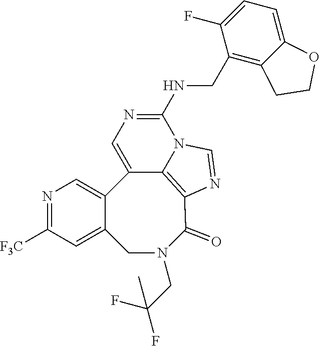 [SMILES: CC(F)(F)CN1Cc2cc(C(F)(F)F)ncc2-c2cnc(NCc3c(F)ccc4c3CCO4)n3cnc(c23)C1=O] | 4-(2,2-difluoropropyl)-12-(((5-fluoro-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| 83 | 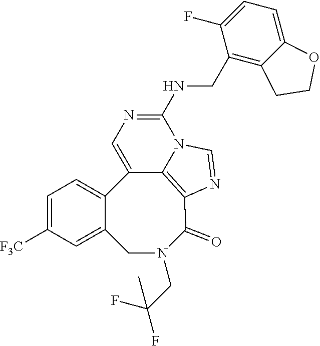 [SMILES: CC(F)(F)CN1Cc2cc(C(F)(F)F)ccc2-c2cnc(NCc3c(F)ccc4c3CCO4)n3cnc(c23)C1=O] | 12-(((5-fluoro-2,3-dihydrobenzofuran-4- yl)methyl)amino)-4-(2,2,2-trifluoroethyl)-7- (trifluoromethyl)-4,5-dihydro-3H-2,4,11,12a- tetraazabenzo[4,5]cycloocta[1,2,3-cd]inden-3-one |
| 84 | 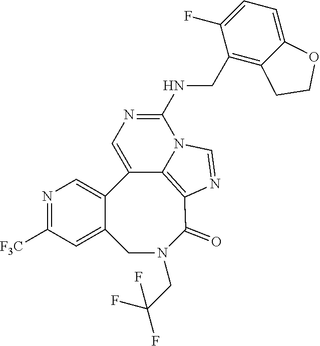 [SMILES: O=C1c2ncn3c(NCc4c(F)ccc5c4CCO5)ncc(c23)-c2cnc(C(F)(F)F)cc2CN1CC(F)(F)F] | 12-(((5-fluoro-2,3-dihydrobenzofuran-4- yl)methyl)amino)-4-(2,2,2-trifluoroethyl)-7- (trifluoromethyl)-4,5-dihydro-3H-2,4,8,11,12a- pentaazabenzo[4,5]cycloocta[1,2,3-cd]inden-3- one |
| 85 | 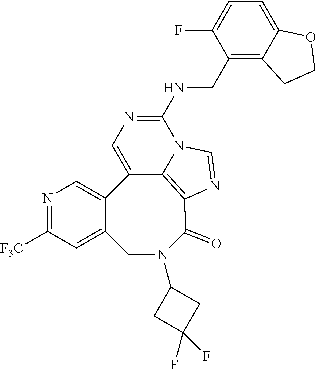 [SMILES: O=C1c2ncn3c(NCc4c(F)ccc5c4CCO5)ncc(c23)-c2cnc(C(F)(F)F)cc2CN1C1CC(F)(F)C1] | 4-(3,3-difluorocyclobutyl)-12-(((5-fluoro-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| 86 | 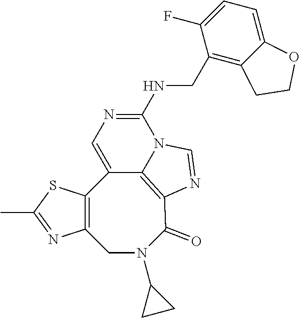 [SMILES: Cc1nc2c(s1)-c1cnc(NCc3c(F)ccc4c3CCO4)n3cnc(c13)C(=O)N(C1CC1)C2] | 4-cyclopropyl-11-(((5-fluoro-2,3- dihydrobenzofuran-4-yl)methyl)amino)-7-methyl- 4,5-dihydro-3H-8-thia-2,4,6,10,11a- pentaazacyclopenta[4,5]cycloocta[1,2,3-cd]inden- 3-one |
| 87 | 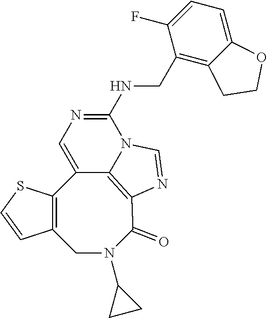 [SMILES: O=C1c2ncn3c(NCc4c(F)ccc5c4CCO5)ncc(c23)-c2sccc2CN1C1CC1] | 4-cyclopropyl-11-(((5-fluoro-2,3- dihydrobenzofuran-4-yl)methyl)amino)-4,5- dihydro-3H-8-thia-2,4,10,11a- tetraazacyclopenta[4,5]cycloocta[1,2,3-cd]inden- 3-one |
| 88 | 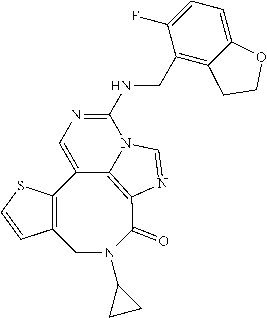 [SMILES: O=C1c2ncn3c(NCc4c(F)ccc5c4CCO5)ncc(c23)-c2sccc2CN1C1CC1] | 4-cyclopropyl-11-(((5-fluoro-2,3- dihydrobenzofuran-4-yl)methyl)amino)-4,5- dihydro-3H-6-thia-2,4,10,11a- tetraazacyclopenta[4,5]cycloocta[1,2,3-cd]inden- 3-one |
| 89 | 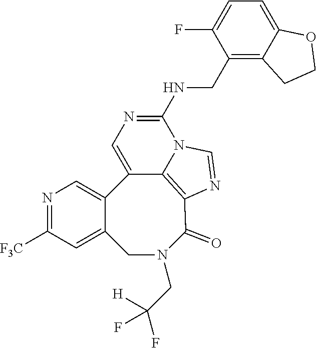 [SMILES: O=C1c2ncn3c(NCc4c(F)ccc5c4CCO5)ncc(c23)-c2cnc(C(F)(F)F)cc2CN1CC(F)F] | 4-(2,2-difluoroethyl)-12-(((5-fluoro-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| 90 | 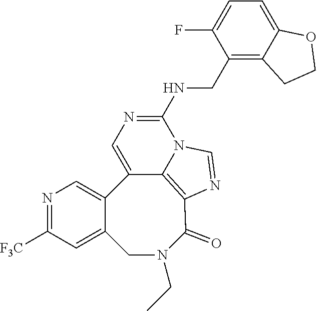 [SMILES: CCN1Cc2cc(C(F)(F)F)ncc2-c2cnc(NCc3c(F)ccc4c3CCO4)n3cnc(c23)C1=O] | 4-ethyl-12-(((5-fluoro-2,3-dihydrobenzofuran-4- yl)methyl)amino)-7-(trifluoromethyl)-4,5- dihydro-3H-2,4,8,11,12a- pentaazabenzo[4,5]cycloocta[1,2,3-cd]inden-3- one |
| 91 | 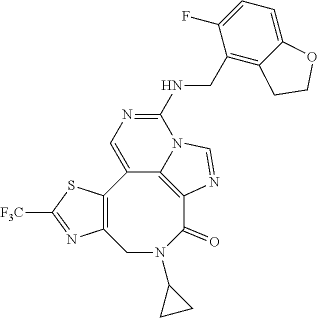 [SMILES: O=C1c2ncn3c(NCc4c(F)ccc5c4CCO5)ncc(c23)-c2sc(C(F)(F)F)nc2CN1C1CC1] | 4-cyclopropyl-11-(((5-fluoro-2,3- dihydrobenzofuran-4-yl)methyl)amino)-7- (trifluoromethyl)-4,5-dihydro-3H-8-thia- 2,4,6,10,11a- pentaazacyclopenta[4,5]cycloocta[1,2,3-cd]inden- 3-one |
| 92 | 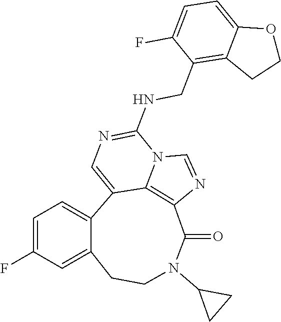 [SMILES: O=C1c2ncn3c(NCc4c(F)ccc5c4CCO5)ncc(c23)-c2ccc(F)cc2CCN1C1CC1] | 4-cyclopropyl-8-fluoro-13-(((5-fluoro-2,3- dihydrobenzofuran-4-yl)methyl)amino)-5,6- dihydro-2,4,12,13a- tetraazabenzo[4,5]cyclonona[1,2,3-cd]inden- 3(4H)-one |
| 93 | 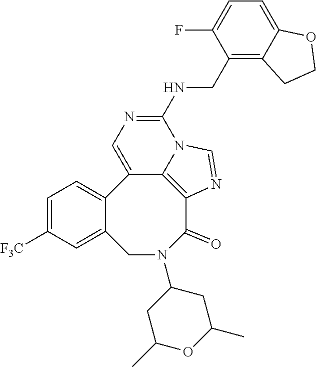 [SMILES: CC1CC(N2Cc3cc(C(F)(F)F)ccc3-c3cnc(NCc4c(F)ccc5c4CCO5)n4cnc(c34)C2=O)CC(C)O1] | 4-(2,6-dimethyltetrahydro-2H-pyran-4-yl)-12- (((5-fluoro-2,3-dihydrobenzofuran-4- yl)methyl)amino)-7-(trifluroomethyl)-4,5- dihydro-3H-2,4,11,12a- tetraazabenzo[4,5]cycloocta[1,2,3-cd]inden-3-one |
| 94 | 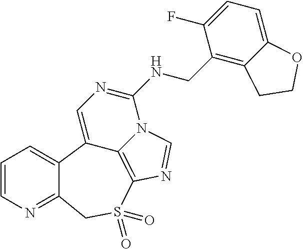 [SMILES: O=S1(=O)Cc2ncccc2-c2cnc(NCc3c(F)ccc4c3CCO4)n3cnc1c23] | 11-(((5-fluoro-2,3-dihydrobenzofuran-4- yl)methyl)amino)-4H-3-thia-2,5,10,11a- tetraazadibenzo[cd,f]azulene 3,3-dioxide |
| 95 | 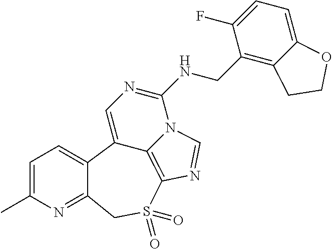 [SMILES: Cc1ccc2c(n1)CS(=O)(=O)c1ncn3c(NCc4c(F)ccc5c4CCO5)ncc-2c13] | 11-(((5-fluoro-2,3-dihydrobenzofuran-4- yl)methyl)amino)-6-methyl-4H-3-thia-2,5,10,11a- tetraazadibenzo[cd,f]azulene 3,3-dioxide |
| 96 | 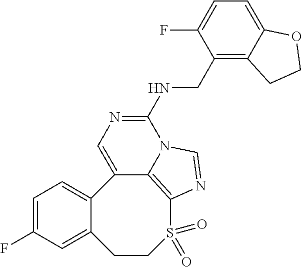 [SMILES: O=S1(=O)CCc2cc(F)ccc2-c2cnc(NCc3c(F)ccc4c3CCO4)n3cnc1c23] | 7-fluoro-12-(((5-fluoro-2,3-dihydrobenzofuran-4- yl)methyl)amino)-4,5-dihydro-3-thia-2,11,12a- triazabenzo[4,5]cycloocta[1,2,3-cd]indene 3,3- dioxide |
| 97 | 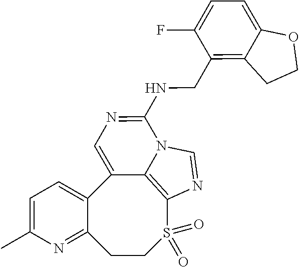 [SMILES: Cc1ccc2c(n1)CCS(=O)(=O)c1ncn3c(NCc4c(F)ccc5c4CCO5)ncc-2c13] | 12-(((5-fluoro-2,3-dihydrobenzofuran-4- yl)methyl)amino)-7-methyl-4,5-dihydro-3-thia- 2,6,11,12a-tetraazabenzo[4,5]cycloocta[1,2,3- cd]indene 3,3-dioxide |
| 98 | 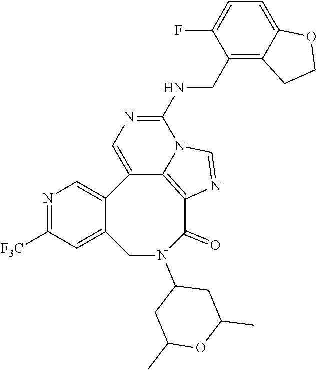 [SMILES: CC1CC(N2Cc3cc(C(F)(F)F)ncc3-c3cnc(NCc4c(F)ccc5c4CCO5)n4cnc(c34)C2=O)CC(C)O1] | 4-(2,6-dimethyltetrahydro-2H-pyran-4-yl)-12- (((5-fluoro-2,3-dihydrobenzofuran-4- yl)methyl)amino)-7-(trifluoromethyl)-4,5- dihydro-3H-2,4,8,11,12a- pentaazabenzo[4,5]cycloocta[1,2,3-cd]inden-3- one |
| 99 | 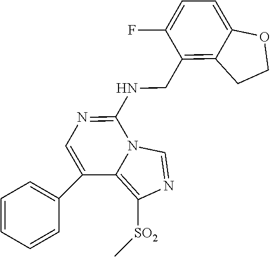 [SMILES: CS(=O)(=O)c1ncn2c(NCc3c(F)ccc4c3CCO4)ncc(-c3ccccc3)c12] | N-((5-fluoro-2,3-dihydrobenzofuran-4-yl)methyl)- 1-(methylsulfonyl)-8-phenylimidazo[1,5- c]pyrimidin-5-amine |
| 100 | 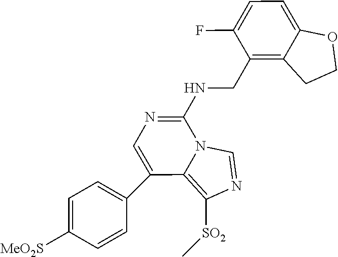 [SMILES: CS(=O)(=O)c1ccc(-c2cnc(NCc3c(F)ccc4c3CCO4)n3cnc(S(C)(=O)=O)c23)cc1] | N-((5-fluoro-2,3-dihydrobenzofuran-4-yl)methyl)- 1-(methylsulfonyl)-8-(4 (methylsulfonyl)phenyl)imidazo[1,5-c]pyrimidin- 5-amine |
| 101 | 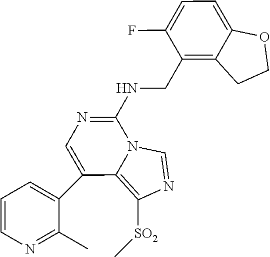 [SMILES: Cc1ncccc1-c1cnc(NCc2c(F)ccc3c2CCO3)n2cnc(S(C)(=O)=O)c12] | N-((5-fluoro-2,3-dihydrobenzofuran-4-yl)methyl)- 8-(2-methylpyridin-3-yl)-1- (methylsulfonyl)imidazo[1,5-c]pyrimidin-5-amine |
| 102 | 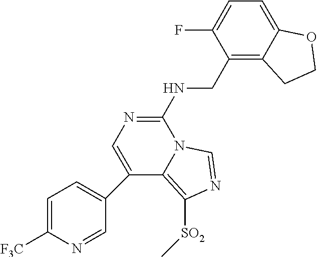 [SMILES: CS(=O)(=O)c1ncn2c(NCc3c(F)ccc4c3CCO4)ncc(-c3ccc(C(F)(F)F)nc3)c12] | N-((5-fluoro-2,3-dihydrobenzofuran-4-yl)methyl)- 1-(methylsulfonyl)-8-(6-(trifluoromethyl)pyridin- 3-yl)imidazo[1,5-c]pyrimidin-5-amine |
| 103 | 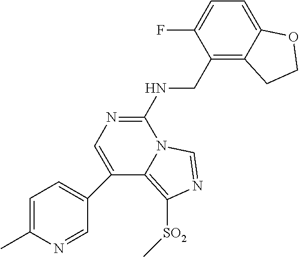 [SMILES: Cc1ccc(-c2cnc(NCc3c(F)ccc4c3CCO4)n3cnc(S(C)(=O)=O)c23)cn1] | N-((5-fluoro-2,3-dihydrobenzofuran-4-yl)methyl)- 8-(6-methylpyridin-3-yl)-1- (methylsulfonyl)imidazo[1,5-c]pyrimidin-5-amine |
| 104 | 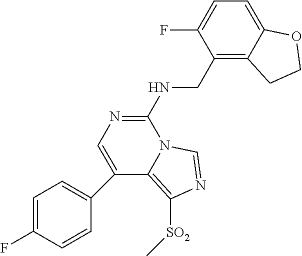 [SMILES: CS(=O)(=O)c1ncn2c(NCc3c(F)ccc4c3CCO4)ncc(-c3ccc(F)cc3)c12] | N-((5-fluoro-2,3-dihydrobenzofuran-4-yl)methyl)- 8-(4-fluorophenyl)-1 (methylsulfonyl)imidazo[1,5-c]pyrimidin-5-amine |
| 105 | 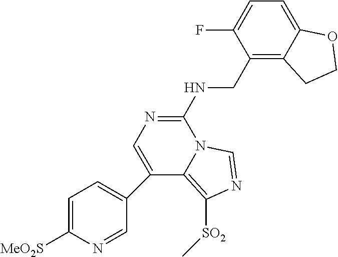 [SMILES: CS(=O)(=O)c1ccc(-c2cnc(NCc3c(F)ccc4c3CCO4)n3cnc(S(C)(=O)=O)c23)cn1] | N-((5-fluoro-2,3-dihydrobenzofuran-4-yl)methyl)- 1-(methylsulfonyl)-8-(6-methylsulfonyl)pyridin- 3-yl)imidazo[1,5-c]pyrimidin-5-amine |
| 106 | 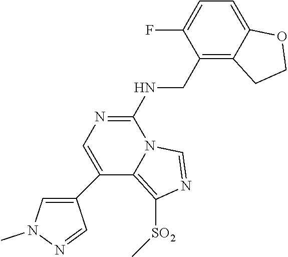 [SMILES: Cn1cc(-c2cnc(NCc3c(F)ccc4c3CCO4)n3cnc(S(C)(=O)=O)c23)cn1] | N-((5-fluoro-2,3-dihydrobenzofuran-4-yl)methyl)- 8-(1-methyl-1H-pyrazol-4-yl)-1- (methylsulfonyl)imidazo[1,5-c]pyrimidin-5-amine |
| 107 | 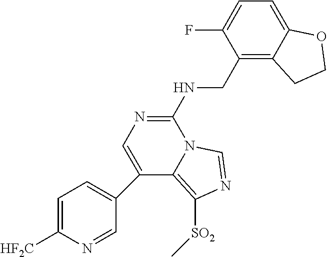 | 8-(6-(difluoromethyl)pyridin-3-yl)-N-((5-fluoro- 2,3-dihydrobenzofuran-4-yl)methyl)-1- (methylsulfonyl)imidazo[1,5-c]pyrimidin-5-amine |
| 108 | 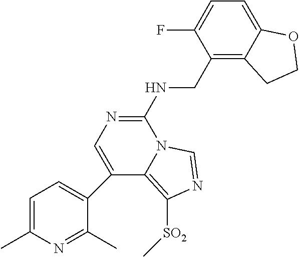 [SMILES: Cc1ccc(-c2cnc(NCc3c(F)ccc4c3CCO4)n3cnc(S(C)(=O)=O)c23)c(C)n1] | 8-(2,6-dimethylpyridin-3-yl)-N-((5-fluoro-2,3- dihydrobenzofuran-4-yl)methyl)-1- (methylsulfonyl)imidazo[1,5-c]pyrimidin-5-amine |
| 109 | 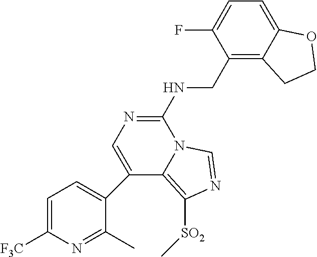 [SMILES: Cc1nc(C(F)(F)F)ccc1-c1cnc(NCc2c(F)ccc3c2CCO3)n2cnc(S(C)(=O)=O)c12] | N-((5-fluoro-2,3-dihydrobenzofuran-4-yl)methyl)- 8-(2-methyl-6-(trifluoromethyl)pyridin-3-yl)-1- (methylsulfonyl)imidazo[1,5-c]pyrimidin-5-amine |
| 110 | 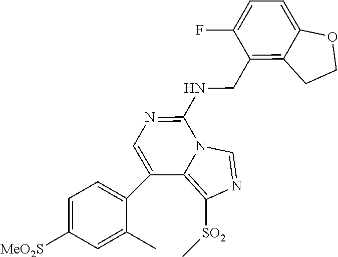 [SMILES: Cc1cc(S(C)(=O)=O)ccc1-c1cnc(NCc2c(F)ccc3c2CCO3)n2cnc(S(C)(=O)=O)c12] | N-((5-fluoro-2,3-dihydrobenzofurna-4-yl)methyl)- 8-(2-methyl-4-(methylsulfonyl)phenyl)-1- (methylsulfonyl)imidazo[1,5-c]pyrimidin-5-amine |
| 111 | 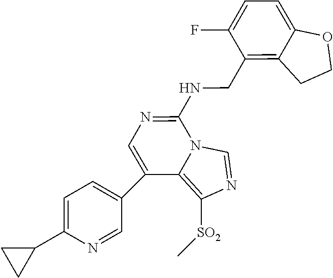 [SMILES: CS(=O)(=O)c1ncn2c(NCc3c(F)ccc4c3CCO4)ncc(-c3ccc(C4CC4)nc3)c12] | 8-(6-cyclopropylpyridin-3-yl)-N-((5-fluoro-2,3- dihydrobenzofuran-4-yl)methyl)-1- (methyslulfonyl)imidazo[1,5-c]pyrimidin-5-amine |
| 112 | 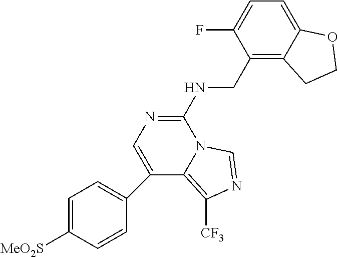 [SMILES: CS(=O)(=O)c1ccc(-c2cnc(NCc3c(F)ccc4c3CCO4)n3cnc(C(F)(F)F)c23)cc1] | N-((5-fluoro-2,3-dihydrobenzofuran-4-yl)methyl)- 8-(4-(methylsulfonyl)phenyl)-1- (trifluoromethyl)imidazo[1,5-c]pyrimdiin-5-amine |
| 113 | 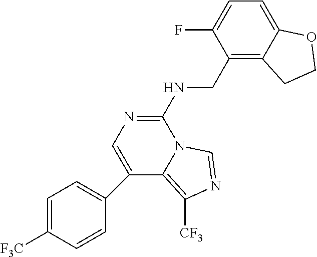 [SMILES: Fc1ccc2c(c1CNc1ncc(-c3ccc(C(F)(F)F)cc3)c3c(C(F)(F)F)ncn13)CCO2] | N-((5-fluoro-2,3-dihydrobenzofuran-4-yl)methyl)- 1-(trifluoromethyl)-8-(4- (trifluoromethyl)phenyl)imidazo[1,5-c]pyrimidin- 5-amine |
| 114 | 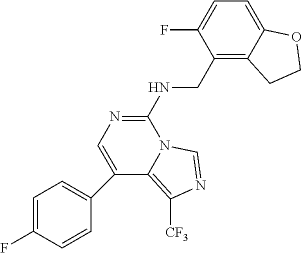 [SMILES: Fc1ccc(-c2cnc(NCc3c(F)ccc4c3CCO4)n3cnc(C(F)(F)F)c23)cc1] | N-((5-fluoro-2,3-dihydrobenzofuran-4-yl)methyl)- 8-(4-fluorophenyl)-1- (trifluoromethyl)imidazo[1,5-c]pyrimidin-5-amine |
| 115 | 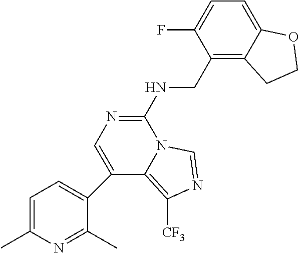 [SMILES: Cc1ccc(-c2cnc(NCc3c(F)ccc4c3CCO4)n3cnc(C(F)(F)F)c23)c(C)n1] | 8-(2,6-dimethylpyridin-3-yl)-N-((5-fluoro-2,3- dihydrobenzofuran-4-yl)methyl)-1- (trifluoromethyl)imidazo[1,5-c]pyrimdiin-5-amine |
| 116 | 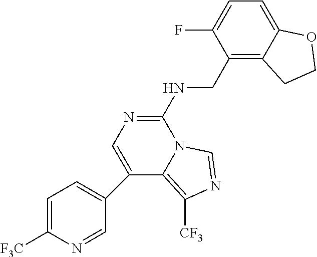 [SMILES: Fc1ccc2c(c1CNc1ncc(-c3ccc(C(F)(F)F)nc3)c3c(C(F)(F)F)ncn13)CCO2] | N-((5-fluoro-2,3-dihydrobenzofuran-4-yl)methyl)- 1-(trifluoromethyl)-8-(6-(trifluoromethyl)pyridin- 3-yl)imidazo[1,5-c]pyrimidin-5-amine |
| 117 | 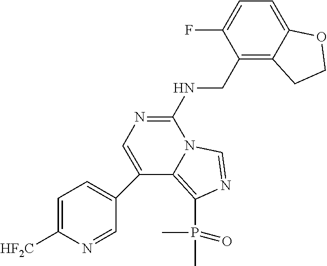 | (8-(6-(difluoromethyl)pyridin-3-yl)-5-(((5-fluoro- 2,3-dihydrobenzofuran-4- yl)methyl)amino)imidazo[1,5-c]pyrimidin-1- yl)dimethylphosphine oxide |
| 118 | 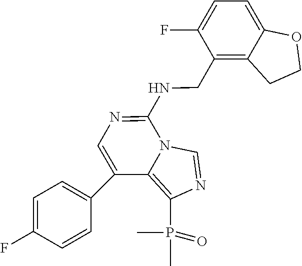 [SMILES: CP(C)(=O)c1ncn2c(NCc3c(F)ccc4c3CCO4)ncc(-c3ccc(F)cc3)c12] | (5-(((5-fluoro-2,3-dihydrobenzofuran-4- yl)methyl)amino)-8-(4-fluorophenyl)imidazo[1,5- c]pyrimidin-1-yl)dimethylphosphine oxide |
| 119 | 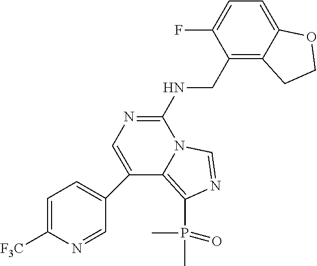 [SMILES: CP(C)(=O)c1ncn2c(NCc3c(F)ccc4c3CCO4)ncc(-c3ccc(C(F)(F)F)nc3)c12] | (5-(((5-fluoro-2,3-dihydrobenzofuran-4- yl)methyl)amino)-8-(6-(trifluoromethyl)pyridin-3- yl)imidazo[1,5-c]pyrimidin-1- yl)dimethylphosphine oxide |
| 120 | 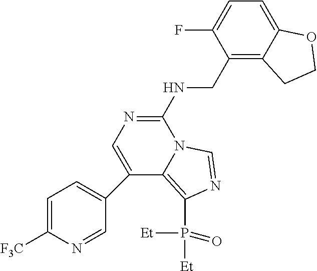 [SMILES: CCP(=O)(CC)c1ncn2c(NCc3c(F)ccc4c3CCO4)ncc(-c3ccc(C(F)(F)F)nc3)c12] | diethyl(5-(((5-fluoro-2,3-dihydrobenzofuran-4- yl)methyl)amino)-8-(6-(trifluoromethyl)pyridin-3- yl)imidazo[1,5-c]pyrimidin-1-yl)phosphine oxide |
| 121 | 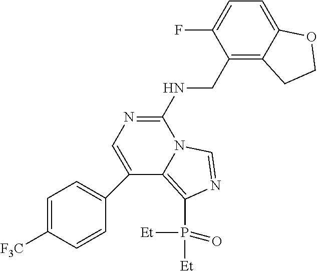 [SMILES: CCP(=O)(CC)c1ncn2c(NCc3c(F)ccc4c3CCO4)ncc(-c3ccc(C(F)(F)F)cc3)c12] | diethyl(5-(((5-fluoro-2,3-dihydrobenzofuran-4- yl)methyl)amino)-8-(4- (trifluoromethyl)phenyl)imidazo[1,5-c]pyrimidin- 1-yl)phosphine oxide |
| 122 | 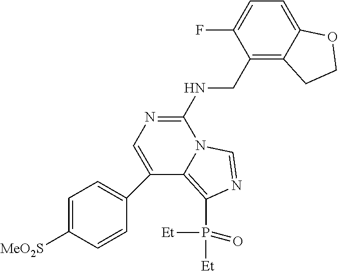 [SMILES: CCP(=O)(CC)c1ncn2c(NCc3c(F)ccc4c3CCO4)ncc(-c3ccc(S(C)(=O)=O)cc3)c12] | diethyl(5-(((5-fluoro-2,3-dihydrobenzofuran-4- yl)methyl)amino)-8-(4- (methylsulfonyl)phenyl)imidazo[1,5-c]pyrimidin- 1-yl)phosphine oxide |
| 123 | 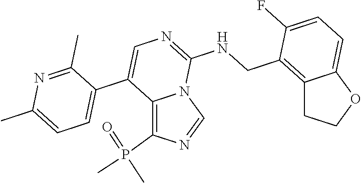 [SMILES: Cc1ccc(-c2cnc(NCc3c(F)ccc4c3CCO4)n3cnc(P(C)(C)=O)c23)c(C)n1] | (8-(2,6-dimethylpyridin-3-yl)-5-(((5-fluoro-2,3- dihydrobenzofuran-4- yl)methyl)amino)imidazo[1,5-c]pyrimidin-1- yl)dimethylphosphine oxide |
| 124 | 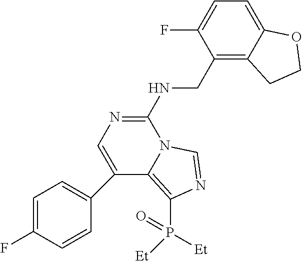 [SMILES: CCP(=O)(CC)c1ncn2c(NCc3c(F)ccc4c3CCO4)ncc(-c3ccc(F)cc3)c12] | diethyl(5-(((5-fluoro-2,3-dihydrobenzofuran-4- yl)methyl)amino)-8-(4-fluorophenyl)imidazo[1,5- c]pyrimidin-1-yl)phosphine oxide |
| 125 | 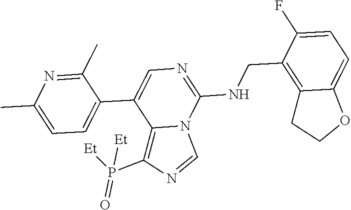 [SMILES: CCP(=O)(CC)c1ncn2c(NCc3c(F)ccc4c3CCO4)ncc(-c3ccc(C)nc3C)c12] | (8-(2,6-dimethylpyriidn-3-yl)-5-(((5-fluoro-2,3- dihydrobenzofuran-4- yl)methyl)amino)imidazo[1,5-c]pyrimidin-1- yl)diethylphosphine oxide |
| 126 | 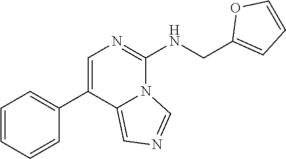 [SMILES: c1ccc(-c2cnc(NCc3ccco3)n3cncc23)cc1] | N-(furan-2-ylmethyl)-8-phenylimidazo[1,5- c]pyrimidin-5-amine |
| 127 | 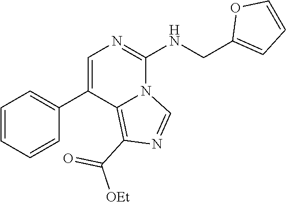 [SMILES: CCOC(=O)c1ncn2c(NCc3ccco3)ncc(-c3ccccc3)c12] | ethyl 5-((furan-2-ylmethyl)amino)-8- phenylimidazo[1,5-c]pyrimidine-1-carboxylate |
| 128 | 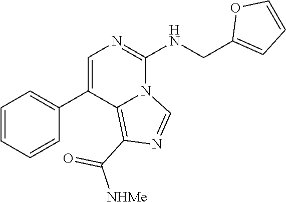 [SMILES: CNC(=O)c1ncn2c(NCc3ccco3)ncc(-c3ccccc3)c12] | 5-((furan-2-ylmethyl)amino)-N-methyl-8- phenylimidazo[1,5-c]pyrimidine-1-carboxamide |
| 129 | 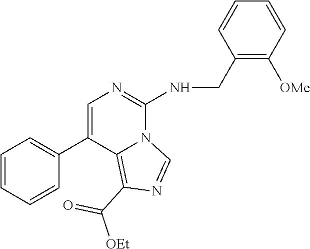 [SMILES: CCOC(=O)c1ncn2c(NCc3ccccc3OC)ncc(-c3ccccc3)c12] | ethyl 5-((2-methoxybenzyl)amino)-8- phenylimidazo[1,5-c]pyrimidine-1-carboxylate |
| 130 | 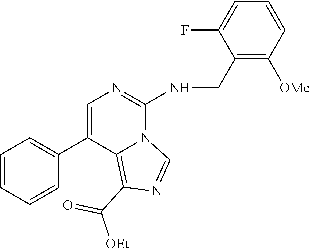 [SMILES: CCOC(=O)c1ncn2c(NCc3c(F)cccc3OC)ncc(-c3ccccc3)c12] | ethyl 5-((2-fluoro-6-methoxybenzyl)amino)-8- phenylimidazo[1,5-c]pyrimidine-1-carboxylate |
| 131 | 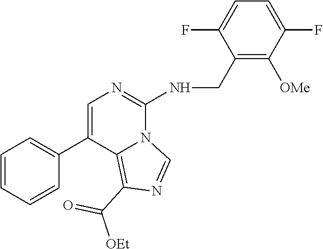 [SMILES: CCOC(=O)c1ncn2c(NCc3c(F)ccc(F)c3OC)ncc(-c3ccccc3)c12] | ethyl 5-((2,6-difluoro-3-methoxybenzyl)amino)-8- phenylimidazo[1,5-c]pyrimidine-1-carboxylate |
| 132 | 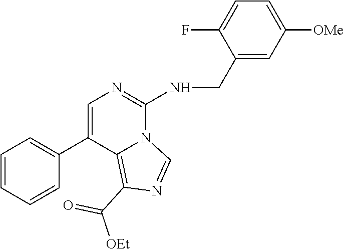 [SMILES: CCOC(=O)c1ncn2c(NCc3cc(OC)ccc3F)ncc(-c3ccccc3)c12] | ethyl 5-((2-fluoro-5-methoxybenzyl)amino)-8- phenylimidazo[1,5-c]pyrimidine-1-carboxylate |
| 133 | 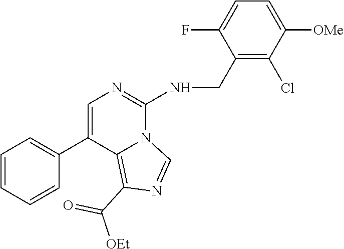 [SMILES: CCOC(=O)c1ncn2c(NCc3c(F)ccc(OC)c3Cl)ncc(-c3ccccc3)c12] | ethyl 5-((2-chloro-6-fluoro-3- methoxybenzyl)amino)-8-phenylimidazo[1,5- c]pyrimidine-1-carboxylate |
| 134 | 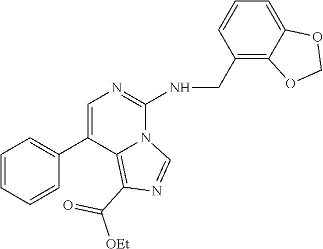 [SMILES: CCOC(=O)c1ncn2c(NCc3cccc4c3OCO4)ncc(-c3ccccc3)c12] | ethyl 5-((benzo[d][1,3]dioxol-4-ylmethyl)amino)- 8-phenylimidazo[1,5-c]pyrimidine-1-carboxylate |
| 135 | 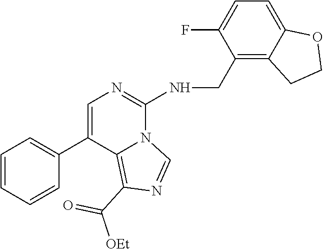 [SMILES: CCOC(=O)c1ncn2c(NCc3c(F)ccc4c3CCO4)ncc(-c3ccccc3)c12] | ethyl 5-(((5-fluoro-2,3-dihydrobenzofuran-4- yl)methyl)amino)-8-phenylimidazo[1,5- c]pyrimidine-1-carboxylate |
| 136 | 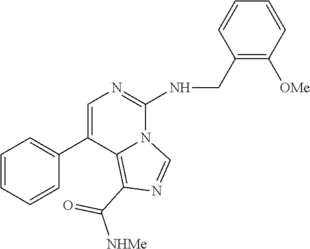 [SMILES: CNC(=O)c1ncn2c(NCc3ccccc3OC)ncc(-c3ccccc3)c12] | 5-((2-methoxybenzyl)amino)-N-methyl-8- phenylimidazo[1,5-c]pyrimidine-1-carboxamide |
| 137 | 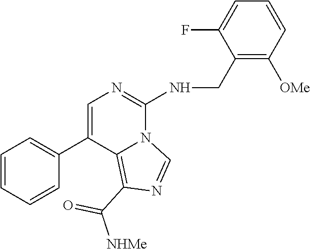 [SMILES: CNC(=O)c1ncn2c(NCc3c(F)cccc3OC)ncc(-c3ccccc3)c12] | 5-((2-fluoro-6-methoxybenzyl)amino)-N-methyl- 8-phenylimidazo[1,5-c]pyrimidine-1-carboxamide |
| 138 | 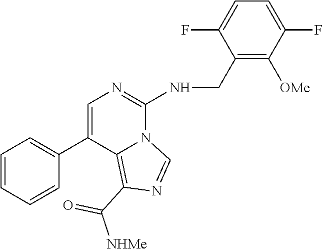 [SMILES: CNC(=O)c1ncn2c(NCc3c(F)ccc(F)c3OC)ncc(-c3ccccc3)c12] | 5-((3,6-difluoro-2-methoxybenzyl)amino)-N- methyl-8-phenylimidazo[1,5-c]pyrimidine-1- carboxamide |
| 139 | 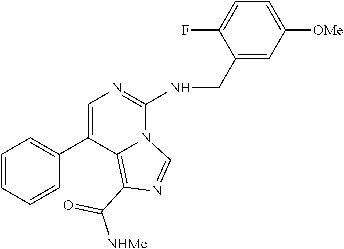 [SMILES: CNC(=O)c1ncn2c(NCc3cc(OC)ccc3F)ncc(-c3ccccc3)c12] | 5-((2-fluoro-5-methoxybenzyl)amino)-N-methyl- 8-phenylimidazo[1,5-c]pyrimidine-1-carboxamide |
| 140 | 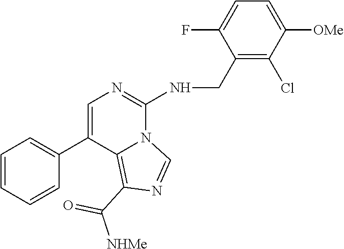 [SMILES: CNC(=O)c1ncn2c(NCc3c(F)ccc(OC)c3Cl)ncc(-c3ccccc3)c12] | 5-((2-chloro-6-fluoro-3-methoxybenzyl)amino)- N-methyl-8-phenylimidazo[1,5-c]pyrimidine-1- carboxamide |
| 141 | 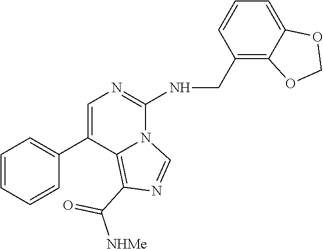 [SMILES: CNC(=O)c1ncn2c(NCc3cccc4c3OCO4)ncc(-c3ccccc3)c12] | 5-((benzo[d][1,3]dioxol-4-ylmethyl)amino)-N- methyl-8-phenylimidazo[1,5-c]pyrimidine-1- carboxamide |
| 142 | 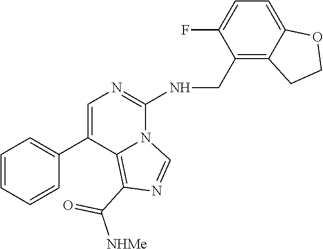 [SMILES: CNC(=O)c1ncn2c(NCc3c(F)ccc4c3CCO4)ncc(-c3ccccc3)c12] | 5-(((5-fluoro-2,3-dihydrobenzofuran-4- yl)methyl)amino)-N-methyl-8-phenylimidazo[1,5- c]pyrimidine-1-carboxamide |
| 143 | 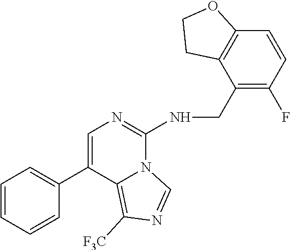 [SMILES: Fc1ccc2c(c1CNc1ncc(-c3ccccc3)c3c(C(F)(F)F)ncn13)CCO2] | N-((5-fluoro-2,3-dihydrobenzofuran-4-yl)methyl)- 8-phenyl-1-(trifluoromethyl)imidazo[1,5- c]pyrimidin-5-amine |
| 144 | 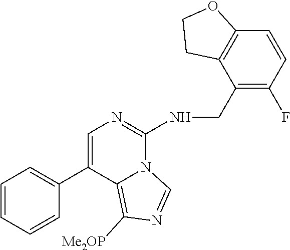 | (5-(((5-fluoro-2,3-dihydrobenzofuran-4- yl)methyl)amino)-8-phenylimidazo[1,5- c]pyrimidin-1-yl)dimethylphosphine oxide |
| 146 | 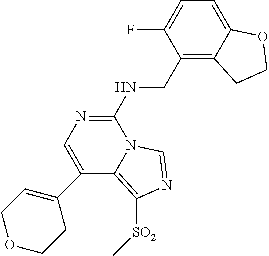 [SMILES: CS(=O)(=O)c1ncn2c(NCc3c(F)ccc4c3CCO4)ncc(C3=CCOCC3)c12] | 8-(3,6-dihydro-2H-pyran-4-yl)-N-((5-fluoro-2,3- dihydrobenzofuran-4-yl)methyl)-1- (methylsulfonyl)imidazo[1,5-c]pyrimidin-5-amine |
| 147 | 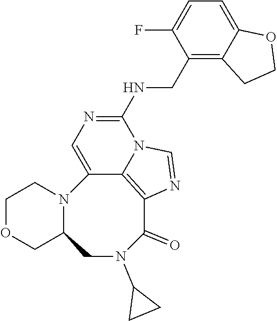 [SMILES: O=C1c2ncn3c(NCc4c(F)ccc5c4CCO5)ncc(c23)N2CCOC[C@@H]2CN1C1CC1] | (S)-4-cyclopropyl-12-(((5-fluoro-2,3- dihydrobenzofuran-4-yl)methyl)amino)- 4,5,5a,6,8,9-hexahydro-3H-7-oxa-2,4,9a,11,12a- pentaazabenzo[4,5]cycloocta[1,2,3-cd]inden-3- one |
| 148 | 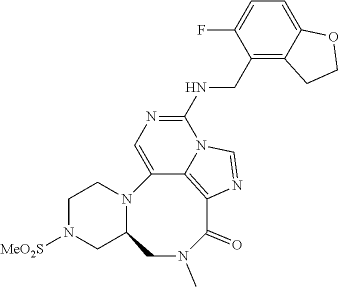 [SMILES: CN1C[C@H]2CN(S(C)(=O)=O)CCN2c2cnc(NCc3c(F)ccc4c3CCO4)n3cnc(c23)C1=O] | (S)-12-(((5-fluoro-2,3-dihydrobenzofuran-4- yl)methyl)amino)-4-methyl-7-(methylsulfonyl)- 5,5a,6,7,8,9-hexahydro-2,4,7,9a,11,12a- hexaazabenzo[4,5]cycloocta[1,2,3-cd]inden- 3(4H)-one |
| 149 | 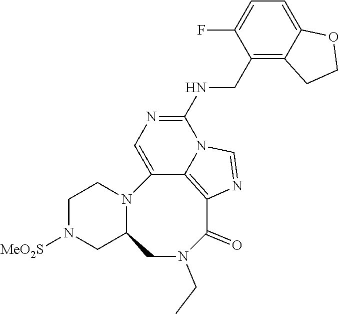 [SMILES: CCN1C[C@H]2CN(S(C)(=O)=O)CCN2c2cnc(NCc3c(F)ccc4c3CCO4)n3cnc(c23)C1=O] | (S)-4-ethyl-12-(((5-fluoro-2,3-dihydrobenzofuran- 4-yl)methyl)amino)-7-(methylsulfonyl)- 5,5a,6,7,8,9-hexahydro-2,4,7,9a,11,12a- hexaazabenzo[4,5]cycloocta[1,2,3-cd]inden- 3(4H)-one |
| 150 | 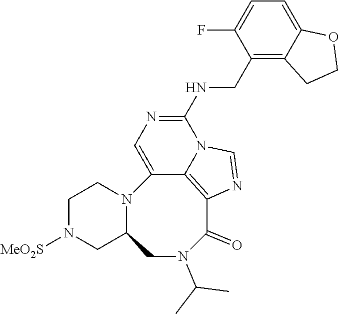 [SMILES: CC(C)N1C[C@H]2CN(S(C)(=O)=O)CCN2c2cnc(NCc3c(F)ccc4c3CCO4)n3cnc(c23)C1=O] | (S)-12-(((5-fluoro-2,3-dihydrobenzofuran-4- yl)methyl)amino)-4-isopropyl-7-(methylsulfonyl)- 5,5a,6,7,8,9-hexahydro-2,4,7,9a,11,12a- hexazabenzo[4,5]cycloocta[1,2,3-cd]inden- 3(4H)-one |
| 151 | 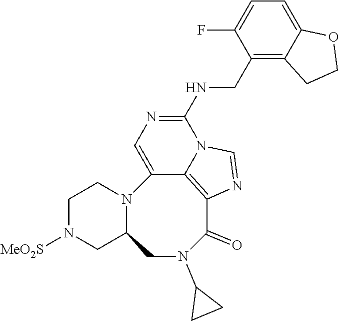 [SMILES: CS(=O)(=O)N1CCN2c3cnc(NCc4c(F)ccc5c4CCO5)n4cnc(c34)C(=O)N(C3CC3)C[C@H]2C1] | (S)-4-cyclopropyl-12-(((5-fluoro-2,3- dihydrobenzofuran-4-yl)methyl)amino)-7- (methylsulfonyl)-5,5a,6,7,8,9-hexahydro- 2,4,7,9a,11,12a- hexaazabenzo[4,5]cycloocta[1,2,3-cd]inden- 3(4H)-one |
| 152 | 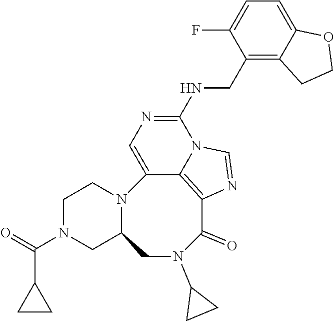 [SMILES: O=C(C1CC1)N1CCN2c3cnc(NCc4c(F)ccc5c4CCO5)n4cnc(c34)C(=O)N(C3CC3)C[C@H]2C1] | (R)-7-(cyclopropanecarbonyl)-4-cyclopropyl-12- (((5-fluoro-2,3-dihydrobenzofuran-4- yl)methyl)amino)-5,5a,6,7,8,9-hexahydro- 2,47,9a,11,12a- hexaazabenzo[4,5]cycloocta[1,2,3-cd]inden- 3(4H)-one |
| 153 | 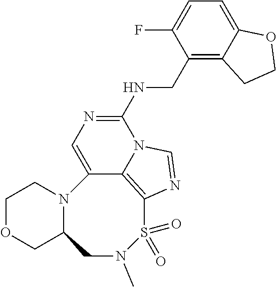 [SMILES: CN1C[C@H]2COCCN2c2cnc(NCc3c(F)ccc4c3CCO4)n3cnc(c23)S1(=O)=O] | (S)-12-(((5-fluoro-2,3-dihydrobenzofuran-4- yl)methyl)amino)-4-methyl-4,5,5a,6,8,9- hexahydro-7-oxa-3-thia-2,4,9a,11,12a- pentaazabenzo[4,5]cycloocta[1,2,3-cd]indene-3,3- dioxide |
| 154 | 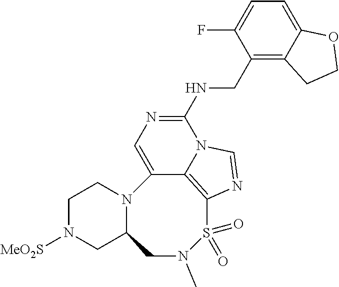 [SMILES: CN1C[C@H]2CN(S(C)(=O)=O)CCN2c2cnc(NCc3c(F)ccc4c3CCO4)n3cnc(c23)S1(=O)=O] | (S)-12-(((5-fluoro-2,3-dihydrobenzofuran-4- yl)methyl)amino)-4-methyl-7-(methylsulfonyl)- 5,5a,6,7,8,9-hexahydro-4H-3-thia-2,4,7,9a,11,12a- hexaazabenzo[4,5]cycloocta[1,2,3-cd]indene 3,3- dioxide |
| 155 | 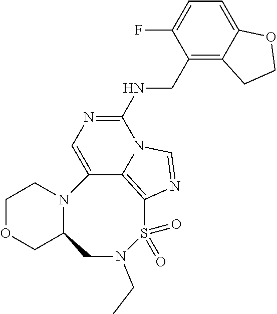 [SMILES: CCN1C[C@H]2COCCN2c2cnc(NCc3c(F)ccc4c3CCO4)n3cnc(c23)S1(=O)=O] | (S)-4-ethyl-12-(((5-fluoro-2,3-dihydrobenzofuran- 4-yl)methyl)amino)-4,5,5a,6,8,9-hexahydro-7- oxa-3-thia-2,4,9a,11,12a- pentaazabenzo[4,5]cycloocta[1,2,3-cd]indene 3,3- dioxide |
| 156 | 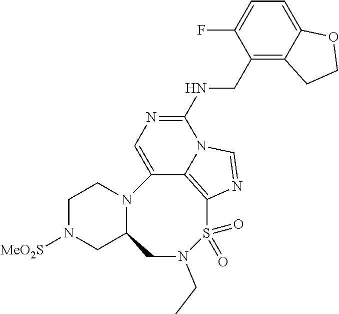 [SMILES: CCN1C[C@H]2CN(S(C)(=O)=O)CCN2c2cnc(NCc3c(F)ccc4c3CCO4)n3cnc(c23)S1(=O)=O] | (R)-4-ethyl-12-(((5-fluoro-2,3-dihydrobenzofuran- 4-yl)methyl)amino)-7-(methylsulfonyl)- 5,5a,6,7,8,9-hexahydro-4H-3-thia-2,4,7,9a,11,12a- hexaazabenzo[4,5]cycloocta[1,2,3-cd]indene 3,3- dioxide |
| 157 | 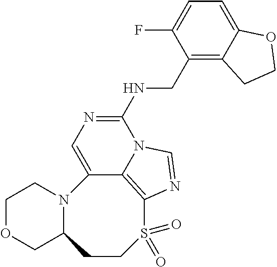 [SMILES: O=S1(=O)CC[C@H]2COCCN2c2cnc(NCc3c(F)ccc4c3CCO4)n3cnc1c23] | (S)-12-(((5-fluoro-2,3-dihydrobenzofuran-4- yl)methyl)amino)-4,5,5a,6,8,9-hexahydro-7-oxa- 3-thia-2,9a,11,12a- tetraazabenzo[4,5]cycloocta[1,2,3-cd]indene 3,3- dioxide |
| 158 | 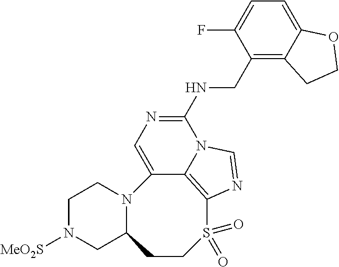 [SMILES: CS(=O)(=O)N1CCN2c3cnc(NCc4c(F)ccc5c4CCO5)n4cnc(c34)S(=O)(=O)CC[C@H]2C1] | (S)-12-(((5-fluoro-2,3-dihydrobenzofuran-4- yl)methyl)amino)-7-(methylsulfonyl)-5,5a,6,7,8,9- hexahydro-4H-3-thia-2,7,9a,11,12a- pentaazabenzo[4,5]cycloocta[1,2,3-cd]indene 3,3- dioxide |
| 169 | 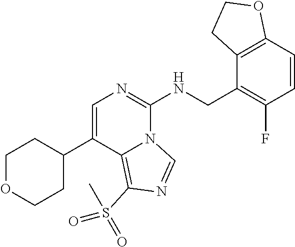 [SMILES: CS(=O)(=O)c1ncn2c(NCc3c(F)ccc4c3CCO4)ncc(C3CCOCC3)c12] | N-((5-fluoro-2,3-dihydrobenzofuran-4-yl)methyl)- 1-(methylsulfonyl)-8-(tetrahydro-2H-pyran-4- yl)imidazo[1,5-c]pyrimidin-5-amine |
| 160 | 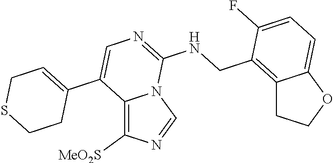 [SMILES: CS(=O)(=O)c1ncn2c(NCc3c(F)ccc4c3CCO4)ncc(C3=CCSCC3)c12] | 8-(3,6-dihydro-2H-thiopyran-4-yl)-N-((5-fluoro- 2,3-dihydrobenzofuran-4-yl)methyl)-1- (methylsulfonyl)imidazo[1,5-c]pyrimidin-5-amine |
| 161 | 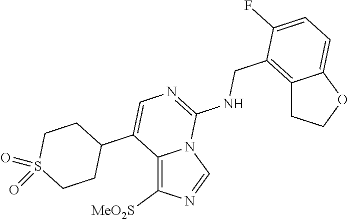 [SMILES: CS(=O)(=O)c1ncn2c(NCc3c(F)ccc4c3CCO4)ncc(C3CCS(=O)(=O)CC3)c12] | 4-(5-(((5-fluoro-2,3-dihydrobenzofuran-4- yl)methyl)amino)-1-(methylsulfonyl)imidazo[1,5- c]pyrimidin-8-yl)tetrahydro-2H-thiopyran 1,1- dioxide |
| 162 | 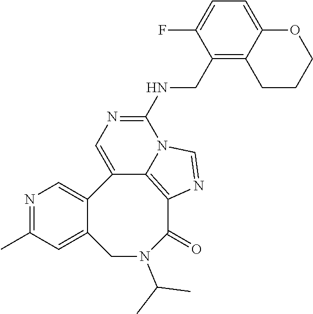 [SMILES: Cc1cc2c(cn1)-c1cnc(NCc3c(F)ccc4c3CCCO4)n3cnc(c13)C(=O)N(C(C)C)C2] | 12-(((6-fluorochroman-5-yl)methyl)amino)-4- isopropyl-7-methyl-4,5-dihydro-3H-2,4,8,11,12a- pentaazabenzo[4,5]cycloocta[1,2,3-cd]inden-3- one |
| 163 | 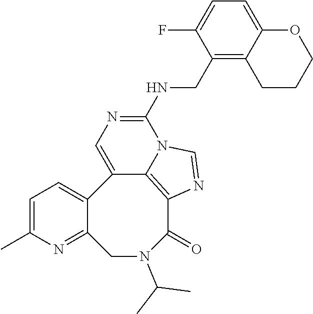 [SMILES: Cc1ccc2c(n1)CN(C(C)C)C(=O)c1ncn3c(NCc4c(F)ccc5c4CCCO5)ncc-2c13] | 12-(((6-fluorochroman-5-yl)methyl)amino)-4- isopropyl-7-methyl-4,5-dihydro-3H-2,4,6,11,12a- pentaazabenzo[4,5]cycloocta[1,2,3-cd]inden-3- one |
| 164 | 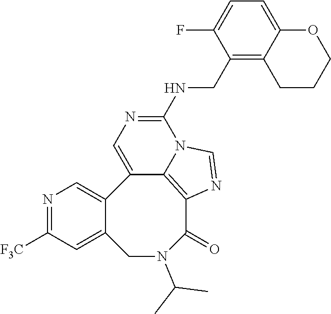 [SMILES: CC(C)N1Cc2cc(C(F)(F)F)ncc2-c2cnc(NCc3c(F)ccc4c3CCCO4)n3cnc(c23)C1=O] | 12-(((6-fluorochroman-5-yl)methyl)amino)-4- isopropyl-7-(trifluoromethyl)-4,5-dihydro-3H- 2,4,8,11,12a-pentaazabenzo[4,5]cycloocta[1,2,3- cd]inden-3-one |
| 165 | 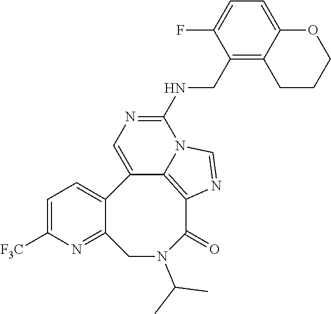 [SMILES: CC(C)N1Cc2nc(C(F)(F)F)ccc2-c2cnc(NCc3c(F)ccc4c3CCCO4)n3cnc(c23)C1=O] | 12-(((6-fluorochroman-5-yl)methyl)amino)-4- isopropyl-7-(trifluoromethyl)-4,5-dihydro-3H- 2,4,6,11,12a-pentaazabenzo[4,5]cycloocta[1,2,3- cd]inden-3-one |
| 166 | 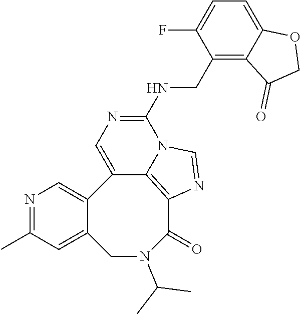 [SMILES: Cc1cc2c(cn1)-c1cnc(NCc3c(F)ccc4c3C(=O)CO4)n3cnc(c13)C(=O)N(C(C)C)C2] | 12-(((5-fluoro-3-oxo-2,3-dihydrobenzofuran-4- yl)methyl)amino)-4-isopropyl-7-methyl-4,5- dihydro-3H-2,4,8,11,12a- pentaazabenzo[4,5]cycloocta[1,2,3-cd]inden-3- one |
| 167 | 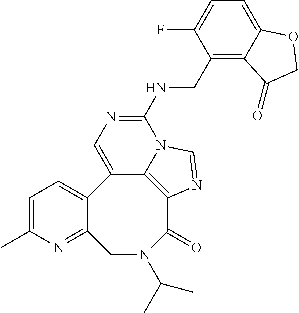 [SMILES: Cc1ccc2c(n1)CN(C(C)C)C(=O)c1ncn3c(NCc4c(F)ccc5c4C(=O)CO5)ncc-2c13] | 12-(((5-fluoro-3-oxo-2,3-dihydrobenzofuran-4- yl)methyl)amino)-4-isopropyl-7-methyl-4,5- dihydro-3H-2,4,6,11,12a- pentaazabenzo[4,5]cycloocta[1,2,3-cd]inden-3- one |
| 168 | 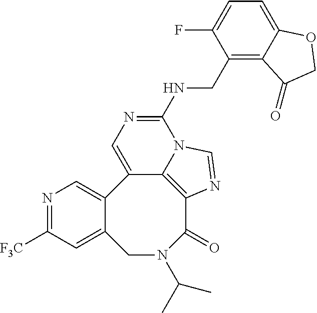 [SMILES: CC(C)N1Cc2cc(C(F)(F)F)ncc2-c2cnc(NCc3c(F)ccc4c3C(=O)CO4)n3cnc(c23)C1=O] | 12-(((5-fluoro-3-oxo-2,3-dihydrobenzofuran-4- yl)methyl)amino)-4-isopropyl-7-(trifluoromethyl)- 4,5-dihydro-3H-2,4,8,11,12a- pentaazabenzo[4,5]cycloocta[1,2,3-cd]inden-3- one |
| 169 | 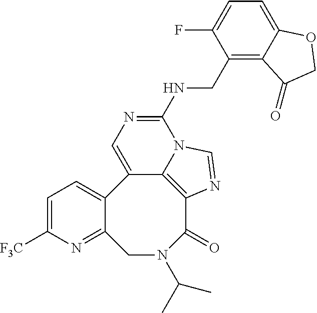 [SMILES: CC(C)N1Cc2nc(C(F)(F)F)ccc2-c2cnc(NCc3c(F)ccc4c3C(=O)CO4)n3cnc(c23)C1=O] | 12-(((5-fluoro-3-oxo-2,3-dihydrobenzofuran-4- yl)methyl)amino)-4-isopropyl-7-(trifluoromethyl)- 4,5-dihydro-3H-2,4,6,11,12a- pentaazabenzo[4,5]cycloocta[1,2,3-cd]inden-3- one |
| 170 | 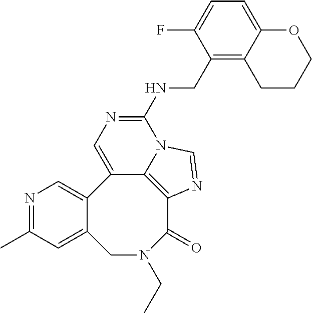 [SMILES: CCN1Cc2cc(C)ncc2-c2cnc(NCc3c(F)ccc4c3CCCO4)n3cnc(c23)C1=O] | 4-ethyl-12-(((6-fluorochroman-5- yl)methyl)amino)-7-methyl-4,5-dihydro-3H- 2,4,8,11,12a-pentaazabenzo[4,5]cycloocta[1,2,3- cd]inden-3-one |
| 171 | 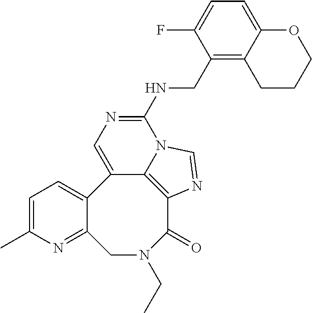 [SMILES: CCN1Cc2nc(C)ccc2-c2cnc(NCc3c(F)ccc4c3CCCO4)n3cnc(c23)C1=O] | 4-ethyl-12-(((6-fluorochroman-5- yl)methyl)amino)-7-methyl-4,5-dihydro-3H- 2,4,6,11,12a-pentaazabenzo[4,5]cycloocta[1,2,3- cd]inden-3-one |
| 172 | 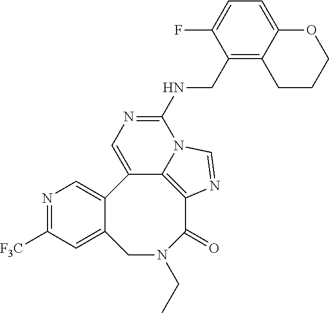 [SMILES: CCN1Cc2cc(C(F)(F)F)ncc2-c2cnc(NCc3c(F)ccc4c3CCCO4)n3cnc(c23)C1=O] | 4-ethyl-12-(((6-fluorochroman-5- yl)methyl)amino)-7-(trifluoromethyl)-4,5- dihydro-3H-2,4,8,11,12a- pentaazabenzo[4,5]cycloocta[1,2,3-cd]inden-3- one |
| 173 | 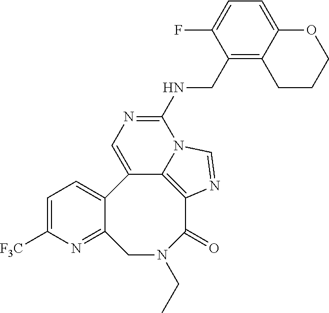 [SMILES: CCN1Cc2nc(C(F)(F)F)ccc2-c2cnc(NCc3c(F)ccc4c3CCCO4)n3cnc(c23)C1=O] | 4-ethyl-12-(((6-fluorochroman-5- yl)methyl)amino)-7-(trifluoromethyl)-4,5- dihydro-3H-2,4,6,11,12a- pentaazabenzo[4,5]cycloocta[1,2,3-cd]inden-3- one |
| 174 | 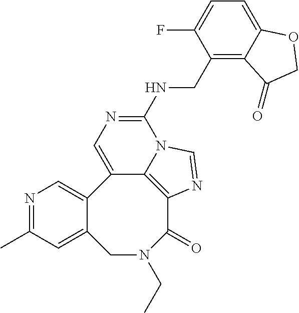 [SMILES: CCN1Cc2cc(C)ncc2-c2cnc(NCc3c(F)ccc4c3C(=O)CO4)n3cnc(c23)C1=O] | 4-ethyl-12-(((5-fluoro-3-oxo-2,3- dihydrobenzofuran-4-yl)methyl)amino)-7-methyl- 4,5-dihydro-3H-2,4,8,11,12a- pentaazabenzo[4,5]cycloocta[1,2,3-cd]inden-3- one |
| 175 | 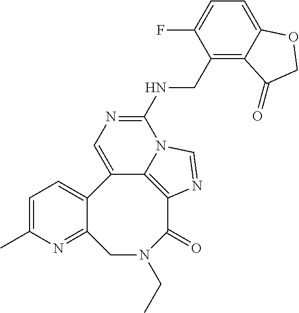 [SMILES: CCN1Cc2nc(C)ccc2-c2cnc(NCc3c(F)ccc4c3C(=O)CO4)n3cnc(c23)C1=O] | 4-ethyl-12-(((5-fluoro-3-oxo-2,3- dihydrobenzofuran-4-yl)methyl)amino)-7-methyl- 4,5-dihydro-3H-2,4,6,11,12a- pentaazabenzo[4,5]cycloocta[1,2,3-cd]inden-3- one |
| 176 | 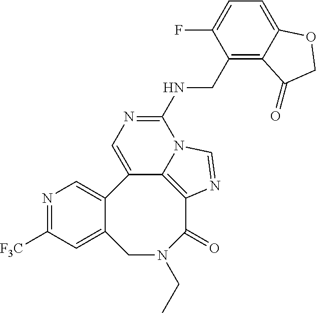 [SMILES: CCN1Cc2cc(C(F)(F)F)ncc2-c2cnc(NCc3c(F)ccc4c3C(=O)CO4)n3cnc(c23)C1=O] | 4-ethyl-12-(((5-fluoro-3-oxo-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| 177 | 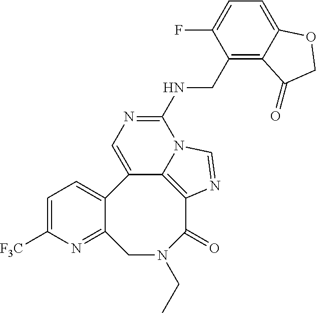 [SMILES: CCN1Cc2nc(C(F)(F)F)ccc2-c2cnc(NCc3c(F)ccc4c3C(=O)CO4)n3cnc(c23)C1=O] | 4-ethyl-12-(((5-fluoro-3-oxo-2,3- dihydrobenzofuran-4-yl)methyl)amino)-7- (trifluoromethyl)-4,5-dihydro-3H-2,4,6,11,12a- pentaazabenzo[4,5]cycloocta[1,2,3-cd]inden-3- one |
| 178 | 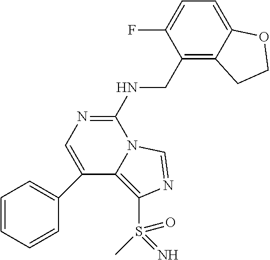 [SMILES: CS(=N)(=O)c1ncn2c(NCc3c(F)ccc4c3CCO4)ncc(-c3ccccc3)c12] | (5-(((5-fluoro-2,3-dihydrobenzofuran-4- yl)methyl)amino)-8-phenylimidazo[1,5- c]pyrimidin-1-yl)(imino)(methyl)-16-sulfanone |
| 179 | 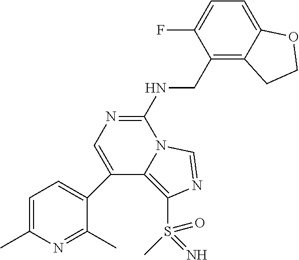 [SMILES: Cc1ccc(-c2cnc(NCc3c(F)ccc4c3CCO4)n3cnc(S(C)(=N)=O)c23)c(C)n1] | (8-(2,6-dimethylpyridin-3-yl)-5-(((5-fluoro-2,3- dihydrobenzofuran-4- yl)methyl)amino)imidazo[1,5-c]pyrimidin-1- yl)(imino)(methyl)-sulfanone |
| 180 | 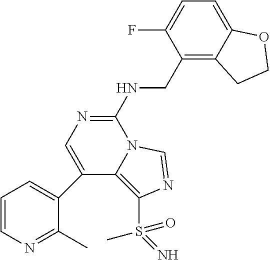 [SMILES: Cc1ncccc1-c1cnc(NCc2c(F)ccc3c2CCO3)n2cnc(S(C)(=N)=O)c12] | (5-(((5-fluoro-2,3-dihydrobenzofuran-4- yl)methyl)amino)-8-(2-methylpyridin-3- yl)imidazo[1,5-c]pyrimidin-1-yl)(imino)(methyl)- sulfanone |
| 181 | 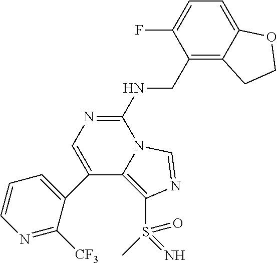 [SMILES: CS(=N)(=O)c1ncn2c(NCc3c(F)ccc4c3CCO4)ncc(-c3cccnc3C(F)(F)F)c12] | (5-(((5-fluoro-2,3-dihydrobenzofuran-4- yl)methyl)amino)-8-(2-(trifluoromethyl)pyridin-3- yl)imidazo[1,5-c]pyrimidin-1-yl)(imino)(methyl)- 16-sulfanone |
| 182 | 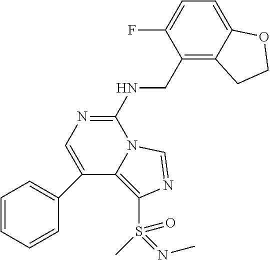 [SMILES: CN=S(C)(=O)c1ncn2c(NCc3c(F)ccc4c3CCO4)ncc(-c3ccccc3)c12] | (5-(((5-fluoro-2,3-dihydrobenzofuran-4- yl)methyl)amino)-8-phenylimidazo[1,5- c]pyrimidin-1-yl)(methyl)(methylimino)-16- sulfanone |
| 183 | 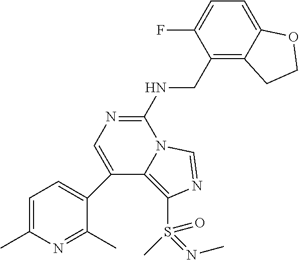 [SMILES: CN=S(C)(=O)c1ncn2c(NCc3c(F)ccc4c3CCO4)ncc(-c3ccc(C)nc3C)c12] | (8-(2,6-dimethylpyridin-3-yl)-5-(((5-fluoro-2,3- dihydrobenzofuran-4- yl)methyl)amino)imidazo[1,5-c]pyrimidin-1- yl)(methyl)(methylimino)-16-sulfanone |
| 184 | 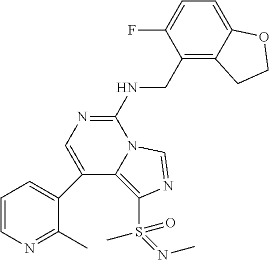 [SMILES: CN=S(C)(=O)c1ncn2c(NCc3c(F)ccc4c3CCO4)ncc(-c3cccnc3C)c12] | (5-(((5-fluoro-2,3-dihydrobenzofuran-4- yl)methyl)amino)-8-(2-methylpyridin-3- yl)imidazo[1,5-c]pyrimidin-1- yl)(methyl)(methylimino)-16-sulfanone |
| 185 | 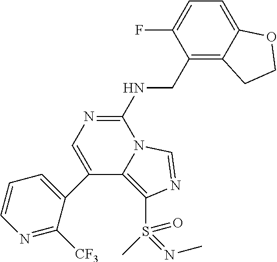 [SMILES: CN=S(C)(=O)c1ncn2c(NCc3c(F)ccc4c3CCO4)ncc(-c3cccnc3C(F)(F)F)c12] | (5-(((5-fluoro-2,3-dihydrobenzofuran-4- yl)methyl)amino)-8-(2-(trifluoromethyl)pyridin-3- yl)imidazo[1,5-c]pyrimidin-1- yl)(methyl)(methylimino)-16-sulfanone |
| 186 | 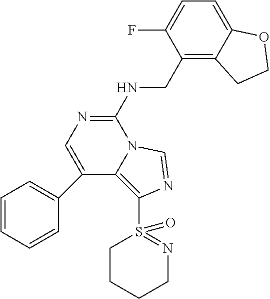 [SMILES: O=S1(c2ncn3c(NCc4c(F)ccc5c4CCO5)ncc(-c4ccccc4)c23)=NCCCC1] | 1-(5-(((5-fluoro-2,3-dihydrobenzofuran-4- yl)methyl)amino)-8-phenylimidazo[1,5- c]pyrimidin-1-yl)-3,4,5,6-tetrahydro-1,2-thiazine 1-oxide |
| 187 | 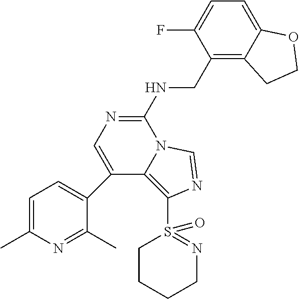 [SMILES: Cc1ccc(-c2cnc(NCc3c(F)ccc4c3CCO4)n3cnc(S4(=O)=NCCCC4)c23)c(C)n1] | 1-(8-(2,6-dimethylpyridin-3-yl)-5-(((5-fluoro-2,3- dihydrobenzofuran-4- yl)methyl)amino)imidazo[1,5-c]pyrimidin-1-yl)- 3,4,5,6-tetrahydro-1,2-thiazine 1-oxide |
| 188 | 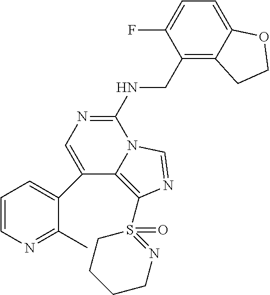 [SMILES: Cc1ncccc1-c1cnc(NCc2c(F)ccc3c2CCO3)n2cnc(S3(=O)=NCCCC3)c12] | 1-(5-(((5-fluoro-2,3-dihydrobenzofuran-4- yl)methyl)amino)-8-(2-methylpyridin-3- yl)imidazo[1,5-c]pyrimidin-1-yl)-3,4,5,6- tetrahydro-1,2-thiazine 1-oxide |
| 189 | 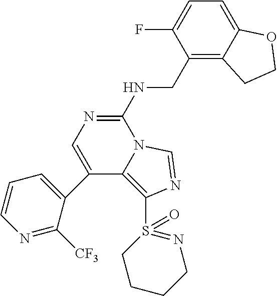 [SMILES: O=S1(c2ncn3c(NCc4c(F)ccc5c4CCO5)ncc(-c4cccnc4C(F)(F)F)c23)=NCCCC1] | 1-(5-(((5-fluoro-2,3-dihydrobenzofuran-4- yl)methyl)amino)-8-(2-(trifluoromethyl)pyridin-3- yl)imidazo[1,5-c]pyrimidin-1-yl)-3,4,5,6- tetrahydro-1,2-thiazine 1-oxide |
| E1 | 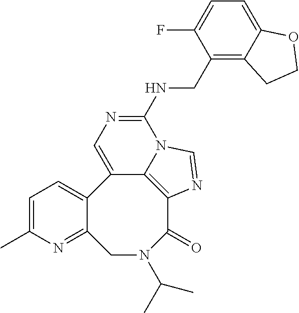 [SMILES: Cc1ccc2c(n1)CN(C(C)C)C(=O)c1ncn3c(NCc4c(F)ccc5c4CCO5)ncc-2c13] | 12-(((5-fluoro-2,3-dihydrobenzofuran-4- yl)methyl)amino)-4-isopropyl-7-methyl-4,5- dihydro-3H-2,4,6,11,12a- pentaazabenzo[4,5]cycloocta[1,2,3-cd]inden-3- one |
| E2 | 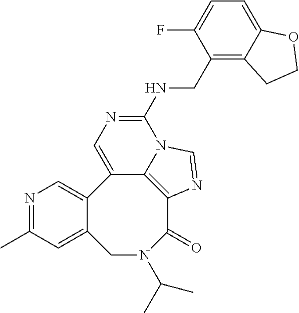 [SMILES: Cc1cc2c(cn1)-c1cnc(NCc3c(F)ccc4c3CCO4)n3cnc(c13)C(=O)N(C(C)C)C2] | 12-(((5-fluoro-2,3-dihydrobenzofuran-4- yl)methyl)amino)-4-isopropyl-7-methyl-4,5- dihydro-3H-2,4,8,11,12a- pentaazabenzo[4,5]cycloocta[1,2,3-cd]inden-3- one |
| E3 | 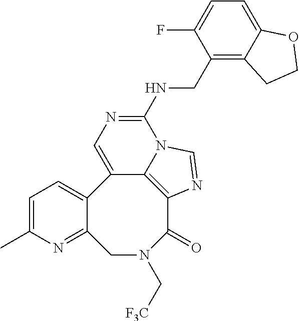 [SMILES: Cc1ccc2c(n1)CN(CC(F)(F)F)C(=O)c1ncn3c(NCc4c(F)ccc5c4CCO5)ncc-2c13] | 12-(((5-fluoro-2,3-dihydrobenzofuran-4- yl)methyl)amino)-7-methyl-4-(2,2,2- trifluoroethyl)-4,5-dihydro-3H-2,4,6,11,12a- pentaazabenzo[4,5]cycloocta[1,2,3-cd]inden-3- one |
| E4 | 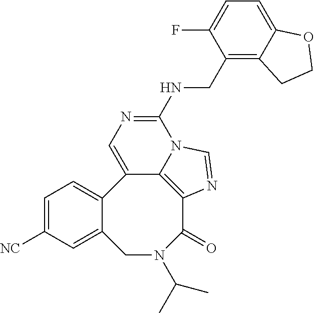 [SMILES: CC(C)N1Cc2cc(C#N)ccc2-c2cnc(NCc3c(F)ccc4c3CCO4)n3cnc(c23)C1=O] | 12-(((5-fluoro-2,3-dihydrobenzofuran-4- yl)methyl)amino)-4-isopropyl-3-oxo-4,5-dihydro- 3H-2,4,11,12a-tetraazabenzo[4,5]cycloocta[1,2,3- cd]indene-7-carbonitrile |
| E5 | 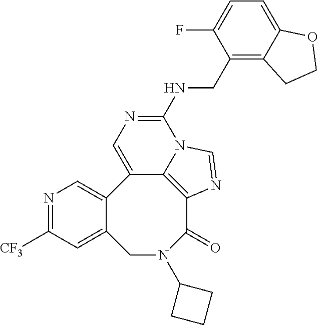 [SMILES: O=C1c2ncn3c(NCc4c(F)ccc5c4CCO5)ncc(c23)-c2cnc(C(F)(F)F)cc2CN1C1CCC1] | 4-cyclobutyl-12-(((5-fluoro-2,3- dihydrobenzofuran-4-yl)methyl)amino)-7- (trifluoromethyl)-4,5-dihydro-3H-2,4,8,11,12a- pentaazabenzo[4,5]cycloocta[1,2,3-cd]inden-3- one |
| E6 | 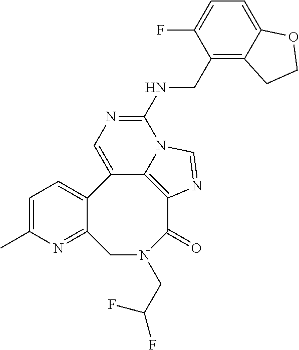 [SMILES: Cc1ccc2c(n1)CN(CC(F)F)C(=O)c1ncn3c(NCc4c(F)ccc5c4CCO5)ncc-2c13] | 4-(2,2-difluoroethyl)-12-(((5-fluoro-2,3- dihydrobenzofuran-4-yl)methyl)amino)-7-methyl- 4,5-dihydro-3H-2,4,6,11,12a- pentaazabenzo[4,5]cycloocta[1,2,3-cd]inden-3- one |
| E7 | 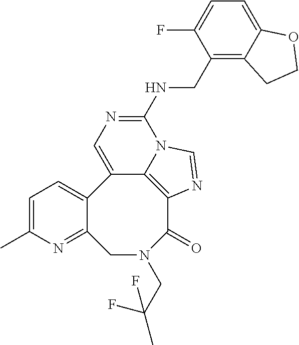 [SMILES: Cc1ccc2c(n1)CN(CC(C)(F)F)C(=O)c1ncn3c(NCc4c(F)ccc5c4CCO5)ncc-2c13] | 4-(2,2-difluoropropyl)-12-(((5-fluoro-2,3- dihydrobenzofuran-4-yl)methyl)amino)-7-methyl- 4,5-dihydro-3H-2,4,6,11,12a- pentaazabenzo[4,5]cycloocta[1,2,3-cd]inden-3- one |
| E8 | 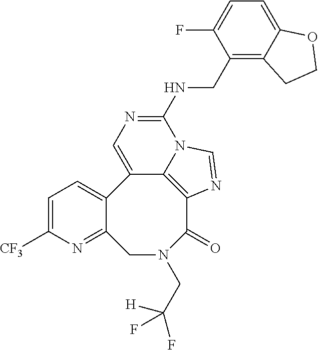 [SMILES: O=C1c2ncn3c(NCc4c(F)ccc5c4CCO5)ncc(c23)-c2ccc(C(F)(F)F)nc2CN1CC(F)F] | 4-(2,2-difluoroethyl)-12-(((5-fluoro-2,3- dihydrobenzofuran-4-yl)methyl)amino)-7- (trifluoromethyl)-4,5-dihydro-3H-2,4,6,11,12a- pentaazabenzo[4,5]cycloocta[1,2,3-cd]inden-3- one |
| E9 | 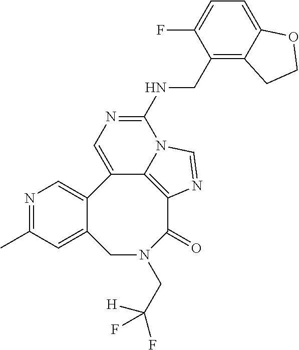 [SMILES: Cc1cc2c(cn1)-c1cnc(NCc3c(F)ccc4c3CCO4)n3cnc(c13)C(=O)N(CC(F)F)C2] | 4-(2,2-difluoroethyl)-12-(((5-fluoro-2,3- dihydrobenzofuran-4-yl)methyl)amino)-7-methyl- 4,5-dihydro-3H-2,4,8,11,12a- pentaazabenzo[4,5]cycloocta[1,2,3-cd]inden-3- one |
| E10 | 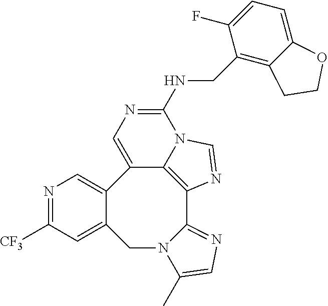 [SMILES: Cc1cnc2n1Cc1cc(C(F)(F)F)ncc1-c1cnc(NCc3c(F)ccc4c3CCO4)n3cnc-2c13] | N-((5-fluoro-2,3-dihydrobenzofuran-4-yl)methyl)- 5-methyl-8-(trifluoromethyl)-6H-2,3,5a,9,12,13a- hexaazabenzo[4,5]cyclopenta[8,9]cycloocta[1,2,3- cd]inden-13-amine |
| E11 | 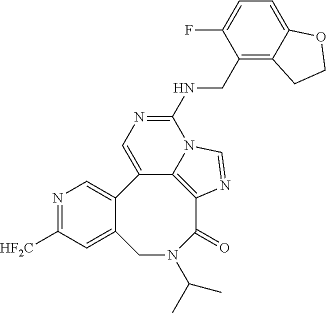 | 7-(difluoromethyl)-12-(((5-fluoro-2,3- dihydrobenzoufran-4-yl)methyl)amino)-4- isopropyl-4,5-dihydro-3H-2,4,8,11,12a- pentaazabenzo[4,5]cycloocta[1,2,3-cd]inden-3- one |
| E12 | 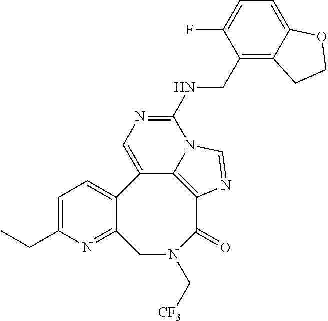 [SMILES: CCc1ccc2c(n1)CN(CC(F)(F)F)C(=O)c1ncn3c(NCc4c(F)ccc5c4CCO5)ncc-2c13] | 7-ethyl-12-(((5-fluoro-2,3-dihydrobenzofuran-4- yl)methyl)amino)-4-(2,2,2-trifluoroethyl)-4,5- dihydro-3H-2,4,6,11,12a- pentaazabenzo[4,5]cycloocta[1,2,3-cd]inden-3- one |
| E13 | 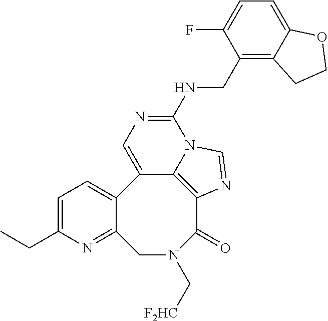 [SMILES: CCc1ccc2c(n1)CN(CC(F)F)C(=O)c1ncn3c(NCc4c(F)ccc5c4CCO5)ncc-2c13] | 4-(2,2-difluoroethyl)-7-ethyl-12-(((5-fluoro-2,3- dihydrobenzofuran-4-yl)methyl)amino)-4,5- dihydro-3H-2,4,6,11,12a- pentaazabenzo[4,5]cycloocta[1,2,3-cd]inden-3- one |
| E14 | 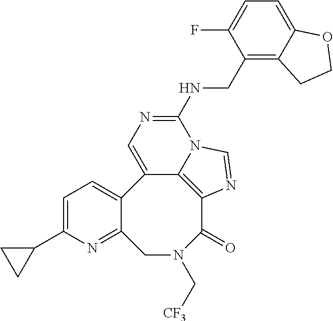 [SMILES: O=C1c2ncn3c(NCc4c(F)ccc5c4CCO5)ncc(c23)-c2ccc(C3CC3)nc2CN1CC(F)(F)F] | 7-cyclopropyl-12-(((5-fluoro-2,3- dihydrobenzofuran-4-yl)methyl)amino)-4-(2,2,2- trifluoroethyl)-4,5-dihydro-3H-2,4,6,11,12a- pentaazabenzo[4,5]cycloocta[1,2,3-cd]inden-3- one |
| E15 | 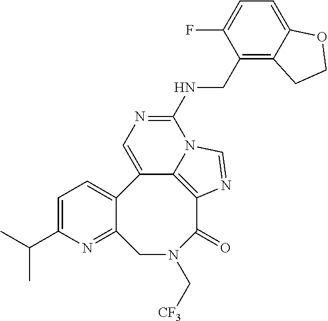 [SMILES: CC(C)c1ccc2c(n1)CN(CC(F)(F)F)C(=O)c1ncn3c(NCc4c(F)ccc5c4CCO5)ncc-2c13] | 12-(((5-fluoro-2,3-dihydrobenzofuran-4- yl)methyl)amino)-7-isopropyl-4-(2,2,2- trifluoroethyl)-4,5-dihydro-3H-2,4,6,11,12a- pentaazabenzo[4,5]cycloocta[1,2,3-cd]inden-3- one |
| E16 | 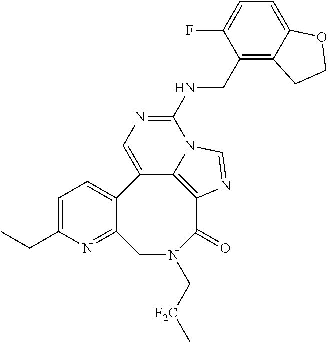 [SMILES: CCc1ccc2c(n1)CN(CC(C)(F)F)C(=O)c1ncn3c(NCc4c(F)ccc5c4CCO5)ncc-2c13] | 4-(2,2-difluoropropyl)-7-ethyl-12-(((5-fluoro-2,3- dihydrobenzofuran-4-yl)methyl)amino)-4,5- dihydro-3H-2,4,6,11,12a- pentaazabenzo[4,5]cycloocta[1,2,3-cd]inden-3- one |
| E17 | 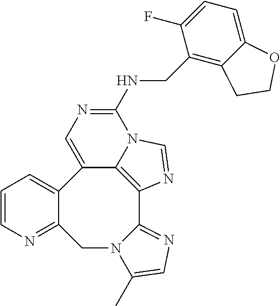 [SMILES: Cc1cnc2n1Cc1ncccc1-c1cnc(NCc3c(F)ccc4c3CCO4)n3cnc-2c13] | N-((5-fluoro-2,3-dihydrobenzofuran-4-yl)methyl)- 5-methyl-6H-2,3,5a,7,12,13a- hexaazabenzo[4,5]cyclopenta[7,8]cycloocta[1,2,3- cd]inden-13-amine |
| E18 | 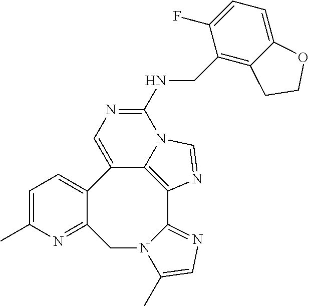 [SMILES: Cc1ccc2c(n1)Cn1c(C)cnc1-c1ncn3c(NCc4c(F)ccc5c4CCO5)ncc-2c13] | N-((5-fluoro-2,3-dihydrobenzofuran-4-yl)methyl)- 5,8-dimethyl-6H-2,3,5a,7,12,13a- hexaazabenzo[4,5]cyclopenta[7,8]cycloocta[1,2,3- cd]inden-13-amine |
| E19 | 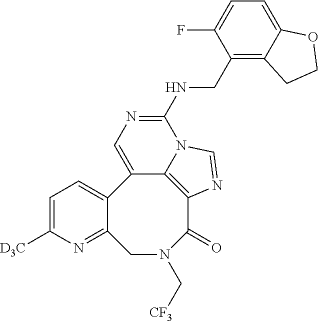 [SMILES: [2H]C([2H])([2H])c1ccc2c(n1)CN(CC(F)(F)F)C(=O)c1ncn3c(NCc4c(F)ccc5c4CCO5)ncc-2c13] | 12-(((5-fluoro-2,3-dihydrobenzofuran-4- yl)methyl)amino)-7-(methyl-d3)-4-(2,2,2- trifluoroethyl)-4,5-dihydro-3H-2,4,6,11,12a- pentaazabenzo[4,5]cycloocta[1,2,3-cd]inden-3- one |
| E20 | 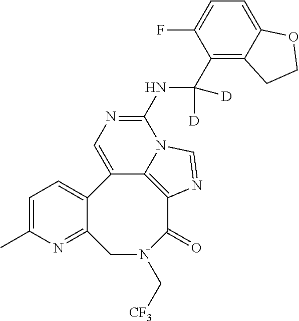 [SMILES: [2H]C([2H])(Nc1ncc2c3c(ncn13)C(=O)N(CC(F)(F)F)Cc1nc(C)ccc1-2)c1c(F)ccc2c1CCO2] | 12-(((5-fluoro-2,3-dihydrobenzofuran-4- yl)methyl-d2)amino)-7-methyl-4-(2,2,2- trifluoroethyl)-4,5-dihydro-3H-2,4,6,11,12a- pentaazabenzo[4,5]cycloocta[1,2,3-cd]inden-3- one |
| E21 | 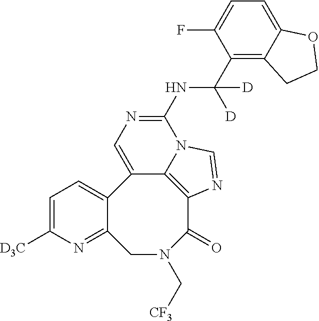 [SMILES: [2H]C([2H])([2H])c1ccc2c(n1)CN(CC(F)(F)F)C(=O)c1ncn3c(NC([2H])([2H])c4c(F)ccc5c4CCO5)ncc-2c13] | 12-(((5-fluoro-2,3-dihydrobenzofuran-4- yl)methyl-d2)amino)-7-(methyl-d3)-4-(2,2,2- trifluoroethyl)-4,5-dihydro-3H-2,4,6,11,12a- pentaazabenzo[4,5]cycloocta[1,2,3-cd]inden-3- one |
| E22 | 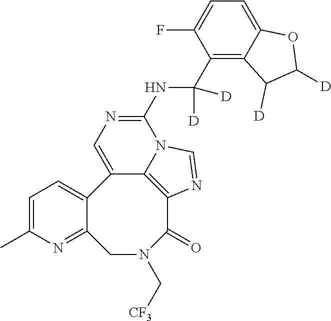 [SMILES: [2H]C1Oc2ccc(F)c(C([2H])([2H])Nc3ncc4c5c(ncn35)C(=O)N(CC(F)(F)F)Cc3nc(C)ccc3-4)c2C1[2H]] | 12-(((5-fluoro-2,3-dihydrobenzofuran-4-yl-2,3- d2)methyl-d2)amino)-7-methyl-4-(2,2,2- trifluoroethyl)-4,5-dihydro-3H-2,4,6,11,12a- pentaazabenzo[4,5]cycloocta[1,2,3-cd]inden-3- one |
| E23 | 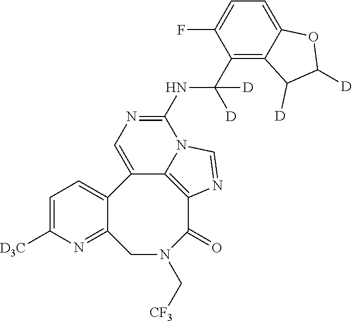 [SMILES: [2H]C1Oc2ccc(F)c(C([2H])([2H])Nc3ncc4c5c(ncn35)C(=O)N(CC(F)(F)F)Cc3nc(C([2H])([2H])[2H])ccc3-4)c2C1[2H]] | 12-(((5-fluoro-2,3-dihydrobenzofuran-4-yl-2,3- d2)methyl-d2)amino)-7-(methyl-d3)-4-(2,2,2- trifluoroethyl)-4,5-dihydro-3H-2,4,6,11,12a- pentaazabenzo[4,5]cycloocta[1,2,3-cd]inden-3- one |
| E24 | 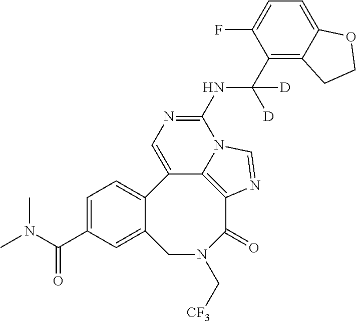 [SMILES: [2H]C([2H])(Nc1ncc2c3c(ncn13)C(=O)N(CC(F)(F)F)Cc1cc(C(=O)N(C)C)ccc1-2)c1c(F)ccc2c1CCO2] | 12-(((5-fluoro-2,3-dihydrobenzofuran-4- yl)methyl-d2)amino)-N,N-dimethyl-3-oxo-4- (2,2,2-trifluoroethyl)-4,5-dihydro-3H-2,4,11,12a- tetraazabenzo[4,5]cycloocta[1,2,3-cd]indene-7- carboxamidee |
| E25 | 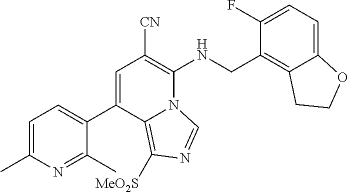 [SMILES: Cc1ccc(-c2cc(C#N)c(NCc3c(F)ccc4c3CCO4)n3cnc(S(C)(=O)=O)c23)c(C)n1] | 8-(2,6-dimethylpyridin-3-yl)-5-(((5-fluoro-2,3- dihydrobenzofuran-4-yl)methyl)amino)-1- (methylsulfonyl)imidazo[1,5-a]pyridine-6- carbonitrile |
| E26 | 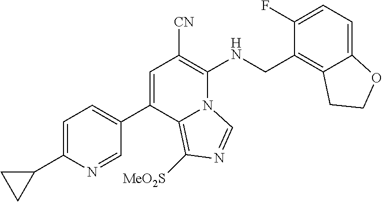 [SMILES: CS(=O)(=O)c1ncn2c(NCc3c(F)ccc4c3CCO4)c(C#N)cc(-c3ccc(C4CC4)nc3)c12] | 8-(6-cyclopropylpyridin-3-yl)-5-(((5-fluoro-2,3- dihydrobenzofuran-4-yl)methyl)amino)-1- (methylsulfonyl)imidazo[1,5-a]pyridine-6- carbonitrile |
| E27 | 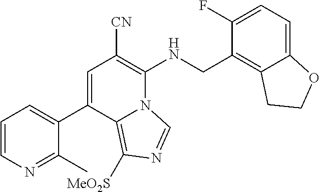 [SMILES: Cc1ncccc1-c1cc(C#N)c(NCc2c(F)ccc3c2CCO3)n2cnc(S(C)(=O)=O)c12] | 5-(((5-fluoro-2,3-dihydrobenzofuran-4- yl)methyl)amino)-8-(2-methylpyridin-3-yl)-1- (methylsulfonyl)imidazo[1,5-a]pyridine-6- carbonitrile |
| E28 | 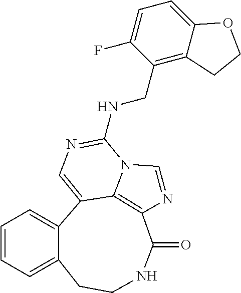 [SMILES: O=C1NCCc2ccccc2-c2cnc(NCc3c(F)ccc4c3CCO4)n3cnc1c23] | 13-(((5-fluoro-2,3-dihydrobenzofuran-4- yl)methyl)amino)-5,6-dihydro-2,4,12,13a- tetraazabenzo[4,5]cyclonona[1,2,3-cd]inden- 3(4H)-one |
| E29 | 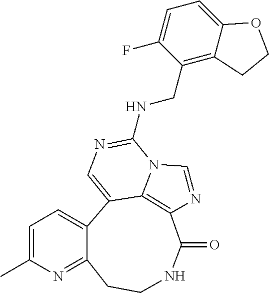 [SMILES: Cc1ccc2c(n1)CCNC(=O)c1ncn3c(NCc4c(F)ccc5c4CCO5)ncc-2c13] | 13-(((5-fluoro-2,3-dihydrobenzofuran-4- yl)methyl)amino)-8-methyl-5,6-dihydro- 2,4,7,12,13a-pentaazabenzo[4,5]cyclonona[1,2,3- cd]inden-3(4H)-one |
| E30 | 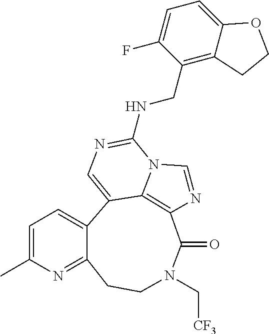 [SMILES: Cc1ccc2c(n1)CCN(CC(F)(F)F)C(=O)c1ncn3c(NCc4c(F)ccc5c4CCO5)ncc-2c13] | 13-(((5-fluoro-2,3-dihydrobenzofuran-4- yl)methyl)amino)-8-methyl-4-(2,2,2- trifluoroethyl)-5,6-dihydro-2,4,7,12,13a- pentaazabenzo[4,5]cyclonona[1,2,3-cd]inden- 3(4H)-one |
| E31 | 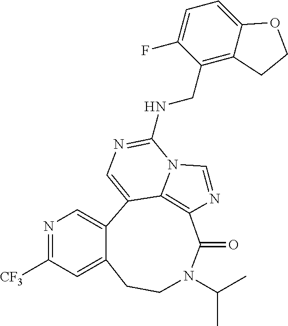 [SMILES: CC(C)N1CCc2cc(C(F)(F)F)ncc2-c2cnc(NCc3c(F)ccc4c3CCO4)n3cnc(c23)C1=O] | 13-(((5-fluoro-2,3-dihydrobenzofuran-4- yl)methyl)amino)-4-isopropyl-8-(trifluoromethyl)- 5,6-dihydro-2,4,9,12,13a- pentaazabenzo[4,5]cyclonona[1,2,3-cd]inden- 3(4H)-one |
| E32 | 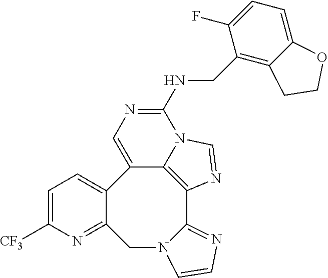 [SMILES: Fc1ccc2c(c1CNc1ncc3c4c(ncn14)-c1nccn1Cc1nc(C(F)(F)F)ccc1-3)CCO2] | N-((5-fluoro-2,3-dihydrobenzofuran-4-yl)methyl)- 8-(trifluoromethyl)-6H-2,3,5a,7,12,13a- hexaazabenzo[4,5]cyclopenta[7,9]cycloocta[1,2,3- cd]inden-13-amine |
| E33 | 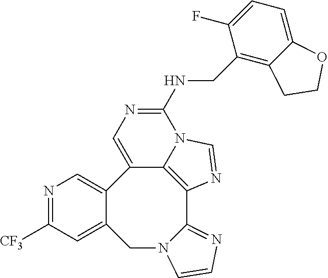 [SMILES: Fc1ccc2c(c1CNc1ncc3c4c(ncn14)-c1nccn1Cc1cc(C(F)(F)F)ncc1-3)CCO2] | N-((5-fluoro-2,3-dihydrobenzofuran-4-yl)methyl)- 8-(trifluoromethyl)-6H-2,3,5a,9,12,13a- hexaazabenzo[4,5]cyclopenta[7,8]cycloocta[1,2,3- cd]inden-13-amine |
| E34 | 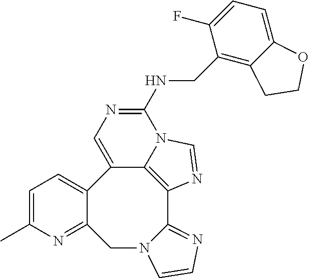 [SMILES: Cc1ccc2c(n1)Cn1ccnc1-c1ncn3c(NCc4c(F)ccc5c4CCO5)ncc-2c13] | N-((5-fluoro-2,3-dihydrobenzofuran-4-yl)methyl)- 8-methyl-6H-2,3,5a,7,12,13a- hexaazabenzo[4,5]cyclopenta[7,8]cycloocta[1,2,3- cd]inden-13-amine |
| E35 | 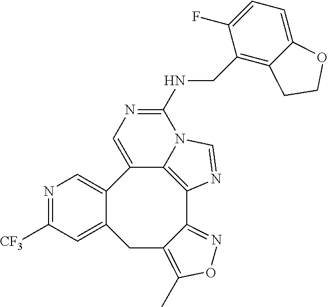 [SMILES: Cc1onc2c1Cc1cc(C(F)(F)F)ncc1-c1cnc(NCc3c(F)ccc4c3CCO4)n3cnc-2c13] | N-((5-fluoro-2,3-dihydrobenzofuran-4-yl)methyl)- 5-methyl-8-(trifluoromethyl)-6H-4-oxa- 2,3,9,12,13a- pentaazabenzo[4,5]cyclopenta[7,8]cycloocta[1,2,3- cd]inden-13-amine |
| E36 | 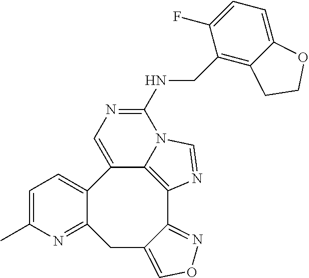 [SMILES: Cc1ccc2c(n1)Cc1conc1-c1ncn3c(NCc4c(F)ccc5c4CCO5)ncc-2c13] | N-((5-fluoro-2,3-dihydrobenzofuran-4-yl)methyl)- 8-methyl-6H-4-oxa-2,3,7,12,13a- pentaazabenzo[4,5]cyclopenta[7,8]cycloocta[1,2,3- cd]inden-13-amine |
| E37 | 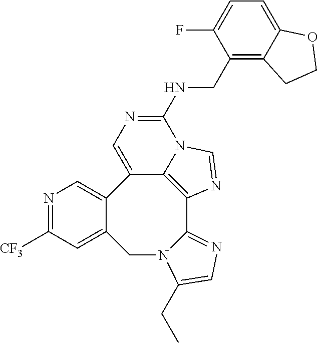 [SMILES: CCc1cnc2n1Cc1cc(C(F)(F)F)ncc1-c1cnc(NCc3c(F)ccc4c3CCO4)n3cnc-2c13] | 5-ethyl-N-((5-fluoro-2,3-dihydrobenzofuran-4- yl)methyl)-8-(trifluoromethyl)-6H- 2,3,5a,9,12,13a- hexaazabenzo[4,5]cyclopenta[7,8]cycloocta[1,2,3- cd]inden-13-amine |
| E38 | 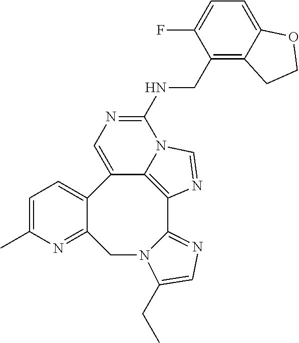 [SMILES: CCc1cnc2n1Cc1nc(C)ccc1-c1cnc(NCc3c(F)ccc4c3CCO4)n3cnc-2c13] | 5-ethyl-N-((5-fluoro-2,3-dihydrobenzofuran-4- yl)methyl)-8-methyl-6H-2,3,5a,7,12,13a- hexaazabenzo[4,5]cyclopenta[7,8]cycloocta[1,2,3- cd]inden-13-amine |
| E39 | 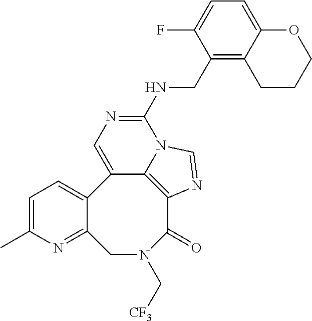 [SMILES: Cc1ccc2c(n1)CN(CC(F)(F)F)C(=O)c1ncn3c(NCc4c(F)ccc5c4CCCO5)ncc-2c13] | 12-(((6-fluorochroman-5-yl)methyl)amino)-7- methyl-4-(2,2,2-trifluoroethyl)-4,5-dihydro-3H- 2,4,6,11,12a-pentaazabenzo[4,5]cycloocta[1,2,3- cd]inden-3-one |
| E40 | 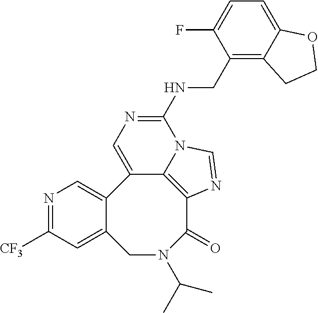 [SMILES: CC(C)N1Cc2cc(C(F)(F)F)ncc2-c2cnc(NCc3c(F)ccc4c3CCO4)n3cnc(c23)C1=O] | 12-(((5-fluoro-2,3-dihydrobenzofuran-4- yl)methyl)amino)-4-isopropyl-7-(trifluoromethyl)- 4,5-dihydro-3H-2,4,8,11,12a- pentaazabenzo[4,5]cycloocta[1,2,3-cd]inden-3- one |
| E41 | 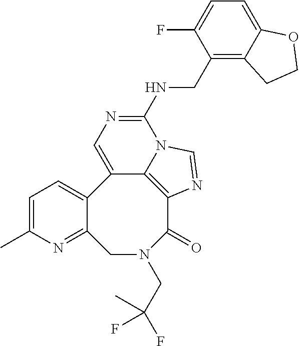 [SMILES: Cc1ccc2c(n1)CN(CC(C)(F)F)C(=O)c1ncn3c(NCc4c(F)ccc5c4CCO5)ncc-2c13] | 4-(2,2-difluoropropyl)-12-(((5-fluoro-2,3- dihydrobenzofuran-4-yl)methyl)amino)-7-methyl- 4,5-dihydro-3H-2,4,6,11,12a- pentaazabenzo[4,5]cycloocta[1,2,3-cd]inden-3- one |
| E42 | 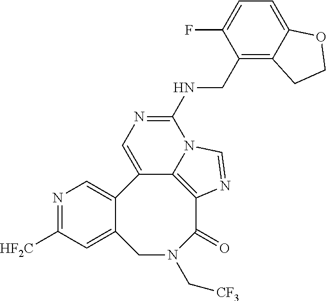 | 7-(difluoromethyl)-12-(((5-fluoro-2,3- dihydrobenzofuran-4-yl)methyl)amino)-4-(2,2,2- trifluoroethyl)-4,5-dihydro-3H-2,4,8,11,12a- pentaazabenzo[4,5]cycloocta[1,2,3-cd]inden-3- one |
| E43 | 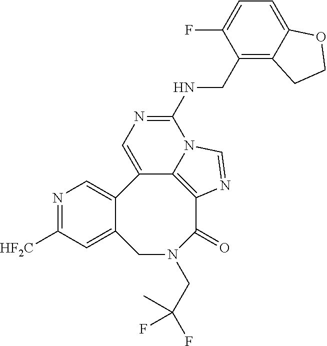 | 7-(difluoromethyl)-4-(2,2-difluoropropyl)-12-(((5- fluoro-2,3-dihydrobenzofuran-4- yl)methyl)amino)-4,5-dihydro-3H-2,4,8,11,12a- pentaazabenzo[4,5]cycloocta[1,2,3-cd]inden-3- one |
| E44 | 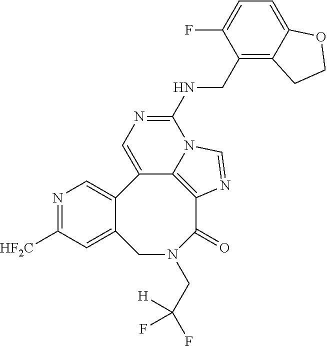 | 4-(2,2-difluoroethyl)-7-(difluoromethyl)-12-(((5- fluoro-2,3-dihydrobenzofuran-4- yl)methyl)amino)-4,5-dihydro-3H-2,4,8,11,12a- pentaazabenzo[4,5]cycloocta[1,2,3-cd]inden-3- one |
| E45 | 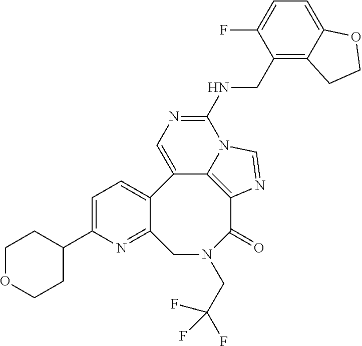 [SMILES: O=C1c2ncn3c(NCc4c(F)ccc5c4CCO5)ncc(c23)-c2ccc(C3CCOCC3)nc2CN1CC(F)(F)F] | 12-(((5-fluoro-2,3-dihydrobenzofuran-4- yl)methyl)amino)-7-(tetrahydro-2H-pyran-4-yl)-4- (2,2,2-trifluoroethyl)-4,5-dihydro-3H- 2,4,6,11,12a-pentaazabenzo[4,5]cycloocta[1,2,3- cd]inden-3-one |
| E46 | 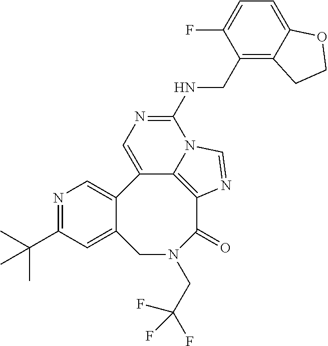 [SMILES: CC(C)(C)c1cc2c(cn1)-c1cnc(NCc3c(F)ccc4c3CCO4)n3cnc(c13)C(=O)N(CC(F)(F)F)C2] | 7-(tert-butyl)-12-(((5-fluoro-2,3- dihydrobenzofuran-4-yl)methyl)amino)-4-(2,2,2- trifluoroethyl)-4,5-dihydro-3H-2,4,8,11,12a- pentaazabenzo[4,5]cycloocta[1,2,3-ced]inden-3- one |
| E47 | 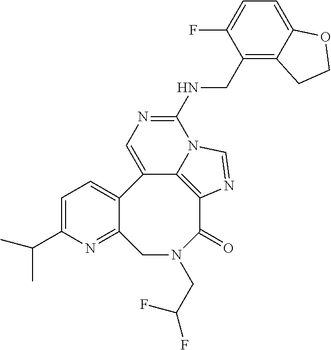 [SMILES: CC(C)c1ccc2c(n1)CN(CC(F)F)C(=O)c1ncn3c(NCc4c(F)ccc5c4CCO5)ncc-2c13] | 4-(2,2-difluoroethyl)-12-(((5-fluoro-2,3- dihydrobenzofuran-4-yl)methyl)amino)-7- isopropyl-4,5-dihydro-3H-2,4,6,11,12a- pentaazabenzo[4,5]cycloocta[1,2,3-cd]inden-3- one |
| E48 | 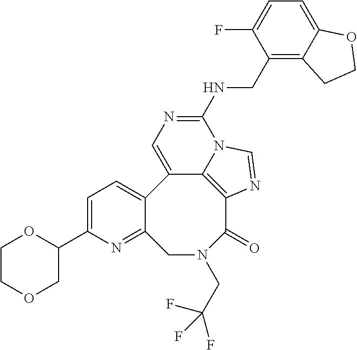 [SMILES: O=C1c2ncn3c(NCc4c(F)ccc5c4CCO5)ncc(c23)-c2ccc(C3COCCO3)nc2CN1CC(F)(F)F] | 7-(1,4-dioxan-2-yl)-12-(((5-fluoro-2,3- dihydrobenzofuran-4-yl)methyl)amino)-4-(2,2,2- trifluoroethyl)-4,5-dihydro-3H-2,4,6,11,12a- pentaazabenzo[4,5]cycloocta[1,2,3-cd]inden-3- one |
| E49 | 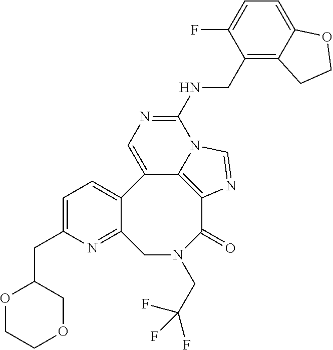 [SMILES: O=C1c2ncn3c(NCc4c(F)ccc5c4CCO5)ncc(c23)-c2ccc(CC3COCCO3)nc2CN1CC(F)(F)F] | 7-((1,4-dioxan-2-yl)methyl)-12-(((5-fluoro-2,3- dihydrobenzofuran-4-yl)methyl)amino)-4-(2,2,2- trifluoroethyl)-4,5-dihydro-3H-2,4,6,11,12a- pentaazabenzo[4,5]cycloocta[1,2,3-cd]inden-3- one |
| E50 | 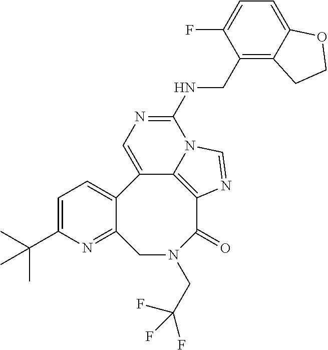 [SMILES: CC(C)(C)c1ccc2c(n1)CN(CC(F)(F)F)C(=O)c1ncn3c(NCc4c(F)ccc5c4CCO5)ncc-2c13] | 7-(tert-butyl)-12-(((5-fluoro-2,3- dihydrobenzofuran-4-yl)methyl)amino)-4-(2,2,2- trifluoroethyl)-4,5-dihydro-3H-2,4,6,11,12a- pentaazabenzo[4,5]cycloocta[1,2,3-cd]inden-3- one |
| E51 | 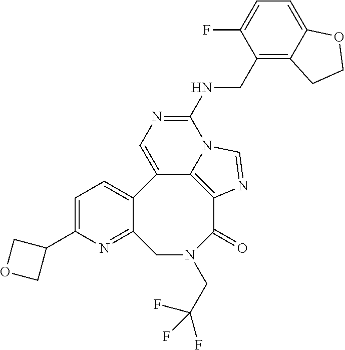 [SMILES: O=C1c2ncn3c(NCc4c(F)ccc5c4CCO5)ncc(c23)-c2ccc(C3COC3)nc2CN1CC(F)(F)F] | 12-(((5-fluoro-2,3-dihydrobenzofuran-4- yl)methyl)amino)-7-(oxetan-3-yl)-4-(2,2,2- trifluoroethyl)-4,5-dihydro-3H-2,4,6,11,12a- pentaazabenzo[4,5]cycloocta[1,2,3-cd]inden-3- one |
| E52 | 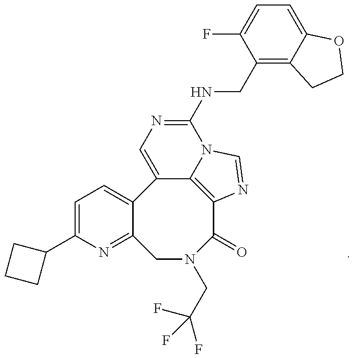 [SMILES: O=C1c2ncn3c(NCc4c(F)ccc5c4CCO5)ncc(c23)-c2ccc(C3CCC3)nc2CN1CC(F)(F)F] | 7-cyclobutyl-12-(((5-fluoro-2,3- dihydrobenzofuran-4-yl)methyl)amino)-4-(2,2,2- trifluoroethyl)-4,5-dihydro-3H-2,4,6,11,12a- pentaazabenzo[4,5]cycloocta[1,2,3-cd]inden-3- one |

39. The method of claim 38, wherein the substance X are selected from group consisting of:
4-ethyl-12-(((5-fluoro-2,3-dihydrobenzofuran-4-yl)methyl)amino)-7-(trifluoromethyl)-4,5-dihydro-3H-2,4,8,11,12a-pentaazabenzo[4,5]cycloocta[1,2,3-cd]inden-3-one;
12-(((5-fluoro-2,3-dihydrobenzofuran-4-yl)methyl)amino)-4-(2,2,2-trifluoroethyl)-7-(trifluoromethyl)-4,5-dihydro-3H-2,4,8,11,12a-pentaazabenzo[4,5]cycloocta[1,2,3-cd]inden-3-one;
4-cyclopropyl-12-(((5-fluoro-2,3-dihydrobenzofuran-4-yl)methyl)amino)-7-(trifluoromethyl)-4,5-dihydro-3H-2,4,8,11,12a-pentaazabenzo[4,5]cycloocta[1,2,3-cd]inden-3-one;
12-(((5-fluoro-2,3-dihydrobenzofuran-4-yl)methyl)amino)-4-isopropyl-7-(trifluoromethyl)-4,5-dihydro-3H-2,4,8,11,12a-pentaazabenzo[4,5]cycloocta[1,2,3-cd]inden-3-one; and
11-(((5-fluoro-2,3-dihydrobenzofuran-4-yl)methyl)amino)-6-methyl-4H-3-thia-2,5,10,11a-tetraazadibenzo[cd,f]azulene 3,3-dioxide,
or a pharmaceutically acceptable salt or solvate thereof.

40. The method of claim 38, wherein the substance X is Cpd. 73 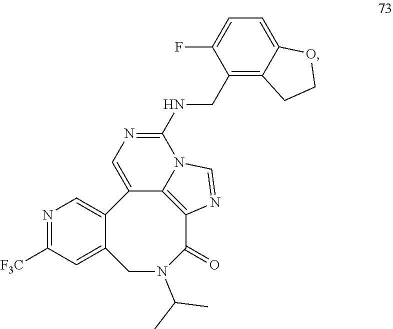 [SMILES: CC(C)N1Cc2cc(C(F)(F)F)ncc2-c2cnc(NCc3c(F)ccc4c3CCO4)n3cnc(c23)C1=O]
or a pharmaceutically acceptable salt or solvate thereof.

41. The method of any one of claims 1-40, wherein the subject is a mammal, preferably, a mice or a human.

42. The method of any one of claims 1-40, wherein the pharmaceutical composition comprises the substance X and an excipient and/or pharmaceutically acceptable carrier.

43. The method of any one of claims 1-40, wherein the substance X or the pharmaceutical composition is administered orally.

44. The method of any one of claims 1-40, wherein the substance X or the pharmaceutical composition is administered one or more times daily or weekly.

45. The method of any one of claims 1-40, wherein the substance X is administered in an amount of 3 mg/kg to 90 mg/kg per time to the subject in need thereof, preferably, an amount of 3 mg/kg, 10 mg/kg, 30 mg/kg or 90 mg/kg per time to the subject in need thereof.

46. The method of any one of claims 1-40, wherein the substance X or the pharmaceutical composition is administered continuously for at least 5 days, at least 1 week, at least 2 weeks, at least 3 weeks or at least 4 weeks.

47. A method of decreasing the H3K27me3 level in erythroid lineage cells or the H3K27me3 level in PBMC monocytes, which comprises administering to a subject in need thereof a therapeutically effective amount of a substance X or a pharmaceutical composition comprising the substance X; the substance X is as defined in any one of claims 1-40; the pharmaceutical composition is as defined in any one of claims 1-40.

48. The method of claim 47, wherein the erythroid lineage cells is the erythroid lineage cells from bone marrow, preferably, the TER119⁺ erythroid lineage cells from bone marrow.

49. The method of claim 47, wherein the subject suffers from a disease or disorder associated with a defect in hemoglobin protein activity or expression.

50. The method of claim 47, wherein the subject is a mammal, preferably, a mice or a human.

51. The method of claim 47, wherein the pharmaceutical composition comprises the substance X and an excipient and/or pharmaceutically acceptable carrier.

52. The method of claim 47, wherein the substance X or the pharmaceutical composition is administered orally.

53. The method of claim 47, wherein the substance X or the pharmaceutical composition is administered one or more times daily or weekly.

54. The method of claim 47, wherein the substance X is administered in an amount of 3 mg/kg to 90 mg/kg per time to the subject in need thereof, preferably, an amount of 3 mg/kg, 10 mg/kg, 30 mg/kg or 90 mg/kg per time to the subject in need thereof.

55. The method of claim 47, wherein the substance X or the pharmaceutical composition is administered continuously for at least 5 days, at least 1 week, at least 2 weeks, at least 3 weeks or at least 4 weeks.

56. A method of increasing Hbb-bh1 mRNA level, HBG gene level (HBG1 gene level, HBG2 gene level), γ hemoglobin level or HbF level, which comprises administering to a subject in need thereof a therapeutically effective amount of a substance X or a pharmaceutical composition comprising the substance X; the substance X is as defined in any one of claims 1-40; the pharmaceutical composition is as defined in any one of claims 1-40.

57. The method of claim 56, wherein the Hbb-bh1 mRNA level is the Hbb-bh1 mRNA level in blood, preferably, peripheral blood.

58. The method of claim 56, wherein the Hbb-bh1 mRNA level is the Hbb-bh1 mRNA level in the blood of CD-1 mice.

59. The method of claim 56, wherein the HBG1 gene level is the HBG1 gene level in blood, preferably, peripheral blood.

60. The method of claim 56, wherein the HBG1 gene level is the HBG1 gene level in the blood of human.

61. The method of claim 56, wherein the HBG2 gene level is the HBG2 gene level in blood, preferably, peripheral blood.

62. The method of claim 56, wherein the HBG2 gene level is the HBG2 gene level in the blood of human.

63. The method of claim 56, wherein the γ hemoglobin level is the γ hemoglobin level in blood, preferably, peripheral blood.

64. The method of claim 56, wherein the γ hemoglobin level is the γ hemoglobin level in the blood of human.

65. The method of claim 56, wherein the HbF level is the HbF level in blood, preferably, peripheral blood.

66. The method of claim 56, wherein the HbF level is the HbF level in the blood of human.

67. The method of claim 56, wherein the subject suffers from a disease or disorder associated with a defect in hemoglobin protein activity or expression.

68. The method of claim 56, wherein the subject is a mammal, preferably, a mice or a human.

69. The method of claim 56, wherein the pharmaceutical composition comprises the substance X and an excipient and/or pharmaceutically acceptable carrier.

70. The method of claim 56, wherein the substance X or the pharmaceutical composition is administered orally.

71. The method of claim 56, wherein the substance X or the pharmaceutical composition is administered one or more times daily or weekly.

72. The method of claim 56, wherein the substance X is administered in an amount of 3 mg/kg to 90 mg/kg per time to the subject in need thereof, preferably, an amount of 3 mg/kg, 10 mg/kg, 30 mg/kg or 90 mg/kg per time to the subject in need thereof.

73. The method of claim 56, wherein the substance X or the pharmaceutical composition is administered continuously for at least 5 days, at least 1 week, at least 2 weeks, at least 3 weeks or at least 4 weeks.

74. The method of any one of claims 1-73, wherein the disease or disorder is a blood disorder.

75. The method of any one of claims 1-73, wherein the disease or disorder is anemia.

76. The method of any one of claims 1-73, wherein the disease or disorder is selected from a group consisting of: Sickle cell disease, β-thalassemia, β-thalessemia intermedia, β-thalessemia major, β-thalessemia minor, chemotherapy-induced anemia, chronic kidney disease related anemia, and Cooley's anemia.

77. The method of any one of claims 1-73, wherein the disease or disorder is Sickle cell disease.

78. The method of any one of claims 1-73, wherein the disease or disorder is β-thalassemia.

79. The method of any one of claims 1-73, wherein the disease or disorder is chemotherapy-induced anemia.

80. The method of any one of claims 1-73, wherein the disease or disorder is chronic kidney disease related anemia.

81. The method of claim 1-80, wherein the method comprising using a second therapeutic agent.

82. The method of claim 81, wherein the second therapeutic agent is Erythropoietin (EPO).

83. The method of claim 1-80, wherein the dosage of substance X is 10-30 mg/kg, preferably 15 mg/kg.

84. The method of claim 82, wherein the dosage of Erythropoietin (EPO) is 10-100 U/kg, preferably 50 U/kg.

85. The method of any one of claims 1-40, wherein the innate immune response, type 1 or type 3 immunity related immune disorder is inflammatory bowel disease.

86. The method of claim 85, wherein the innate immune response, type 1 or type 3 immunity related immune disorder is Crohn's disease.

87. The method of claim 85, wherein the innate immune response, type 1 or type 3 immunity related immune disorder is ulcerative colitis.

88. The method of any one of claims 1-40, wherein the innate immune response, type 1 or type 3 immunity related immune disorder is selected from the group consisting of Nonorgan specific systemic autoimmune disorders: acute disseminated encephalomyelitis (ADEM), Addison disease, ankylosing spondylitis, antiphospholipid syndrome (APGS), aplastic anemia, American Industrial Hygiene Association (AIHA), autoimmune hepatitis (AIH), autoimmune hypoparathyroidism, Autoimmune hypophysitis, autoimmune myocardioptis, autoimmune oophoritis, autoimmune orchitis, Autoimmune thrombocytopenic purpura (AITP), Behcet's disease, bullous pemphigoid, Chronic inflammatory demyelinating polyneuropathy, Churg-Strauss syndrome, Crohn's disease, dermatomyositis, familial dysautonomia, epidermolysis bullosa, Pemphigoid during pregnancy, giant cell arteritis, Goodpasture syndrome, Granulomatous disease with polyvasculitis, Graves' disease, Guillain-barre syndrome, Hashimoto Disease, Immunoglobulin A (IgA) neurological disease, ulcerative colitis, interstitial cystitis (IC), Kawasaki Disease, Lambert-Eaton myasthenic syndrome (LEMS), Chronic Lyme disease, Mooren's ulcer, morphea, myasthenia gravis, neuromyotonia, multiple sclerosis, Clonic syndrome of strabismus, optic neuritis, Ord thyroiditis, pemphigus, pernicious anemia, polyarteritis, polyarthritis, Polyglandular autoimmune syndrome, primary biliary cirrhosis, psoriasis, Reiter's syndrome, Sarcoidosis, rheumatic arthritis, Sjogren's syndrome, stiff-man syndrome, systemic lupus erythematosus, Takayasu arthritis, and Vogt-Kovangai-Harada disease.

89. The method of any one of claims 85-88, wherein the dosage of substance X is 10-100 mg/kg, preferably 50 mg/kg or 90 mg/kg.

90. A pharmaceutical composition, which comprise (i) the substance X according to any one of claims 1-40; (ii) erythropoietin.
